SEQUENCE LISTING (26 sequences)

SEQ ID NO: 1 - DNA, 206 bp, Artificial Sequence
  HBV target
gacaagaatcctcacaataccgcagagtctagactcgtggtggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcc

SEQ ID NO: 2 - DNA, 20 bp, Artificial Sequence
  HBV F3 primer
tcctcacaataccgcagagt

SEQ ID NO: 3 - DNA, 20 bp, Artificial Sequence
  HBV B3 primer
gcatagcagcaggatgaaga

SEQ ID NO: 4 - DNA, 42 bp, Artificial Sequence
  HBV FIP primer
gttggggactgcgaattttggcctcgtggtggacttctctca

SEQ ID NO: 5 - DNA, 42 bp, Artificial Sequence
  HBV BIP primer
tcaccaacctcctgtcctccaaataaaacgccgcagacacat

SEQ ID NO: 6 - DNA, 17 bp, Artificial Sequence
  HBV LF primer
cacgggtgatcccccta

SEQ ID NO: 7 - DNA, 20 bp, Artificial Sequence
  HBV LB primer
tttgtcctggttatcgctgg

SEQ ID NO: 8 - DNA, 300 bp, Artificial Sequence
  Hinf target
tggtacgccaatacattcaacaagaaattaatccaaaagaaaaatttgcgtttgttgaattctgggggcgaggctatacacaagatacctttggtcgtctgctaaatgatgcctttggtaaagaagtaaaaaacccattctattatgtcagaagttttactgatgatatgggtacatctgttcgccataacttcatcttagcaccacaaaacttctcattcttcgagcctatttttgcacaaaccccatacgacagtattcctgattactacgaagaaaaaggcagaattgaaccaatta

SEQ ID NO: 9 - DNA, 22 bp, Artificial Sequence
  Hinf LF primer
gcagacgaccaaaggtatcttg

SEQ ID NO: 10 - DNA, 17 bp, Artificial Sequence
  Hinf B3 primer
cgtatggggtttgtgca

SEQ ID NO: 11 - DNA, 20 bp, Artificial Sequence
  Hinf F3 primer
cgccaatacattcaacaaga

SEQ ID NO: 12 - DNA, 47 bp, Artificial Sequence
  Hinf BIP primer
ctgatgatatgggtacatctgttcgcgaagaatgagaagttttgtgg

SEQ ID NO: 13 - DNA, 49 bp, Artificial Sequence
  Hinf FIP primer sequence
acttctttaccaaaggcatcattttgcgtttgttgacgccaaattctgg

SEQ ID NO: 14 - DNA, 34 bp, Artificial Sequence
  RPA Primer
cgccaatacattcaacaagaaattaatccaaaag

SEQ ID NO: 15 - DNA, 10 bp, Artificial Sequence
  RPA Blocker
cttttggatt

SEQ ID NO: 16 - DNA, 12 bp, Artificial Sequence
  RPA Blocker
cttttggattaa

SEQ ID NO: 17 - DNA, 15 bp, Artificial Sequence
  RPA Blocker
cttttggattaattt

SEQ ID NO: 18 - DNA, 17 bp, Artificial Sequence
  RPA Blocker
cttttggattaatttct

SEQ ID NO: 19 - DNA, 19 bp, Artificial Sequence
  RPA Blocker
cttttggattaatttcttg

SEQ ID NO: 20 - DNA, 21 bp, Artificial Sequence
  RPA Blocker
cttttggattaatttcttgtt

SEQ ID NO: 21 - DNA, 48 bp, Artificial Sequence
  LAMP Primer
acttctttaccaaaggcatcatttaatttgcgtttgttgaattctggg

SEQ ID NO: 22 - DNA, 50 bp, Artificial Sequence
  LAMP Primer
ttttactgatgatatgggtacatctgggctcgaagaatgagaagttttgt

SEQ ID NO: 23 - DNA, 20 bp, Artificial Sequence
  LAMP Primer
tggtacgccaatacattcaa

SEQ ID NO: 24 - DNA, 23 bp, Artificial Sequence
  LAMP Primer
cgccataacttcatcttagcacc

SEQ ID NO: 25 - DNA, 15 bp, Artificial Sequence
  LAMP Blocker
cccagaattcaacaa

SEQ ID NO: 26 - DNA, 16 bp, Artificial Sequence
  LAMP Blocker
acaaaacttctcattc

(19) United States
(12) Patent Application Publication
(10) Pub. No.: US 2022/0073975 A1
(43) Pub. Date: Mar. 10, 2022

(54) ISOTHERMAL AMPLIFICATION WITH ELECTRICAL DETECTION

(71) Applicant: Alveo Technologies, Inc., Alameda, CA (US)

(72) Inventors: Rixun Fang, Menlo Park, CA (US); Joseph Carl Gaiteri, Emeryville, CA (US); Brenna Hearn Lord, San Francisco, CA (US); Yuh-Min Chiang, Sunnyvale, CA (US); Ronald Phillip Chiarello, Orinda, CA (US)

(21) Appl. No.: 17/416,110

(22) PCT Filed: Dec. 18, 2019
(86) PCT No.: PCT/US2019/067077

§ 371 Date: Jun. 18, 2021

Related U.S. Application Data
(60) Provisional application No. 62/783,117, filed on Dec. 20, 2018.

(51) Int. Cl. C12Q 1/6844; G01N 27/02

(52) U.S. Cl. CPC C12Q 1/6844; G01N 27/02

(57) ABSTRACT

Some embodiments of the methods provided herein relate to amplifying and detecting a target nucleic acid. Some such embodiments include performing a recombinase polymerase amplification (RPA) and, optionally, a second isothermal amplification reaction. In some embodiments, the second isothermal amplification reaction includes loop-mediated isothermal amplification (LAMP). In some embodiments, the second isothermal amplification reaction is performed in conjunction with the RPA. In some embodiments, the second isothermal amplification reaction is performed on amplification products of the RPA. Some embodiments also include detecting the presence of amplification products by measuring a modulation of an electoral signal such as impedance.

### RELATED APPLICATIONS

This application claims priority to U.S. Prov. App. 62/783,117 filed Dec. 20, 2018 entitled “ISOTHERMAL AMPLIFICATION WITH ELECTRICAL DETECTION” which is hereby expressly incorporated by reference in its entirety.

### REFERENCE TO SEQUENCE LISTING

The present application is being filed along with a Sequence Listing in electronic format. The Sequence Listing is provided as a file entitled ALVEO021WOSEQLISTING, created Dec. 7, 2019, which is approximately 5 Kb in size. The information in the electronic format of the Sequence Listing is incorporated herein by reference in its entirety.

### FIELD

Some embodiments of the methods provided herein relate to amplifying and detecting a target nucleic acid. Some such embodiments include performing a recombinase polymerase amplification (RPA) and, optionally, a second isothermal amplification reaction, such as Loop-Mediated Isothermal Amplification (LAMP). Some embodiments also include detecting the presence of amplification products generated by RPA with or without a second isothermal amplification, such as LAMP, by measuring or analyzing a modulation of an electrical signal, such as impedance, which is desirably compared to a control.

### BACKGROUND

Pathogens in a sample may be identified by detecting specific genomic material (DNA or RNA). Beyond pathogen detection, many other biomarkers are available for testing, including molecules that provide early detection of cancer, vital prenatal information, or a greater understanding of a patient's microbiome. In conventional nucleic acid testing (“NAT”), genomic material in a sample may first be exponentially copied using a molecular amplification process known as the polymerase chain reaction (“PCR”) until the quantity of DNA present is great enough to be measurable. In the case of RNA, the genomic material of many viruses, an additional step can be included to first transcribe the RNA into DNA before amplifying by PCR.

### SUMMARY

Some embodiments include a method of amplifying and detecting a target nucleic acid, comprising: providing a recombinase polymerase amplification (RPA) reagent solution comprising a target nucleic acid, primers each being complementary to a region of said target nucleic acid, a buffer, dNTPs, a recombinase, a single-stranded DNA-binding protein (SSB), and a strand-displacing DNA polymerase, preferably, in a single vessel; combining the RPA reagent solution with a second reagent solution configured for a second isothermal amplification reaction, which does not utilize a recombinase, preferably in said single vessel, such as LAMP, so as to produce an amplification reaction solution; conducting RPA in said amplification reaction solution and, optionally, said second isothermal amplification, preferably in said single vessel, to produce an amplified target nucleic acid; optionally, conducting spiral RPA using a pair of primers with the forward and reverse primer sequences reverse complementary to each other at their 5′ end and their 3′ end sequences complementary to the target sequences; and detecting the presence of the amplified target nucleic acid by measuring a modulation of an electrical signal, such as impedance, in the amplification reaction solution when said amplification reaction is subjected to an electrical field, as compared to a control.

In some embodiments, the detecting of the amplified target nucleic acid is performed in a device comprising a test well, which comprises an excitation electrode and a sensor electrode, and wherein said detecting further comprises: applying an excitation signal from a reader device to the excitation electrode; sensing a signal from the test well using the excitation electrode, wherein the signal represents the impedance of the amplification reaction solution; and transmitting the signal to the reader device, wherein the reader device analyzes the signal.

In some embodiments, the recombinase comprises UvsX or RecA. In some embodiments, the SSB comprises gp32 or E. coli SSB. In some embodiments, the strand-displacing DNA polymerase comprises a Bst DNA polymerase large fragment, a Bst 2.0 polymerase, a Bst 3.0 polymerase, a Gsp polymerase, a Sau polymerase, a Bsu DNA polymerase large fragment, a Deep VentR DNA Polymerase, a Deep VentR (exo−) DNA Polymerase, a Klenow Fragment (3′→5′ exo−), a DNA Polymerase I Large (Klenow) Fragment, a phi29 DNA polymerase, a VentR DNA polymerase, or a VentR (exo−) DNA polymerase or any combination thereof.

In some embodiments, the RPA reagent solution further comprises a reagent selected from: Tris-Acetate, polyethylene glycol, trehalose, potassium acetate, creatine kinase, phosphocreatine, adenosine triphosphate, or a recombinase loading protein, such as uvsY, or wherein said RPA reagent solution or said amplification reaction solution does not comprise a primer having a detectable label or dye. In some embodiments, the amplification reaction solution comprises a concentration of 0.5-1 mM DTT, 0.25-0.5 mM DTT, about 0.25 mM DTT, 0.25 mM DTT, 0-0.25 mM DTT, or 0 mM DTT. In some embodiments, the amplification reaction solution comprises a concentration of 1-10 mM DTT, about 5 mM DTT or 5 mM DTT, and 5-15 mM calcium chloride; about 0.9 mM calcium chloride, 0.9 mM calcium chloride, 5-15 mM Ca2+, about 0.9 mM Ca2+, or 0.9 mM Ca2+. In some embodiments, the amplification reaction solution comprises magnesium or magnesium ions at a concentration of 1-20 mM, 1-5 mM, 5-10 mM, 7-9 mM, 8 mM, about 8 mM, 10-20 mM, about 13 mM, or 13 mM. In some embodiments, the amplification reaction solution comprises a dNTP mixture at a concentration of 1-10 mM, 1-3 mM, about 1.8 mM, 1.8 mM, 5-6 mM, about 5.6 mM or 5.6 mM. In some embodiments, the amplification reaction solution further comprises a blocker oligonucleotide comprising a nucleic acid sequence that is a reverse complement to part of the nucleic acid sequence of one or more of said primers.

In some embodiments, the RPA comprises leading strand RPA (QsRPA), simultaneous leading and lagging strand synthesis, or nested RPA. In some embodiments, the second isothermal amplification comprises self-sustaining sequence replication reaction (3SR), 90-I, BAD Amp, cross priming amplification (CPA), isothermal exponential amplification reaction (EXPAR), isothermal chimeric primer initiated amplification of nucleic acids (ICAN), isothermal multi displacement amplification (IMDA), ligation-mediated SDA; multi displacement amplification; polymerase spiral reaction (PSR), restriction cascade exponential amplification (RCEA), smart amplification process (SMAP2), single primer isothermal amplification (SPIA), transcription-based amplification system (TAS), transcription meditated amplification (TMA), ligase chain reaction (LCR), or multiple cross displacement amplification (MCDA), rolling circle replication (RCA), Nicking Enzyme Amplification Reaction (NEAR) or Nucleic acid sequence based amplification (NASBA).

In some embodiments, the second isothermal amplification comprises Loop-Mediated Isothermal Amplification (LAMP). In some embodiments, the amplification reaction solution comprises a primer oligonucleotide compatible with LAMP. In some embodiments, the amplification reaction solution comprises FIP, BIP, LF, or LB primer oligonucleotides compatible with LAMP, and primers compatible with RPA. In some embodiments, the amplification reaction solution comprises FIP, BIP, LF, LB, F3, or B3 primer oligonucleotides compatible with LAMP.

In some embodiments, the RPA and the second isothermal amplification, such as LAMP, are conducted at the same or substantially the same temperature. In some embodiments, the RPA or the second isothermal amplification, such as LAMP, are conducted at 25° C., 30° C., 37° C., 40° C., 45° C., 50° C., 60° C., 65° C., 70° C., 75° C., or at a temperature that is within a range defined by any two of the aforementioned temperatures. In some embodiments, the RPA is conducted at a lower or higher temperature than the second isothermal amplification, such as LAMP. In some embodiments, the RPA is conducted at 37° C., about 37° C., 40° C., or about 40° C., and the second isothermal amplification, such as LAMP, is conducted at 60° C., about 60° C., 65° C., or about 65° C.

In some embodiments, the primers do not comprise a label, marker, or a dye. In some embodiments, the amplification and detecting are performed in the absence of a detection reagent such as a dye, a turbidity agent, a fluorophore, a double-stranded nucleic acid intercalating agent, a sequencing index, or a nanoparticle.

Some embodiments include a method of amplifying and detecting a target nucleic acid, comprising: providing a recombinase polymerase amplification (RPA) reagent solution comprising a target nucleic acid, primers each being complementary to a region of said target nucleic acid, a buffer, dNTPs, a recombinase, a single-stranded DNA-binding protein (SSB), and a strand-displacing DNA polymerase, preferably, in a single vessel; conducting RPA in the RPA reagent solution to produce an amplified target nucleic acid; combining the amplified target nucleic acid with a second reagent solution configured for a second isothermal amplification reaction, such as LAMP, which does not utilize a recombinase, preferably in said single vessel, to produce a second amplification reaction solution; conducting a second isothermal amplification of said second amplification reaction solution to produce a further amplified target nucleic acid; and detecting the presence of the amplified target nucleic acid by measuring a modulation of an electrical signal, such as impedance, in the amplification reaction solution when said amplification reaction is subjected to an electrical field, optionally, as compared to a control.

In some embodiments, combining the amplified target nucleic acid with a second reagent solution configured for a second isothermal amplification reaction comprises adding the second reagent solution to the RPA reagent solution after conducting RPA to produce an amplified target nucleic acid. In some embodiments, combining the amplified target nucleic acid with a second reagent solution configured for a second isothermal amplification reaction comprises adding the RPA reagent solution or a portion of the RPA reagent solution to a second reagent solution after conducting RPA to produce an amplified target nucleic acid.

In some embodiments, the recombinase comprises UvsX or RecA. In some embodiments, the SSB comprises gp32 or E. coli SSB. In some embodiments, the strand-displacing DNA polymerase comprises a Bst DNA polymerase large fragment, a Bst 2.0 polymerase, a Bst 3.0 polymerase, a Gsp polymerase, a Sau polymerase, a Bsu DNA polymerase large fragment, a Deep VentR DNA Polymerase, a Deep VentR (exo−) DNA Polymerase, a Klenow Fragment (3′→5′ exo−), a DNA Polymerase I Large (Klenow) Fragment, a phi29 DNA polymerase, a VentR DNA polymerase, or a VentR (exo−) DNA polymerase. In some embodiments, the RPA reagent solution further comprises a reagent selected from: Tris-Acetate, polyethylene glycol, trehalose, potassium acetate, creatine kinase, phosphocreatine, a recombinase loading protein, such as uvsY, or wherein said RPA reagent solution or said amplification reaction solution does not comprise a primer having a detectable label, marker, or dye.

In some embodiments, the amplification reaction solution comprises a concentration of 0.5-1 mM DTT, 0.25-0.5 mM DTT, about 0.25 mM DTT, 0.25 mM DTT, 0-0.25 mM DTT, or 0 mM DTT. In some embodiments, the amplification reaction solution comprises a concentration of 1-10 mM DTT, about 5 mM DTT or 5 mM DTT, and 5-15 mM calcium chloride; about 0.9 mM calcium chloride, 0.9 mM calcium chloride, 5-15 mM Ca2+, about 0.9 mM Ca2+, or 0.9 mM Ca2+. In some embodiments, the amplification reaction solution comprises magnesium or magnesium ions at a concentration of 1-20 mM, 1-5 mM, 5-10 mM, 7-9 mM, 8 mM, about 8 mM, 10-20 mM, about 13 mM, or 13 mM. In some embodiments, the amplification reaction solution comprises a dNTP mixture at a concentration of 1-10 mM, 1-3 mM, about 1.8 mM, 1.8 mM, 5-6 mM, about 5.6 mM or 5.6 mM. In some embodiments, the amplification reaction solution further comprises a blocker oligonucleotide comprising a nucleic acid sequence that is a reverse complement to part of the nucleic acid sequence of one or more of said primers. In some embodiments of the methods described herein, an amplification reaction solution comprises TCEP. In some embodiments, the TCEP in the amplification reaction solution is at 5 mM, about 5 mM, 4-5 mM, 5-6 mM, 4-6 mM, 3-7 mM, 2-8 mM, 1-9 mM or 1-10 mM. In some embodiments of the methods described herein, an amplification reaction solution comprises a reducing agent such as DTT or TCEP. In some embodiments, the reducing agent in the amplification reaction solution is at 5 mM, about 5 mM, 4-5 mM, 5-6 mM, 4-6 mM, 3-7 mM, 2-8 mM, 1-9 mM or 1-10 mM.

In some embodiments, the RPA comprises leading strand RPA (QsRPA), simultaneous leading and lagging strand synthesis, or nested RPA. In some embodiments, the second isothermal amplification comprises self-sustaining sequence replication reaction (3SR), 90-I, BAD Amp, cross priming amplification (CPA), isothermal exponential amplification reaction (EXPAR), isothermal chimeric primer initiated amplification of nucleic acids (ICAN), isothermal multi displacement amplification (IMDA), ligation-mediated SDA; multi displacement amplification; polymerase spiral reaction (PSR), restriction cascade exponential amplification (RCEA), smart amplification process (SMAP2), single primer isothermal amplification (SPIA), transcription-based amplification system (TAS), transcription meditated amplification (TMA), ligase chain reaction (LCR), or multiple cross displacement amplification (MCDA), rolling circle replication (RCA), Nicking Enzyme Amplification Reaction (NEAR) or Nucleic acid sequence based amplification (NASBA).

In some embodiments, the second isothermal amplification comprises Loop-Mediated Isothermal Amplification (LAMP). In some embodiments, the amplification reaction solution comprises a primer oligonucleotide compatible with LAMP. In some embodiments, the amplification reaction solution comprises FIP, BIP, LF, or LB primer oligonucleotides compatible with LAMP, and primers compatible with RPA. In some embodiments, the amplification reaction solution comprises FIP, BIP, LF, LB, F3, or B3 primer oligonucleotides compatible with LAMP.

In some embodiments, the RPA and the second isothermal amplification, such as LAMP, are conducted at the same or substantially the same temperature. In some embodiments, the RPA or the second isothermal amplification, such as LAMP, are conducted at 25° C., 30° C., 37° C., 40° C., 45° C., 50° C., 60° C., 65° C., 70° C., 75° C., or at a temperature that is within a range defined by any two of the aforementioned temperatures. In some embodiments, the RPA is conducted at a lower or higher temperature than the second isothermal amplification, such as LAMP. In some embodiments, the RPA is conducted at 37° C., about 37° C., 40° C., or about 40° C., and the second isothermal amplification, such as LAMP, is conducted at 60° C., about 60° C., 65° C., or about 65° C.

In some embodiments, the primers do not comprise a label, marker, or a dye. In some embodiments, the amplification and detecting are performed in the absence of a detection reagent such as a dye, a turbidity agent, a fluorophore, a double-stranded nucleic acid intercalating agent, a sequencing index, or a nanoparticle.

Some embodiments include a method of amplifying and detecting a target nucleic acid, comprising: performing a recombinase polymerase amplification (RPA) and a Loop-Mediated Isothermal Amplification (LAMP) on a target nucleic acid in a single vessel to produce an amplified target nucleic acid, preferably without isolating or purifying the amplified target nucleic acid between said RPA amplification and said LAMP amplification, such as in a single vessel; and detecting the presence of the amplified target nucleic acid by measuring a modulation of an electrical signal, such as impedance, in the amplification reaction solution when said amplification reaction is subjected to an electrical field, optionally, as compared to a control.

In some embodiments, the RPA and the LAMP amplifications are conducted at the same or substantially the same temperature. In some embodiments, the RPA or LAMP amplifications are conducted at 25° C., 30° C., 37° C., 40° C., 45° C., 50° C., 60° C., 65° C., 70° C., 75° C., or at a temperature that is within a range defined by any two of the aforementioned temperatures. In some embodiments, the RPA is conducted at a lower or higher temperature than the LAMP amplification. In some embodiments, the RPA is conducted at 37° C., about 37° C., 40° C., or about 40° C., and the LAMP amplification is conducted at 60° C., about 60° C., 65° C., or about 65° C.

In some embodiments, the primers in the RPA or LAMP amplifications do not comprise a label, marker, or a dye. In some embodiments, the RPA and LAMP amplifications and the detecting are performed in the absence of a detection reagent such as a dye, a turbidity agent, a fluorophore, a double-stranded nucleic acid intercalating agent, a sequencing index, or a nanoparticle.

In some embodiments, the detecting of the amplified target nucleic acid is performed in a device comprising a test well, which comprises an excitation electrode and a sensor electrode, and wherein said detecting further comprises: applying an excitation signal from a reader device to the excitation electrode; sensing a signal from the test well using the excitation electrode, wherein the signal represents the impedance of the amplification reaction solution; and in some embodiments, transmitting the signal to the reader device, wherein the reader device analyzes the signal.

### BRIEF DESCRIPTION OF THE DRAWINGS

FIGS. 1A-1D depict an example cartridge for detection of a target.

FIG. 2 depicts another example cartridge for detection of a target.

FIGS. 3A and 3B depict another example cartridge for detection of a target.

FIGS. 4A-4G depict various examples of electrodes that can be used in a test well of the cartridges of FIGS. 1A-3B, FIGS. 51A-53B or in the test well or channel of another suitable target detection cartridge as described herein.

FIG. 5A depicts a first electrode or excitation electrode and a second electrode or signal electrode that may be spaced apart from one another within a test well of the cartridges of FIGS. 1A-3B, FIGS. 51A-53B or in the test well or channel of another suitable target detection cartridge as described herein.

FIG. 5B depicts an example signal that can be extracted from the signal electrode of FIG. 5A.

FIG. 5C depicts the resistance and reactance components extracted from a signal as shown in FIG. 5B generated based on an example positive test.

FIG. 5D depicts the resistance and reactance components extracted from signals as shown in FIG. 5B from example tests of positive and negative controls.

FIG. 5E depicts the resistance and reactance components extracted from a signal as shown in FIG. 5B generated based on another example positive test.

FIG. 6 depicts a schematic block diagram of an example reader device that can be used with the cartridges described herein.

FIG. 7A depicts a flowchart of an example process for operating a reader device during a test as described herein.

FIG. 7B depicts a flowchart of an example process for analyzing test data to detect a target as described herein.

FIG. 8 depicts an amplification immunoassay scheme.

FIG. 9 depicts a bead-based amplification immunoassay scheme.

FIG. 10 depicts a magnetic bead-based amplification immunoassay scheme.

FIG. 11 depicts a first electrode or excitation electrode and a second electrode or signal electrode that may be spaced apart from one another along a channel.

FIG. 12 is a graph showing the impedance of a signal is dependent on the excitation frequency and changes after a LAMP reaction occurs in a channel in which the left inequality may define a frequency region.

FIG. 13 is a graph showing that in both extremal regions the impedance is capacitor-like and is out of phase (approaching 90°) with the excitation voltage.

FIG. 14 is a graph depicting the measured impedance of a sample chip with respect to excitation frequency.

FIG. 15 is a graph depicting a synchronous detector response plotted with respect to non-dimensional conductivity.

FIG. 16 is a graph depicting results of a model demonstrating agreement with a detector output for a wide range of conductivities and for a given steps in frequencies.

FIG. 17A and FIG. 17B depict an embodiment of a detection system that may be used to detect presence or absence of a particular nucleic acid and/or a particular nucleotide in a sample. FIG. 17A is a top view of the system, while FIG. 17B is a cross-sectional side view of the system.

FIG. 18 is a process flow chart illustrating an implementation of device for detecting a target.

FIG. 19 is a process flow chart illustrating an implementation of a device for detecting a target.

FIG. 20 depicts an example fluidics cartridge.

FIG. 21 is a plan view of the example fluidic cartridge of FIG. 20.

FIG. 22 depicts an example configuration for electrodes.

FIG. 23 depicts an example channel.

FIG. 24 is a graph depicting sensor voltage over time for pre-amplification (− control), and post-amplification (+ control).

FIG. 25 is a graph depicting sensor voltage over time for pre-amplification (− control), and post-amplification (+ control) for 0% whole blood.

FIG. 26 is a graph depicting sensor voltage over time for pre-amplification (− control), and post-amplification (+ control) for 1% whole blood.

FIG. 27 is a graph depicting sensor voltage over time for pre-amplification (− control), and post-amplification (+ control) for 5% whole blood.

FIG. 28 is a graph depicting sensor voltage over time for pre-amplification (− control), and post-amplification (+ control) with 0% whole blood for unfiltered sample.

FIG. 29 is a graph depicting sensor voltage over time for pre-amplification (− control), and post-amplification (+ control) with 0% whole blood for filtered sample.

FIG. 30 depicts a graph of time over target load with error bars showing standard deviation.

FIG. 31 depicts a graph of conductivity for various samples from pre-amplification vial (− control), and post-amplification vial (+ control).

FIG. 32 depicts a magnetic bead-based amplification immunoassay scheme for the detection of HBsAg.

FIG. 33 depicts a graph illustrating detection of HBsAg.

FIG. 34 depicts a graph illustrating detection of HBsAg with a low ionic buffer (T 10).

FIG. 35 depicts a graph illustrating impedance characteristics of a fluidics cartridge.

FIG. 36A depicts a graph for out of phase signals for LAMP carried on a cartridge at 65° C.

FIG. 36B depicts a graph for in phase signals for LAMP carried on a cartridge at 65° C.

FIG. 36C depicts a graph for out of phase signals for LAMP carried on a cartridge at 67° C.

FIG. 36D depicts a graph for in phase signals for LAMP carried on a cartridge at 67° C.

FIG. 36E depicts a graph for out of phase signals for LAMP carried on a cartridge at 67° C.

FIG. 36F depicts a graph for in phase signals for LAMP carried on a cartridge at 67° C.

FIG. 37 depicts an example of RecA/Primer Loading.

FIG. 38A is a schematic depicting an example of steps of Leading Strand Recombinase Polymerase Amplification (lsRPA).

FIG. 38B is a schematic depicting an example of steps of Leading Strand Recombinase Polymerase Amplification (lsRPA).

FIG. 39A is a schematic depicting an example of steps of Leading and Lagging Strand Recombinase Polymerase Amplification.

FIG. 39B is a schematic depicting an example of steps of Leading and Lagging Strand Recombinase Polymerase Amplification.

FIG. 39C is a schematic depicting an example of steps of Leading and Lagging Strand Recombinase Polymerase Amplification.

FIG. 39D is a schematic depicting an example of steps of Leading and Lagging Strand Recombinase Polymerase Amplification.

FIG. 40 depicts an example of nested primers chosen for nested RPA.

FIG. 41 is a diagram of an example of a method described herein.

FIG. 42 is a diagram of an example of a method described herein.

FIG. 43 is a diagram of an example of a method described herein.

FIG. 44 is a plot depicting cartridge test results.

FIG. 45A is a plot showing amplification curves according to some embodiments.

FIG. 45B is a plot showing melt curves according to some embodiments.

FIG. 45C is a plot showing amplification curves according to some embodiments.

FIG. 45D is a plot showing melt curves according to some embodiments.

FIG. 45E is a table with Ct data according to some embodiments.

FIG. 46A is an amplification plot for RPA according to some embodiments.

FIG. 46B is an amplification plot for RPA according to some embodiments.

FIG. 47A is an amplification plot for an RPA stage according to some embodiments.

FIG. 47B is an amplification plot for an RPA stage according to some embodiments.

FIG. 48A is an amplification plot for a LAMP stage according to some embodiments.

FIG. 48B is a melt curve plot for a LAMP stage according to some embodiments.

FIG. 48C is an amplification plot for a LAMP stage according to some embodiments.

FIG. 48D is a melt curve plot for a LAMP stage according to some embodiments.

FIG. 48E is an amplification plot for a LAMP stage according to some embodiments.

FIG. 48F is a melt curve plot for a LAMP stage according to some embodiments.

FIG. 49A is an amplification plot according to some embodiments.

FIG. 49B is an amplification plot according to some embodiments.

FIG. 49C is an amplification plot according to some embodiments.

FIG. 49D is an amplification plot according to some embodiments.

FIG. 50A is an amplification plot according to some embodiments.

FIG. 50B is an amplification plot according to some embodiments.

FIG. 50C is an amplification plot according to some embodiments.

FIGS. 51A-51C depict an example handheld system for detection of a target.

FIGS. 52A-52F depict an example cartridge for detection of a target that can be used in the handheld system of FIGS. 51A-51C.

FIGS. 53A-53E depict a mechanical fluid transfer mechanism of the example cartridge of FIGS. 52A-52F.

FIG. 54 depicts a flowchart of an example process for operating a reader device during a test as described herein.

FIGS. 55A-55D depict an example user interface of a user device implementing an example testing process in communication with a reader device as described herein.

FIGS. 56A and 56B depict an example handheld system for detection of a target.

FIGS. 57A-57K depict an example cartridge for detection of a target that can be used in the handheld system of FIGS. 56A and 56B.

FIGS. 58A-58D depict a mechanical fluid transfer mechanism of the example cartridge of FIGS. 57A-57J.

FIG. 59 depicts a schematic block diagram of an example reader device that can be used with the cartridges described herein.

FIGS. 60A-60I depict an example cartridge for detection of a target that can be used in conjunction with the handheld systems disclosed herein.

### DETAILED DESCRIPTION

Aspects of the disclosure herein concern the use of amplification and contactless electrical sensing to detect the presence of a target in a sample. Such a diagnostic platform may replace the complex optical systems and expensive fluorescent labels used for optical detection and the electrodes and electroactive agents used in existing electrochemical and FET techniques with common electronic components. In some aspects, the amplification can be isothermal, such as utilizing RPA with or without LAMP. The platform described herein is inexpensive, robust, portable, and consumes less power than traditional diagnostic systems. In some aspects, the diagnostic platform is small enough to fit in the palm of a consumer's hand and capable of performing in the field, for example, a diagnosis in a doctor's office, in the home, in a location remote from a medical facility.

Certain embodiments provided herein include aspects disclosed in: U.S. 62/782,610 filed on Dec. 20, 2018 entitled “METHODS AND COMPOSITIONS TO REDUCE NONSPECIFIC AMPLIFICATION IN ISOTHERMAL AMPLIFICATION REACTIONS”; U.S. 62/783,104 filed on Dec. 20, 2018 entitled “HANDHELD IMPEDANCE-BASED DIAGNOSTIC TEST SYSTEM FOR DETECTING ANALYTES”; and U.S. 62/783,051 filed on Dec. 20, 2018 entitled “METHODS AND COMPOSITIONS FOR DETECTION OF AMPLIFICATION PRODUCTS”, the entire contents of which are each expressly incorporated by reference in its entirety. Certain embodiments provided herein also include aspects disclosed in U.S. 2016/0097740, U.S. 2016/0097741, U.S. 2016/0097739, U.S. 2016/0097742, U.S. 2016/0130639, and U.S. 2019/0232282, the entire contents of which are each expressly incorporated by reference in its entirety.

Many commercially available nucleic acid detection platforms utilize traditional PCR, thereby requiring temperature cycling, fluorescent labels and optical detection instrumentation. These factors result in expensive, lab-based instrumentation which employ delicate, vibration sensitive detectors, costly fluorescent markers, and have a large footprint. The equipment requires operation, and frequent calibration, by highly trained personnel.

These large, unwieldy platforms make routine use of conventional NAT challenging to use in the clinic, much less in the home. NAT remains a costly and slow strategy closely tied to centralized laboratory facilities. The presently disclosed technology, in contrast, avoids these challenges.

A hurdle to point of care (“POC”) testing is the potential inhibition of amplification by interferents often encountered in crude, unprocessed clinical samples such as whole blood or mucus. The mitigation of amplification inhibitors may challenge the direct detection of target nucleic acids from clinically relevant biologic samples.

Traditional detection strategies commonly rely on fluorescence detection techniques. Such techniques may be complex, more expensive, and require precision optical systems. The present disclosure however, generally relies on electrical detection systems. Such electrical detection systems may leverage microelectronics that consume relatively low power and can be manufactured at a reduced cost due to high volume manufacturing. Thus, electrical detection of genomic material may transfer the advances of the computer industry to bioassay sensing.

Existing electronic methods for monitoring amplification may require the binding of an electrochemically active label or the selective binding of the amplified material to a surface. However, when used in real world clinical applications, these techniques often suffer from slow response times, biofouling of the electrode or binding surfaces resulting in poor signal to noise ratios, and limitations on the lifetime and reliability of the device. While potentially enabling great sensitivity, the use of electrochemical or field effect transistor “FET” detection adds a layer of complexity to the detection. This can result in more expensive and less robust strategies than POC and other consumer applications typically dictate. Accordingly, the need for additional diagnostic devices is manifest.

The platform disclosed herein relies on measurement of the change in electrical conductivity that occurs during nucleic acid amplification. In sum, during biochemical synthesis of DNA from nucleotide triphosphates, the number and the mobility of electrically charged molecules are altered. This, in turn, results in a change in the solution conductivity as amplification progresses. This change in solution electrical conductivity may be sensed using frequency-dependent capacitively coupled contactless conductivity detection (“ƒC⁴D”).

In some implementations, ƒC⁴D uses a pair of electrodes in close proximity to, but not in contact with, a fluid disposed in an amplification chamber to measure the solution's electrical properties. The ability to measure the properties of the solution in this way, without direct contact, avoids the challenges of surface fouling common to other electrical measurement methods.

In some implementations, utilizing ƒC⁴D, a high-frequency alternating current (“AC”) signal is applied to the excitation electrode. This signal is capacitively coupled through the solution where it is detected at the signal electrode. By comparing the excitation signal with the signal at the signal electrode, the solution's conductivity can be determined.

Informed by high-resolution finite element models and empirical studies, specific tolerances of ƒC⁴D based technology may achieve the optimal detection sensitivity and dynamic sensing range for particular implementations of the platform. Such calculated and empirically determined parameters of microfluidic dimensions, capacitive coupling characteristics, and the applied frequency can enable the determination of the effective parameters for detecting solution conductivity changes. In some embodiments, the parameters corresponding to optimal detection can be interdependent variables. According to the following equation, the measured impedance is a function of the solution resistance, capacitance and the applied frequency:

Z=R−(1/pi*f*C)*j

As the thickness of the electrode passivation layer increases, a parasitic capacitance due to this layer consequently increases. The optimal AC frequency with which to measure solution conductivity by ƒC⁴D therefore can be chosen with respect to the capacitance of the passivation layer.

### Overview of Example Cartridges, Readers, and Signal Processing

In some aspects, a system for detecting a target in a sample includes a removable fluidics cartridge that is couplable to a companion reader device. A user can apply a sample to the cartridge and then insert it into the reader device. The reader device is configured for performing the testing procedures using the cartridge and analyzing the test data to determine the presence, absence, or quantity of a target in the sample. For example, the cartridge can be provided with the desired agents, proteins, or other chemical matter for an amplification process by which a target initially present in the sample is amplified. Specifically, some cartridges can be provided with the desired chemical matter for nucleic acid testing, wherein genomic material in the sample is exponentially copied using a molecular amplification process, as described herein. The cartridge can also include a test well for containing the amplification process, where a test well refers to a well, chamber, channel, or other geometry configured for containing (or substantially containing) test fluid and constituents of the amplification process. The reader device may maintain a desired temperature or other test environment parameters for the cartridge to facilitate the amplification process and can electronically monitor a test well of the cartridge throughout some or all of the amplification process. The reader device can thus gather signal data representing the impedance of the test well over time during the amplification process and can analyze the impedance as described herein to ascertain the presence, absence, or quantity of the target in the sample. As an example, the amplification process can range from five minutes to sixty minutes, with some examples ranging from ten minutes to thirty minutes. Preferably, in some embodiments, the amplification products are detected while being suspended or while being mobilized in the fluid within the wells such that the amplification products are not attached or sequestered to the wells or fixed or bound to probes, which are bound to the wells or particles within the wells. In other embodiments, the amplification products are detected as they are attached or sequestered to the wells or particles within the wells e.g., fixed or bound to probes, which are bound to the wells or particles present in the wells.

Such systems can beneficially provide target detection performable in a clinical setting or even the home of a user, rather than requiring the sample to be sent to a laboratory for amplification and analysis. In the clinical setting, this can avoid the delays of conventional nucleic acid testing thereby enabling clinicians to determine diagnoses within the typical timeframe of a patient's office visit. As such, the disclosed systems enable clinicians to develop treatment plans for patients during their initial office visit, rather than requiring the clinician to wait for hours or even days to receive test results back from a laboratory. For example, when a patient visits a clinic a nurse or other healthcare practitioner can collect a sample from the patient and begin testing using the described system. The system can provide the test result by the time the patient consults with their doctor or clinician to determine a treatment plan. Particularly when used to diagnose pathologies that progress quickly, the disclosed systems can avoid the delays associated with laboratory testing that can negatively impact the treatment and outcome of the patient.

As another benefit, the disclosed systems can be used outside of the clinical setting (e.g., in the field, in rural settings without easy access to an established healthcare clinic) to detect health conditions such as contagious diseases (e.g., Ebola), thus enabling the appropriate personnel to take immediate action to prevent or mitigate the spread of a contagious disease. Similarly, the disclosed systems can be used in the field or at the site of a suspected hazardous contaminant (e.g., anthrax) to quickly determine whether a sample contains the hazardous contaminant, thus enabling the appropriate personnel to take immediate action to prevent or mitigate human exposure to the contaminant. Additionally, the disclosed systems can be used to detect contaminants in the blood or plasma supply or in the food industry. It will be appreciated that the disclosed systems can provide similar benefits in other scenarios in which real-time detection of a target enables more effective action than delayed detection through sending a sample to an off-site laboratory.

Another benefit of such systems is their use of low-cost, disposable single use cartridges together with a reusable reader device that can be used many times with different cartridges and/or for tests with different targets.

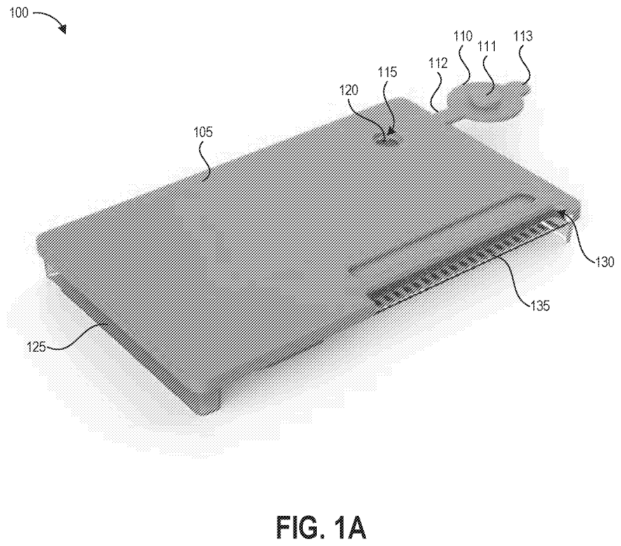

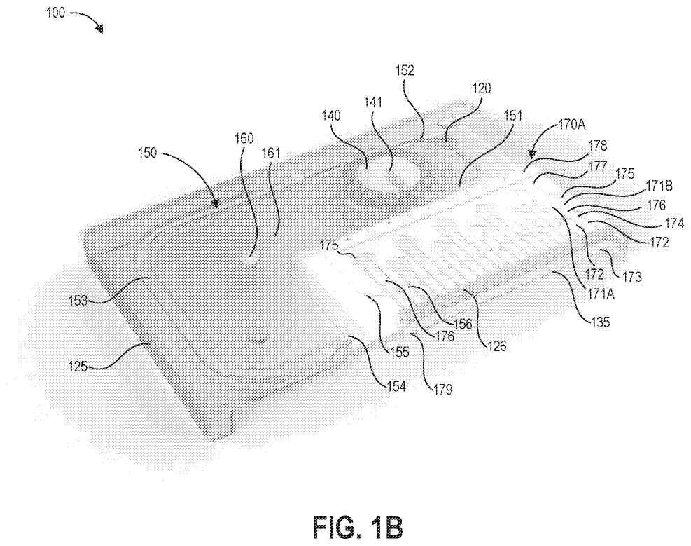

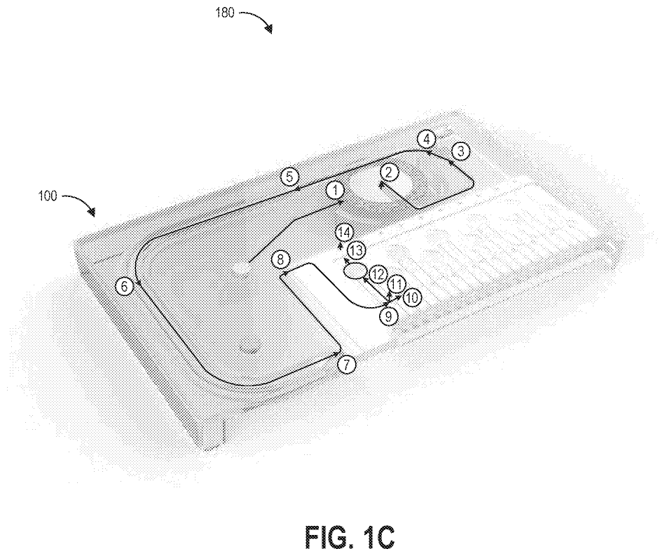

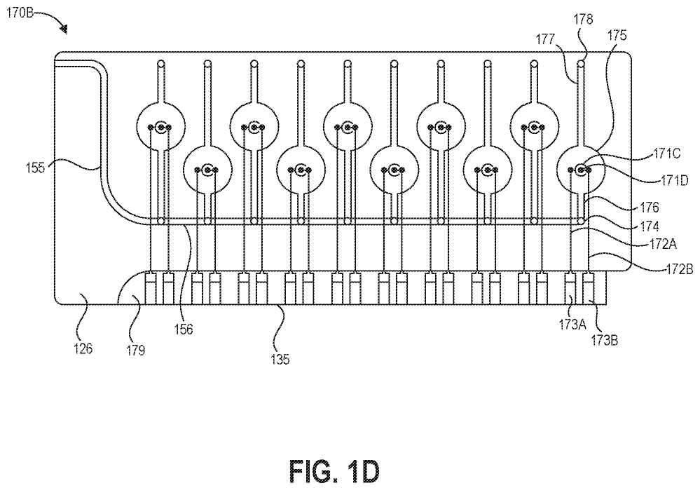

FIGS. 1A-1D depict an example cartridge 100 configured for detection of a target. As described herein, the target may be a viral target, bacterial target, antigen target, parasite target, microRNA target, or agricultural analyte. Some embodiments of the cartridge 100 can be configured for testing for a single target, while some embodiments of the cartridge 100 can be configured for testing for multiple targets.

FIG. 1A depicts the cartridge 100 with cover 105 provided over its base 125. In use, the cover 105 can operate to seal a provided sample within the cartridge 100, thereby preventing exposure of test operators to the sample and preventing any liquid from escaping into the electronics of an associated reader device. The cover 105 may be permanently affixed to the base 125 or may be removable in certain embodiments. The cover 105 can be formed from suitable materials such as plastic and may be opaque as depicted or in other examples may be translucent or transparent.

The cover 105 includes an aperture 115 positioned over a sample introduction area 120 of the base 125. Over, as used here, refers to the aperture 115 being above the sample introduction area 120 when the cartridge 100 is viewed from a top-down perspective orthogonally to the planar surface of the cover 105 including the aperture 115. The cover 105 also includes a cap 110 configured to fluidically seal the aperture 115 before and after provision of a sample through the aperture 115. The cap 110 includes a cylindrical protrusion 111 that plugs the aperture 115 when the cap 110 is sealed with the aperture 115, a release tab 113 configured to assist a user in pulling the cap 110 out of the aperture 115 when the cap 110 is sealed with the aperture 115, and a hinge 112 configured to enable the cap 110 to be moved away from the aperture 115 and out of a sample provision path while keeping the cap 110 secured to the cover 105. It will be appreciated that other variations of the shape of the cap 110 can similarly be used to achieve the sealing of aperture 115, and in some embodiments the hinge 112 and/or release tab 113 can be modified or omitted. In the illustrated embodiment, the cover 105 and cap 110 are formed integrally as a single piece of material, however in other embodiments the cap 110 can be a separate structure from the cover 105.

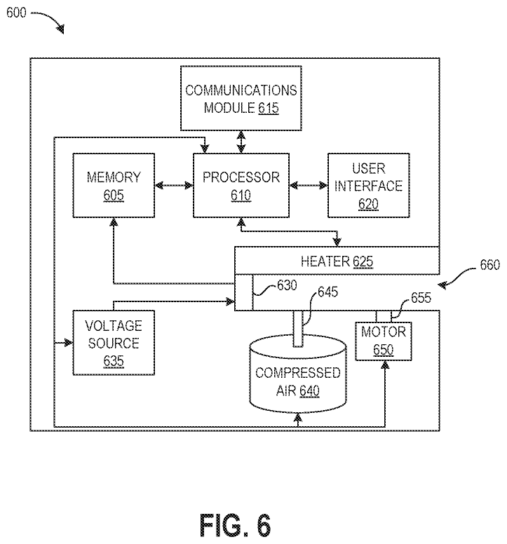

In use, a user opens the cap 110 and applies a sample potentially containing the target(s) to the sample introduction area 120 of the base 125 through the aperture 115 in the cover. For example, a user can prick a finger and apply a whole blood sample to the sample introduction area 120, for example through a capillary. The cartridge 100 can be configured to accept one or more of liquid, semi-solid, and solid samples. After applying the sample, the user can close the cap 110 to seal the aperture 115. Beneficially, sealing the entrance to the fluid path of the base 125 allows the sample (and other liquids) to be moved through the fluid path of the base 125 to a test well. For example, the user can insert the sealed cartridge 100 containing the sample into a reader device as described herein, and the reader device can activate an optional pneumatic interface for moving the sample to the test well. The fluid path and test well are described in more detail with respect to FIGS. 1B and 1C, and an example reader device is described with respect to FIG. 6.

The cover 105 also includes a recess 130 for exposing an electrode interface 135 of the base 125, described in more detail below. In some embodiments, the cover 105 can include a movable flap or removable sheath for protecting the electrode interface 135 prior to use.

FIG. 1B depicts the cartridge 100 of FIG. 1A with the cover removed to expose the features of the base 125. The base 125 can be formed from a fluid impermeable material, for example injection molded or milled acrylic or plastic. The base 125 includes sample introduction area 120, a blister pack 140, pneumatic interface 160, test region 170A including test wells 175, and a fluid path 150 configured for mixing the applied sample with the liquids contained in the blister pack 140 and for carrying this mixed liquid to the test wells 175. It will be appreciated that the particular geometric configurations or relative arrangements of these features may be varied in other embodiments.

Blister pack 140 includes a film, for example a thermoformed plastic, forming a sealed chamber containing liquids for mixing with the applied sample. These liquids can include amplification reagents, buffer solutions, water, or other desired liquid constituents for the testing process. The particular selection and chemistry of these liquids can be tailored to a particular target or targets for which the cartridge 100 is designed to test. Some embodiments of the blister pack 140 can additionally include non-liquid compounds dissolved or suspended in the enclosed liquid. The blister pack 140 can be secured to the base 125, for example within a fluid-tight chamber having a pneumatic fluid path 161 leading into the chamber and aperture 141 leading out of the chamber into the fluid path 150. For example, a ring of pressure-sensitive adhesive disposed along the outer edge of one or both surfaces of the blister pack 140 can be used to secure the blister pack 140 in place.

In use, a user or reader device can mechanically actuate a sharp (e.g., a needle or other body having a sharp point) to puncture the blister pack 140 and release its liquid contents through aperture 141 and into the first segment 151 of the fluid path 150. The sharp may be incorporated into the cartridge 100, for example located in a chamber containing the blister pack 140 with the chamber in fluidic communication with the first segment 151 of the fluid path. As used herein, fluidic communication refers to the capability to transfer fluids (e.g., liquid gas gas). In another embodiment, the user or reader device can press on a lower surface of the blister pack 140 (though not illustrated, the lower surface opposes the surface visible in FIG. 1B) to push it upward into the sharp and puncture the blister pack 140. In other embodiments, the sharp can be omitted, and the blister pack 140 can be compressed by the user or reader device until the pressure of its liquid contents causes the blister pack 140 to rupture. Though described as a rupturable blister pack, other embodiments can implement mechanically openable chambers configured to similarly release the enclosed liquids into the first segment 151 of the fluid path 150.

As described above, after application of the sample the user seals the aperture 115 of the cover, thereby sealing the fluid path 150 within the cartridge 100. The pneumatic interface 160 is configured to provide a fluid or medium such as air into the sealed fluid path 150 through the blister pack chamber in order to promote flow of fluid in the desired direction along the fluid path 150 to the test wells 175. Pneumatic interface 160 can be an aperture leading into and in fluidic communication with a pneumatic fluid path 161 that in turn leads into and is in fluidic communication with the blister pack 140 or the chamber containing the blister pack 140. In some embodiments, the pneumatic interface 160 can be a compressible one-way valve that forces ambient air into the pneumatic fluid path 161 when compressed and takes in ambient air from its environment as it decompresses. In such embodiments, repeated compression of the pneumatic interface 160 can force the fluid in the cartridge along the fluid path.

The fluid path 150 includes segments 151, 152, 153, 154, 155, and 156 as well as sample introduction area 120, test well 175, test well inlet path 176, and test well outlet path 177. The first segment 151 of the fluid path 150 leads from the blister pack 140 to the sample introduction area 120. The second segment 152 of the fluid path 150 leads from the sample introduction area 120 to the mixing chamber 153. The mixing chamber 153 is the third segment of the fluid path 150 and is widened relative to the second segment 152 and fourth segment 154. The fourth segment 154 of the fluid path 150 leads from the mixing chamber 153 to the fifth segment 155 of the fluid path. The fifth segment 155 of the fluid path 150 is formed in the test region 170A. The fifth segment 155 of the fluid path 150 leads into both the first test well inlet path 176 and into the sixth segments 156 of the fluid path 150. The sixth segments 156 of the fluid path 150 each form a continuation of the fluid path 150 between adjacent test well inlets until the last test well inlet 176. A test well inlet path 176 fluidically connects a test well 175 to the fluid path 150, and may closed off by a valve 174, for example to prevent cross-amplification between the test wells. A test well outlet path 177 leads from a test well 175 to an outlet aperture 178 that allows gas to escape from the test well 175 and out of the cartridge 100.

Even or homogenous mixing of the liquid from the blister pack 140 with the applied sample can yield more accurate test results in some embodiments. As such, the mixing chamber 153 is configured to promote even mixing of the liquid from the blister pack 140 with the applied sample, for example by including curved regions and/or a cross-sectional shape that promote turbulent flow rather than laminar flow of the liquids within the mixing chamber 153. Turbulent flow is a flow regime in fluid dynamics characterized by chaotic changes in pressure and flow velocity of a fluid. Turbulent flow is in contrast to laminar flow, which occurs when fluid flows in parallel layers, with no disruption between those layers.

The segments 151, 152, 153, 154 of the fluid path 150 can be entirely encased within the material of the base 125 or can have three surfaces formed from the material of the base 125 with the cover 105 forming an upper surface that seals these channels. The segments 155, 156 of the fluid path 150 and the test well inlet path 176 and test well outlet path 177 can be entirely encased within the material of the base 125, can have three surfaces formed from the material of the base 125 with the cover 105 forming an upper surface that seals these features, or can have two surfaces formed from the material of the base 125 with the circuit board 179 forming a lower surface of these features and the cover 105 forming an upper surface of these features.

FIG. 1C illustrates the direction of flow along the fluid path 150 with encircled numbers shown as labels for certain points along the fluid path. The encircled numbers are discussed below as example steps of a progression of fluid 180 as it travels through the fluid path 150 within the cartridge 100, with each step including a directional arrow showing the direction of fluid travel at that step.

Prior to step (1), a user applies a sample at the sample introduction area 120. For clarity and simplicity of FIG. 1C, the components labeled with reference numbers in FIG. 1B are not labeled in FIG. 1C. Also prior to step (1), the blister pack 140 is ruptured so that its liquid contents are released from its previously sealed chamber.

At step (1), air or other fluid flowing from the pneumatic interface 160 travels in the illustrated direction along pneumatic fluid path 161 towards the ruptured blister pack 140.

At step (2), the liquid released from the ruptured blister pack 140 (referred to herein as a “master mix”) travels through the aperture 141 in the illustrated direction and into the first segment 151 of the fluid path 150. The master mix continues flowing along the first segment 151 until step (3), when it enters the sample introduction area 120 and begins to carry the sample with itself further along the fluid path.

At step (4), the master mix and sample leave the sample introduction area 120 and flow along the second segment 152 of the fluid path 150 in the illustrated direction. The volume of the master mix can be pre-selected to completely or substantially completely flush the applied sample from the sample introduction area 120 and/or to at least fill the test wells 175 and their respective inlet paths 176.

At step (5), the master mix and sample flow in the illustrated direction into the entrance to the wider third segment 153 of the fluid path 150, and at step (6) the master mix and sample are mixed into a homogenous solution in which the sample is evenly distributed throughout the master mix. As described above, the third segment 153 includes curved segments and a planar mixing chamber configured to promote mixing of the master mix and the sample. The rate of fluid provided by the pneumatic interface 160 can be selected to further facilitate this mixing in some embodiments.

At step (7), the mixed master mix and sample (referred to as the “test fluid”) leave the mixing chamber 153 and enter the fourth segment 154 of the fluid path 150 that leads into the test region 170A.

At step (8), the test fluid travels along the fifth segment 155 of the fluid path 150 in the illustrated direction through the test region 170A towards the test wells 175.

At step (9), the test fluid reaches the first test well inlet path 176 and its flow is directed along the three possible paths shown trifurcating from the arrow of the fluid path of step (9).

The path of step (10) shows the flow of the test fluid further along the segment 156 of the fluid path 150 to subsequent test well inlet paths 176. Optionally, the valve 174 at the test well inlet path 176 may be closed, preventing the flow of the test fluid to step (10).

The path of step (11) shows the optional flow of a gas portion of the test fluid through the valve 174. In some embodiments, the valve 174 can include a liquid impermeable, gas permeable filter to allow any gas present in the test fluid to vent through the valve 174 prior to entering the test well 175. In some embodiments the valve 174 may not be configured to vent gas.

The path of step (12) shows the direction of the flow of the test fluid into the test well 175. In some embodiments, the valve 174 can be closed to seal off the test well 175 upon occurrence of a predetermined trigger. The trigger can occur after a predetermined volume of liquid corresponding to the volume of at least the test well 175 (and additionally the inlet and outlet paths 176, 177) has flowed along the path of step (12). Another example of the valve closing trigger can occur after a predetermined amount of time has elapsed corresponding to the time expected for this volume of liquid to flow along the path of step (12). In another embodiment, the trigger can be the deactivation of the pneumatic interface 160, at which point fluid may begin to flow backward along the illustrated paths, causing cross-contamination of the amplification processes occurring in different test wells. In some embodiments, the depicted location of the valve 174 may instead be a gas outlet aperture optionally covered with a liquid impermeable, gas permeable filter, and the described valve can be located along the test well inlet path 176 or along the fluid path segment 156.

The path of step (13) shows the direction of the flow of the test fluid or a gas component thereof out of the test well 175 through the outlet path 177. The outlet path 177 can be a channel leading out of the test well 175, and the test fluid can be pushed into the outlet path 177 by the pressure provided by the pneumatic interface 160. In some embodiments, a liquid impermeable, gas permeable filter can be provided at the interface of the test well 175 and the outlet path 177 so that only a gas component of the test fluid flows through the outlet path 177.

At step (14), gas from the test fluid is vented from the cartridge 100 through the outlet aperture 178. Outlet aperture 178 can be covered by a liquid impermeable, gas permeable filter to allow gas to escape and prevent liquid from escaping the cartridge 100. Beneficially, allowing and facilitating the venting of gas from the test fluid can minimize the amount of gas that remains in the test well, maximizing the amount of liquid in the test well. As described below, minimizing the potential for gas bubbles to form in the path between electrodes can beneficially lead to more reliable signals and more accurate test results.

Returning to FIG. 1B, the test region 170A includes the segments 155, 156 of the fluid path 150, the test wells 175, the test well inlet paths 176, the test well outlet paths 177, the apertures/valves 176, 178, and a circuit board 179. The circuit board 179 includes the electrodes 171A, 171B of the test wells, the conductors 172 for carrying current or other electric signals, and the electrode interface 135. The electrode interface 135 includes contact pads 173; half of the contact pads 173 are configured for coupling an excitation electrode of a test well with a voltage or current source of a reader device and the other half of the contact pads 173 are configured for electrically coupling a signal electrode of the test well with a signal reading conductor of the test device. For clarity of FIG. 1B, only certain ones of the repeated features of the test region 170A are labeled with reference numbers.

The circuit board 179 can be a printed circuit board, for example a screen-printed or silkscreen printed circuit board having multiple layers. The circuit board 179 can be a printed onto a flexible plastic substrate or semiconductor substrate. The circuit board 179 can be formed at least partly from a separate material from the base 125 and secured to the underside of the base 125, with an overlying region 126 of the base 125 including the segments 155, 156 of the fluid path 150, the test wells 175, the test well inlets 176, the test well outlets 178, and the apertures/valves 176, 178. For example, the circuit board 179 can be a multilayered printed circuit board adhered, affixed, or laminated to the acrylic of the overlying region 126. The electrode interface 135 can extend beyond the edge of the overlying region 126. The test wells 175 can be formed as openings in the material of the overlying region 126 such that the electrodes 171A, 171B of the circuit board 179 are exposed within a well 175. As such, the electrodes 171A, 171B can be in direct contact with fluid that flows into the well 175. The circuit board 179 can be butter coated by having a resin on its upper surface in order to create a smooth, flat surface for the bottoms of the test wells.

The test wells 175 can be provided with solid dried constituents for the testing process, for example primers and proteins. The particular selection and chemistry of these dried constituents can be tailored to a particular target or targets for which the cartridge 100 is designed to test. The test wells 175 can be provided with the same or different dried constituents. These dried constituents can be hydrated with the liquid that flows into the test well (e.g., the liquid from the blister pack 140 mixed with the applied sample) and thus activated for the test procedure. Beneficially, providing the liquid constituents in the blister pack 140 separately from the dried solid constituents in the test wells 175 enables the cartridge 100 to be stored before use containing the components needed for the amplification process, while also delaying initiation of amplification until after the sample has been applied.

The test wells 175 are depicted as circular wells arranged in two rows at staggered distances from the electrode interface 135. The test wells 175 can be generally cylindrical, for example formed as circular openings in the material of the overlying region 126 and bounded by planar surfaces at their upper (e.g., cover 105 or a portion of the overlying region 126) and lower (e.g., circuit board 179) sides. Each test well 175 contains two electrodes 171A, 171B, with one electrode being an excitation electrode configured to apply current to the sample in the test well 175 and the other electrode being a signal electrode configured to detect current flowing from the excitation electrode through the liquid sample. In some embodiments, one or more test wells can be provided with a thermistor in place of the electrodes in order to provide for monitoring of the temperature of the fluid within the cartridge 100.

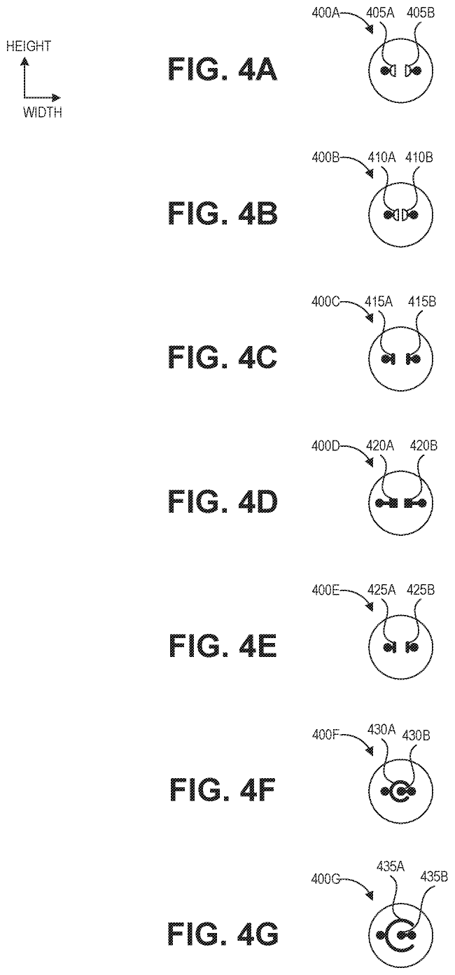

Each test well can be monitored independently of the other test wells, and thus each test well can constitute a different test. The depicted electrodes 171A, 171B within each test well are linear electrodes positioned parallel to one another. The depicted arrangement of the test wells 175 provides a compact test region 170A with access from the fluid path 150 to each test well 175. Some embodiments can include only a single test well, and various embodiments can include two or more test wells arranged in other configurations. Further, the shape of the test wells can be varied in other embodiments, and the electrode shapes can be any of the electrodes shown in FIGS. 4A-4G.

In some embodiments, gas bubbles within a test well 175, particularly if positioned along the current path between the electrodes 171A, 171B, can create noise in the signal picked up by the signal electrode. This noise can reduce the accuracy of test results determined based on the signal from the signal electrode. A desired high-quality signal may be obtained when only liquid is present along the current path or when minimal gas bubbles are present along the current path. As described above, any air initially present in the fluid flowing along the fluid path 150 can be pushed out through the outlet aperture 178. In addition, the electrodes 171A, 171B and/or test well 175 can be shaped to mitigate or prevent nucleation of the liquid sample in which air or gas bubbles form in the liquid sample and collect along the electrodes.

For example, the electrodes 171A, 171B are positioned at the bottom of the test well 175. This can allow any air or gas to rise to the top of the fluid in the test well and away from the path between the electrodes. As used herein, the bottom of the test well refers to the portion of the test well in which heavier liquid settles due to gravity, and the top of the test well refers to the portion of the test well in which lighter gas rises above the heavier liquids. Further, the electrodes 171A, 171B are positioned away from the perimeter or edges of the test well 175 which is a location at which bubble nucleation typically occurs.

Further, the electrodes 171A, 171B can be formed from a thin, flat layer of material that has minimal height relative to the underlying circuit board layer that forms the bottom of the test well. In some embodiments, the electrodes 171A, 171B can be formed using electrodeposition and patterning to form a thin layer of metal film, for example around 300 nm in height. This minimal height can help prevent or mitigate air bubbles from becoming trapped along the interface between the electrode and the underlying layer. In some embodiments, a layer of conductive material can be deposited on top of each electrodes to create a smoother transition between the edge of the electrode and the bottom of the test well. For example, a thin polyimide layer (e.g., around 5 microns in height) can be deposited on top of the electrode or the circuit board can be butter coated. Additionally or alternatively, the electrodes can be positioned in grooves in the underlying layer with the grooves having a depth approximately equal to the height of the electrode. These and other suitable methods can achieve an electrode that is approximately flat or flush with the bottom surface of the well.

Beneficially, the above-described features can help to keep the electrodes 171A, 171B surrounded by liquid and prevent or reduce gas bubbles from becoming positioned along the current path between the electrodes 171A, 171B.

FIG. 1D is a line drawing depicting a top plan view of test region 170B of the cartridge 100. As with FIG. 1B, certain repeated features are labeled with reference numbers in only one location for simplicity and clarity of the drawing of FIG. 1D.

The test region 170B is an alternate embodiment of the test region 170A, with the difference between the two embodiments being a different electrode configuration within the test wells 175. In the embodiment of the test region 170B, the test wells are provided with annular electrodes 171C and 171D. With the linear electrodes 171A, 171B of the test region 170A, either electrode can be the excitation electrode or the signal electrode. In the embodiment of the test region 170B, the inner electrode 171D is the excitation electrode and the outer electrode 171C is the signal electrode.

The inner electrode 171D can be a disc or circular-shaped electrode coupled to the current providing conductor 172B, which is in turn coupled to a current providing pad 173 of the electrode interface 135 that transmits current (e.g., AC current at a specified frequency) to the inner electrode 171D from a reader device. The inner electrode 171D can be positioned in the center of the test well 175. The outer electrode 171C is a semicircular electrode formed concentrically around the inner electrode 171D and separated from the inner electrode 171D by a gap. A break in the semicircle of the outer electrode 171C occurs where a conductive lead connects the inner electrode 171D to the current providing conductor 172B. The outer electrode 171C is coupled to the current sensing conductor 172B, which is in turn coupled to a current sensing pad 173 of the electrode interface 135 that transmits the sensed current to the reader device.

The cartridge 100 of FIGS. 1A-1D provides a self-contained, easy to use device for performing an amplification-based test for a target, for example nucleic acid testing wherein genomic material in the sample is exponentially copied using a molecular amplification process. Beneficially, the user only needs to apply the sample and insert the cartridge 100 into a reader device in order to ascertain the result of the test in some embodiments, as the liquid and solid constituents of the amplification process are pre-provided within the cartridge and automatically mixed with the sample. In some embodiments, one or both of the cartridge or reader may include a heater and a controller configured to operate the heater to maintain the cartridge at the desired temperature for amplification. In some embodiments, one or both of the cartridge or reader may include a motor to impart vibrations to or otherwise agitate the cartridge to cause any trapped gas to rise to the top of the liquid and vent from the test wells.

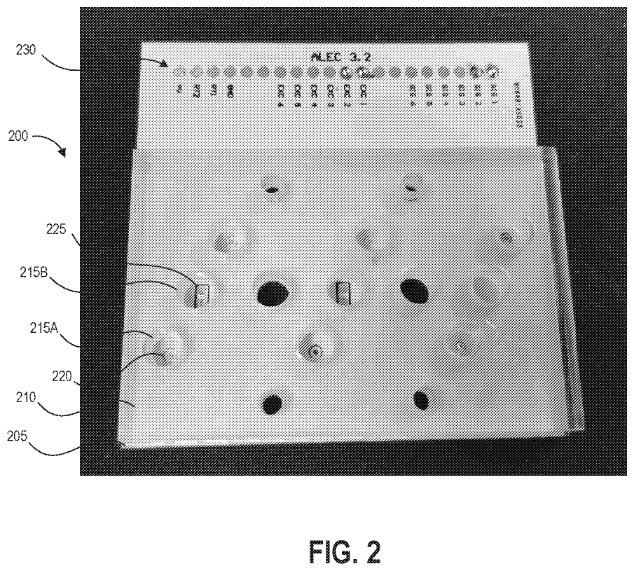

FIG. 2 depicts a photograph of another example cartridge 200 configured for detection of a target. The cartridge 200 was used to generate some of the test data described herein and represents an alternate configuration of some of the components described with respect to the cartridge 100.

Cartridge 200 includes a printed circuit board layer 205 and an acrylic layer 210 overlying and adhered to a portion of the printed circuit board layer 205 using a pressure-sensitive adhesive. The acrylic layer 210 includes a plurality of test wells 215A and a plurality of temperature monitoring wells 215B formed as circular apertures extending through the height of the acrylic layer 210. The printed circuit board layer 205 can be formed similarly to the circuit board 179 described above and includes a pair of electrodes 220 positioned within each test well 215A and a thermistor 225 positioned within each temperature monitoring well 215B. The electrodes 220 and thermistors 225 are each coupled to conductors terminating at a number of leads 230 of the printed circuit board. As illustrated, six of the leads are labeled “SIG” followed by a number 1-6 for the signal electrodes, six of the leads are labeled “EXC” followed by a number 1-6 for the excitation electrodes, and two leads are labeled RT1 and RT2 for the thermistors.

During some of the tests described herein, the following example protocol was followed. First, the user filled the wells 215A with a test fluid and capped the fluid with mineral oil. The test fluid can have no primer control, allowing for a definitive negative control as there is no primer to cause amplification.

Next, the user heated the cartridge 200 to 65 degrees Celsius for ten minutes to expand any trapped air in the test fluid and cause it to rise as bubbles to the top of the liquid. During this initial heating, bubbles formed in the wells 215A.

At the next step, the user scraped the bubbles from the surface of the liquid in the wells 215A using a pipette or other tool. As described above, elimination of air bubbles can promote more accurate test results.

After eliminating the bubbles, the user allowed the cartridge 200 to cool to room temperature. Next, the user injected loop mediated isothermal amplification (LAMP) positive control (PC) into the bottom of each of the test wells 215A, placed the cartridge 200 on a heat block, and began performing the LAMP tests. The signals detected from the signal electrodes were analyzed as described herein to identify a positive signal cliff.

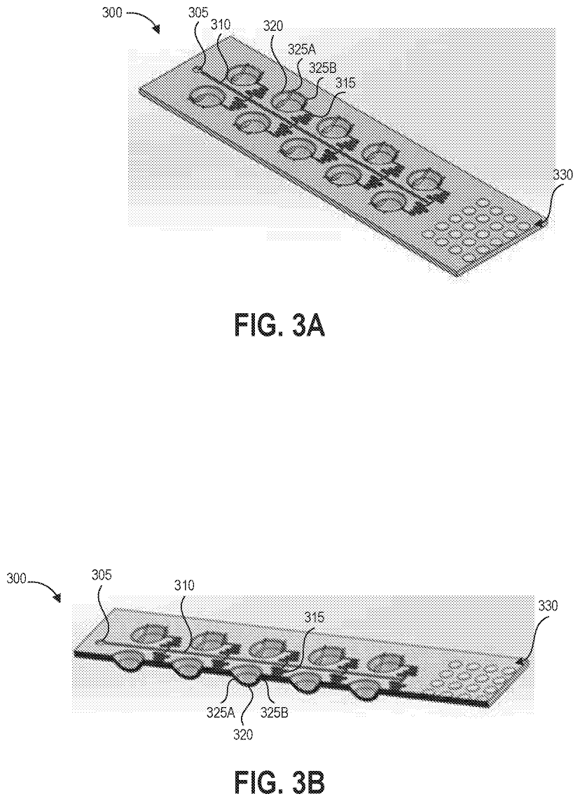

FIGS. 3A and 3B depict another example cartridge 300 configured for detection of a target. FIG. 3A depicts a top, front, and left perspective view of the cartridge 300 and FIG. 3B depicts a perspective cutaway view showing the contour of the wells 320 of the cartridge 300. The cartridge 300 represents an alternate configuration of some of the components described with respect to the cartridge 100.

The cartridge 300 includes sample introduction area 305, central channel 310, test wells 320, branches 315 fluidically connecting the test wells 320 to the central channel 310, electrodes 325A, 325B positioned within each test well 320, and an electrode interface 320 including contact pads coupled to conductors that are in turn coupled to respective ones of the electrodes 325A, 325B and configured to receive or send signals from or to a reader device. As shown in FIG. 3B, the wells 320 can have a curved bottom surface such that each well is generally hemispherical. The cartridge 300 is depicted as having an open top for purposes of revealing its interior components, however in use a cover or other upper layer can be provided to seal the fluid pathways of the cartridge 300. The cover can include vents to allow gas to escape from the cartridge 300, for example provided with liquid impermeable gas permeable filters, as described above with respect to FIGS. 1A-1D.

The fluid sample applied at the sample introduction area 305 flows down the central channel 310, for example in response to pressure from a reader device injecting the sample into the cartridge 300 through a port coupled above the sample introduction area 305. Such a reader device can be provided with a set of cartridges in some embodiments, for example positioned in a stack, and can provide the same or different sample to each cartridge. The fluid sample can be predominantly liquid with dissolved or trapped gas (e.g., air bubbles). The fluid can flow from the central channel 310 through the branched channels 315 into the test wells 320. The branched channels 315 can inlet into the top of the well and can be tortuous (e.g., including a number of turns having small radii) in order to prevent or mitigate backflow of fluid that could lead to cross-contamination of the amplification processes between the various wells.

FIGS. 4A-4G depict various examples of electrode configurations that can be used in a test well of the cartridges of FIG. 1A-3B or 51A-53B, or in the test well or channel of another suitable target detection cartridge as described herein. The test wells shown in FIGS. 4A-4G are depicted as circular, however the electrodes can be used in test wells of other geometries in other examples. Unless otherwise noted, the solid circles in FIGS. 4A-4G represent contacts between the disclosed electrodes and conductors leading to or from the electrode. “Width” as used below refers to a dimension along the horizontal direction of the page of FIGS. 4A-4G, and “height” as used below refers to a dimension along the vertical direction of the page of FIGS. 4A-4G. Though depicted in a particular orientation, the illustrated electrodes of FIGS. 4A-4G can be rotated in other implementations. Further, the disclosed example dimensions represent certain potential implementations of the electrode configurations 400A-400G, and variations can have different dimensions that follow the same ratios between the provided example dimensions. The electrodes shown in FIGS. 4A-4G can be made from suitable materials including platinum, gold, steel, or tin. In experimental testing, tin and platinum performed similarly and suitable for certain test setups and test targets.

FIG. 4A depicts a first electrode configuration 400A wherein the first and second electrodes 405A, 405B are each formed as a semicircular perimeter. The straight edge of the first electrode 405A is positioned adjacent to the straight edge of the second electrode 405B and separated by a gap along the width of the configuration 400A. The gap is larger than the radius of the semicircle of the electrodes. Thus, the first and second electrodes 405A, 405B are positioned as mirrored semicircular perimeters. In one example of the first electrode configuration 400A, the gap between the closest portions of the first and second electrodes 405A, 405B spans approximately 26.369 mm, the height (along the straight edge) of each of the electrodes 405A, 405B is approximately 25.399 mm, and the radius of the semicircle of each of the electrodes 405A, 405B is approximately 12.703 mm.

FIG. 4B depicts a second electrode configuration 400B. Similar to the first electrode configuration 400A, the first and second electrodes 410A, 410B of the second electrode configuration 400B are each formed as a semicircular perimeter and are positioned as mirrored semicircles with their straight edges facing one another. The first and second electrodes 410A, 410B of the second electrode configuration 400B can be the same size as the first and second electrodes 405A, 405B of the first configuration 400A. In the second electrode configuration 400B, the gap along the width of the configuration 400B between the first and second electrodes 410A, 410B is smaller than in the first configuration 400A, and the gap is smaller than the radius of the semicircle of the electrodes 410A, 410B. In one example of the second electrode configuration 400B, the gap between the closest portions of the first and second electrodes 410A, 410B spans approximately 10.158 mm, the height (along the straight edge) of each of the electrodes 410A, 410B is approximately 25.399 mm, and the radius of the semicircle of each of the electrodes 410A, 410B is approximately 12.703 mm.

FIG. 4C depicts a third electrode configuration 400C having first and second linear electrodes 415A, 415B separated by a gap along the width of the configuration 400C, where the gap is approximately equal to the height of the electrodes 415A, 415B. The width of the electrodes 415A, 415B is approximately one half to one third of the height of the electrodes. In one example of the third electrode configuration 400C, the gap between the closest portions of the first and second electrodes 415A, 415B spans approximately 25.399 mm, the height of each of the electrodes 415A, 415B is also approximately 25.399 mm, and the width of each of the electrodes 415A, 415B is approximately 10.158 mm. The ends of the first and second electrodes 415A, 415B can be radiused, for example having a radius of around 5.078 mm.

FIG. 4D depicts a fourth electrode configuration 400D having first and second rectangular electrodes 420A, 420B separated by a gap along the width of the configuration 400D, where the gap is approximately equal to the width of the electrodes 420A, 420B. In one example of the fourth electrode configuration 400D, the gap between the closest portions of the first and second electrodes 420A, 420B spans approximately 20.325 mm, the height of each of the electrodes 420A, 420B is also approximately 23.496 mm, and the width of each of the electrodes 420A, 420B is approximately 17.777 mm.

FIG. 4E depicts a fifth electrode configuration 400E having first and second linear electrodes 425A, 425B separated by a gap along the width of the configuration 400E, where the gap is approximately equal to the height of the electrodes 425A, 425B. The fifth electrode configuration 400E is similar to the third electrode configuration 400C, with the width of the electrodes 425A, 425B reduced to around one half to two thirds of the width of the electrodes 415A, 415B while having the same height. In one example of the fifth electrode configuration 400E, the gap between the closest portions of the first and second electrodes 425A, 425B spans approximately 25.399 mm, the height of each of the electrodes 425A, 425B is also approximately 25.399 mm, and the width of each of the electrodes 425A, 425B is approximately 5.078 mm. The ends of the first and second electrodes 425A, 425B can be radiused, for example having a radius of around 2.542 mm.

FIG. 4F depicts a sixth electrode configuration 400F having concentric annular electrodes 430A, 430B. The sixth electrode configuration 400F is the configuration shown in the test wells 175 of FIG. 1D. The inner electrode 430B can be a disc or circular-shaped electrode and can be positioned in the center of the test well. The outer electrode 430A can be a semicircular electrode formed concentrically around the inner electrode 430B and separated from the inner electrode 430B by a gap. In the sixth electrode configuration 400F, the gap is approximately equal to the radius of the inner electrode 430B. A break in the semicircle of the outer electrode 430A occurs where a conductive lead connects the inner electrode 430B to the current providing conductor. In one example of the sixth electrode configuration 400F, the gap between the inner edge of the annular first electrode 430A and the outer perimeter of the circular second electrode 430B spans approximately 11.430 mm, the radius of the circular second electrode 430B is approximately 17.777 mm, and the thickness of the annulus of the annular first electrode 430A is approximately 5.080 mm. The ends of the first electrode 430A can be radiused, for example having a radius of around 2.555 mm, and the gap between the open ends of the annulus of the first electrode 435A can be around 28.886 mm from vertex to vertex.

FIG. 4G depicts a seventh electrode configuration 400G having concentric annular electrodes 435A, 435B. Similar to the embodiment of FIG. 4F, the inner electrode 435B can be a disc or circular-shaped electrode having the same radius as inner electrode 430B and can be positioned in the center of the test well. The outer electrode 435A can be a semicircular electrode formed concentrically around the inner electrode 435A and separated from the inner electrode 435A by a gap. In the seventh electrode configuration 400G, the gap is greater than the radius of the inner electrode 435B, for example two to three times greater. Correspondingly, the outer electrode 435B has a larger radius than the outer electrode 430B. In one example of the seventh electrode configuration 400G, the gap between the inner edge of the annular first electrode 435A and the outer perimeter of the circular second electrode 435B spans approximately 24.131 mm, the radius of the circular second electrode 435B is approximately 17.777 mm, and the thickness of the annulus of the annular first electrode 435A is approximately 5.080 mm. The ends of the first electrode 435A can be radiused, for example having a radius of around 2.555 mm, and the gap between the open ends of the annulus of the first electrode 435A can be around 46.846 mm from vertex to vertex.

In the embodiments of FIGS. 4A-4E, either electrode can be used as the excitation electrode and the other electrode can be used as the signal electrode. In the embodiments of FIGS. 4F and 4G, the inner electrode 430B, 435B is configured to be used as the excitation electrode (e.g., coupled to a current source) and the outer electrode 430A, 435A is configured to be used as the signal electrode (e.g., provides its signal to a memory or processor). In some example tests, the sixth electrode configuration 400F exhibited the best performance of the configurations shown in FIGS. 4A-4G.

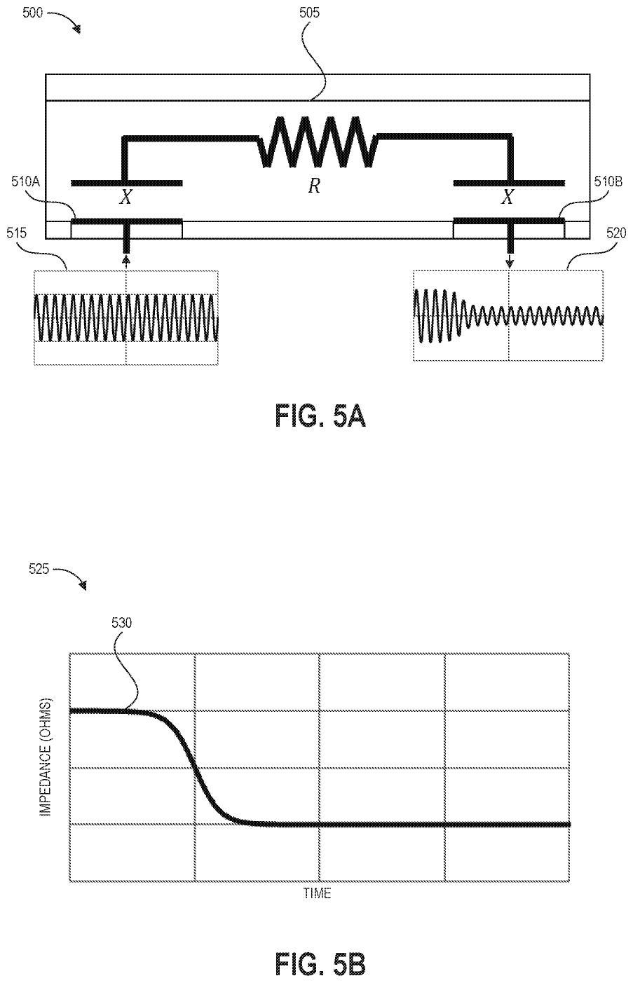

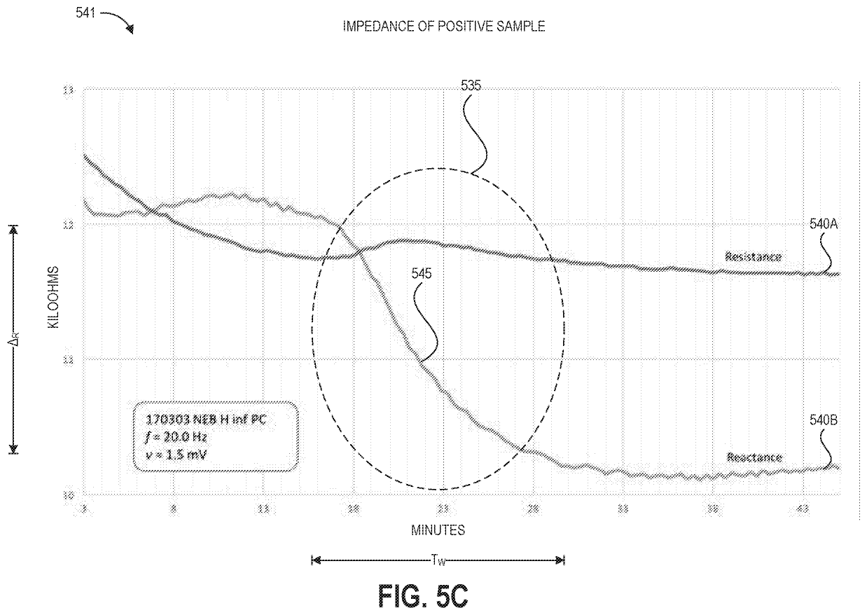

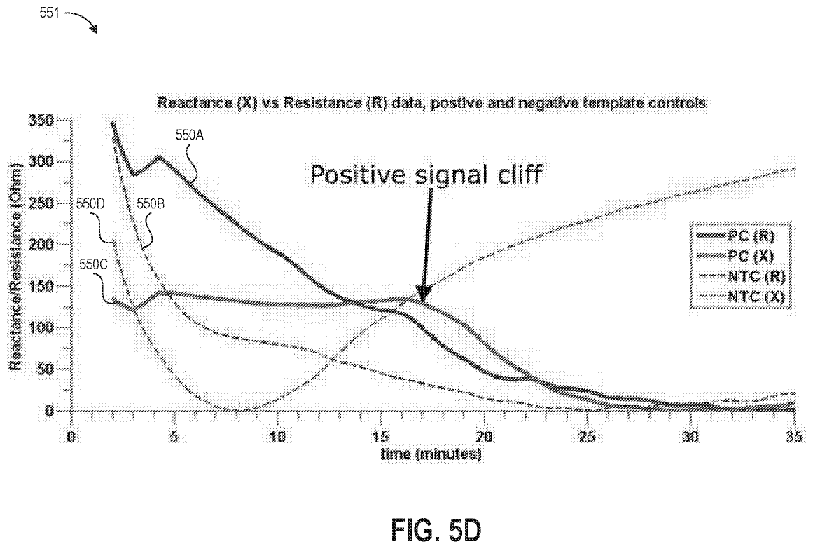

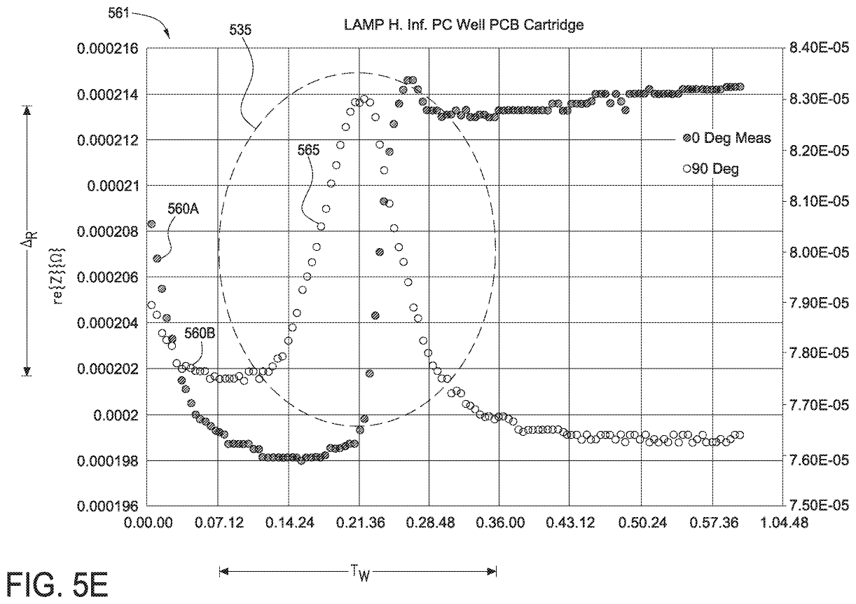

FIG. 5A depicts a first electrode or excitation electrode and a second electrode or signal electrode that may be spaced apart from one another within a test well of the cartridges of FIG. 1A-3B or 51A-53B, or in the test well or channel of another suitable target detection cartridge as described herein.

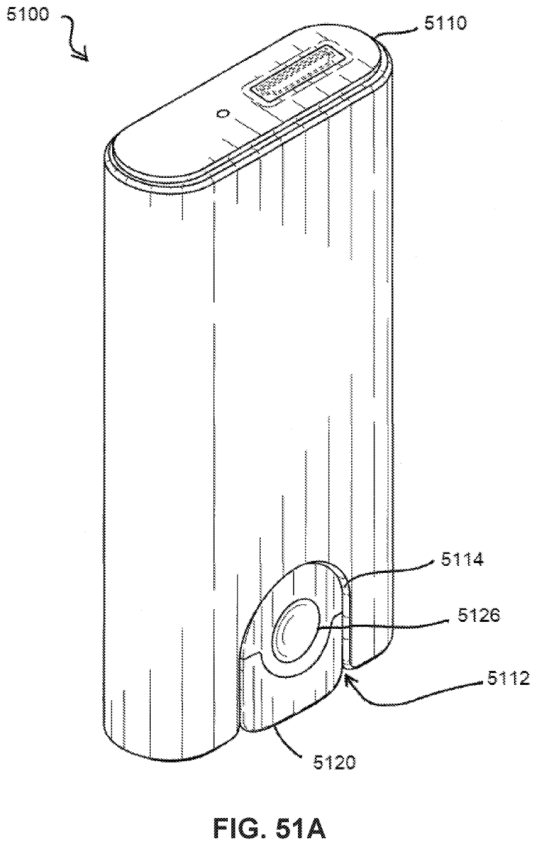

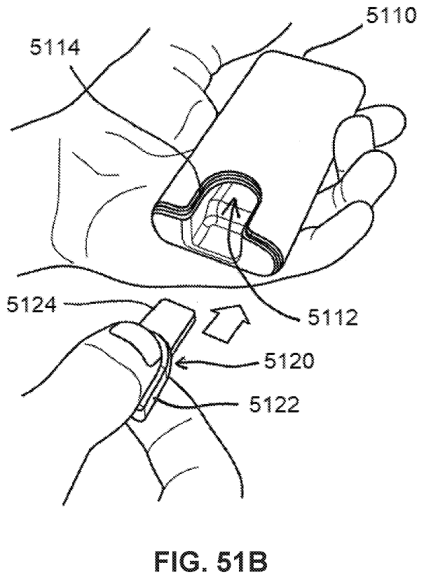

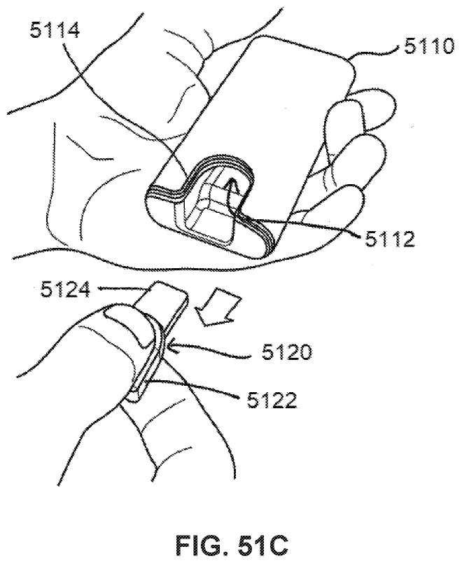

The formation of an aggregate, nucleic acid complex, or polymer, for example during an amplification process in the test wells of cartridges of FIGS. 1A-3B or of FIGS. 51A-53B, can affect waveform characteristics of one or more electrical signals that are sent through a channel. As shown in FIG. 5A, a first electrode or excitation electrode 510A is spaced apart from a second electrode or sensing electrode 510B within test well 505. The test well 505 can contain a test solution undergoing an amplification process. During some of all of that process, an excitation voltage 515 can be provided to the excitation electrode 510A, from which the excitation voltage 515 is transmitted into the fluid (preferably all or substantially all liquid) within the well 505.

After passage through and attenuation by the liquid sample (represented schematically by the resistance R and reactance X), the attenuated excitation voltage is sensed or detected at the sensing electrode 510B. The fluid acts as a resistor R in series with the excitation electrode 510A and the sensing electrode 510B. The fluid also acts as in series capacitor(s), shown by the reactance X. The raw sensed signal during some or all of the duration of a test can be represented over time as a sinusoidal curve with varying amplitudes, similar to that shown in plot 520.

The excitation voltage 515 can be an alternating current at a predetermined drive frequency. The particular frequency selected can depend for example upon the particular target sought to be detected, the medium of the test sample, the chemical makeup of the amplification process constituents, the temperature of the amplification process, and/or the excitation voltage. In some embodiments of the cartridges of FIGS. 1A-3B or of FIGS. 51A-53B, the excitation drive frequency can be between 1 kHz and 10 kHz at as low an excitation voltage as possible. As one example, in tests performed to identify a target of H. Influenzae (10⁶ copies/reaction) spiked into 5% whole blood, excitation sensor drive frequency was varied from 100 Hz to 100,000 Hz at 0.15 Volts. These tests revealed that the desired “signal cliff,” an artifact in a portion of the signal indicative of a positive test sample described in more detail below, becomes more easily detectable below 100 Hz and is most easily detectable between 1 kHz and 10 kHz. Further, with frequencies in the range between 1 kHz and 10 kHz, the signal cliff advantageously could be identified before 12 minutes of test time had elapsed. Beneficially, faster identification of the signal cliff can result in shorter test times, in turn resulting in quicker provision of test results and the ability to perform more tests per day. At frequencies lower than 1 kHz, the reactance component of the signal (in which the signal cliff may be found in a positive sample) decreased monotonically. The sensor drive frequency can be similarly fine-tuned for other tests to optimize performance, that is, to optimize the detectability of a signal cliff. Detectability of a signal cliff refers to the ability to consistently differentiate between a positive sample and a negative sample.

FIG. 5B depicts an example plot 525 showing an impedance signal 530 that can be extracted from the raw signal 520 provided by the sensing electrode 510B. The impedance signal 530 represents the electrical impedance Z of the test well over time. The impedance Z can be represented by a Cartesian complex number equation as follows:

Z=R+jX

where R represents the resistance of the test well and is the real part of the above equation and the X represents the reactance of the test well and is the imaginary part of the above equation (denoted by j). Thus, the impedance of the test well can be parsed into two components, the resistance R and the reactance X.

Initially, the value of the resistance R can be determined by taking a baseline measurement of the test well prior to or at the outset of the amplification process. Although the resistance of the test fluid can drift away from this baseline value throughout the duration of the test, the current sensed by the sensing electrode 510B due to the resistance of the test fluid can be in phase with the signal provided through the excitation electrode 510A. Thus, changes or drift in the resistance can be identified by values of the in phase component of the signal 520 over time. The reactance can arise from the effect of inductance in the test fluid, capacitance in the test fluid, or both; this effect can cause the fluid to retain current (e.g., electrons provided by excitation electrode 510A) temporarily. After some time this retained current flows out of the test fluid into the sensing electrode 510B. Due to this delay, the current sensed by the sensing electrode 510B due to the reactance of the test fluid can be out of phase with the current sensed from the resistance of the test fluid. Thus, values of the reactance of the test fluid can be identified by values of the out of phase component of the signal 520 over time. The reactance can fluctuate throughout the duration of the test based on changes to the chemical constituents of the test fluid due to the amplification process. The signal cliff (e.g., a rise or drop in the reactance at or greater than a threshold rate or magnitude and/or during a predetermined window of time) indicative of a positive sample can be found in the reactance X.

During a test, the excitation electrode 510A can be sinusoidally excited with some amplitude and voltage. The excitation electrode 510A is in series with the test liquid in the well, which can be considered as a resistor R. The resistor (e.g., the test fluid) and electrode form a voltage divider, which has a voltage determined by the ratio of the resistor and electrode chemistry/impedances. The resulting voltage waveform sensed at the sensing electrode 510B represents the complex impedance signal 530. In some embodiments, a curve such as the impedance signal 530 may not be generated, but rather the raw sensed signal 520 can be parsed into its resistance and reactance components as described herein. The impedance signal 530 is provided as an example representation of a combined curve representing both the resistance of the test fluid and the reactance of the test fluid over time. The complex impedance signal 530 can be interpreted as a quadrature-modulated waveform (e.g., a combination of an in-phase waveform resulting from the resistance of the test fluid and an out-of-phase waveform resulting from the reactance of the test fluid), where the in-phase and out-of-phase components change on a timescale much greater than the modulation frequency. The in-phase waveform is in-phase with the composite waveform of the complex impedance. Some implementations can use a synchronous detector, for example having multipliers and low pass filters implemented in a field programmable gate array (FPGA), to extract the in-phase and out-of-phase components from the raw signal 520 and compute their amplitude and phase.

In order to parse the impedance signal 530 (or the raw sensed signal 520) into its constituent resistance and reactance components, the voltage waveform 520 at the sensing electrode 510B is sampled faster than its Nyquist frequency (e.g., two times the highest frequency of the excitation voltage) and then decomposed into an in-phase component (resistance) and an out-of-phase component (reactance). The in-phase and out-of-phase voltage components can be computed using the known series resistance (e.g., the value of R) to calculate the real component of the impedance (the resistance) and the imaginary component of the impedance (the reactance).

FIG. 5C depicts a plot 541 of the resistance 540A and reactance components 540B over time (t=3 minutes to t=45 minutes) extracted from a raw signal 520 generated based on an example positive test. As illustrated, the signal cliff 545 represents a change Δ_{R} in the reactance 540B during a particular window of time T_{W}. The signal cliff 545 indicates a positive sample. At times occurring prior to the signal cliff 545, the reactance curve 540B is relatively flat or stable, and again after the signal cliff 545 the reactance curve 540B is relatively flat or stable. Thus, in this embodiment the signal cliff 545 for the particular test parameters represented by the plot 541 occurs as a drop of Δ_{R} in the expected region 535.

The magnitude of the change Δ_{R} in the reactance that corresponds to a positive sample signal cliff 545, as well as the position and/or duration of the particular window of time T_{W} at which the signal cliff 545 is expected to occur, can vary depending on a number of parameters of the test. These parameters include the particular target of the test (e.g., the rate at which that target amplifies), the frequency of the excitation voltage, the configuration of the excitation and sensor electrodes (e.g., their individual shapes and dimensions, the gap separating the electrodes, and the material of the electrodes), the sampling rate, the quantity of amplification agents provided at the start of the test, the temperature of the amplification process, and the amount of target present in the sample. In some embodiments, the expected characteristics of a signal cliff of a positive sample, predetermined for example through experimentation, can be used for differentiating between positive samples and negative samples. In some embodiments, the expected characteristics of a signal cliff can be used for determining the severity or progress of a medical condition, for example via correlations between particular signal cliff characteristics and particular initial quantities of the target in the sample. The predetermined expected characteristics can be provided to, stored by, and then accessed during test result determination by a reader device configured to receive signals from the sensing electrode(s) of a test cartridge.

For a given test, the expected magnitude of the change Δ_{R} in the reactance and the expected window of time T_{W} of a signal cliff 545 for a positive sample can be determined experimentally based on monitoring and analyzing the reactance curves generated by positive control samples (and optionally negative control samples). In some embodiments, the test parameters influencing the signal cliff can be varied and fine-tuned to identify the parameters that correspond to an accurately distinguishable signal cliff. A reader and cartridge as described herein can be configured to match the tested configuration and provided with expected signal cliff characteristics for that test.

For example, in a set of experimental tests for H. influenzae, the test fluid initially included amplification primers and 1,000,000 added target copies, the excitation voltage was 200 mV P2P, the test parameters included a 10 kHz sweep start and a 10 MHz sweep stop for the frequency of the excitation current, and close and far electrode gaps were configured at 2.55 mm and 5 mm respectively. The amplification temperature was set to 65.5 degrees Celsius, and the two electrode setups (one for each of the close and far gaps) included platinum electrodes. At low frequencies (10 kHz-100 kHz), detectable signal cliffs were identified beginning around 23 minutes into amplification around 10 kHz and around 30 minutes around 100 kHz using the 5 mm gap electrode configuration, with the magnitude of change in reactance being around 3.5-4 Ohms at 10 kHz and dropping to around 3.25-3.5 Ohms at 100 kHz. At low frequencies (10 kHz-100 kHz), detectable signal cliffs were identified beginning around 25 minutes into amplification around 10 kHz and around 30 minutes around 100 kHz using the 2.5 mm gap electrode configuration, with the magnitude of change in reactance being around 3.5-4 Ohms. At higher frequencies, the drop in reactance of the signal cliff decreased, and the time at which these smaller signal cliffs were identified was shifted to later in the amplification process. Accordingly, in this example a test well in a test cartridge may be configured with the 5 mm gap electrodes and a reader device may be configured to provide 10 kHz excitation current to the test cartridge during amplification. The reader device can be provided with instructions to provide this current and monitor the resulting reactance of the test well throughout amplification or for a window of time around the expected signal cliff time (here, 23 minutes), for example between 20 and 35 minutes. The reader device can also be provided with instructions to identify a positive sample based on the reactance exhibiting around a 3.5-4 Ohm change around 23 minutes into amplification, or within the window of time around the expected signal cliff time.

Once identified, the values for Δ_{R} and T_{W} can be provided to reader devices for use in distinguishing between positive and negative samples for that particular test. In some examples, such devices can determine whether the reactance curve 540B has the required value and/or slope at the identified window of time T_{W} to correspond to the signal cliff. In other embodiments, the reader device can analyze the shape of the reactance curve over time to determine whether it contains a signal cliff. In some embodiments, a reader can modify its testing procedures based on the identified window of time T_{W} at which the signal cliff 545 is expected to occur, for example by only providing the excitation voltage and monitoring the resultant signal within this window, advantageously conserving power and processing resources compared to continuous monitoring during an entire test time.

FIG. 5D depicts a plot 551 of the resistance and reactance components extracted from the raw sensor data of a sensing electrode 510B during example tests of positive and negative controls. Specifically, the plot 551 shows a curve 550A of the resistance of the positive sample, a curve 550B of the reactance of the positive sample, a curve 550C of the resistance of the positive sample, and a curve 550D of the reactance of the positive sample over the 35 minute duration of the test. As shown by FIG. 5D, the positive sample signal cliff occurs around 17 minutes into the test, with a relatively flat and stable reactance curve 550B leading up to the signal cliff. In contrast, at this same time the negative sample reactance curve 550D exhibits no signal cliff, but rather maintains a quadratic curvature from around t=8 minutes through the end of the test.

FIG. 5E depicts a plot 561 of the resistance 560A and reactance components 560B over time (t=0 minutes to t=60 minutes since the start of amplification) extracted from a raw signal 520 generated based on an example positive test. As illustrated, the signal cliff 565 represents a change Δ_{R} in the reactance 560B during a particular window of time T_{W}. The signal cliff 565 indicates a positive sample. At times occurring prior to the signal cliff 565, the reactance curve 560B is relatively flat or stable, and again after the signal cliff 565 the reactance curve 560B is relatively flat or stable with slight concavity. The signal cliff 565 for the particular test parameters represented by the plot 561 occurs as a peak, spike, or bell curve in the expected region 535, during which the reactance values rise and fall by the Δ_{R} value in an approximately parabolic curve. As described herein, varying of certain test parameters (e.g., test well configuration, chemistry and initial quantity of amplification constituents, target, and excitation current characteristics) can vary the geometry of the signal cliff yielded from a positive sample. Thus, in some embodiments the geometry of a “signal cliff” in the reactance values vs time curve can vary from test to test, though for a particular test the curve geometry and/or timing signal cliff remains consistent within reactance change and/or timing parameters across positive samples for that test. FIG. 6 depicts a schematic block diagram of an example reader device 600 that can be used with the cartridges described herein, for example the cartridges 100 or 300. The reader device 600 includes a memory 605, processor 610, communications module 615, user interface 620, heater 625, electrode interface 630, voltage source 635, compressed air storage 640, motor 650, and a cavity 660 into which a cartridge can be inserted.

When test cartridge 100 is inserted into the reader device, the electrode interface 135 of the cartridge couples with the electrode interface 630 of the reader device 600. This can allow the reader device 600 to detect that a cartridge is inserted, for example by testing whether a communication path is established. Further, such communications can enable the reader device 600 to identify a particular inserted test cartridge 100 and access corresponding testing protocols. Testing protocols can include the duration of the test, the temperature of the test, the characteristics of a positive sample impedance curve, and the information to output to the user based on various determined test results. In other embodiments, the reader device 600 can receive an indication via user interface 620 that a cartridge is inserted (e.g., by a user inputting a “begin testing” command and optionally a test cartridge identifier).

The memory 605 includes one or more physical electronic storage devices configured for storing computer-executable instructions for controlling operations of the reader device 600 and data generated during use of the reader device 600. For example, the memory 605 can receive and store data from sensing electrodes coupled to the electrode interface 630.

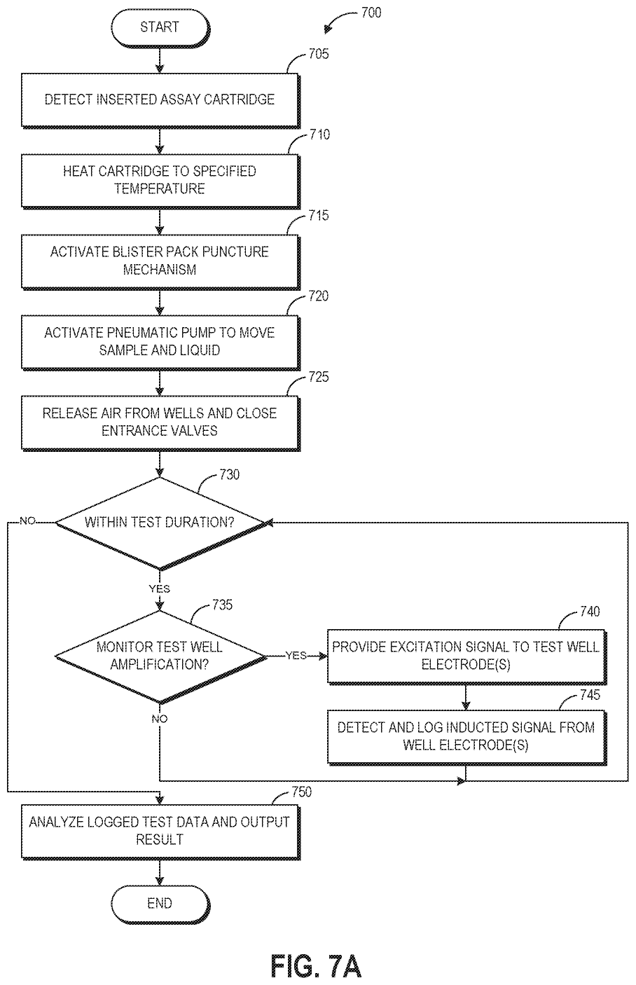

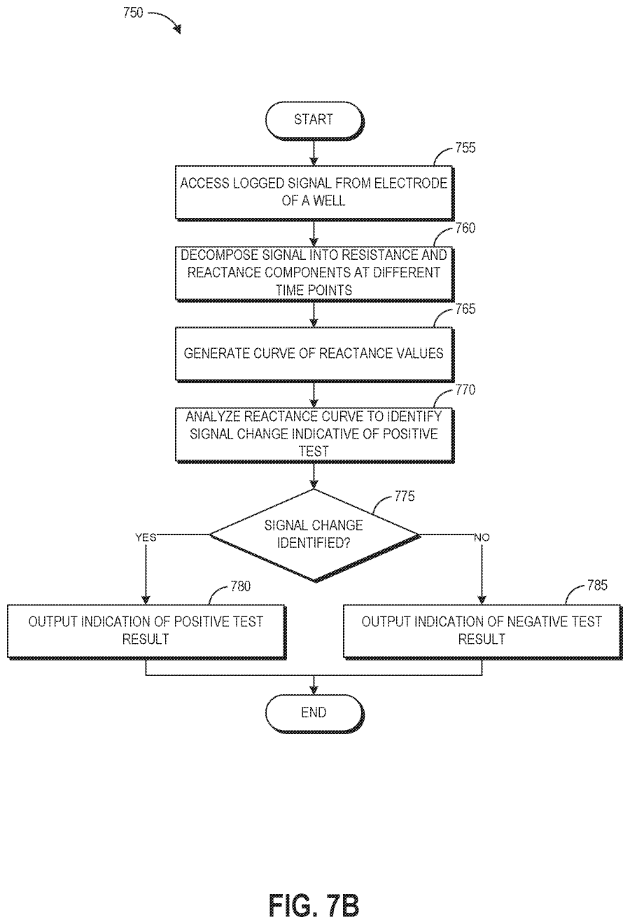

The processor 610 includes one or more hardware processors that execute the computer-executable instructions to control operations of the reader device 600 during a test, for example by managing the user interface 620, controlling the heater 625, controlling the communications module 615, and activating the voltage source 635, compressed air 640, and motor 650. One example of testing operations is described with respect to FIG. 7A below. The processor 610 can be also be configured by the instructions to determine test results based on data received from the excitation electrodes of an inserted test cartridge, for example by performing the process of FIG. 7B described below. The processor 610 can be configured to identify different targets in the same test sample based on signals received from different test wells of a single cartridge or can identify a single target based on individual or aggregate analysis of the signals from the different test wells.

The communications module 615 can optionally be provided in the reader device 600 and includes network-enabled hardware components, for example wired or wireless networking components, for providing networked communications between the reader device 600 and remote computing devices. Suitable networking components include WiFi, Bluetooth, cellular modems, Ethernet ports, USB ports, and the like. Beneficially, networking capabilities can enable the reader device 600 to send test results and other test data over a network to identified remote computing devices such as hospital information systems and/or laboratory information systems that store electronic medical records, national health agency databases, and the computing devices of clinicians or other designated personnel. For example, a doctor may receive the test results for a particular patient on their mobile device, laptop, or office desktop as the test results are determined by the reader device, enabling them to provide faster turnaround times for diagnosis and treatment plans. In addition, the networking capabilities can enable the reader device 600 to receive information over the network from remote computing devices, for example updated signal cliff parameters for existing test, new signal cliff parameters for new tests, and updated or new testing protocols.

The user interface 620 can include a display for presenting test results and other test information to users, as well as user input devices (e.g., buttons, a touch sensitive display) that allow the user to input commands or test data into the reader device 600.

The heater 625 can be positioned adjacent to the cavity 660 for heating an inserted cartridge to the desired temperature for an amplification process. Though depicted on a single side of the cavity 660, in some embodiments the heater 625 can surround the cavity.

As described herein, the voltage source 635 can provide an excitation signal at a predetermined voltage and frequency to each excitation electrode of an inserted test cartridge. The compressed air storage 640 can be used to provide pneumatic pressure via channel 645 to the pneumatic interface 160 of the test cartridge 100 to promote flow of the liquid within the test cartridge. Compressed air storage 640 can store previously compressed air or generate compressed air as needed by the reader device 600. Other suitable pneumatic pumps and pressure-providing mechanisms may be used in place of stored or generated compressed air in other embodiments. The motor 650 can be operated to move actuator 655 towards and away from the blister pack 140 of an inserted cartridge in order to rupture the blister pack as described above.

FIG. 7A depicts a flowchart of an example process 700 for operating a reader device during a test as described herein. The process 700 can be performed by the reader device 600 described above.

At block 705, the reader device 600 can detect that an assay cartridge 100, 200, 300 has been inserted, for example in response to user input or in response to establishing a signal path with the inserted cartridge. In some embodiments, the cartridge 100, 200, 300 can include an information element that identifies the particular test(s) to be performed to the reader device 600 and optionally includes test protocol information.

At block 710, the reader device 600 can heat the cartridge 100, 200, 300 to a specified temperature for amplification. For example, the temperature can be provided by information stored on the cartridge 100, 200, 300 or accessed in the internal memory of the reader device 600 in response to identification of the cartridge 100, 200, 300.

At block 715, the reader device 600 can active a blister pack puncture mechanism, for example motor 650 and actuator 655. Puncturing the blister pack can cause its liquid contents, including chemical constituents for facilitating amplification, to be released from its previously sealed chamber.

At block 720, the reader device 600 can activate a pneumatic pump to move the sample and liquid from the blister pack through a fluid path of the cartridge towards the test well. As described above, the test wells can include vents that enable the pushing of liquid through the fluid path of the cartridge and also allow any trapped air to escape. The pneumatic pump can include compressed air 640 or another suitable source of pressure and can fluidically communicate with the pneumatic interface 160.

At block 725, the reader device 600 can release any trapped air from the test wells, for example by pushing the fluid through the fluid path of the cartridge until a certain resistance is sensed (e.g., the liquid of the fluid path is pushed against the liquid impermeable, gas permeable filter of a vent). Block 725 may optionally include agitating the inserted cartridge to promote movement of any trapped air or gas bubbles up through the liquid and out through the vents. Further, at block 725 the reader device 600 optionally can provide signals to the cartridge that cause closure of valves positioned between test wells in order to avoid mixing of the amplification processes.

At decision block 730, the reader device 600 can determine whether the test is still within its specified test duration. For example, where the expected window of time in which a signal cliff should appear in a positive sample is known, the duration of the test may end at or some predetermined period of time after the end of the window. If so, the process 700 transitions to optional decision block 735 or, in embodiments omitting block 735, to block 740.

At optional decision block 735, the reader device 600 determines whether to monitor the test well amplification by logging data from the test well sensing electrode. For example, the reader 600 may be provided with instructions to only monitor the impedance of the test well during a particular window or windows of a test. If the reader device 600 determines not to monitor the test well amplification, the process 700 loops back to decision block 730.

If the reader device 600 determines to monitor the test well amplification, the process 700 transitions to block 740. At block 740, the reader device 600 provides an excitation signal to the excitation electrode of the test well(s) of the inserted cartridge. As described above, this can be an alternating current at a particular frequency and voltage.

At block 745, the reader device 600 detects and logs data from the sensing electrode of the test well(s) of the inserted cartridge. In some embodiments, this data can be stored for later analysis, for example after completion of the test. In some embodiments, the reader device 600 can analyze this data in real time (e.g., as the test is still occurring) and may stop the test once a positive sample signal cliff is identified.

When the reader device 600 determines at block 730 that the test is not still within its specified duration, the process 700 moves to block 750 to analyze the test data and output the test result. The test result can include an indication that the sample tested positive or negative for the target or can more specifically indicate an estimated quantity of the target in the tested sample.

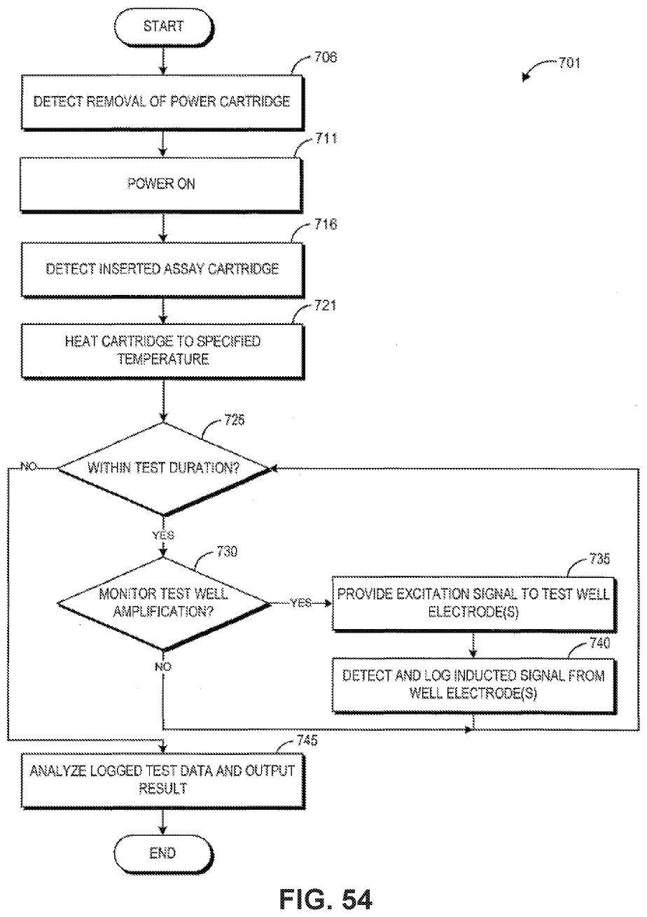

FIG. 7B depicts a flowchart of an example process for analyzing test data to detect a target as described herein that can be performed by the reader device 600 as block 750 of FIG. 7A or of FIG. 54.

At block 755, the reader device 600 can access logged signal data received from the electrode of a well. Even if a cartridge has multiple wells, the data from each well can be analyzed individually. The test results from the wells may later be analyzed in aggregate to determine a single test result for aa single target based on all tests performed within the cartridge, or to determine multiple test results for multiple targets.

At block 760, the reader device 600 can decompose the signal into resistance and reactance components across some or all of the different time points of the test. For example, as described above, at each time point the reader device 600 can determine in phase and out of phase components of the raw sampled voltage waveform and can then deconvolute these components using known series resistance of the electrode circuit to calculate the in-phase (resistance) and out-of-phase (reactance) portions of the impedance of the test well.

At block 765, the reader device 600 can generate a curve of the reactance values over time. Also at block 765, the reader device 600 can optionally generate a curve of the resistance values over time.

At block 770, the reader device 600 can analyze the reactance curve to identify a signal change indicative of a positive test. As described above with respect to the signal cliff of FIG. 5C, the reader device 600 can look for greater than a threshold change in reactance, can look for such a change within a predetermined window of time, can analyze the slope of the reactance curve at a predetermined time, or can analyze the overall shape of the reactance curve in order to determine whether a signal cliff (e.g., a rise or drop in the signal preceded and followed by relatively more stable values) is present.

At decision block 775, based on the analysis performed at block 770, the reader device 600 can determine whether the sought-after signal change was identified in the reactance curve. If so, the process 750 transitions to block 780 to output an indication of a positive test result to the user. If not, the process 750 transitions to block 785 to output an indication of a negative test result to the user. The result can be output locally, for example on the display of the device, or output over a network to a designated remote computing device.

### Overview of Additional Example Cartridges, Readers, and Signal Processing

The embodiments described in the section are exemplary and are not intended to limit the scope of the present disclosure. Some embodiments are directed to systems, methods, and/or devices for diagnostics for sensing and/or identifying pathogens, genomic materials, proteins, and/or other small molecules or biomarkers. In some implementations, a small, portable, low-power device provides rapid and robust sensing and identification. Such a device may utilize microfluidics, biochemistry, and electronics to detect one or more targets in the field and closer to or at the point of care.

FIGS. 51A-51C depict an example handheld detection system 5100 for detection of a target. The system 5100 includes a reader device 5110 and a cartridge 5120 configured to fit within a cavity 5112 of the reader device 5110. The cartridge 5120 generally includes an external section 5122 and an internal section 5124. When the cartridge 5120 is inserted within the reader device 5110, some or all of the internal section 5124 is contained within the reader device 5110. The external section 5122 is sized and shaped to be gripped by a user and may include one or more three-dimensional surface features such as an indentation 5126 to facilitate insertion and/or removal of the cartridge 5120 from the reader device 5110.

As shown in FIGS. 51B and 51C, the reader device 5110 and the cartridge 5120 are sized and shaped such that one or more interchangeable cartridges 5120 can be inserted and/or removed by hand at the cavity 5112. As will be described in greater detail, the reader device 5110 can include one or more heating components configured to heat at least a portion of the internal section 5124 of the cartridge 5120. The reader device 5110 can further include circuitry configured to connect with circuitry of the cartridge 5120 to detect one or more electrical properties of a sample contained within the cartridge.

In some embodiments, some of the cartridges 5120 can be power cartridges. The reader device 5110 can be powered on and powered off by a power cartridge 5120, instead of or in addition to a conventional power switch or button on the exterior of the reader device 5110. A power cartridge 5120 may have a size and shape similar to other cartridges for use with the reader device 5110. In operation, the power cartridge 5120 may be kept engaged within the cavity 5112 when the reader device 5110 is powered off. Circuitry of the power cartridge 5120 can be in contact with internal circuitry of the reader device 5110 such that removal of the power cartridge 5120 from the reader device 5110 causes the reader device 5110 to power on for testing. After completion of one or more tests, or at any other time when the reader device 5110 is to be powered off, the power cartridge 5120 is inserted into the cavity 5112. As the power cartridge 5120 is inserted, the circuitry of the power cartridge 5120 again comes into contact with the internal circuitry of the reader device 5110 such that insertion of the power cartridge 5120 causes the reader device 5110 to power off. Power cartridge applications are discussed in greater detail with reference to FIG. 6.

In some embodiments, one or more external status indicators can be provided on an exterior portion of the reader device 5110 to provide status indications to a user. For example, in one particular implementation the status indicator may include a light ring 5114 disposed about the cavity 5112. In other implementations, the optional status indicators may be located at any suitable location on the reader device 5110. The light ring 5114 or other status indicator may include one or more light sources, such as light emitting diodes (LEDs) or the like. The light ring may also be configured to indicate e.g., when the device is in use or not in use, or when different stages of the detection method using the device have been reached, completed, or are being performed, such as sample being received by the device or in the well(s), amplification being performed, detection of aggregates in the well(s), or transmission of the results to a receiver. Different colored lights can be used to indicate different stages of the detection method using the device such as those mentioned above.

In some embodiments, a plurality of differently colored LEDs may be provided within the light ring 5114 or other status indicator in order to display a variety of status indications. For example, light ring 5114 may include a combination of two or more colors (e.g., white, blue, and red), each of which may be independently activated. Each light source may be operated in a number of modes, such as a “solid” mode characterized by continuous activation of the light source (e.g., a steady “on” state), a “blinking” mode characterized by repeated activation and deactivation of the light source, a “flash” mode characterized by a single activation and deactivation of the light source, a “breathing” mode characterized by repeated gradual brightening and dimming of the light source, etc.

Combinations of colors and activation modes may be used to indicate the status of the reader device 5100. For example, in some embodiments, the light ring 5114 or other status indicator may display a first indication such as a solid white light when the reader device 5100 is powered up and ready to receive an assay cartridge 5120 (e.g., when a power cartridge is removed). Other examples of device status that may be indicated by the status indicator include, for example, a cartridge 5120 is inserted into the reader device 5110, a test has been started and is running, a test is complete, a cartridge is removed after completion of a test, an error (e.g., a test malfunction, premature removal of the cartridge 5120, etc.), Bluetooth pairing, or any other status of the reader device 5110. In one non-limiting example, a solid white light ring 5114 indicates that a power cartridge has been removed and the device is powered up or that a test cartridge has been removed after completion of a test, a solid blue light ring 5114 indicates that a test cartridge has been inserted into the reader device 5110, a breathing blue light ring 5114 indicates that a test has been started and is running, a breathing white light ring 5114 indicates that a test is completed and the cartridge may be removed, a solid, breathing, or blinking red light ring indicates an error, a flash of blue and red at the light ring 5114 indicates Bluetooth pairing in progress, and a steady, flashing, or blinking blue light ring 5114 indicates Bluetooth pairing complete. It will be understood that other implementations may include any combination or subcombination of the status indicator modes listed above, and/or may include further status indications, light colors, operation modes, or the like.

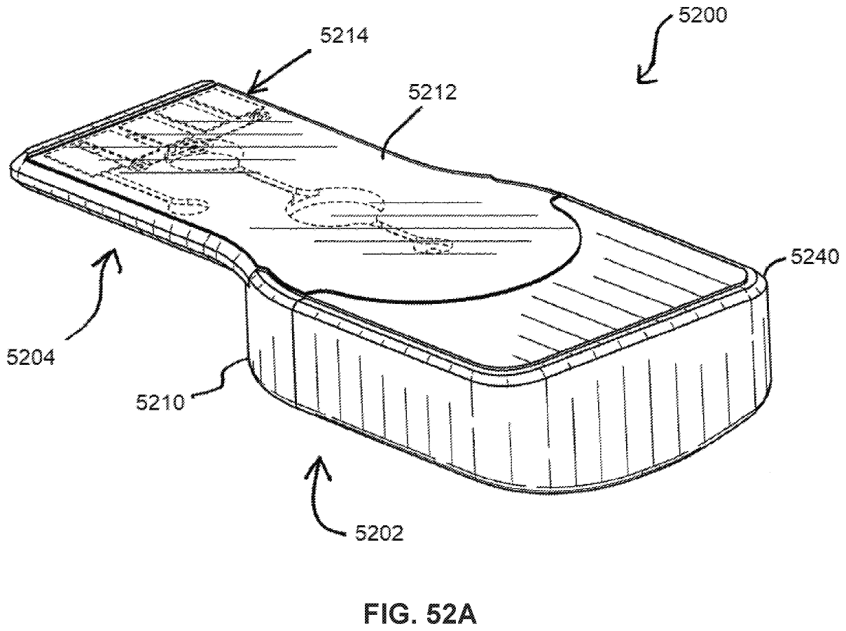

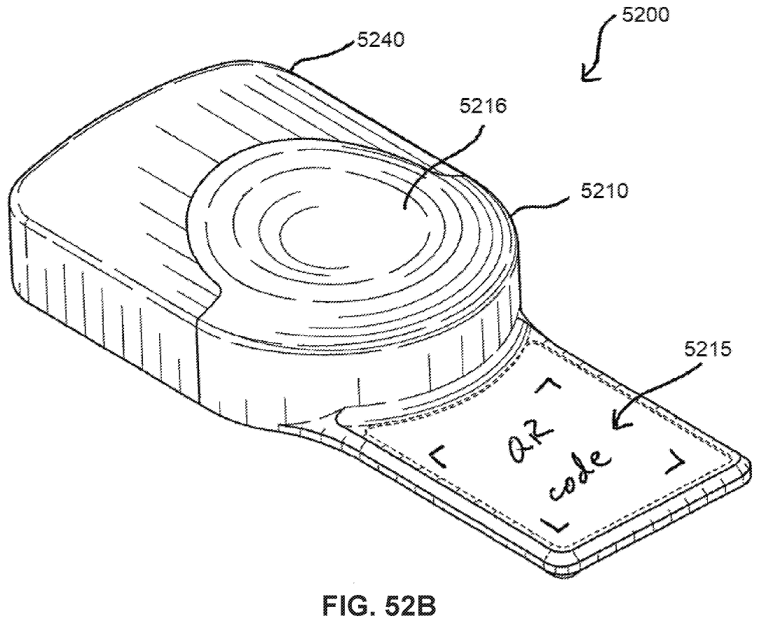

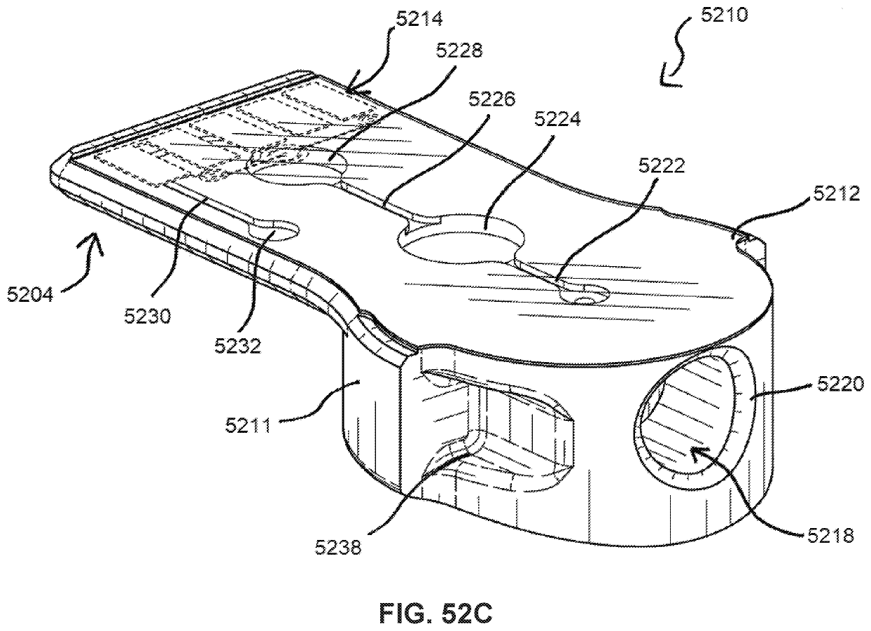

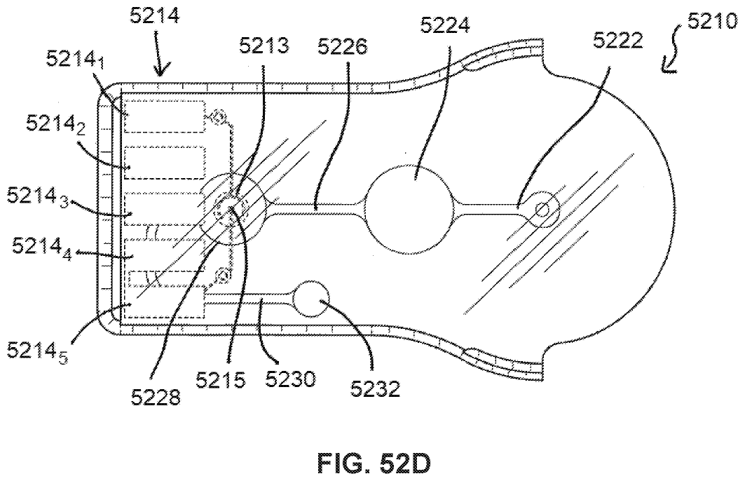

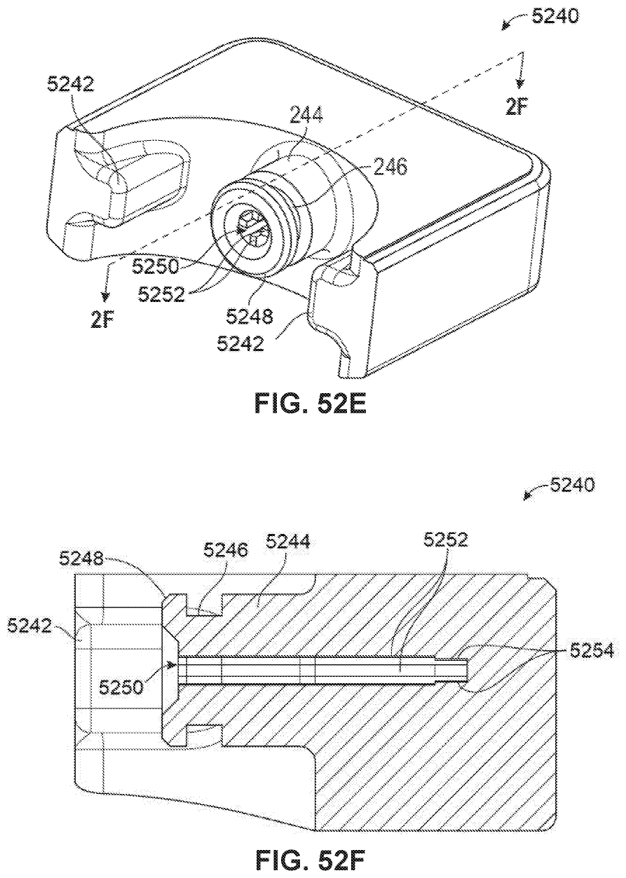

FIGS. 52A-52F depict an example cartridge 5200 configured for detection of a target. As described herein, the target may be a viral target, bacterial target, antigen target, parasite target, microRNA target, or agricultural analyte. Some embodiments of the cartridge 5200 can be configured for testing for a single target, while some embodiments of the cartridge 5200 can be configured for testing for multiple targets. The cartridge 5200 includes a cartridge body 5210 and a cap 5240 configured to be mechanically coupled to the cartridge body 5210. When the cartridge body 5210 and the cap 5240 are coupled together, the cartridge body 5210 forms the internal section 5204 of the cartridge 5200 and a portion of the external section 5202. The cap 5240 forms a remaining portion of the external section 5202.

FIGS. 52A and 52B depict a complete cartridge 5200 including the cartridge body 5210 and the cap 5240 coupled together. In use, the cap 5240 and cartridge body 5210 can operate to seal a provided sample within the cartridge 5200, thereby preventing exposure of test operators to the sample and preventing any liquid from escaping into the electronics of an associated reader device. The cartridge body 5210 and the cap 5240 may be coupled by a friction fit, a snap fit, and/or one or more mechanical or chemical securing means.

The cartridge body 5210 and the cap 5240 can be formed from suitable fluid-impermeable materials such as plastic, and may be opaque, translucent, or transparent. The cartridge body 5210 can also include a translucent or transparent cover 5212 partially defining a fluid flow path within the cartridge body 5210, and one or more electrode interfaces 5214. The cover 5212, fluid flow paths, and electrode interfaces 5214 are discussed in greater detail with reference to FIGS. 52C and 52D. The cartridge body 5210 and/or the cap 5240 can further include a cartridge identifier 5215. The cartridge identifier 5215 may include human-readable and/or machine-readable information, such as text, a barcode, a QR code, or the like. The cartridge identifier 5215 can include any suitable information associated with the cartridge, such as information specifying a type of test, a target agent, a sample type, a cartridge serial number or other individual cartridge identifier, etc. In addition to serving as an identifier for a user of the type of test associated with the cartridge 5200, the cartridge identifier 5215 may also be scanned by a user (e.g., using a user interface device in communication with a reader device) to communicate one or more test protocols to the reader device. The cartridge body 5210 and/or the cap 5240 can include ergonomic features such as an indentation 5216 to facilitate handling of the cartridge 5200.

FIGS. 52C and 52D depict the cartridge body 5210 component of the cartridge 5200 of FIGS. 52A and 52B. The cartridge body 5210 includes a base 5211 and a cover 5212. The base 5211 can be formed from a fluid-impermeable material, for example injection molded or milled acrylic or plastic. The base 5211 includes a receiving well 5218 and components of a cartridge body flow path, including a first segment 5222, a mixing well 5224, a second segment 5226, a test well 5228, a third segment 5230, and a vent 5232. It will be appreciated that the particular geometric configurations or relative arrangements of these features may be varied in other embodiments. As used herein, fluidic communication refers to the capability to transfer fluids (e.g., liquid or gas). The cover 5212 can be formed from a fluid-impermeable material. In some embodiments, the cover 5212 is a translucent or transparent material, such as glass, plastic, or the like. The cover 5212 is sealed to the base 5211 to form the cartridge body 5210 and to serve as a boundary confining fluids within the cartridge body flow path components described above. In some embodiments, a translucent or transparent cover 5212 advantageously allows for visual inspection of a fluid within the cartridge body flow path (e.g., to verify that the test well is full prior to testing, etc.). One or more conductive components of an electrode interface 5214 are disposed on the cover 5212. Mating features 238 are sized and shaped to receive corresponding mating features of the cap 5240. The receiving well 5218 optionally includes a chamfer 5220 to facilitate coupling of the cap 5240 to the cartridge body 5210.

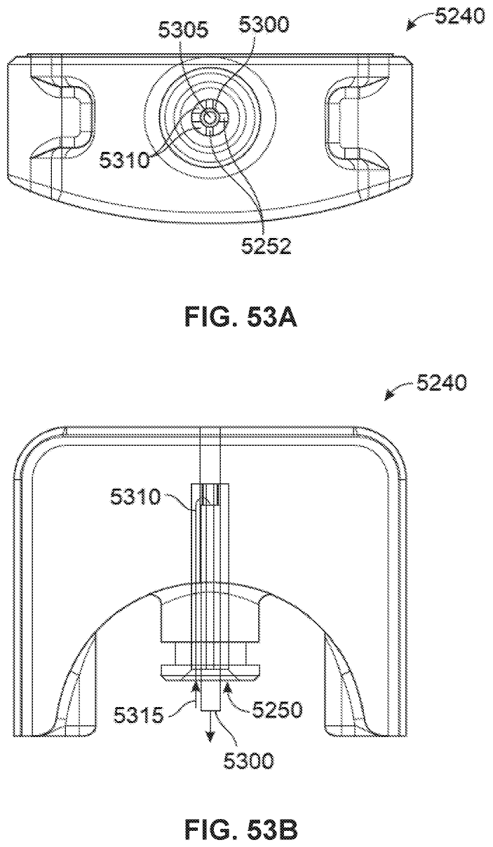

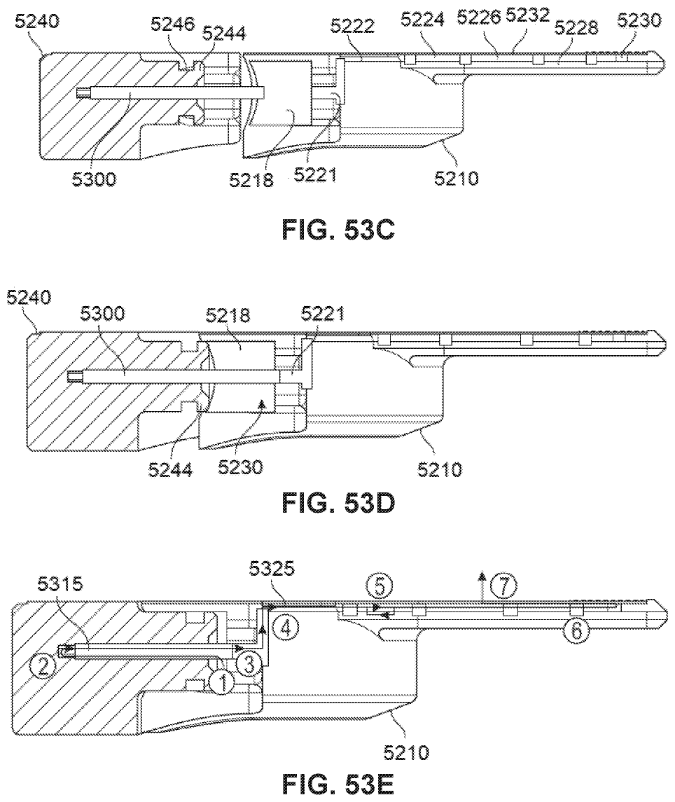

The cartridge body flow path includes segments 5222, 5226, and 5230, as well as an inlet (FIG. 53C) fluidically coupling the receiving well 5218 to the first segment 5222, the mixing well 5224, and the test well 5228. The first segment 5222 of the cartridge body flow path leads from the inlet to the mixing well 5224. The second segment 226 of the cartridge body flow path leads from the mixing well 5224 to the test well 5228. The third segment 5230 is a test well outlet path leading from the test well 5228 to a vent 5232 that allows gas to escape from the test well 5228 and out of the cartridge 5200.

The mixing well 5224 may include one or more reagents in a dry form (e.g., a powder). Powdered reagents and/or other dry reagents may be hydrated by a fluid sample when the fluid sample enters the mixing well 5224. The reagents provided in the mixing well 5224 can be selected based on one or more protocols of the intended test associated with the cartridge 5200. Even or homogenous mixing of the reagents with the fluid sample can yield more accurate test results in some embodiments. As such, the mixing well 5224 is configured to promote even mixing of the reagent with the fluid sample, for example by including curved regions and/or a cross-sectional shape that promote turbulent flow rather than laminar flow of the liquids within the mixing well 5224. Turbulent flow is a flow regime in fluid dynamics characterized by chaotic changes in pressure and flow velocity of a fluid. Turbulent flow is in contrast to laminar flow, which occurs when fluid flows in parallel layers, with no disruption between those layers.

The segments 5222, 5226, and 5230 of the cartridge body flow path, the mixing well 224, and/or the test well 5228 can be entirely encased within the material of the base 5211, or can have three surfaces formed from the material of the base 5211 with the cover 5212 forming an upper surface that seals these channels.

The internal section 5204 or test region of the cartridge body 5210 includes the segments 5226, 5230 of the cartridge body flow path, the test well 5228, the valve 5232, electrodes 5213, 5215, and an electrode interface 5214. The electrode interface 5214 includes a plurality of contact pads 5214₁-5214₅. Although five contact pads 5214₁-5214₅ are depicted, the cartridge body 5210 may equally include more or fewer than five contact pads. A first contact pad 5214₁ is electrically connected to a first electrode 5213 of the test well 5228, and a second contact pad 5214₅ is electrically connected to a second electrode 5215 of the test well 5228. One of the contact pads 5214₁, 5214₅ is configured for coupling an excitation electrode of a test well with a voltage or current source of a reader device and the other of the contact pads 5214₁, 5214₅ is configured for electrically coupling a signal electrode of the test well with a signal reading conductor of the test device. Additional ones of the contact pads 5214₁-5214₅ may serve other purposes in conjunction with the reader device. For example, one or more of the contact pads 5214₁-5214₅ may couple to circuitry of the electrode interface of the reader device to indicate one or more test protocols to the reader. In another example, a power cartridge, as described above with reference to FIGS. 51A-51C, may include a similar set of contact pads 5214₁-5214₅ configured to connect to circuitry of the reader device's electrode interface to activate a power circuit of the reader device.

The mixing well 5224 can be provided with solid dried or lyophilized constituents for the testing process, for example primers and proteins. The particular selection and chemistry of these dried or lyophilized constituents can be tailored to a particular target or targets for which the cartridge 5200 is designed to test. These dried or lyophilized constituents can be hydrated with the liquid e.g., a buffer or liquid sample that flows into the test well (e.g., the fluid sample within the cartridge 5200) and thus activated for the test procedure. Beneficially, providing the dried or lyophilized solid constituents in the mixing well 5224 enables the cartridge 5200 to be stored before use containing the components needed for the amplification process, while also delaying initiation of amplification until after the sample has been applied.

The test well 5224 is depicted as a generally cylindrical well formed as a circular opening in the material of the base 5211 and bounded by the planar surface of the cover 5212. The test well 5224 contains two electrodes 5213, 5215, with one electrode being an excitation electrode configured to apply current to the sample in the test well 5224 and the other electrode being a signal electrode configured to detect current flowing from the excitation electrode through the liquid sample. In some embodiments, one or more test wells can be provided with a thermistor in place of the electrodes in order to provide for monitoring of the temperature of the fluid within the cartridge 5100.

In some embodiments, gas bubbles within the test well 5224, particularly if positioned along the current path between the electrodes 5213, 5215, can create noise in the signal picked up by the signal electrode. This noise can reduce the accuracy of test results determined based on the signal from the signal electrode. A desired high-quality signal may be obtained when only liquid is present along the current path or when minimal gas bubbles are present along the current path. As described above, any air initially present in the fluid flowing along the cartridge body flow path can be pushed out through the vent 5232. In addition, the electrodes 5213, 5215 and/or test well 5224 can be shaped to mitigate or prevent nucleation of the liquid sample in which air or gas bubbles form in the fluid sample and collect along the electrodes 5213, 5215.

For example, the electrodes 5213, 5215 may be positioned at the bottom of the test well 5224 in some embodiments. This can allow any air or gas to rise to the top of the fluid in the test well and away from the path between the electrodes. As used herein, the bottom of the test well 5224 refers to the portion of the test well in which heavier liquid settles due to gravity, and the top of the test well refers to the portion of the test well in which lighter gas rises above the heavier liquids. Further, the electrodes 5213, 5215 are positioned away from the perimeter or edges of the test well 5224 which is a location at which bubble nucleation typically occurs.

Further, the electrodes 5213, 5215 can be formed from a thin, flat layer of material that has minimal height relative to the underlying circuit board layer that forms the bottom of the test well 5224. In some embodiments, the electrodes 5213, 5215 can be formed using electrodeposition and patterning to form a thin layer of metal film, for example around 5300 nm in height. This minimal height can help prevent or mitigate air bubbles from becoming trapped along the interface between the electrode and the underlying layer. In some embodiments, a layer of conductive material can be deposited on top of each electrodes to create a smoother transition between the edge of the electrode and the bottom of the test well. For example, a thin polyimide layer (e.g., around 5 microns in height) can be deposited on top of the electrode or the circuit board can be butter coated. Additionally or alternatively, the electrodes can be positioned in grooves in the underlying layer with the grooves having a depth approximately equal to the height of the electrode. These and other suitable methods can achieve an electrode that is approximately flat or flush with the bottom surface of the well.

Beneficially, the above-described features can help to keep the electrodes 5213, 5215 surrounded by liquid and prevent or reduce gas bubbles from becoming positioned along the current path between the electrodes 5213, 5215.

FIGS. 52E and 52F depict the cap 5240 component of the cartridge 5200. FIG. 52F is a cross-sectional view taken about the line 2F-2F in FIG. 2E to illustrate internal structures of the cap 5240. The cap 5240 is sized and shaped to mate with or otherwise mechanically couple to the cartridge body 5210 to form a complete cartridge 5200. The cap 5240 includes mating features 5242 configured to interlock with corresponding mating features 5238 of the cartridge body when the cartridge 5200 is assembled. The cap further includes a plunger 5244 disposed about a retaining well 5250 for retaining a capillary tube therein.

The plunger 5244 is sized and shaped to sealingly engage with the receiving well 5218 of the cartridge body 5210 (FIGS. 52C-52D). The plunger 5244 optionally includes a groove 246 configured to receive an O-ring or other gasket to eve and/or enhance the seal between the plunger 5244 and the receiving well 5218. An optional chamfer 5248 at a distal end of the plunger 5244 may facilitate the engagement of the plunger 5244 with the receiving well 5218, alone or in combination with the chamfer 5200 of the receiving well 5218 (FIGS. 52C-52D). As will be described in greater detail with reference to FIGS. 53A-53E, the plunger 5244 thus sealingly engages with the receiving well 5218 to propel a fluid sample into the cartridge body 5210.

The retaining well 5250 is configured to partially surround a capillary tube containing a fluid sample for testing. The retaining well 5250 preferably has an interior diameter larger than the exterior diameter of the capillary tube to be inserted. A plurality of retaining structures 5252 extend inward from the interior walls of the retaining well 5250 to hold the capillary tube at a central location within the retaining well 5250. Preferably, the distance between opposing retaining structures 5252 is approximately equal to or slightly larger than the exterior diameter of the capillary tube. As shown in FIG. 52F, a rear portion 5254 of each retaining structure 5252 extends further inward relative to the remaining portion of the retaining structure 5252. The distance between opposing rear portions 5254 is small enough that the capillary tube cannot fit between the rear portions 5254. Accordingly, the rear portions 5254 of the retaining structures 5252 block the movement of the capillary tube along the retaining well 5250 and maintain a space between the capillary tube and the rear wall of the retaining well 5250. As will be described in greater detail with reference to FIGS. 53A-53E, this spaced location of the capillary tube within the cap 5240 allows air or other fluid to flow into the retaining well 5250 around the sides of a capillary tube between the retaining structures and into the rear of the capillary tube.

The cartridge 5200 of FIGS. 52A-52F provides a self-contained, easy to use device for performing an amplification-based test for a target, for example nucleic acid testing wherein genomic material in the sample is exponentially copied using a molecular amplification process. Beneficially, the user only needs to apply the sample and insert the cartridge 5200 into a reader device in order to ascertain the result of the test in some embodiments, as the solid constituents of the amplification process are pre-provided within the cartridge and automatically mixed with the sample. In some embodiments, one or both of the cartridge or reader may include a heater and a controller configured to operate the heater to maintain the cartridge at the desired temperature for amplification. In some embodiments, one or both of the cartridge or reader may include a motor to impart vibrations to or otherwise agitate the cartridge to cause any trapped gas to rise to the top of the liquid and vent from the test wells.

FIGS. 53A-53E illustrate mechanical fluid transfer aspects of the cartridges 5120, 5200 described herein. As will be described in greater detail, the cartridge body 5210 and cap 5240 are configured to create air pressure when coupled together, such that the air pressure propels a fluid sample through the fluid flow path of the cartridge body 5210. FIGS. 53A-53E illustrate the cap 5240 with translucency to reveal interior features of the cap 5240. The cartridge body 5210 is illustrated in a cutaway view in FIGS. 3C-3E to reveal interior features of the cartridge body 5210.

With reference to FIGS. 53A and 53B, a fluid sample may be received in a capillary tube 300, for example, within an inner lumen 5305 of the capillary tube 5300. The cap 5240 is sized and shaped to receive the capillary tube 5300 as described above with reference to FIGS. 52E and 52F. The fluid sample may be introduced into the capillary tube 5300 while the capillary tube 5300 is within the cap 5240, or the capillary tube 5300 may contain the fluid sample when it is placed into the cap 5240.

FIG. 53A is a front view of the cap 5240 of the cartridge 5200. While the capillary tube 300 is disposed within the retaining well 5250 of the cap 5240, the retaining structures 5252 hold the capillary tube 5300 in a position spaced from the walls of the retaining well 5250. Thus, a plurality of air channels 5310 are formed between the interior of the retaining well 5250 and the exterior of the capillary tube 5300.

FIG. 53B is a top view of the cap 5240 of FIG. 53A. A rear portion 5310 of some or all of the retaining structures 5310 (e.g., the rear portions 5254 of FIG. 52F) cause the capillary tube 5300 to remain spaced from the rear of the retaining well 5250. This arrangement forms a cap fluid flow path 5315 such that air or other fluids can flow into the retaining well 5250 through the air channels 5310, around the rear of the capillary tube 5300, and out of the retaining well 5250 through the inner lumen 5305 of the capillary tube 5300. Accordingly, application of a relatively high pressure at the air channels 5310 can cause a fluid within the inner lumen 5305 to flow out of the capillary tube 5300 along the cap fluid flow path 5315.

FIGS. 53C-53E illustrate various stages in a process of coupling the cap 5240 to the cartridge body 5210, together with associated fluid flow paths for effecting sample movement into the test well 5228 and other components of a cartridge body flow path 5325. FIG. 53C depicts the cap 5240 adjacent but not coupled to the cartridge body 5210, FIG. 53D depicts the cap 5240 being coupled to the cartridge body 5210, and FIG. 53E depicts the cap 5240 fully coupled with the cartridge body 5210.

As shown in FIG. 53C, the plunger 5244, retaining well 5250, and capillary tube 5300 are aligned with the receiving well 5218 of the cartridge body 5210. The plunger 5244 is sized and shaped to sealingly engage the receiving well 5218. An O-ring or other seal (not shown) can be positioned in the groove 5246 of the plunger 5244 to achieve and/or enhance the seal between the plunger 5244 and the receiving well 5218. The receiving well 5218 is fluidically coupled to the mixing well 5224 by an inlet 5221 sized and shaped to sealingly receive an end of the capillary tube 5300. When the cap 5240 is aligned with the cartridge body 5210, the process continues to the configuration of FIG. 53D.

As shown in FIG. 53D, the plunger 5244 engages the walls of the receiving well 5218. As the plunger 5244 (and/or an O-ring disposed on the plunger 5244) engages the walls of the receiving well 5218, a volume of ambient air is trapped within the receiving well 5218. This trapped air 5320 has a volume defined by the portion of the receiving well 5218 not occupied by the plunger 5244. As the cap 5240 is pressed further onto the cartridge body 210, an outer end of the capillary tube 5300 enters and sealingly engages with the inlet 5221. Thus, as cap 5240 and the cartridge body 5210 are pressed further together, the trapped air 5320 is compressed within the shrinking volume of the portion of the receiving well 5218 not occupied by the plunger 5214. Because the inlet 5221 is blocked by the capillary tube 5300, the compression of the trapped air 5320 causes the trapped air 5320 to flow along the cap flow path 5315 of FIG. 53B.

Referring now to FIG. 53E, the fluid transfer effected by coupling the cap 5240 and the cartridge body 5210 will be described. FIG. 53E illustrates the flow along the cap flow path 5315 and the cartridge body flow path 5325 with encircled numbers shown as labels for certain points along the fluid path. The encircled numbers are discussed below as example steps of a progression of trapped air 5320 and a fluid sample as they travel through the cap flow path 5315 and the cartridge body flow path 5325 within the cartridge 5200, with each step including a directional arrow showing the direction of fluid travel at that step. For clarity and simplicity of FIG. 53E, some components labeled with reference numbers in FIGS. 52A-53D are not labeled in FIG. 53E.

Prior to step (1), a user provides a fluid sample within a capillary tube 5300. Also prior to step (1), the capillary tube 5300 is placed within the retaining well 5250 of the cap 5240 between the retaining structures 5252 to form the cap flow path 5315.

At step (1), as the plunger 5244 compresses the trapped air 5320, the trapped air 5320 is forced into the air channels 5310. The trapped air 5320 flows along the cap flow path 5315 through the air channels 5310 between the retaining structures 5252 and along the exterior of the capillary tube 5300.

At step (2), the trapped air 5320 reaches the rear of the retaining well 5218. The trapped air 5320 continues along the cap flow path 5315 into the inner lumen 5305 of the capillary tube 5300. Upon entering the inner lumen 5305, the trapped air 5320 contacts and exerts a pressure upon the fluid sample contained within the capillary tube 5300. The pressure is directed along the length of the capillary tube 5300 toward the cartridge body 5210.

At step (3), the fluid sample flows out of the capillary tube 5300 and into the inlet 5221 of the cartridge body 5210. The fluid sample is propelled into the inlet 5221 by the pressured exerted at the opposite end of the capillary tube 5300 by the trapped air 5320. Capillary action or wicking may also propel the fluid sample into the inlet 5221, for example, where the inlet and fluidically connected segments along the cartridge body flow path 5325 are suitably narrow to cause wicking. At step (4), the fluid sample travels through the first segment 5222 of the cartridge body flow path 5325.

At step (5), the fluid sample enters the mixing well 5224. The mixing well may include one or more reagents. Agitation caused by the flow of the fluid sample within the relatively larger space of the mixing well 5224 causes the reagent and the sample to be mixed. In some embodiments, the reagent and the fluid sample are mixed into a homogenous solution in which the reagent is evenly distributed throughout the fluid sample. The depth, width, and/or cross-sectional profile of the mixing well 5224 may be selected to facilitate mixing of the reagent and the fluid sample.

At step (6), the mixed reagent and fluid sample (referred to as the “test fluid”) leave the mixing well 5224 and travel along the second segment 5226 of the cartridge body flow path 5325 into the test well 5228.

At step (7), a portion of the test fluid continues along the third segment 5230 of the cartridge body flow path 5325 to fill any remaining open volume within the cartridge body flow path 5325. The path of step (7) shows the optional flow of a gas (e.g., a gas portion of the test fluid or ambient air present within the cartridge body 5210) through the valve 5232. In some embodiments, the valve 5232 can include a liquid-impermeable, gas-permeable filter to allow any gas present in the test fluid or within the cartridge body 5210 to vent through the valve 5232 as the test fluid fills the space within the cartridge body flow path 5325. The valve 5232 may further minimize the occurrence of air bubbles within the test well 5228. In some embodiments the valve 5232 may not present and/or may not be configured to vent gas.

Following the completion of steps (1)-(7), the cartridge 5200 is sealed and contains the test fluid within the cartridge body 5210 and the cap 5240. The sealed cartridge 5200 may then be placed into a reader device such as the reader devices 5110, 600, 6000 described herein, for testing to detect one or more target agents within the test fluid. In various embodiments, the size of the fluid sample and/or the quantity of the reagent may preferably be selected to provide sufficient test fluid to substantially fill the fluid space enclosed within the cartridge 5200 along the cap flow path 5315 and the cartridge body flow path 5325. The volume of the receiving well 5218, and the corresponding size of the plunger 5244, may preferably be selected so that the receiving well 5218 contains sufficient air for transporting the fluid sample along the length of the fluid path and into the test well 5328. It will be understood that the propulsion of the fluid sample through the capillary tube 5300 into and along the cartridge body flow path 5325, as described above with reference to FIGS. 53A-53E, may occur due to capillary action or wicking, fluid pressure due to the compression of a trapped liquid or gas (e.g., air) within the receiving well 5218, or both.

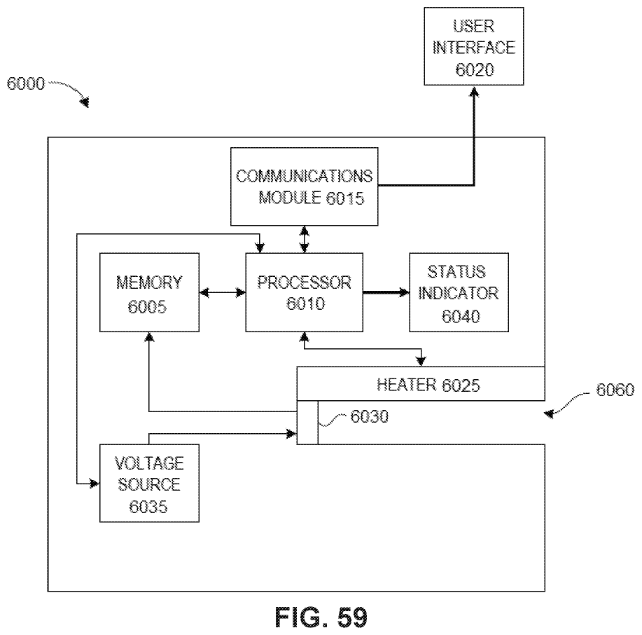

FIG. 59 depicts a schematic block diagram of an example reader device 6000 that can be used with the cartridges described herein, for example the cartridges 5120 or 5200. The schematically illustrated reader device 6000 may be, for example, the reader device 5110 of FIGS. 51A-51C. The reader device 6000 includes a memory 6005, processor 6010, communications module 6015, heater 6025, electrode interface 6030, voltage source 6035, and a cavity 6060 into which a cartridge can be inserted. The reader device 6000 may further include a status indicator 6040. The reader device 6000 is in communication with a user interface 6020, which may include a user interface of a remote computing device such as a smartphone, tablet, or other device having a testing control application executing thereon.

When test cartridge 5120, 5200 is inserted into the cavity 6060 of the reader device 6000, the electrode interface 5214 of the cartridge couples with the electrode interface 6300 of the reader device 6000. This can allow the reader device 6000 to detect that a cartridge is inserted, for example by testing whether a communication path is established. In some embodiments, the optional power cartridges described above with reference to FIGS. 51B and 51C may activate a power supply circuit of the reader device 6000 when the electrode interface 5214 of the cartridge couples with the electrode interface 6030 of the reader device 6000. Further, such communications can enable the reader device 6000 to identify a particular inserted test cartridge 5120, 5200 and access corresponding testing protocols. Testing protocols can include the duration of the test, the temperature of the test, the characteristics of a positive sample impedance curve, and the information to output to the user based on various determined test results. In other embodiments, the reader device 6000 can receive an indication via user interface 6020 that a cartridge is inserted (e.g., by a user inputting a “begin testing” command and optionally a test cartridge identifier).

The memory 6005 includes one or more physical electronic storage devices configured for storing computer-executable instructions for controlling operations of the reader device 6000 and data generated during use of the reader device 6000. For example, the memory 6005 can receive and store data from sensing electrodes coupled to the electrode interface 6030.

The processor 6010 includes one or more hardware processors that execute the computer-executable instructions to control operations of the reader device 6000 during a test, for example by controlling the heater 6025, controlling the communications module 6015 to interact with the user interface 6020, and activating the voltage source 6035. One example of testing operations is described with respect to FIG. 7A below. The processor 6010 can be also be configured by the instructions to determine test results based on data received from the excitation electrodes of an inserted test cartridge, for example by performing the process of FIG. 7B described below.

The communications module 6015 includes network-enabled hardware components, for example wired or wireless networking components, for providing networked communications between the reader device 6000 and remote computing devices. Suitable networking components include WiFi, Bluetooth, cellular modems, Ethernet ports, or USB ports, and the like. Beneficially, networking capabilities can enable the reader device 6000 to interact with and be controlled by remote computing devices such as one or more additional handheld computing devices (e.g., smartphones, tablets, etc.). In some embodiments, remote devices may be in communication additional remote computing systems such as hospital information systems and/or laboratory information systems that store electronic medical records, national health agency databases, and the computing devices of clinicians or other designated personnel. In addition, the networking capabilities can enable the reader device 6000 to receive information over the network from remote computing devices, for example updated signal cliff parameters for existing test, new signal cliff parameters for new tests, and updated or new testing protocols.

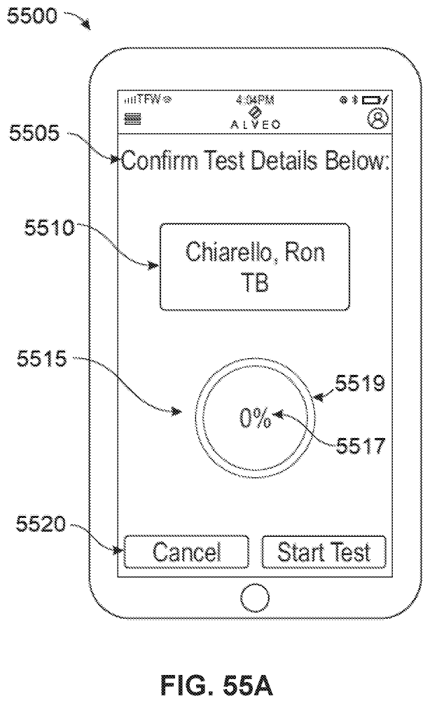

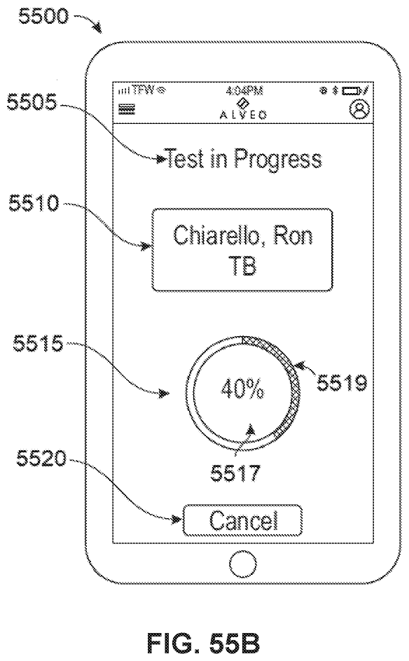

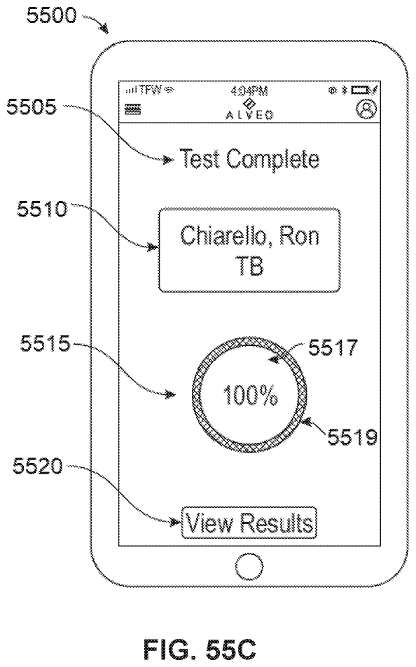

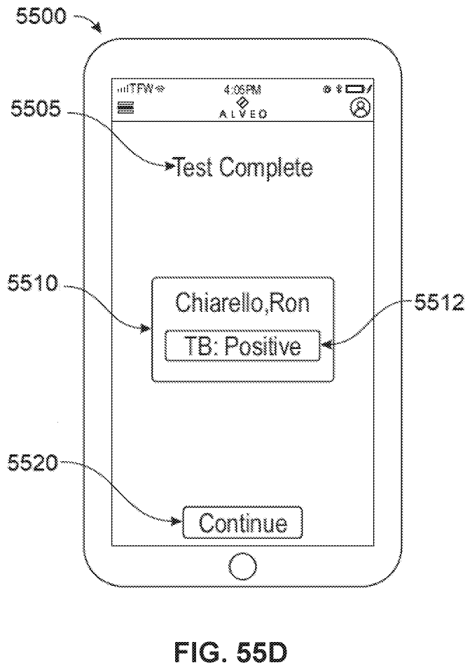

The user interface 6020 can be implemented within a remote device connected to the communications module 6015 via WiFi, or Bluetooth, or the like. The remote device may have a testing control application installed thereon to provide a testing system user interface, for providing control options and/or presenting test results and other test information to users, on a display of the remote device. Further details of the user interface 6020 are described with reference to FIGS. 55A-55D.

The heater 6025 can be positioned adjacent to the cavity 6060 for heating an inserted cartridge to the desired temperature for an amplification process. Though depicted on a single side of the cavity 6060, in some embodiments the heater 6025 can surround the cavity.

As described herein, the voltage source 6035 can provide an excitation signal at a predetermined voltage and frequency to the excitation electrode of an inserted test cartridge.

The status indicator 6040 may include any suitable notification device, such as one or more lights, sound generators, or the like. Operation of a light-based status indicator is described in greater detail with reference to FIGS. 51A-51C.

FIG. 54 depicts a flowchart of an example process 701 for operating a reader device during a test as described herein. The process 701 can be performed by the reader device 600 or 6000 described above.

At block 706, the reader device 600 or 6000 can detect that a power cartridge has been removed from the reader device 600 or 6000. In some embodiments, the detection of block 706 can occur based on the disconnection of a signal path between the electrode interface 630 or 6030 of the reader device 600 or 6000 and one or more contact pads 5214₁-5214₅ (FIG. 52D) of the power cartridge.

At block 711, the reader device 600 or 6000 automatically powers on in response to detecting the removal of the power cartridge at block 706. In some embodiments, the reader device may transmit a notification to a user interface 620 or 6020 device and/or illuminate one or more status lights of a status indicator 640 or 6040 to indicate that the reader device 600 or 6000 is powered on and ready to receive an assay cartridge 120, 200.

At block 716, the reader device 600 or 6000 can detect that an assay cartridge 5120, 5200, has been inserted, for example in response to user input or in response to establishing a signal path with the inserted cartridge. In some embodiments, the cartridge 5120, 5200 can include an information element that identifies the particular test(s) to be performed to the reader device 600 or 6000 and optionally includes test protocol information.

At block 721, the reader device 600 or 6000 can heat the cartridge 5120, 5200 to a specified temperature for amplification. For example, the temperature can be provided by information stored on the cartridge 5120, 5200 or accessed in the internal memory of the reader device 600 or 6000 in response to identification of the cartridge 5120, 5200.

At decision block 725, the reader device 600 or 6000 can determine whether the test is still within its specified test duration. For example, where the expected window of time in which a signal cliff should appear in a positive sample is known, the duration of the test may end at or some predetermined period of time after the end of the window. If so, the process 701 transitions to optional decision block 730 or, in embodiments omitting block 730, to block 735.

At optional decision block 730, the reader device 600 or 6000 determines whether to monitor the test well amplification by logging data from the test well sensing electrode. For example, the reader 600 or 6000 may be provided with instructions to only monitor the impedance of the test well during a particular window or windows of a test. If the reader device 600 or 6000 determines not to monitor the test well amplification, the process 701 loops back to decision block 725.

If the reader device 600 or 6000 determines to monitor the test well amplification, the process 701 transitions to block 735. At block 735, the reader device 600 or 6000 provides an excitation signal to the excitation electrode of the test well(s) of the inserted cartridge. As described above, this can be an alternating current at a particular frequency and voltage.

At block 740, the reader device 600 or 6000 detects and logs data from the sensing electrode of the test well(s) of the inserted cartridge. In some embodiments, this data can be stored for later analysis, for example after completion of the test. In some embodiments, the reader device 600 or 6000 can analyze this data in real time (e.g., as the test is still occurring) and may stop the test once a positive sample signal cliff is identified.

When the reader device 600 or 6000 determines at block 725 that the test is not still within its specified duration, the process 701 moves to block 745 to analyze the test data and output the test result. The test result can include an indication that the sample tested positive or negative for the target, or can more specifically indicate an estimated quantity of the target in the tested sample. Following the conclusion of the test, further tests may be performed by returning to block 715 for a new assay cartridge. Alternatively, the reader device 600 or 6000 may detect insertion of a power cartridge and power off in response.

FIGS. 55A-55D depict screens of an example graphical user interface 5500 of a user device implementing an example testing process in communication with a reader device as described herein. The user interface 5500 may be, for example, the user interface 620 illustrated in connection with the reader device 600 of FIG. 6 or 6000 of FIG. 59. The user interface 5500 may be implemented with any of the reader devices 5110, 600 and/or assay cartridges 5120, 5200 described herein. The screens depicted in FIGS. 55A-55D may be displayed, for example, by an application executing on a smartphone or other user interface device paired to the reader device 5110, 600, 6000 (e.g., by WiFi, Bluetooth, or the like) so as to allow a user to control and/or monitor the reader device 5110, 600, 6000 from the user interface device.

FIG. 55A depicts an initial pre-test screen which may be displayed after an inserted assay cartridge 5120, 5200 has been detected. In one example, a user scans a cartridge identifier (e.g., cartridge identifier 5215 of FIG. 52B) of a cartridge before inserting the cartridge into the reader device. When the device is inserted, the paired reader device detects the inserted cartridge and sends a message to the user interface device that the cartridge has been inserted. The application then displays the initial pre-test screen depicted in FIG. 55A.

The initial pre-test screen includes a status indication area 5505, a test identifying area 5510, a progress indication area 5515 including a numeric progress indication 5517 and a graphical progress indication 5519, and an input area 5520. The status indication area 5505 may include an instruction, such as a request for the user to confirm the information in the test identifying area 5510. The test identifying area 5510 includes information associated with the test to be performed, such as a name or other identifier of a test subject, a condition or target agent to be detected, or the like. In the initial pre-test screen of FIG. 55A, the input area 5520 includes user-selectable “cancel” and “start test” options to allow the user to cancel the test or confirm the details and start the test.

FIG. 55B depicts a mid-test screen that may be displayed while the reader device is conducting the test on the fluid sample within the cartridge. The status indication area 5505 indicates that the test is in progress. As the test progresses, the numeric progress indication 5517 and the graphical progress indication 5519 are updated to display the current progress of the test. A user-selectable option to cancel the test is provided in the input area to allow a user to stop the test if desired.

FIG. 55C depicts an initial test completion screen that may be displayed when the reader device has completed the test and has analyzed the logged test data to determine a test result. The status indication area 5505, numerical progress indication 5517, and/or the graphical progress indication 5519 may indicate that the test is complete. In the input area 5520, a user-selectable option to view the test results is provided.

FIG. 55D depicts a test result display screen for communicating the results of the test to a user. The test identifying area 5510 may still display some or all of the originally displayed test identifying information. The test identifying area 5510 may additionally display an outcome 5512, such as positive or negative, or other condition associated with the test results. The input area 5520 may provide a user-selectable option to continue (e.g., to conduct additional tests, or transmit results, etc.).

FIGS. 55A-55D depict screens of an example graphical user interface 5500 of a user device implementing an example testing process in communication with a reader device as described herein. The user interface 5500 may be, for example, the user interface 6020 illustrated in connection with the reader device 5500 of FIG. 55. The user interface 5500 may be implemented with any of the reader devices 5110, 6000 and/or assay cartridges 5120, 5200 described herein. The screens depicted in FIGS. 55A-55D may be displayed, for example, by an application executing on a smartphone or other user interface device paired to the reader device 5110, 6000 (e.g., by WiFi, Bluetooth, or the like) so as to allow a user to control and/or monitor the reader device 5110, 6000 from the user interface device.

FIG. 55A depicts an initial pre-test screen which may be displayed after an inserted assay cartridge 5120, 5200 has been detected. In one example, a user scans a cartridge identifier (e.g., cartridge identifier 5215 of FIG. 52B) of a cartridge before inserting the cartridge into the reader device. When the device is inserted, the paired reader device detects the inserted cartridge and sends a message to the user interface device that the cartridge has been inserted. The application then displays the initial pre-test screen depicted in FIG. 55A.

The initial pre-test screen includes a status indication area 5505, a test identifying area 5510, a progress indication area 5515 including a numeric progress indication 5517 and a graphical progress indication 5519, and an input area 5520. The status indication area 5505 may include an instruction, such as a request for the user to confirm the information in the test identifying area 5510. The test identifying area 5510 includes information associated with the test to be performed, such as a name or other identifier of a test subject, a condition or target agent to be detected, or the like. In the initial pre-test screen of FIG. 55A, the input area 5520 includes user-selectable “cancel” and “start test” options to allow the user to cancel the test or confirm the details and start the test.

FIG. 55B depicts a mid-test screen that may be displayed while the reader device is conducting the test on the fluid sample within the cartridge. The status indication area 5505 indicates that the test is in progress. As the test progresses, the numeric progress indication 5517 and the graphical progress indication 5519 are updated to display the current progress of the test. A user-selectable option to cancel the test is provided in the input area to allow a user to stop the test if desired.

FIG. 55C depicts an initial test completion screen that may be displayed when the reader device has completed the test and has analyzed the logged test data to determine a test result. The status indication area 5505, numerical progress indication 5517, and/or the graphical progress indication 5519 may indicate that the test is complete. In the input area 5520, a user-selectable option to view the test results is provided.

FIG. 55D depicts a test result display screen for communicating the results of the test to a user. The test identifying area 5510 may still display some or all of the originally displayed test identifying information. The test identifying area 5510 may additionally display an outcome 5512, such as positive or negative, or other condition associated with the test results. The input area 5520 may provide a user-selectable option to continue (e.g., to conduct additional tests, transmit results, etc.).

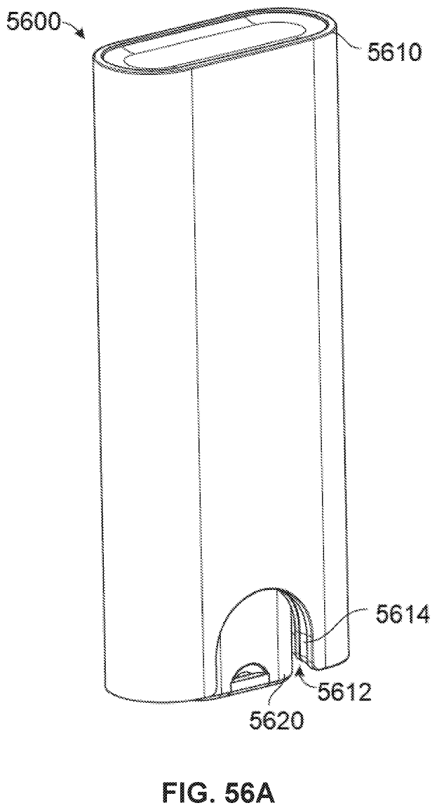

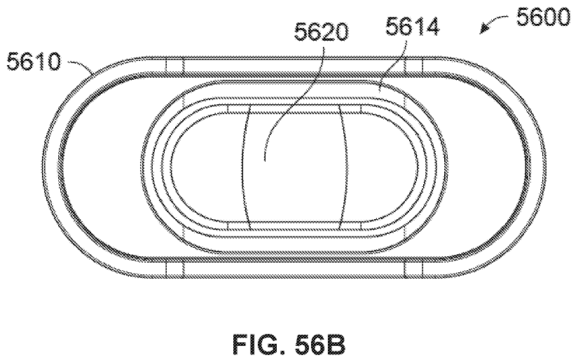

FIGS. 56A and 56B depict a further example of a handheld detection system 5600 for detection of a target. Similar to the system 5100 of FIGS. 51A-51C, the system 5600 may be implemented in conjunction with any of the target detection processes, systems, and devices described herein. The system 5600 includes a reader device 5610 and a cartridge 5620 configured to fit within a cavity 5612 of the reader device 5610. The cartridge 5620 is sized and shaped to be gripped by a user to facilitate insertion and/or removal of the cartridge 5620 from the reader device 5610. The reader device 5610 may further include a light ring 5614 disposed about the cavity 5612. The light ring 5614 may include any or all of the light sources, colors, operation modes, etc., described above with reference to the light ring 5114 of FIGS. 51A-51C.

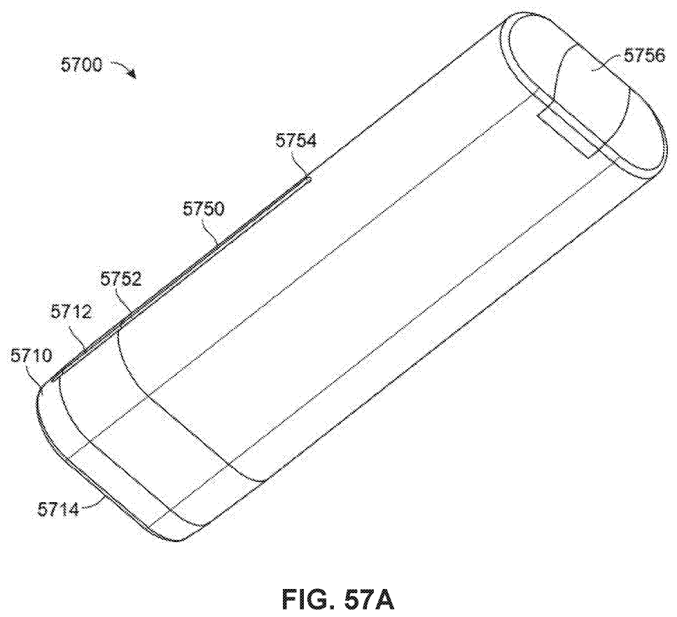

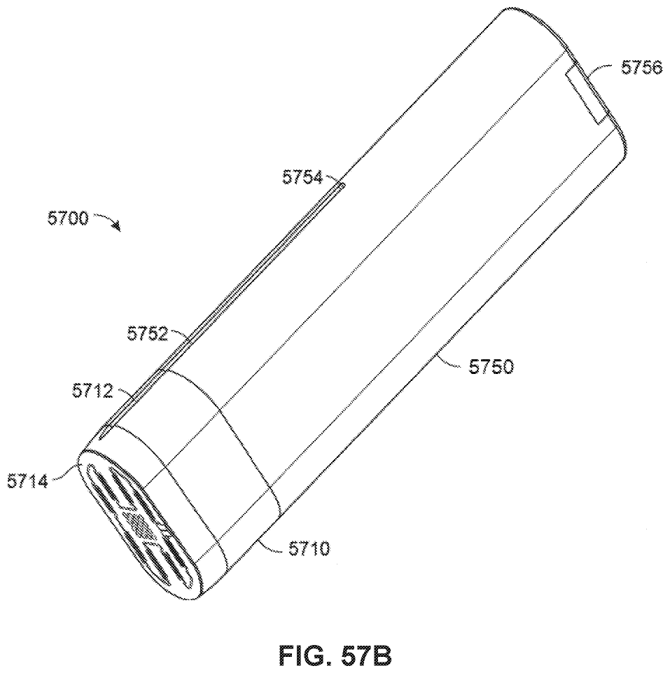

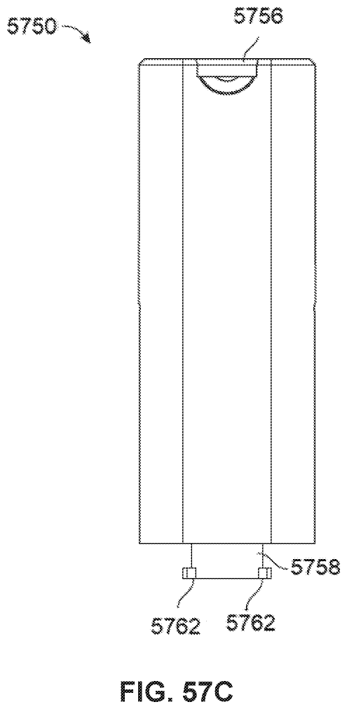

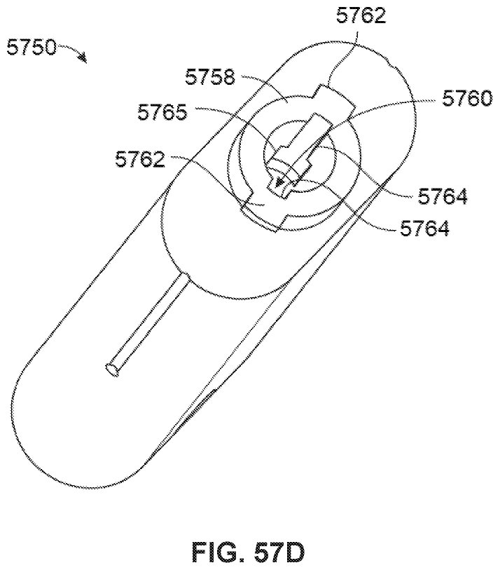

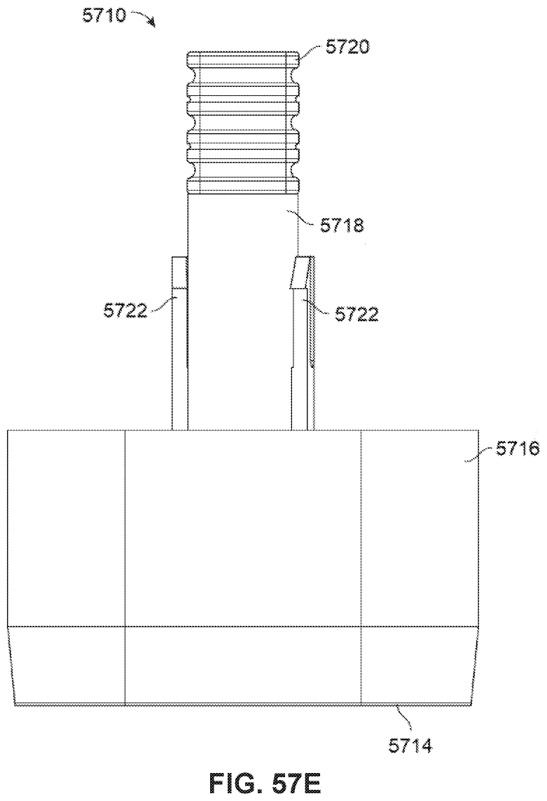

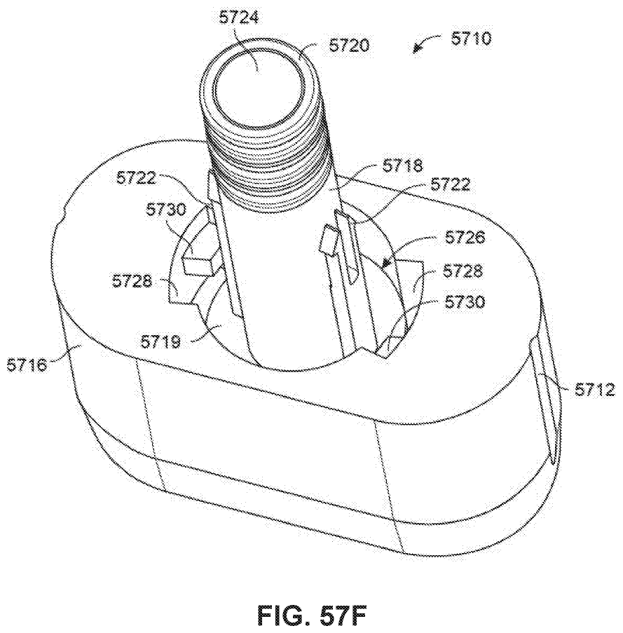

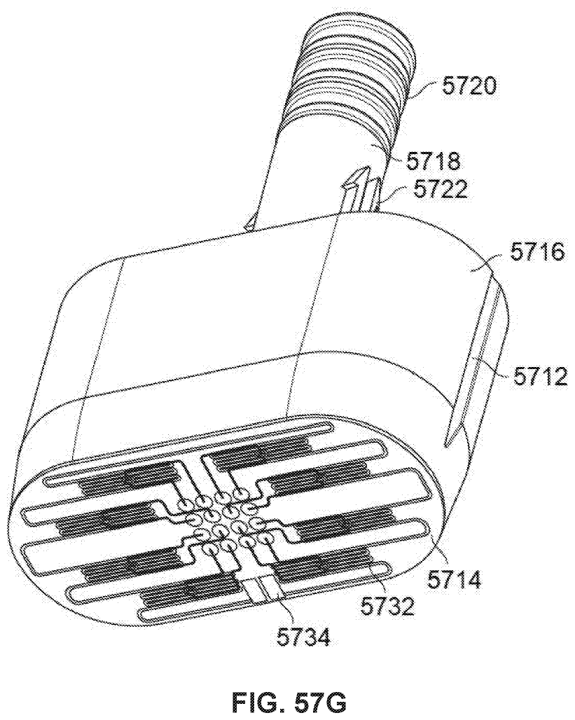

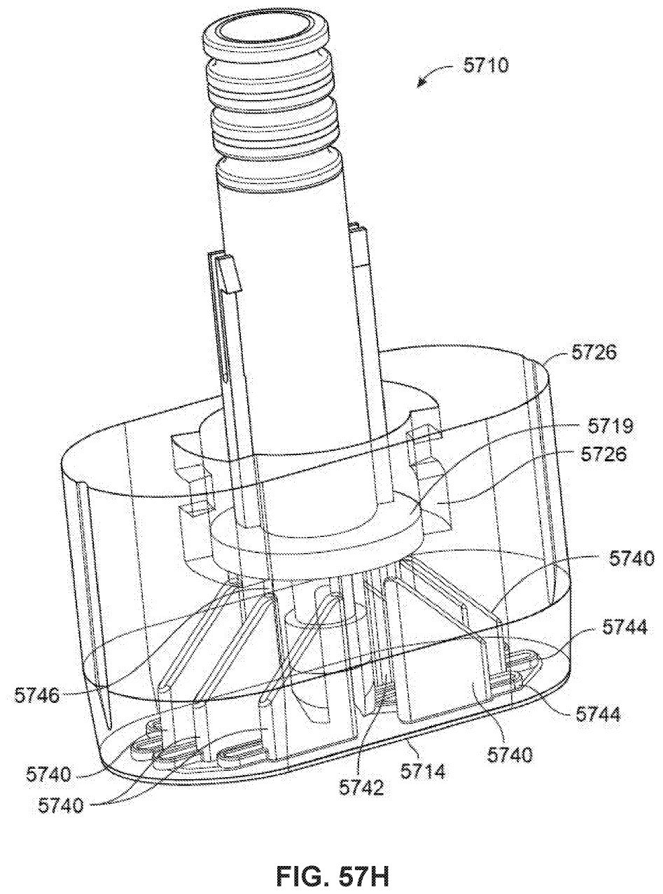

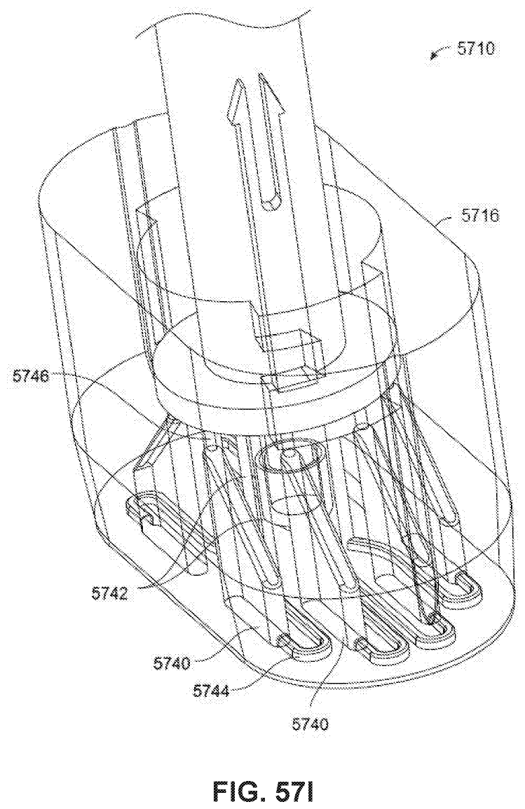

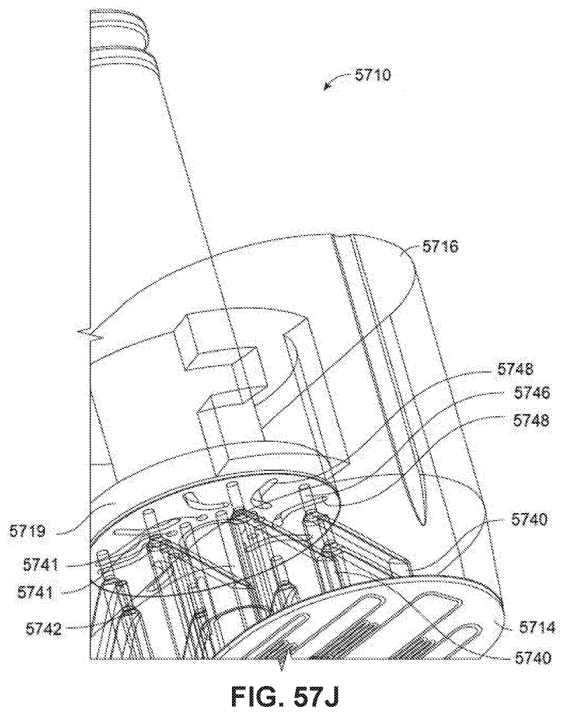

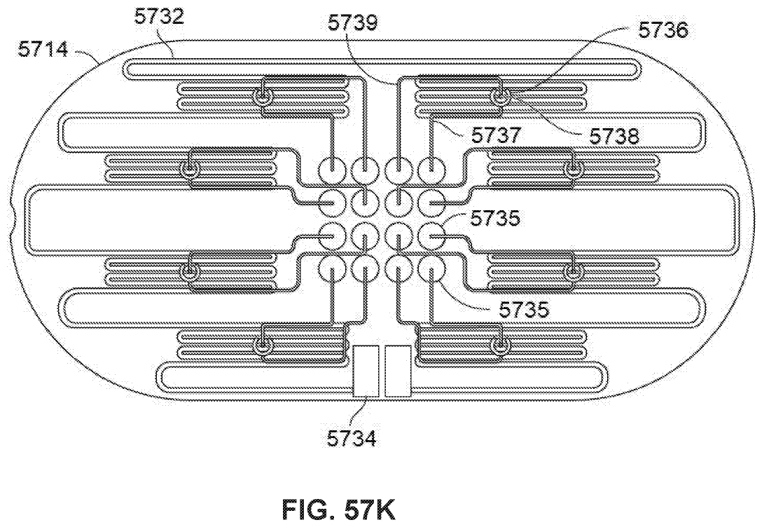

FIGS. 57A-57J depict an example cartridge 5700 configured for detection of a target. As described herein, the target may be a viral target, bacterial target, antigen target, parasite target, microRNA target, agricultural analyte, nucleic acid target, DNA target, human DNA target, genome target, or human genome target. Some embodiments of the cartridge 5700 can be configured for testing for a single target, while some embodiments of the cartridge 5700 can be configured for testing for multiple targets. The cartridge 5700 includes a cartridge body 5710 and a cap 5750 configured to be mechanically coupled to the cartridge body 5710. The cartridge body 5710 and the cap 5750, when coupled together, can form an assembled cartridge 5700 for insertion into a reader device such as the reader device 5600 of FIGS. 56A and 56B. As will be described in greater detail below, the cartridge body 5710 may include a plurality of test wells therein, such that a single cartridge 5700 can be configured for testing a single sample for multiple targets.

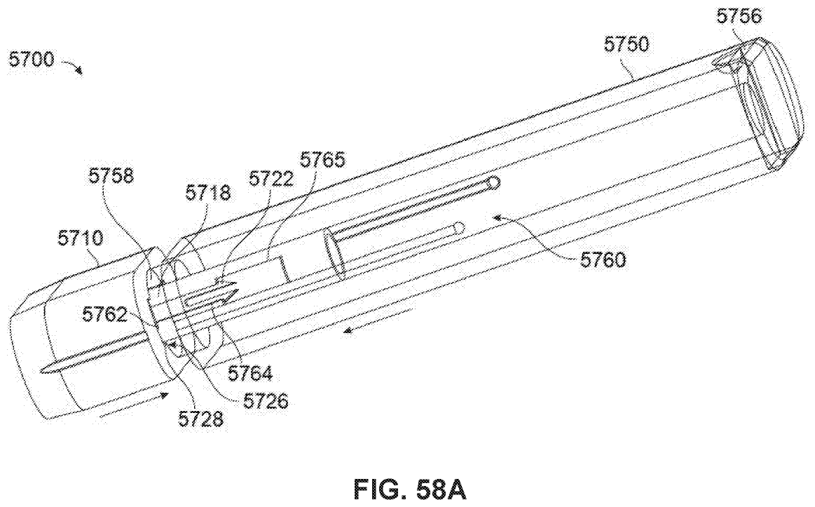

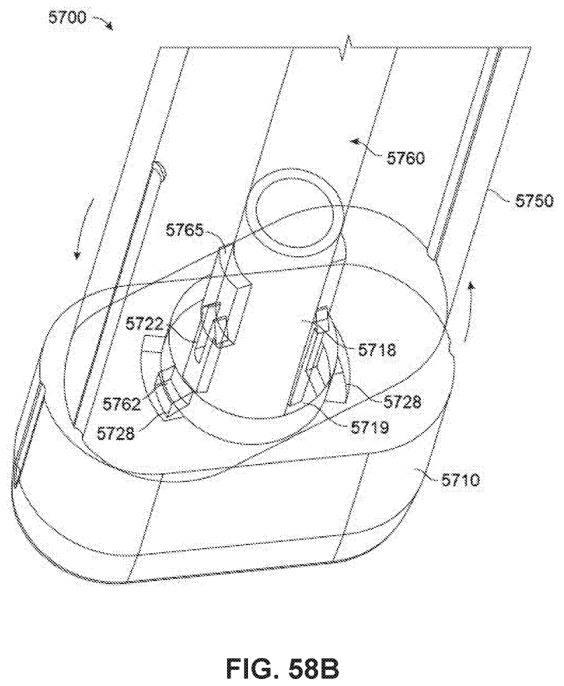

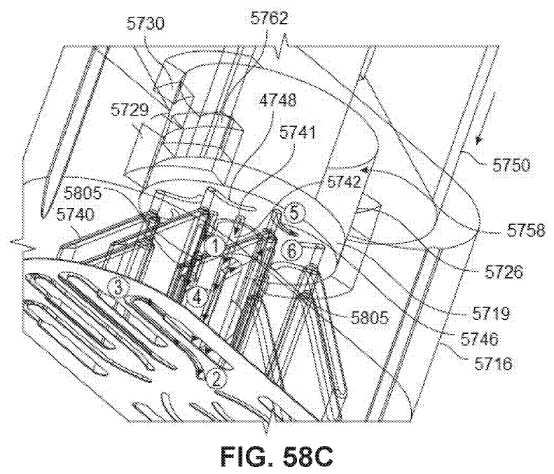

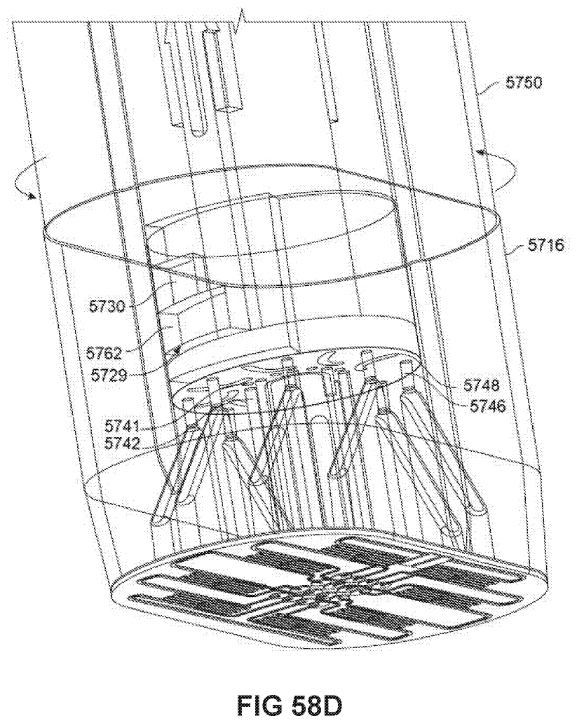

FIGS. 57A and 57B depict a complete cartridge 5700 including the cartridge body 5710 and the cap 5750 coupled together. In use, the cap 5750 and the cartridge body 5710 can operate to seal a provided sample within the cartridge 5700, thereby preventing exposure of test operators to the sample and preventing any liquid from escaping into the electronics of an associated reader device. The cartridge body 5710 and the cap 5750 may be coupled by a friction fit, a snap fit, and/or one or more mechanical or chemical securing means. Coupling of the cartridge body 5710 and the cap 5750 is discussed in greater detail with reference to FIGS. 58A-58D.

The cartridge body 5710 and the cap 5750 can be formed from suitable fluid-impermeable materials such as plastic, metals, or the like, and may be opaque, translucent, or transparent. The cartridge body 5710 can also include a transparent, translucent, or opaque cover surface such as a printed circuit board (PCB) 5714 or other surface partially defining a fluid path within the cartridge body 5710, and one or more electrode interfaces 5735. The PCB 5714, fluid paths, and electrode interfaces 5735 are discussed in greater detail with reference to FIGS. 57E-57J. The cartridge body 5710 and/or the cap 5750 can further include a cartridge identifier 5711. The cartridge identifier 5711 may include human-readable and/or machine-readable information, such as text, a barcode, a QR code, or the like. The cartridge identifier 5711 can include any suitable information associated with the cartridge, such as information specifying a type of test, a target agent, a sample type, a cartridge serial number or other individual cartridge identifier, etc. In addition to serving as an identifier for a user of the type of test associated with the cartridge 5700, the cartridge identifier 5711 may also be scanned by a user (e.g., using a user interface device in communication with a reader device) to communicate one or more test protocols to the reader device.

The cartridge body 5710 and/or the cap 5750 can include ergonomic features such as an indentation or the like to facilitate handling of the cartridge 5700. In the example cartridge 5700 depicted, the cartridge body 5710 further includes an alignment groove 5712 located to align with an alignment groove 5752 of the cap 5750. The alignment groove 5752 of the cap 5750 terminates at a stop 5754 configured to engage a protrusion within a corresponding reader device (e.g., the reader device 5610 of FIGS. 56A and 56B) to define a fully inserted position of the cartridge 5700 within the reader device. The cap 5750 can further include a sample receiving area cap 5756 sized and shaped to sealingly close an opening in the cap 5750 for receiving a swab or other sample carrying holding a sample to be analyzed.

FIGS. 57C and 57D depict the cap 5750 component of the cartridge 5700 of FIGS. 57A and 57B. The cap 5750 comprises an elongate body which is at least partially hollow to receive a sample carrier such as a swab or the like. An opening in the cap 5750 for receiving the sample carrier may be sealed by the sample receiving area cap 5756, which may include one or more O-rings or other resilient structures to sealingly block the opening in the cap 5750.

The cap 5750 further includes a collar 5758 protruding from the cap 5750. The collar 5758 is sized and shaped to facilitate coupling with the cartridge body 5710. The collar 5758 generally comprises a hollow cylindrical body defining a plunger receiving well 5760 through which the fluid sample may pass from the cap 5750 into the cartridge body 5710. The collar 5758 includes interlocking fins 5762 extending radially outward from an exterior surface of the collar 5758, and receiving channels 5764 within the an interior surface of the collar 1058. Each receiving channel 5764 terminates in a widened section 5765 such that the receiving channels 5764 are configured to receive and retain one or more snap-fit connectors of the cartridge body 5710, as will be described with reference to FIGS. 58A-58D.

The cap 5750 may further include one or more liquid constitutents therein to be mixed with a received sample. For example, liquid constituents may include one or more amplification reagents, buffer solutions, water, mucin mitigating agents, or other desired liquid constituents for the testing process. The particular selection and chemistry of these liquids can be tailored to a particular target or targets for which the cartridge 5700 is designed to test. In some embodiments, the liquid constituents may be contained within a blister pack within the cap 5750. The blister pack may be punctured by, for example, insertion of a sample carrier, coupling of the cap 5750 to the cartridge body 5710, etc.

FIGS. 57E-57J depict the cartridge body 5710 component of the cartridge 5700 of FIGS. 57A and 57B. FIGS. 57E-57G are exterior views of the cartridge body 5710. FIGS. 57H-57J depict the cartridge body 5710 with partial translucency to illustrate fluid paths integrally formed therein. Referring to FIGS. 57E-57G, the cartridge body 5710 includes a base 5716 and a hollow plunger 5718 rotatably coupled within a receiving well 5726 of the base such that the plunger 1018 can rotate about its longitudinal axis while being retained within the receiving well 5726.

The plunger 5718 comprises a generally cylindrical body sized and shaped to fit within the plunger receiving well 5760 of the cap 5750 (FIGS. 57C and 57D). A sealing portion 5720 of the plunger 5718 is disposed at a distal end of the plunger 5718 and may include one or more resilient structures (e.g., one or more O-rings, integrally formed elastomeric structures, etc.) having an appropriate diameter to sealingly engage with the interior walls of the plunger receiving well 5760 of the cap 5750. A sacrificial seal 5724 such as a layer of a metallic foil or other thin material may be provided to prevent exposure of the interior of the cartridge body 5710 to the atmosphere prior to use. The plunger 5718 additionally includes one or more snap-fit clips 5722 extending along the exterior of the plunger 5718 parallel to the longitudinal axis of the plunger. The snap-fit clips 5722 are sized and shaped to engage within and be retained by the receiving channels 5764 of the plunger receiving well 5760 of the cap 5750. A plunger baseplate 5719 is rotationally fixed to the plunger 5718 (e.g., may be integrally formed with the plunger 5718). The diameter of the plunger baseplate 5719 may be substantially equal to or slightly larger than the outer diameter of the collar 5758 of the cap 5750.

In some embodiments, one or more liquid constituents may be included within the plunger 5718, instead of or in addition to liquid constituents included within the cap 5750. For example, the sacrificial seal 5724 may contain the liquid constituents within the plunger 5718 and/or the liquid constituents may be contained within a blister pack within the plunger 5718. Liquid constituents contained within the plunger may include one or more amplification reagents, buffer solutions, water, mucin mitigating agents, or other desired liquid constituents for the testing process. The particular selection and chemistry of these liquids can be tailored to a particular target or targets for which the cartridge 5700 is designed to test. The blister pack may be punctured by, for example, insertion of a sample carrier, coupling of the cap 5750 to the cartridge body 5710, etc.

The receiving well 5726 is coaxial with the plunger 5718 and has a generally cylindrical profile with a diameter substantially equal to or slightly larger than the plunger baseplate 5719 and/or the collar 5758 of the cap 5750. The receiving well 5726 further includes cutouts 5728 sized to receive the interlocking fins 5762 of the cap 5750. Stops 5730 within the cutouts 5728 are disposed within the cutouts 5728 to block longitudinal motion of the interlocking fins 5762 in certain rotational positions, as will be described in greater detail with reference to FIGS. 58A-58D.

A printed circuit board (PCB) 5714 or other generally planar layer is disposed along a surface of the cartridge body 5710 opposite the receiving well 5726. In some embodiments, the PCB 5714 may perform heating and/or electrode interface functions, and may further serve as a boundary for one or more fluid paths within the cartridge body 5710. Although the example PCB 5714 depicted herein includes heating and electrode interface functionality, these functions may equally be performed by two or more discrete elements in the cartridge body 5710. In some embodiments, heating may be achieved by heating elements located within a corresponding reader device instead of or in addition to heating elements disposed on or in the cartridge 5700.

The PCB 5714 comprises a generally planar surface having one or more traces disposed thereon in one or more layers. For example, the PCB 5714 may include one or more flex circuits, rigid printed circuit boards, or any other suitable circuitry including one or more current paths disposed on a generally planar substrate. One or more heating traces 5732 electrically connect test well heating elements 5733 to heating current pads 5734. The heating current pads 5734 may come into contact with contacts of a current source of a reader device when the cartridge 5700 is inserted into the reader device, such that a current may be provided to the test well heating elements 5733 to heat fluid samples in one or more test wells of the cartridge body 5710.

The PCB 5714 further comprises a pair of electrodes 5736, 5738 (e.g., an excitation electrode and a sensor electrode) corresponding to each test well. In some embodiments, the electrodes 5736, 5738 may be in direct contact with the fluid sample in each test well if the PCB 5714 serves as a boundary for the test wells. Each electrode 5736, 5738 is electrically connected to an electrode interface pad 5735 by electrode traces 5737, 5739 of the PCB 5714.

In various embodiments, the PCB 5714 may include one or more layers. For example, in some embodiments the PCB is a flex circuit comprising an electrode layer and a heating layer separated from the electrode layer. The electrode layer may include the electrodes 5736, 5738 as well as the electrode traces 5737, 5739 and/or the electrode interface pads 5735. The heating layer may include the heating elements 5733, heating traces 5732, and/or heating current pads 5734. In some aspects, the electrode layer and the heating layer may be disposed on opposite sides of a common substrate, or may be provided on separate substrates. Preferably, the PCB 5714 may be disposed such that the electrode layer including the electrodes 5736, 5738 is adjacent to the cartridge body 5710 and the electrodes 1036, 1038 are fluidically connected to the test wells 5740.

FIGS. 57H-57J depict additional views of the cartridge body 5710 in which the base 5716 is illustrated with transparency to show the fluid paths contained therein. The base 5716 may comprise any suitable liquid-impermeable material, such as plastic or metal. The base 5716 may be formed by one or more processes such as injection molding, die casting, milling, or the like, such that the depicted fluid paths can be integrally formed therein.

The base 5716 of FIGS. 57H-57J includes eight substantially identical fluid paths, each fluid path including a test well 5740. Various embodiments may include fewer than eight or more than eight fluid paths and test wells 5740 without departing from the scope of the present disclosure. For example, a base 5716 may include 1, 2, 3, 4, 5, 6, 7, 9, 10, 11, 12, or more fluid paths. Multiple identical or similar fluid paths within the base may accommodate simultaneous testing for a plurality of different targets and/or a plurality of simultaneous tests for the same target (e.g., to improve reliability of results).

Each fluid path includes an inlet channel 5742, a lateral channel 5744, a test well 5740, and an outlet channel 5746. Each inlet channel 5742 extends vertically through the base 5716 to fluidically connect a first end adjacent to the plunger baseplate 5719 to an opposite end adjacent to the PCB 5714. Each lateral channel 5744 fluidically connects an inlet channel 5742 to the corresponding test well 5740. Each outlet channel 5746 extends vertically from a test well 5740 to fluidically connect the test well 5740 to the bottom of the plunger baseplate 5719. In the cartridge body 5710 of FIGS. 57H-57J, the PCB 5714 forms a boundary partially defining each lateral channel 5744 and each test well 5740. In this example embodiment, the PCB 5714 may be oriented with the electrodes 5736, 5738 on the side adjacent to the cartridge body 5710 such that the electrodes 5736, 5738 are in contact with the interior of the test wells 5740.

As shown in FIG. 57J, each inlet channel 5742 is disposed radially outward from the longitudinal axis of the plunger baseplate 5719 at substantially the same distance as an array of sample inlets 5741 of the plunger baseplate 5719. Similarly, each outlet channel 5746 is disposed radially outward from the longitudinal axis of the plunger baseplate 5719 at substantially the same distance as the outer ends of an array of J-shaped sample outlets 5748 of the plunger baseplate 5719. The sample inlets 5741 and the inner ends of the J-shaped sample outlets 5748 are fluidically connected through the plunger baseplate 5719 to one or more interior spaces within the plunger 5718. Accordingly, when the plunger baseplate 5719 is rotated to an engaged position, as will be described with reference to FIGS. 58B and 58C, the sample inlets 5741 align with the inlet channels 5742, and the sample outlets 5748 align with the outlet channels 5746, such that a fluid sample within the plunger 5718 can flow into and fill the fluid paths within the base 5716 of the cartridge body 5710.

FIGS. 58A-58D illustrate mechanical fluid transfer aspects of the cartridges 5620, 5700 described herein. Similar to the fluid transfer aspects described with reference to FIGS. 53A-53E, the cartridge body 5710 and cap 5750 are configured to create air pressure when coupled together, such that the air pressure propels a fluid sample through the fluid paths of the cartridge body 5710. The cap 5750 is illustrated with translucency in FIGS. 58A-58D, and the cartridge body 5710 is illustrated with translucency in FIGS. 58C and 58D, to reveal interior features of the cap 5750 and cartridge body 5710. The cartridge body 5710 is depicted with the PCB 5714 removed in FIG. 58C.

With reference to FIG. 58A, a fluid sample may be received within the cap 5750. For example, a swab or other sample carrier may be inserted into an opening of the cap 5750 opposite the cartridge body 5710, and the opening may then be sealed by the sample receiving area cap 5756 to contain the sample within the cap (e.g., within the plunger receiving well 5760 or other internal space within the cap 5750. When the fluid sample has been sealed within the cap 5750, the process of FIGS. 58A-58D may be used to mechanically couple the cartridge body 5710 to the cap 5750 and move the fluid sample into the cartridge body 5710.

As shown in FIG. 58A, the mechanical coupling of the cartridge body 5710 and the cap 5750 begins by inserting the plunger 5718 of the cartridge body 5710 into the plunger receiving well 5760 of the cap 5750. The sealing portion 5720 of the plunger 5718 can sealingly engage with the interior of the plunger receiving well 5760 to trap and begin compressing a volume of air within the plunger receiving well 5760. As the plunger 5718 slides into the plunger receiving well 5760, the snap-fit clips 5722 slide within the receiving channels 5764 until they pass into the widened sections 5765 of the receiving channels 5764, where they are longitudinally retained. Retention of the snap-fit clips 5722 within the receiving channels 5764 prevents removal of the cap 5750 from the cartridge body 5710, and further locks the cap 5750 rotationally with the plunger 5718, thereby allowing a user to rotate the plunger 5718 and plunger baseplate 5719 by rotating the cap 5750, which may be relatively large and easy to manipulate manually. The cap 5750 and cartridge body 5710 may slide together until the collar 5758 of the cap 5750 is partially within the receiving well 5726 of the cartridge body 5710 and the interlocking fins 5762 of the collar 5758 contact the stops 5730 (FIG. 57F) within the cutouts 5728.

As shown in FIG. 58B, the cap 5750, plunger 5718, and plunger baseplate 5719 may then be rotated about the longitudinal axis. Because the snap-fit clips 5722 rotationally fix the plunger 5718 to the cap 5750, rotation of the plunger 5718 and plunger baseplate 5719 may be achieved by rotating the cap 5750. The cap 5750 may rotate until the interlocking fins 5762 are blocked by a lateral side of the cutouts 5728 of the receiving well 5726. In the example cartridge 5700, the cutouts 5728 and interlocking fins 5762 are sized to allow a total rotation of approximately 22.5° while the interlocking fins 5762 are within the cutouts 5728. However, other example cartridges may function with a different range of rotational motion, such as between approximately 5° and approximately 90°, between approximately 10° and approximately 45°, between approximately 150 and approximately 30°, between approximately 20° and approximately 25°, or any angle or subrange of angles therebetween. In some embodiments in which liquid constituents are contained in a blister pack within the cap 5750 and/or the plunger 5718, the rotation of components in FIG. 58B may cause the blister pack to be punctured so as to release the liquid constituents to mix with the sample.

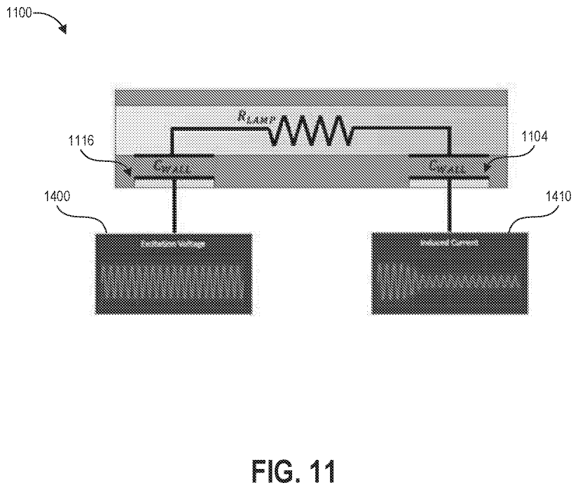

With reference to FIG. 58C, the cap 5750 may be moved downward along the longitudinal axis, such that the collar 5758 moves further into the receiving well 5726. Because the stops 5730 only extend along a portion of the cutouts 5728, the rotational motion described with reference to FIG. 58B moves the interlocking fins 5762 clear of the stops 5730 such that the interlocking fins 5762 can move to an interior portion 5729 of the cutouts 5728. As shown in FIG. 58C, the rotated position of the plunger baseplate 5719 substantially aligns the inlet channels 5742 and the outlet channels 5746 with the sample inlets 5741 and the sample outlets 1048, respectively. When the cap 5750 is pressed further onto the cartridge body 5710 to the position of FIG. 11C, the sample carrier and/or one or more internal structures within the cap 5750 may mechanically contact and rupture a seal on or within the plunger 5718 (e.g., the sacrificial seal 5724 of FIG. 57F), thereby allowing the trapped air compressed by the plunger 5718 to flow into the interior of the plunger and propel the fluid sample through the plunger baseplate 5719 and into the fluid paths of the cartridge body 5710. In some embodiments in which liquid constituents are contained in a blister pack within the cap 5750 and/or the plunger 5718, the longitudinal motion of components in FIG. 58C may cause the blister pack to be punctured so as to release the liquid constituents to mix with the sample.

The flow of the fluid sample through the fluid paths of the cartridge body 5710 will now be described with continued reference to FIG. 58C. FIG. 58C illustrates the flow of a portion of a fluid sample through a single example fluid path 5805 within the cartridge body 5710 with encircled numbers shown as labels for certain points along the fluid path. The encircled numbers are discussed below as example steps of a progression of a fluid sample as it travels through the flow path 5805 within the cartridge body 5710, with each step including a directional arrow showing the direction of fluid travel at that step.

At step (1), as the compressed air is allowed to flow into the plunger 5718, the fluid sample is forced through the sample inlet 5741 into the inlet channel 5742. The fluid sample travels along the inlet channel 5742 toward the lateral channel 5744.

At step (2), the fluid sample reaches the PCB 5714 boundary of the fluid path, and begins traveling parallel to the PCB 5714 within the lateral channel 5744. At step (3), the fluid sample continues through the curved lateral channel 5744 toward the test well 5740.

At step (4), the fluid sample enters the test well 5740. The test well 5740 may contain one or more reagents. Agitation caused by the turbulent flow of the fluid sample within the relatively larger space of the test well 5740 causes the reagent and the sample to be mixed. In some embodiments, the reagent and the fluid sample are mixed into a homogeneous solution in which the reagent is evenly distributed throughout the fluid sample. The depth, width, shape, and/or cross-sectional profile of the test well 5740 may be selected to facilitate mixing of the reagent and the fluid sample.

At step (5), any excess fluid sample is pushed from the test well 5740 into the outlet channel 5746 and enters the outer end of the J-shaped sample outlet 5748. As the excess fluid sample reaches the inner end the sample outlet 5748, it passes through a corresponding opening in the plunger baseplate 5719 at step (6) and is vented into the plunger 5718. In some embodiments, the interior volume of the plunger 5718 and/or the cap 5750 is separated from the interior volume that is fluidically connected to the sample inlet 5741, so as to create a directional flow of fluid sample along the fluid path 5805.

After the cap 5750 is pressed fully onto the cartridge body 5710 as shown in FIG. 58C, initiating the flow of the fluid sample through the fluid path within the cartridge body, the cartridge 5700 may reach a pressure equilibrium as the compressed air forces the fluid sample into the fluid path 5805, the fluid path 5805 is filled with the fluid sample, and a portion of the sample is vented back into the plunger 5718 and/or the cap 5750. The pressure equilibrium may be reached relatively quickly, for example, within 10 seconds, 5 seconds, 2 seconds, 1 second, or less.

Referring now to FIG. 58D, when the fluid path 5705 is filled, the cap 5750, plunger 5718, and plunger baseplate 5719 may again be rotated relative to the cartridge body 5710. In the example process of FIGS. 58A-58D, the above components are rotated by the same angular displacement, but in the opposite direction, relative to the rotation of FIG. 58B. Thus, as shown in FIG. 58D, the plunger baseplate 5719 rotates relative to the fluid path 5805 such that the inlet channels 5742 and outlet channels 5746 are no longer aligned with the sample inlets 5741 and sample outlets 5748, thereby sealing the fluid path 5805 and retaining the fluid sample within the test wells 5740 for testing. This final rotation step additionally causes the interlocking fins 5762 of the cap 5750 to be retained under the stops 5730 within the interior portion 5729 of the receiving well cutouts 5728, completing and securing the mechanical coupling of the cartridge body 5710 and cap 5750. Moreover, the final rotation step substantially aligns the exterior profiles of the cartridge body 5710 and the cap 5750 such that the assembled cartridge 5700 can be inserted into a reader device to perform one or more tests on the fluid sample contained therein.

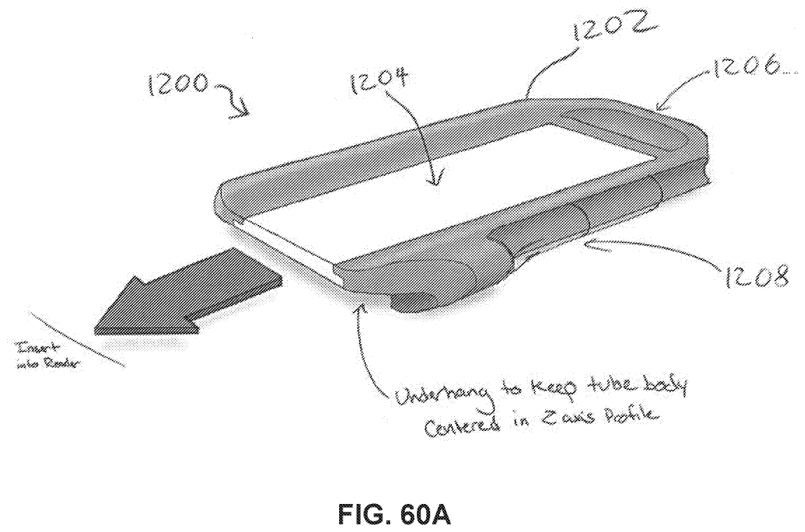

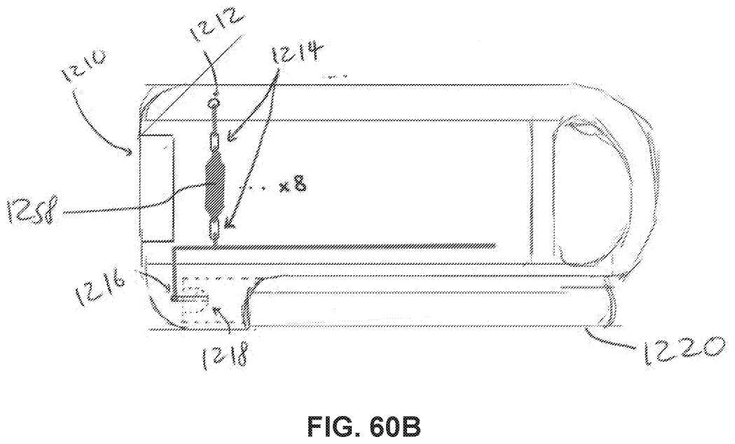

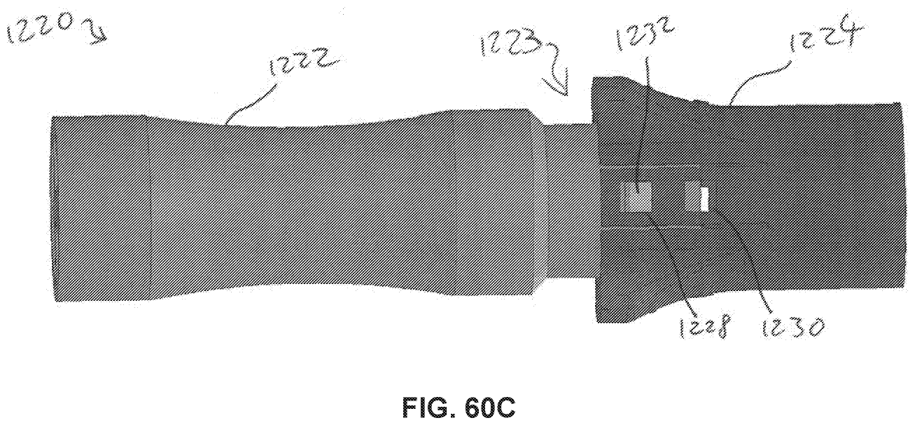

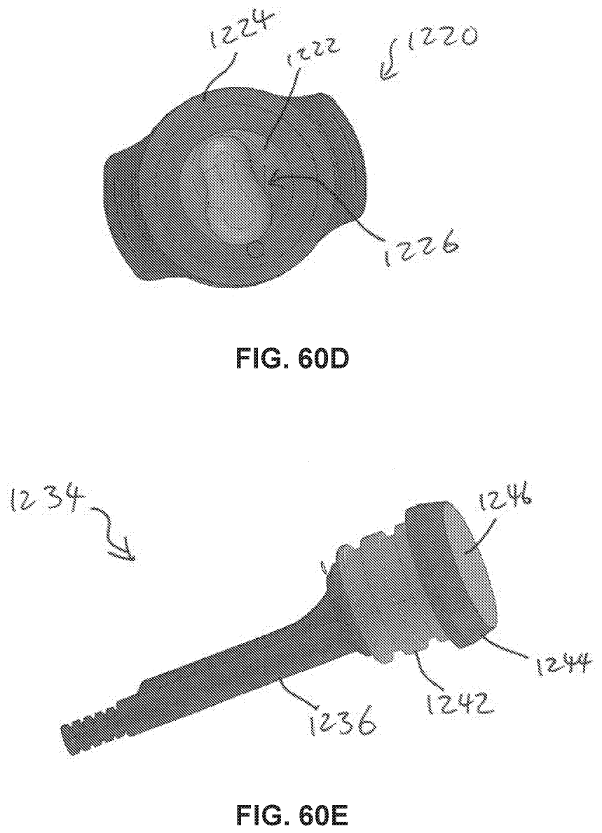

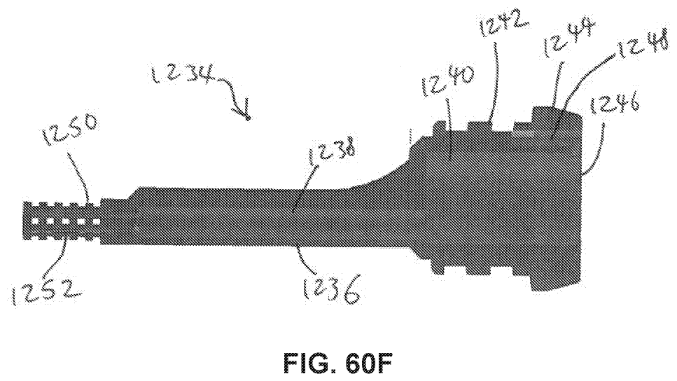

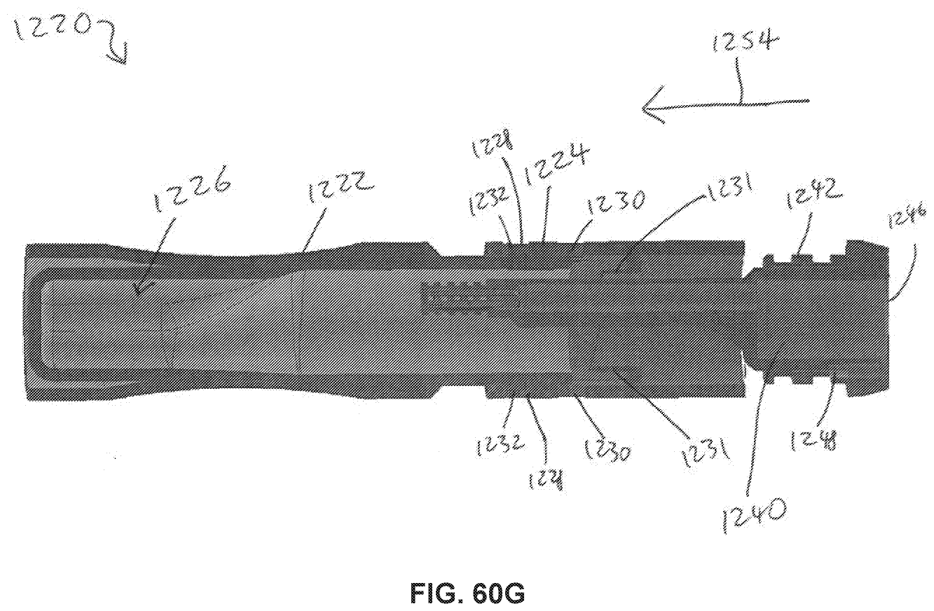

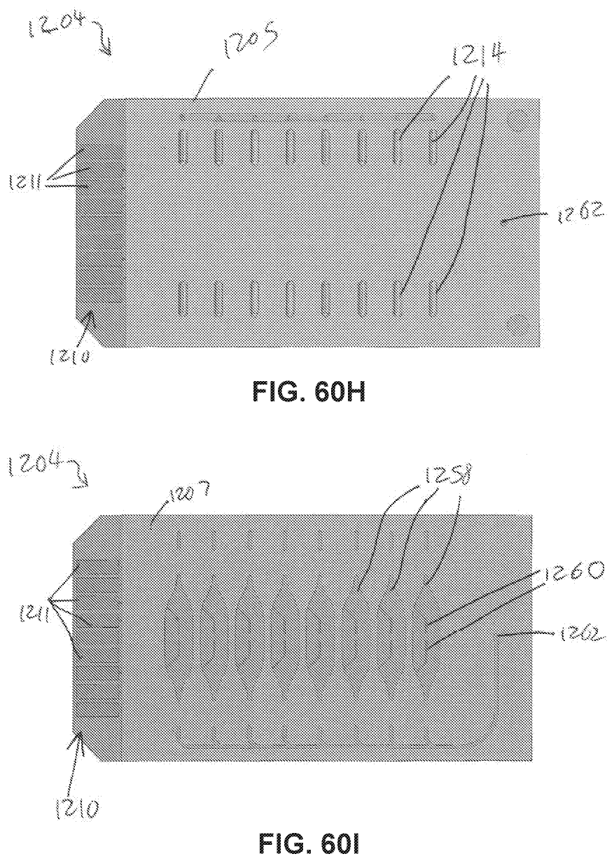

FIGS. 60A-60I illustrate a further embodiment of a cartridge 1200 configured for detection of a target. As described herein, the target may be a viral target, bacterial target, antigen target, parasite target, microRNA target, or agricultural analyte. Some embodiments of the cartridge 1200 can be configured for testing a single target, while some embodiments of the cartridge can be configured for testing for multiple targets. The cartridge 1200 includes a cartridge body 1202 and a swab assembly 1220 configured to be mechanically coupled to the cartridge body 1202 at a swab assembly insertion point 1208.

The cartridge body 1202 includes a thin film testing assembly 1204 and an ergonomic frame 1206 configured to be grasped by a user. The thin film testing assembly 1204 generally includes a plurality of test wells 1258, pinch valves 1214 for isolating fluid within the test wells 1258, a gas permeable filter 1212 such as a membrane or the like, and an electrode interface 1210 for electrically connecting electrodes at the test wells to circuitry of a reader device. The cartridge further includes a fluidic piston 1218 and a transition point 1216 for introducing a fluid sample from the swab assembly 1220 into the thin film testing assembly 1204. The features of the thin film testing assembly 1204 are discussed in greater detail with reference to FIGS. 60H and 60I.

Referring now to FIGS. 60C-60G, the swab assembly 1220 includes a tube 1222, a slider 1224, and a cap 1234 configured to fit together to form a substantially sealed swab assembly 1220. The tube 1222 includes a tube channel 1226 sized and shaped to receive a shaft 1236 of the cap 1234. The tube channel 1226 may be sealed, such as with a foil seal or the like, to contain one or more liquid reagents, buffers, etc., during shipping and/or prior to use of the swab assembly 1220. The tube channel 1226 may have an hourglass profile, a dual lobe profile, or other shape configured to facilitate mixing of fluids therein. In some embodiments, the tube channel 1226 may further include interior threading or other protruding features further configured to facilitate mixing of fluids with in the tube channel 1226. The slider 1224 comprises a hollow structure configured to fit around an engagement end 1223 of the tube 1222. The tube 1222 further includes one or more snap-fit clips 1232 configured to interlock with first and second snap-fit openings 1228 and 1230 of the slider 1224.

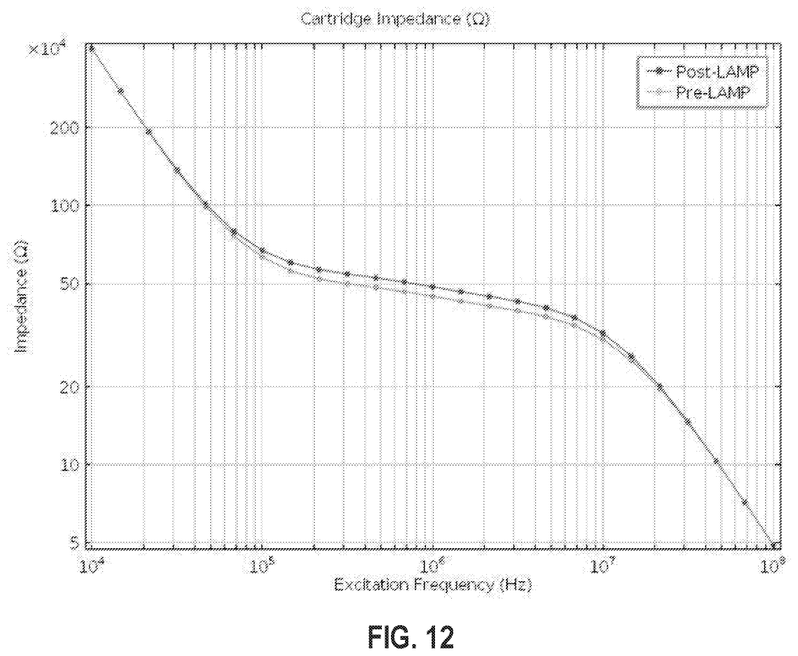

FIGS. 60E and 60F illustrate the cap 1234. FIG. 60E is a perspective view of the cap. FIG. 12F is a cross-sectional view of the cap illustrating internal components thereof. The cap 1234 comprises a hollow shaft 1236 surrounding a cap channel 1238, and a hollow upper section 1244 surrounding a metered volume 1240. The upper section 1244 may further include a sealing portion 1242 comprising a resilient material (e.g., rubber, a resilient plastic, or other elastomeric material) sized and shaped to sealingly engage the interior of the slider 1224. A foil seal 1246 may seal one or more liquid or dried reagents (such as an amplification reaction solution, RPA reagent solution, or reagent solution configured for a first or second isothermal amplification reaction) within the cap 1234. A vent 1248 may be fluidically connected to the metered volume 1240 to allow any gas trapped within the cap 1234 to be vented. The shaft 1236 terminates at a cap inlet 1250 fluidically connected to the cap channel 1238, which may include one or more sections of a filter 1252 configured to allow fluid flow into the cap channel 1238. In some embodiments, an additional seal may be provided over the cap inlet 1250 to seal any liquid or dried reagents within the cap 1234 prior to use.

Referring jointly to FIGS. 60C-60G, and particularly with reference to FIG. 60G, an example process of introducing a sample to the swab assembly 1220 will now be described. Prior to introduction of the sample, the cap 1234 is separate from the tube 1222 and the slider 1224. The tube 1222 includes a liquid comprising one or more liquid reagents, buffers, or the like, sealed within the tube 1222 by a seal at the engagement end 1223 of the tube 1222. The cap 1234 includes one or more additional liquid reagents, buffers, or the like, sealed within the cap 1234 by the foil seal 1246. In the initial configuration, the snap-fit clips 1232 are engaged in first snap-fit openings 1228.

A sample (e.g., a nasal swab or other swab-collected sample) may be received on a swab. Prior to inserting the swab into the swab assembly 1220, the slider 1224 is moved along a first direction 1254 relative to the tube 1222 such that the snap-fit clips 1232 of the tube 1222 engage with the second snap-fit openings 1230. This motion may cause an internal structure 1231 of the slider 1224 to break the foil seal at the opening of the tube to expose the liquid reagents or buffers contained within the tube channel 1226.

When the tube channel 1226 is exposed, the swab may be introduced into the tube channel 1226 such that the sample on the swab mixes with the liquid reagents within the tube channel 1226. The interior profile and/or other mixing features of the tube channel 1226 may facilitate mixing of the sample with the liquid reagents to form a test fluid. In some embodiments, the swab may be broken off from a handle such that the portion of the swab containing the sample remains within the tube channel 1226.

After the sample has been introduced to the tube channel 1226 to form the test fluid, the cap 1234 may be mechanically coupled to the tube 1222 and slider 1224 to complete the swab assembly 1220. If a seal is provided around the cap inlet 1250, the seal may be removed. The shaft 1236 of the cap is inserted through the slider 1224, and the cap 1234 is pushed into the slider 1224 and the tube channel 1222 such that the sealing portion 1242 sealingly engages with the interior of the slider 1242. As the cap 1234 continues to move along the direction 1254, air and fluid are compressed within the tube channel 1222 to drive the mixed test fluid through the cap inlet 1250 and the cap channel 1238 into the metered volume 1240. Any gas, such as air, present within the metered volume 1240 may be vented externally through the vent 1248. The filter 1252 at the cap inlet 1250 prevents solids, such as solids within the swab sample or pieces of the swab itself, from entering the cap 1234. The location of the cap inlet 1250 at an end of the tube channel 1226 distal from the metered volume 1240 may advantageously cause the inlet 1250 to receive an optimal portion of the test fluid in the event that the test fluid has not quite achieved a homogeneous mixture. When the cap 1234 has been sealingly inserted into the tube 1222 and slider 1224, the swab assembly 1220 is fully assembled, and any liquid therein is retained within the swab assembly 1220 by the foil seal 1246 of the cap 1234. The swab assembly 1220 may then be placed into the swab assembly insertion point 1208 of the cartridge 1200 to introduce the test fluid to the thin film testing assembly 1204.

Referring now to FIGS. 60B, 60H, and 60I, the thin film testing assembly 1204 includes a substrate 1207 surrounding a plurality of test wells 1258. One boundary of the test wells 1258 is formed by a cover film 1205 including a plurality of pinch valves 1214 which form a portion of a fluid flow path into the thin film testing assembly 1204. A pair of electrodes 1260, which may be formed consistent with any of the electrodes described elsewhere herein, are provided within each test well 1258 and electrically connected to electrode connection pads 1211 of the electrode interface 1210 for connection to a reader device.

When the swab assembly 1220 is inserted into the swab assembly insertion point 1208 of the cartridge 1200, the fluid within the metered volume 1240 of the swab assembly 1220 flows through the transition point 1216 and along a fluid path within the thin film testing assembly 1204 to fill the test wells 1258. Any gas such as air within the thin film testing assembly 1204 may be displaced from the test wells 1258 and vented at a vent 1262 and/or at the gas permeable filter 1212.

The cartridge 1200 may then be inserted into a reader device sized and shaped to receive the cartridge 1200. As the cartridge 1200 is inserted into the reader device, electrical contacts within the reader device come into contact with the electrode connection pads 1211 of the cartridge 1200. In addition, the opening within the reader device for receiving the cartridge 1200 has a width selected such that an interior surface of the reader device compresses and/or crushes the pinch valves 1214, preventing fluid flow therethrough after the cartridge 1200 has been inserted into the reader device. In some embodiments, the pinch valves 1214 may comprise a thermoformed plastic or other material selected such that the pinch valves 1214 can be compressed and closed off without breaking and allowing the test fluid to escape. When the pinch valves 1214 are compressed and/or crushed, the test fluid within each test well 1258 is fluidically isolated within the test well 1258 for testing. Heating and testing of the test fluids in the test wells 1258 may then proceed as described above with reference to the cartridges 200, 1000. In some embodiments, the test wells 1258 may be pre-loaded with one or more primers (e.g., spot-dried, powdered, or other non-liquid primers) or other reagents corresponding to the tests to be performed and/or target agents to be detected in each test well 1258. Some or all of the test wells 1258 may include the same or different primers as the primers present in the other test wells 1258, depending on the individual test to be performed and/or target agents to be detected in each test well 1258.

### Overview of Example Devices

Some embodiments of the methods, systems and compositions provided herein include devices comprising an excitation electrode and a sensor electrode. In some embodiments, the excitation electrode and the sensor electrode measure electrical properties of a sample. In some embodiments, the electrical properties comprise complex admittance, impedance, conductivity, resistivity, resistance, or a dielectric constant.

In some embodiments, the electrical properties are measured on a sample having electrical properties that do not change during the measurement. In some embodiments, the electrical properties are measured on a sample having dynamic electrical properties. In some such embodiments, the dynamic electrical properties are measured in real-time.

In some embodiments, an excitation signal is applied to the excitation electrode. The excitation signal can include direct current or voltage, and/or alternating current or voltage. In some embodiments, the excitation signal is capacitively coupled to/through a sample. In some embodiments, the excitation electrode and/or the sensor electrode is passivated to prevent direct contact between the sample and the electrode.

In some embodiments, parameters are optimized for the electric properties of a sample. In some such embodiments, parameters can include the applied voltage, applied frequency, and/or electrode configuration with respect to the sample volume size and/or geometry.

In some embodiments, the voltage and the frequency of the excitation voltage may be fixed or varied during the measurement. For example, measurement may involve sweeping voltages and frequencies during detection, or selecting a specific voltage and frequency which may be optimized for each sample. In some embodiments, the excitation voltage induces a current on the signal electrode that is can vary with the admittance of the device and/or sample characteristics.

In some embodiments, the detection parameters are optimized by modeling the admittance, device and sample by the lumped-parameter equivalent circuit consisting of electrode-sample coupling impedances, sample impedance, and inter-electrode parasitic impedance. Parameters of the lumped-parameter equivalent circuit is determined by measuring the admittance of the electrode-sample system at one or many excitation frequencies for a device. In some embodiments, the complex (number having both real and imaginary components) admittance of the electrode-sample system is measured using both magnitude- and phase-sensitive detection techniques. In some embodiments, the detection parameters are optimized by determining the frequencies corresponding to the transitions between the frequency regions by measuring the admittance across a wide range of frequencies. In some embodiments, the detection parameters are optimized by determining the frequencies corresponding to the transitions between the frequency regions by computing from the values given lumped-parameter model.

In some embodiments, the admittance of a capacitively-coupled electrode-sample system comprises three frequency regions: a low frequency region dominated by the electrode-sample coupling impedance, a mid-frequency region dominated by the sample impedance, and a high frequency region dominated by parasitic inter-electrode impedance. The admittance in the electrode-sample coupling region is capacitive in nature and is characterized by a magnitude that increases linearly with frequency, whose phase is ninety degrees. The admittance in the sample region is conductive in nature and is characterized by an admittance that does not vary significantly with respect to frequency, whose phase is approximately zero degrees. The admittance inter-electrode region is capacitive in nature and is characterized by a magnitude that increases linearly with frequency and a phase of ninety degrees.

In some embodiments, an induced current at the pick-up electrode is related to the excitation voltage and complex admittance by the relation:

current=(complex admittance)×(voltage)

In some embodiments, the device measures both the excitation voltage magnitude and induced current magnitude to determine the magnitude of the complex admittance. In some embodiments, the device is calibrated to known excitation voltages and measure the magnitude of the induced current. In order to determine the phase of complex admittance, the device may measure the relative phase difference between the excitation voltage and the induced current.

In some embodiments, the magnitude and phase are measured directly.

In some embodiments, the magnitude and phase are measured indirectly e.g., by using both synchronous and asynchronous detection. The synchronous detector gives the in-phase component of the induced current. The asynchronous detector gives the quadrature component of the induced current. Both components can be combined to determine the complex admittance.

In some embodiments, the electrodes are not passivated.

In some embodiments, the excitation and/or detection electrodes are passivated. The excitation and/or detection electrodes may be passivated to prevent e.g., undesirable adhesion, fouling, adsorption or other detrimental physical interactions between the electrode with the sample or components therein. In some embodiments, the passivation layer comprises a dielectric material. In some embodiments, passivation enables efficient capacitive coupling from the electrodes to the sample. The efficiency of the coupling is determined by measuring the characteristics of the electrode/sample system, for example, which may include: the dielectric properties of the passivation layer, the thickness of the passivation layer, the area of the passivation/sample interface, the passivation surface roughness, the electric double layer at the sample/passivation interface, temperature, applied voltage and applied frequency, the electrical properties of the sample, the electric and/or chemical properties of the electrode materials.

In some embodiments, the electrode configuration and fabrication is optimized to mitigate undesirable parasitic coupling between electrodes. This may be accomplished through electric field shielding, the use of a varying dielectric constant electrode substrate, layout optimization, and/or grounding layers.

Overview of Example Devices for detection of biomolecules

Some embodiments of the methods, systems and compositions provided herein include devices for the detection of a target, such as a biomolecule. In some such embodiments, measurement of the electrical properties of a sample is used as a detection strategy for biomolecular assays.

In some embodiments, the target is a nucleic acid, protein, small molecule, drug, metabolite, toxin, parasite, intact virus, bacteria, or spore or any other antigen, which may be recognized and/or bound by a capture and/or detection probe moiety. In some embodiments, the carbohydrate is detectable by a carbohydrate binding protein such as galectin or lectin.

In some embodiments, the target is a nucleic acid. In some embodiments, methods comprise nucleic acid amplification. In some embodiments, amplification comprises isothermal amplification, such as LAMP. In some embodiments, a nucleic acid amplification reaction is quantified by measuring the electric properties, or change therein, of the reaction solution. In some embodiments, the electrical properties of the amplification reaction are measured in real-time over the course of the reaction, or comparison measurements are made using before and after reaction electrical property measurements.

In some embodiments, a target antigen is detected via the specific binding of a detection probe such as e.g., an antibody, aptamer or other molecular recognition and/or binding moiety to the antigen. In an example embodiment, a detection antibody is linked to a nucleic acid sequence to form an antibody-nucleic acid chimeric complex. The chimeric complex is synthesized prior to the assay for the purpose of detecting the antigen. Many different nucleic acids may be conjugated to a single antibody thereby increasing the sensitivity for detection of binding of the chimeric complex to the antigen. After removing any excess chimeric complex not bound to antigen, the nucleic acid portion of the chimeric complex is amplified and the amplification reaction is quantified via the measurement of the electric properties (or changes therein) of the reaction solution as described herein. In this way, the degree of amplification of the nucleic acids, which are bound to the antigen through the chimeric complex signifies the presence of the target antigen and permits quantitation of antigen. The use of secondary amplification representative of antigen recognition, in combination with electrical detection, allows for greater ease, sensitivity and dynamic range than other antigen detection methods.

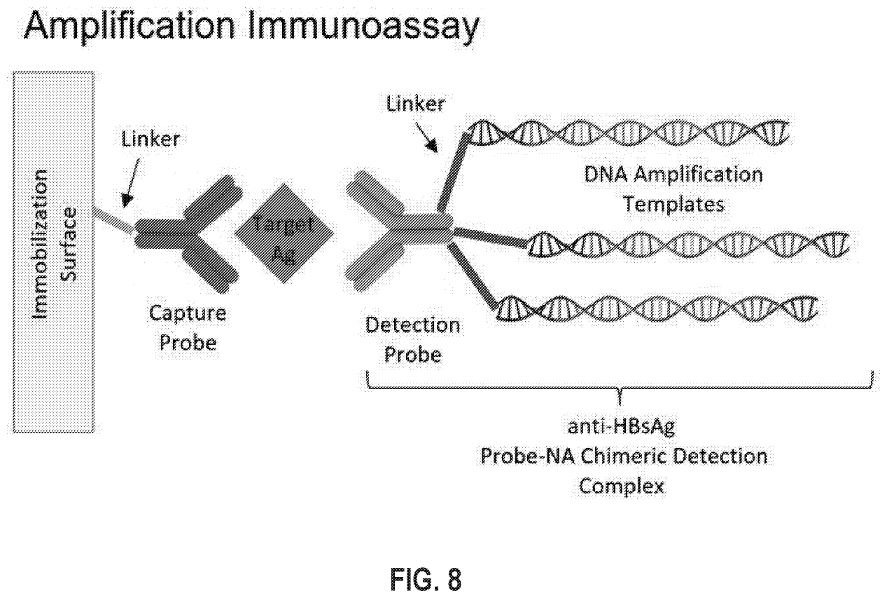

In some embodiments, a capture probe such as an antibody, aptamer or other molecular recognition and/or binding moiety to an antigen is bound to a surface by a conjugation or linkage. The immobilization of the capture probe onto a surface allows for the removal of excess, unbound reagents and/or antigen through washing. The chimeric complex is bound to the surface captured antigen enabling unbound chimera complex to be removed by washing. In this way, only captured antigen is retained for detection by the chimera complex. An example embodiment is depicted in FIG. 8. In some embodiments, the capture probe and the detection antibody are the same.

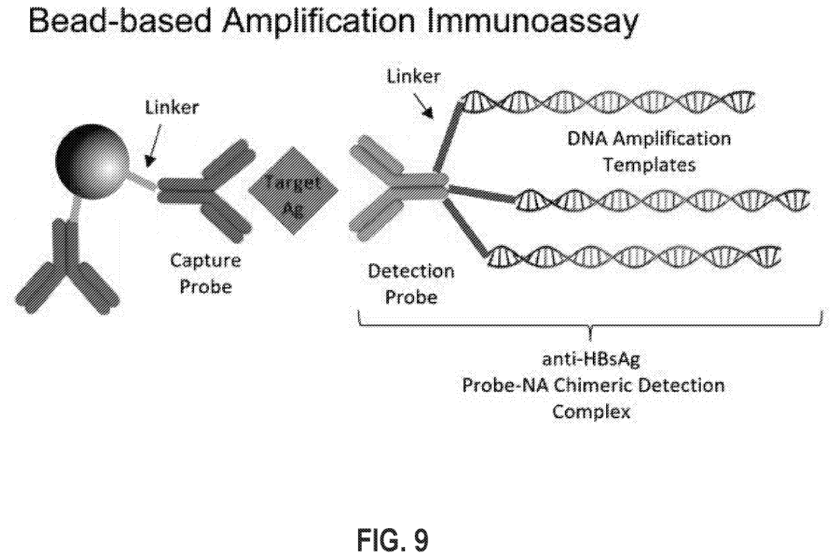

In some embodiments, the capture probe is immobilized onto a surface by covalent conjugation, the use of streptavidin-biotin linkages, or other bioconjugation and molecular immobilization methodologies as are commonly employed and familiar to those in the field. In some embodiments, the surface is a planar surface, a scaffold, a filter, a microsphere, a particle of any shape, a nanoparticle, or a bead or the like. An example embodiment is depicted in FIG. 9.

### Overview of Example Magnetic Beads

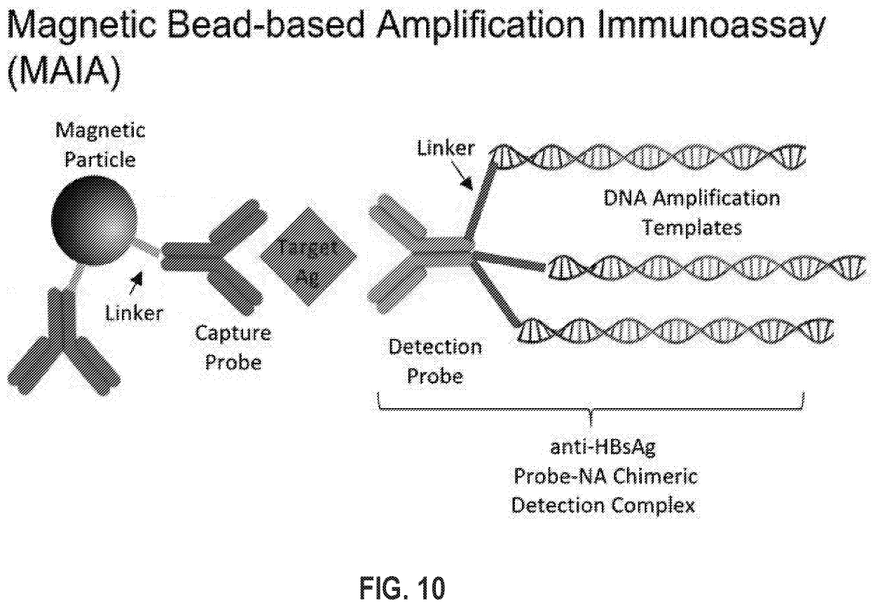

Some embodiments of the methods, systems and compositions provided herein include magnetic beads or the use thereof. In some embodiments, the microsphere, particle or bead is magnetic and/or magnetizable. The use of a magnetic support in such embodiments can facilitate the washing of the beads to remove excess, antigen and/or non-specifically adsorbed chimeric complex from the surfaces. A method, which includes the use of a magnetic particle support, may comprise a magnetic amplification immunoassay (MAIA). An example embodiment is depicted in FIG. 10.

In some embodiments, magnetic beads are useful to capture targets, and are used for magnetophoretic manipulation within the context of a purely electrical (MEMS) sample processing and/or amplification/detection cartridge and reduce or eliminate reliance on flow/pressure driven mobility within the fluidics. In some embodiments, magnetic beads are used to extract, and/or concentrate target genomic material from a sample. See e.g., Tekin, H C., et al., Lab Chip DOI: 10.1039/c3lc50477h, which is hereby incorporated by reference in its entirety. An automated microfluidic processing platform useful for embodiments provided herein is described in Sasso, L A., et al., Microfluid Nanofluidics. 13:603-612, which is hereby incorporated by reference in its entirety. Examples of beads useful with embodiments provided herein include Dynabeads® for Nucleic Acid IVD (ThermoFisher Scientific), or Dynabeads® SILANE Viral NA Kit (ThermoFisher Scientific).

### Overview of Example ƒC⁴D Excitation and Detection

In some implementations, the disclosed devices, systems, and/or methods utilize a ƒC⁴D based approach to monitor nucleic acid amplification in real-time. Thus, one or more phase-sensitive electrical conductivity measurements may be indicative of one or more targets within a sample.

In some aspects, a method includes rapidly sweeping frequencies at specific drive voltage values to determine an optimal excitation frequency (ƒₒₚₜ) where the sample conductivity linked to amplification is maximal. At ƒₒₚₜ the sensor output corresponds to a minimum in the relative phase difference between the excitation voltage and the induced current, thereby enabling high-sensitivity biomolecule quantification through conductivity measurements.

In some implementations, a ƒC⁴D detection system employs at least two electrodes. The two electrodes are placed in relatively close proximity to a microchannel where nucleic acid amplification is performed. An AC signal is applied to one of the two electrodes. The electrode to which the signal is applied to may be capacitively coupled through the microchannel to the second of the two electrodes. Thus, in some aspects the first electrode is a signal electrode and the second electrode is a signal electrode.

In general, the detected signal at the signal electrode is of an identical frequency as the AC signal that is applied to the signal electrode but is smaller in magnitude and has a negative phase shift. The pickup current may subsequently be amplified. In some aspects, the pickup current is converted to a voltage. In some aspects, the voltage is rectified. In some aspects, the rectified voltage is converted to a DC signal using a low-pass filter. The signal may be biased to zero before it is sent to a DAQ system for further processing.

The above-described system may be represented by a series of capacitors and resistors. Changes in electrical conductivity that occurs during nucleic acid amplification within the channel may cause the total impedance of the system to decrease and thus cause an increase in the level of the pickup signal that is produced. Such changes in the level of the resultant output signal may appear as one or more peaks on the DAQ system.

The signal generation and demodulation electronics is implemented with circuitry. For example, a printed circuit board (“PCB”), ASIC device, or other integrated circuitry (“IC”) is made using traditional manufacturing and fabrication techniques. In some aspects, such electronics are fully or partially designed to be single-use and/or disposable components. The physical geometry and electrical characteristics (passivation layer thickness, electrode pad area, channel cross sectional area and length, and dielectric strength) of such circuits is varied to achieve the desired results.

An example nucleic acid detection system includes at least one channel, and detects one or more physical properties, such as pH, optical properties, electrical properties and/or characteristics, along at least a portion of the length of the channel to determine whether the channel contains a particular nucleic acid of interest and/or a particular nucleotide of interest.

An example detection system is configured to include one or more channels for accommodating a sample and one or more sensor compounds (e.g., one or more nucleic acid probes), one or more input ports for introduction of the sample and the sensor compounds into the channel and, in some embodiments, one or more output ports through which the contents of the channel may be removed.

One or more sensor compounds (e.g., one or more nucleic acid probes) may be selected such that direct or indirect interaction among the nucleic acid and/or nucleotide of interest (if present in the sample) and particles of the sensor compounds results in formation of an aggregate that alters one or more physical properties, such as pH, optical properties, or electrical properties and/or characteristics, of at least a portion of the length of the channel.

In certain cases, formation of an aggregate, nucleic acid complex, or polymer inhibits or blocks fluid flow in the channel, and therefore causes a measurable drop in the electrical conductivity and electrical current measured along the length of the channel. Similarly, in these cases, formation of the aggregate, nucleic acid complex, or polymer causes a measurable increase in the resistivity along the length of the channel. In certain other cases, the aggregate, nucleic acid complex, or polymer is electrically conductive, and formation of aggregate, nucleic acid complex, or polymer enhances an electrical pathway along at least a portion of the length of the channel, thereby causing a measurable increase in the electrical conductivity and electrical current measured along the length of the channel. In these cases, formation of an aggregate, nucleic acid complex, or polymer causes a measurable decrease in the resistivity along the length of the channel.

In certain cases, formation of an aggregate, nucleic acid complex, or polymer affects waveform characteristics of one or more electrical signals that are sent through a channel. As shown, for example in FIG. 11, a first electrode or excitation electrode 1116 and a second electrode (a ‘pickup’ or ‘sensor’ electrode) 118 are spaced apart from one another along a channel 1104. FIG. 11 represents an alternate or complementary approach to that described above with respect to FIGS. 5A-5D. The first and second electrodes 1116, 1118 may not be in contact with the measured solution that is contained within the channel 1104. In this sense the first and second electrodes 1116, 1118 are capacitively-coupled to the solution within the channel 1104. The strength of the capacitive coupling depends on the electrode geometry, passivation layer thickness, and the passivation layer material (specifically its relative dielectric strength).

In some aspects, the solution is confined to the channel 1104. The channel may have a micron-scale cross-sectional area. As such, the solution behaves as a resistor whose resistance depends on the solution's conductivity and the channel 1104 geometry.

In some implementations, an alternating current/voltage is applied to the excitation electrode 1116 and the induced current is measured at the signal electrode 1118. The induced current is proportional to the inter-electrode impedance, which may change with the solution's conductivity. As shown, an excitation voltage 1400 is applied to the excitation electrode 1116 and an induced current 1410 is detected by the signal electrode 1118.

In some implementations, detector sensitivity is at least partially dependent on excitation frequency. Thus, in some aspects a maximal sensitivity occurs when the absolute value of the phase of the induced current is at a minimum. In this region, chip impedance is dominated by fluid impedance. Fluid impedance is a function of fluid conductivity and chip geometry. Complex impedance information is important for ensuring maximal detector sensitivity and correct detector operation

An analysis of lumped parameter model for the equivalent circuit has shown that detector sensitivity is intimately related to the strength of coupling capacitance, C_{WALL}, the solution resistance, R_{LAMP}, and the parasitic capacitance, C_{X}. Specifically, the change in inter-electrode impedance with respect to conductivity change is maximal when the excitation frequency, ƒ, satisfies the following:

1/(πR_{LAMP}C_{WALL})<<ƒ<<1/(πR_{LAMP}C_{X})

As shown in FIG. 12, the impedance of the signal is dependent on the excitation frequency and changes after a LAMP reaction occurs in the channel 1104. As also seen in FIG. 12, the left inequality may define a frequency region below which the coupling impedance dominates and changes in the solution's impedance become practically invisible. The right inequality may define a frequency region above which parasitic effects dominate, and the electrodes 1116, 1118 are in effect shunted together.

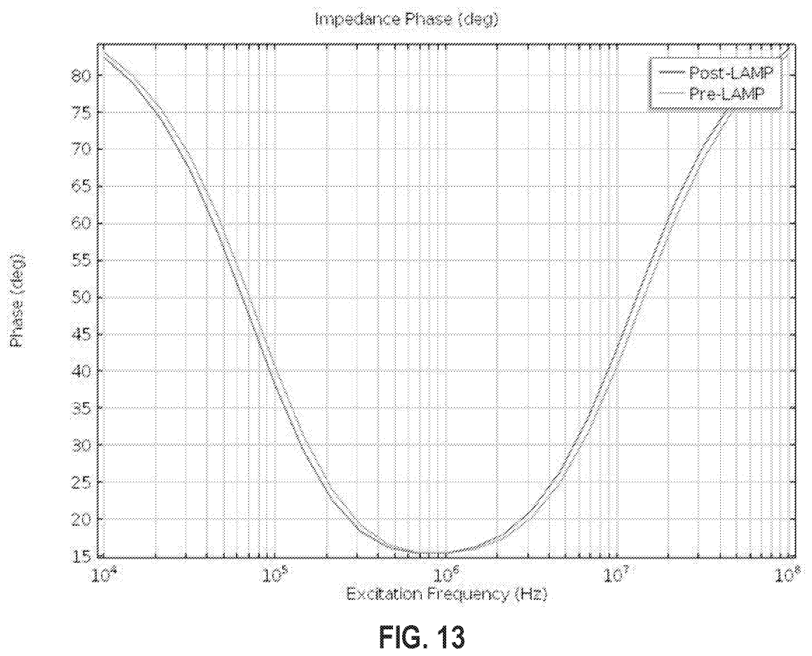

As shown in FIG. 13, in both extremal regions, the impedance is capacitor-like, and is out of phase (approaching 90°) with the excitation voltage. Between the two regions, the impedance begins to approach the limit of a simple resistor, and the impedance versus frequency response flattens out. In fact, maximal detector sensitivity corresponds to the phase minimum of the impedance.

To elucidate the need for synchronous detection, one may consider two parallel paths for current in a simplified model: current through the chip via the fluidic channel and parasitic or geometric capacitance. Given an excitation signal, V, at a given frequency, ƒ, the induced current, I, will be:

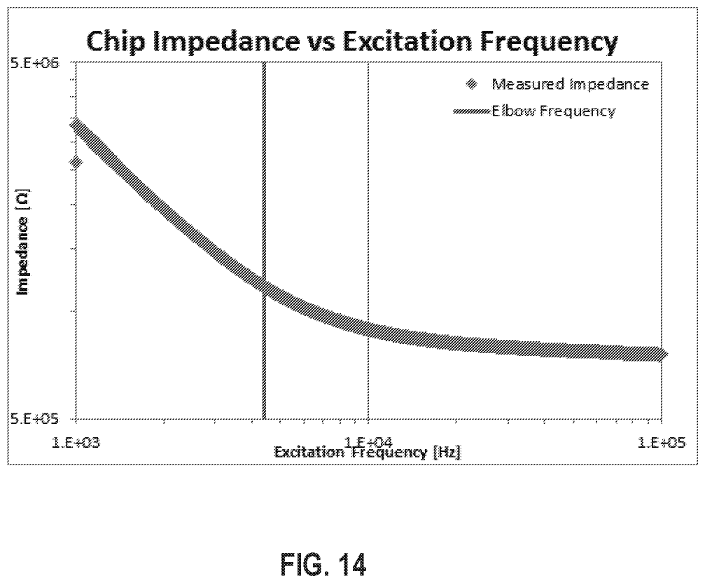

I(t)=(Y+2πƒCₓj)V(t)

where Y is the admittance of the chip due to coupling to the fluidic path, Cₓ is the parasitic capacitance, and j is the imaginary unit. Multiplication by j means the current through the parasitic path is 90° out of phase with the excitation voltage. The measured impedance of a sample chip with respect to excitation frequency is shown in FIG. 14.

In a synchronous detector, the pickup current is multiplied by an in-phase square wave, m, then low-pass filtered.

$$m ( t ) = sgn ( sin ( 2 π ft ) ) = \frac{4}{π} {\sum }_{k = 1}^{∞} \frac{sin ( 2 π ( 2 k - 1 ) ft )}{2 k - 1}$$

It is straightforward to show that the contribution of signals 90° out of phase with the modulating signal will be zero, so we may ignore the parasitic capacitance in this analysis. To see the effect synchronous detection on the current through the fluidic path, one can multiply the induced current (minus the parasitic contribution), with the modulating wave

$$ml = mYV = \frac{4}{π} \vert V \vert \vert Y \vert sin ( 2 π ft + φ ) sin ( 2 π ft ) + H . F . T . = \begin{matrix}2 \\ π\end{matrix} \vert V \vert \vert Y \vert cos ( φ ) \begin{matrix}2 \\ π\end{matrix} \vert V \vert \vert Y \vert cos ( 2 π ( 2 f ) t \vert φ ) \vert H . F . T .$$

where |Y| is the magnitude of the admittance, and φ=arg(Y), and H.F.T. means high frequency terms (e.g., greater than ƒ). After low pass filtering, one may be left with the DC term of the synchronous output:

$$s = \frac{2}{π} \vert V \vert \vert Y \vert cos ( φ )$$

This expression can be simplified this by noting that:

$$cos ( φ ) = \frac{Re \{ Y \}}{\vert Y \vert}$$

resulting in:

$$s = \frac{2}{π} \vert V \vert Re \{ Y \}$$

Alternatively, one can express this in terms of impedance by Z, by noting that

$$Y = \frac{1}{Z} = \frac{\overline{Z}}{{\vert Z \vert}^{2}}$$

where the bar denotes complex conjugation. The synchronous detector output thus becomes

$$s = \frac{2}{π} \vert V \vert \frac{Re \{ Z \}}{{\vert Z \vert}^{2}}$$

Given the simple circuit models for the chip, the impedance is computed explicitly, and the output of the synchronous detector is predicted.

A simple equivalent circuit model comprises two capacitors, C, in series with a resistor, R. As discussed above, the resistance R is primarily a function of the microfluidic geometry and solution conductance. The capacitance is primarily a function of the electrode area, the dielectric used for the passivation layer and the passivation layer thickness. The impedance, Z, of the simplified circuit is given by:

$$Z = R - ( \frac{1}{π fC} ) j$$

The square of the magnitude of the impedance is:

|Z|²=R²+(πƒC)⁻²

and the output of the synchronous detector is:

$$s = \frac{2}{π} \vert V \vert \frac{R}{{R}^{2} + {( π fC )}^{- 2}} = \frac{2}{π} \vert V \vert \frac{G}{1 + {( \frac{G}{π fC} )}^{2}} =$$

where the numerator and denominator is multiplied by the square of the conductance, G=1/R.

For conductivity meters, a cell constant, k, may be defined to be:

$$R = \frac{k}{σ}$$

where k has units of inverse length. The cell constant k, primarily depends on electrode placement, area, and fluidic path, and may not be a simple linear relationship. The synchronous detector output is then:

$$s = \frac{2}{π} \vert V \vert \frac{σ / k}{1 + {( \frac{σ}{π kfC} )}^{2}}$$

To aid in the analysis, one may introduce a dimensionless conductivity parameter, \tilde{σ}, where:

$$\tilde{σ} = \frac{σ}{π kfC}$$

So that:

$$s = \frac{2}{π} \vert V \vert fC \frac{\tilde{σ}}{1 + {\tilde{σ}}^{2}}$$

The dependence of the detector output on the non-dimensional conductivity, \tilde{σ}, is of note.
1) The detector response is asymptotically proportional to for \tilde{σ} for \tilde{σ}<<1
2) The detector response reaches a local maximum of Eₘₐₓ=|V|ƒC at \tilde{σ}=1
3) The detector response is asymptotically proportional to 1/\tilde{σ} for \tilde{σ}>>1.

Given the dependence of the detector response on the non-dimensional conductance, it is important to tightly couple the design the chip and detector. Translating the previously-stated points in terms of the actual conductance result in the following:
1) The detector response is asymptotically proportional to σ for

$$f ⪢ \frac{σ}{π kC}$$

2) The detector response is asymptotically proportional to

$$\frac{1}{σ}$$

for

$$f ⪡ \frac{σ}{π kC}$$

3) The detector response becomes non-monotonic at σ=πkƒC

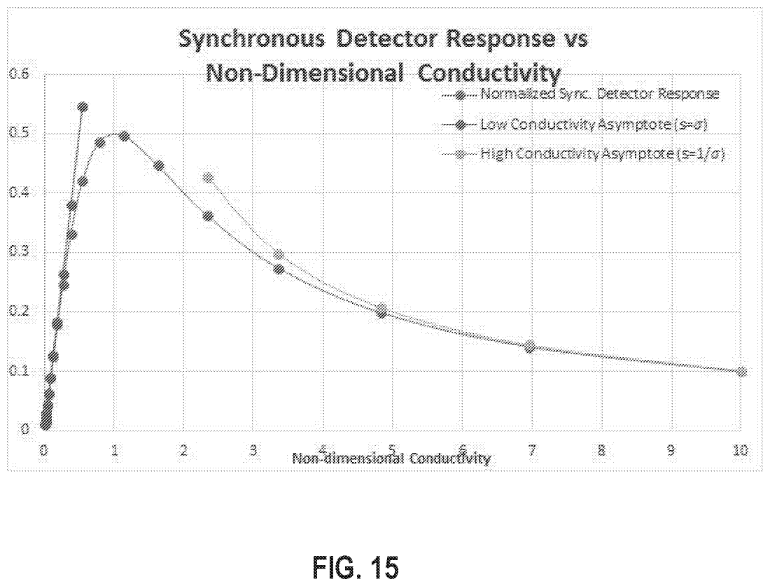

In other words, increasing the excitation frequency expands the range of conductivities for which the synchronous detector output is linear. A synchronous detector response is plotted with respect to non-dimensional conductivity in FIG. 15.

To evaluate the lumped parameter model's validity, the detector response for known conductivity solutions of KCl was measured. The chip's channel was 2 mm with 0.01 mm² cross-sectional area. The two electrodes were each 9 mm², passivated with a 10 um layer of SU8 photoresist. The cell constant and capacitance were estimated and an excitation frequency was chosen so that the conductivity corresponding to the non-linearity in the detector output would be approximately 5 mS/cm. The experiment was repeated at excitation frequencies of 10, 15, and 20 kHz.

The conductivity of pre-LAMP chemistries has been measured to be approximately 10 mS/cm. TABLE 1 below, presents the estimates for the minimal detector frequency governed by the constraint found earlier, namely:

$$f ⪢ \frac{G}{π C}$$

TABLE 1

| Geometry | A_{E} [mm²] | t [μm] | εᵣ | C [pF] | A_{F} [mm²] | t [mm] | G [mS] | f [MHz] |
|---|---|---|---|---|---|---|---|---|
| Restrictive | 9 | 10 | 3 | 24 | 0.01 | 3 | 0.003 | 0.044 |
| Channel | | | | | | | | |
| Bulk Well, | 0.8 | 0.3 | 3 | 71 | 0.8 | 1 | 0.8 | 3.6 |
| Planar | | | | | | | | |
| Electrodes | | | | | | | | |
| Parallel | 16 | 300 | 2.8 | 1.3 | 16 | 1.5 | 10.5 | 2500 |
| Plate, | | | | | | | | |
| Non-integrated | | | | | | | | |
| Electrodes | | | | | | | | |

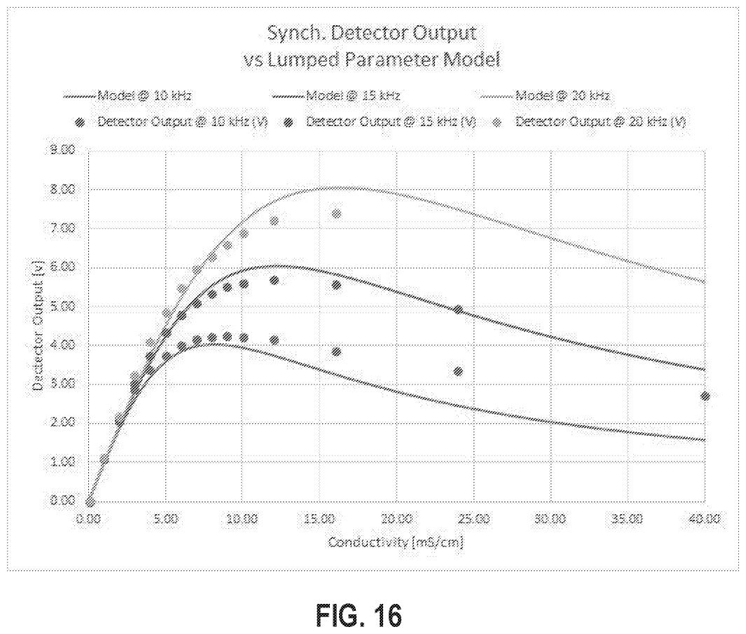

The results of the model, shown in FIG. 16, demonstrate good agreement with the detector output for a wide range of conductivities and for the given steps in frequencies. It is important to note that the same two parameters, k and C, are used at each frequency. The model predicts the qualitative behavior of the detector response. Namely, the functional form the response, the dependence of the critical conductivity at which the nonlinearity occurs on the excitation frequency. The model overestimates the divergence of the frequency-dependent behavior for conductivities past the critical conductivity.

As a tool to quickly estimate the conductance and wall capacitance, one may ignore surface conductivity and capacitance effects in addition to fringe fields effects. A geometry-specific finite element model can be used to further refine this crude estimate.

The electrode is modeled as a parallel plate capacitor of area A_{E}, separated by a dielectric of relative dielectric strength εᵣ, and thickness t. The capacitance is then approximated as:

$$C = \frac{{ɛ}_{0} {ɛ}_{r} {A}_{E}}{t}$$

where ε₀ is the dielectric constant.

The fluid may be modeled as a simple resistor of cross-sectional area A_{F}, length l, and conductivity σ. Thus, the conductance of the fluidic path may be approximated as

$$G = \frac{σ {A}_{F}}{t}$$

From this, the cell constant is also approximated.

In some aspects, the device is configured to determine “impedance spectrum” after the chip is introduced. The device may include a digitally controlled excitation frequency. The device may have quick frequency sweeping ability. The device may include in-phase and quadrature components of the induced signal, from which complex impedance can be determined. The fitness of impedance spectrum is determined, at least in part, based on curve fit or other heuristic to determine proper chip insertion and/or proper sample introduction. In some aspects, the device is first tested by exciting at a frequency determined by initial sweep. In some implementations, the device includes a detector that utilizes synchronous detection. In this way, measured induced currents attributable to the fluidic path (at phase minimum) may be detected in real time.

### Overview of Example Channels

In some embodiments, a channel or conduit has the following dimensions: a length measured along its longest dimension (y-axis) and extending along a plane parallel to the substrate of the detection system; a width measured along an axis (x-axis) perpendicular to its longest dimension and extending along the plane parallel to the substrate; and a depth measured along an axis (z-axis) perpendicular to the plane parallel to the substrate. An example channel may have a length that is substantially greater than its width and its depth. In some cases, example ratios between the length:width may be: 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1 or within a range defined by any two of the aforementioned ratios.

In some embodiments, a channel or conduit is configured to have a depth and/or a width that is substantially equal to or smaller than the diameter of an aggregate, nucleic acid complex, or polymer formed in the channel, preferably while in suspension in the channel, due to interaction between the nucleic acid of interest and particles of the sensor compounds (e.g., one or more nucleic acid probes) used to detect the nucleic acid of interest.

In some embodiments, a channel is configured to have a width taken along the x-axis ranging from about 1 nm to about 50,000 nm or a width that is within a range defined by any two numbers within the aforementioned range but is not limited to these example ranges. An example channel or conduit has a length taken along the y-axis ranging from about 10 nm to about 2 cm, or a length that is within a range defined by any two numbers within the aforementioned range but is not limited to these example ranges. An example channel has a depth taken along the z-axis ranging from about 1 nm to about 1 micron, or a depth that is within a range defined by any two numbers within the aforementioned range but is not limited to these example ranges.

In some embodiments, a channel or conduit has any suitable transverse cross-sectional shape (e.g., a cross-section taken along the x-z plane) including, but not limited to, circular, elliptical, rectangular, square, D-shaped (due to isotropic etching), and the like.

In some embodiments, a channel or conduit has a length in a range from 10 nm to 10 cm, such as e.g., at least or equal to 10 nm, 50 nm, 100 nm, 200 nm, 400 nm, 600 nm, 800 nm, 1 μm, 10 μm, 50 μm, 100 μm, 300 μm, 600 μm, 900 μm, 1 cm, 3 cm, 5 cm, 7 cm, or 10 cm or a length that is within a range defined by any two of the aforementioned lengths. In some embodiments, a channel has a depth in a range from 1 nm to 1 μm, such as e.g., at least or equal to 1 nm, 5 nm, 7 nm, 10 nm, 50 nm, 100 nm, 200 nm, 400 nm, 600 nm, 800 nm, 1 μm, 10 μm, 20 μm, 30 μm, 40 μm, 50 μm, 100 μm, 500 μm, or 1 mm or a depth that is within a range defined by any two of the aforementioned depths. In some embodiments, a channel has a width in a range from 1 nm to 50 μm, such as e.g., 1 nm, 5 nm, 7 nm, 10 nm, 50 nm, 100 nm, 200 nm, 400 nm, 600 nm, 800 nm, 1 μm, 10 μm, 20 μm, 30 μm, 40 μm, 50 μm, 100 μm, 500 μm, or 1 mm or a width that is within a range defined by any two of the aforementioned widths.

In some implementations, the channels or conduits are formed in a cartridge that is later inserted into a device. In some aspects, the cartridge may be a disposable cartridge. In some aspects, the cartridge is made of cost-effective plastic materials. In some aspects, at least a portion of the cartridge is made from paper and laminate-based materials for fluidics.

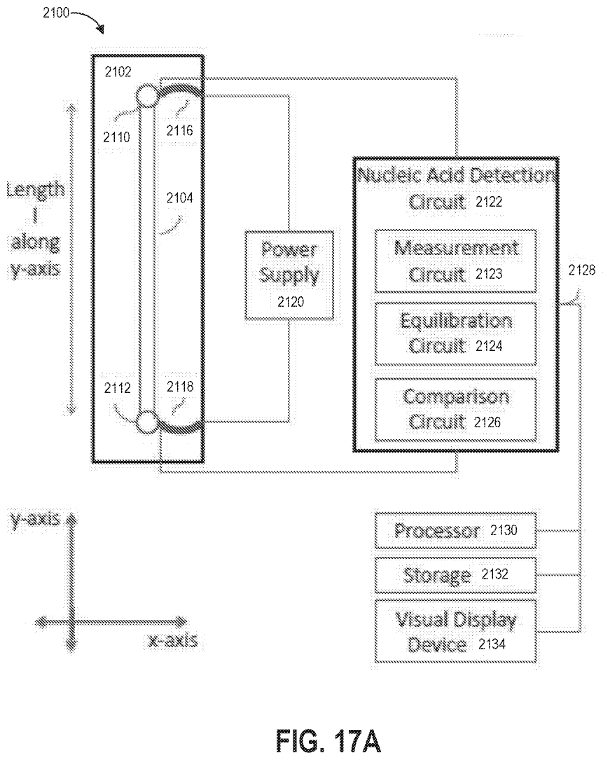

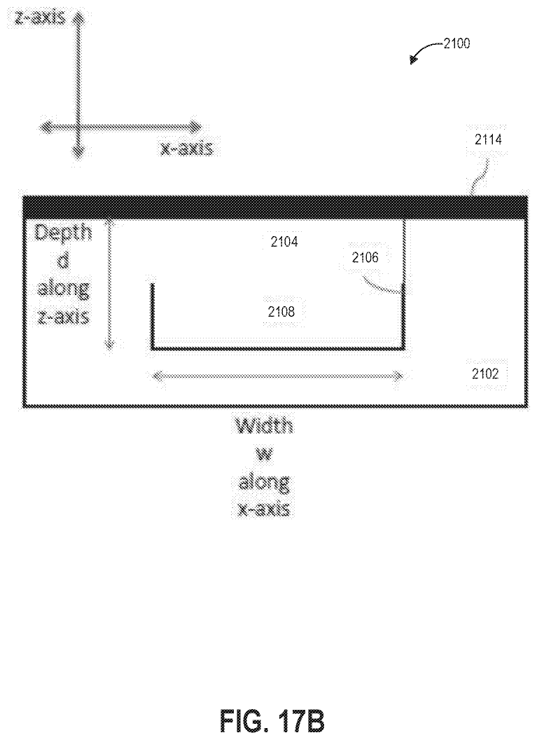

An embodiment of a detection system 2100 that is used to detect presence or absence of a particular nucleic acid and/or a particular nucleotide in a sample is illustrated in FIGS. 17A-17B. FIG. 17A is a top view of the system, while FIG. 17B is a cross-sectional side view of the system. The detection system 2100 includes a substrate 2102 that extends substantially along a horizontal x-y plane. In some embodiments, the substrate 2102 may be formed of a dielectric material, for example, silica. Other example materials for the substrate 2102 include, but are not limited to, glass, sapphire, or diamond.

The substrate 2102 supports or includes a channel 2104 having at least an inner surface 2106 and an inner space 2108 for accommodating a fluid. In some cases, the channel 2104 is etched in a top surface of the substrate 2102. Example materials for the inner surfaces 2106 of the channel 2104 include, but are not limited to, glass or silica.

The channel 2104 and the substrate 2102 are formed of glass in certain embodiments. Biological conditions represent a barrier to the use of glass-derive implantations due to the slow dissolution of glass into biological fluids and adhesion of proteins and small molecules to the glass surface. In certain non-limiting embodiments, surface modification using a self-assembled monolayer offers an approach for modifying glass surfaces for nucleic acid detection and analysis. In certain embodiments, at least a portion of the inner surface 2106 of the channel 2104 is pre-treated or covalently modified to include or be coated with a material that enables specific covalent binding of a sensor compound to the inner surface. In certain embodiments, a cover slip 2114 covering the channel may also be covalently modified with a material.

Exemplary materials that are used to modify the inner surface 2106 of the channel 2104 include, but are not limited to, a silane compound (e.g., trichlorosilane, alkylsilane, triethoxysilane, perfluoro silane), zwitterionic sultone, poly(6-9)ethylene glycol (Peg), perfluorooctyl, fluorescein, an aldehyde, or a graphene compound. The covalent modification of the inner surface of the channel decreases non-specific absorption of certain molecules. In one example, covalent modification of the inner surface may enable covalent bonding of sensor compound molecules to the inner surface while preventing nonspecific absorption of other molecules to the inner surface. For example, poly(ethylene glycol) (Peg) is used to modify the inner surface 2106 of the channel 2104 to reduce nonspecific adsorption of materials to the inner surface.

In some embodiments, the channel 2104 is nano or micro-fabricated to have a well-defined and smooth inner surface 2106. Exemplary techniques for fabricating a channel and modifying the inner surface of a channel are taught in Sumita Pennathur and Pete Crisallai (2014), “Low Temperature Fabrication and Surface Modification Methods for Fused Silica Micro- and Nanochannels,” MRS Proceedings, 1659, pp 15-26. doi:10.1557/opl.2014.32, the entire contents of which are hereby expressly incorporated herein by reference.

A first end section of the channel 2104 includes or is in fluid communication with an input port 2110, and a second end section of the channel 2104 includes or is in fluid communication with an output port 2112. In certain non-limiting embodiments, the ports 2110 and 2112 are provided at terminal ends of the channel 2104.

The top surface of the substrate 2102 having the channel 2104 and the ports 2110, 2112 is covered and sealed with a cover slip 2114 in some embodiments. In some embodiments, a rigid plastic is used to define the channels, including the top, and a semipermeable membrane may also be used.

A first electrode 2116 is electrically connected at the first end section of the channel 2104, for example, at or near the input port 2110. A second electrode 2118 is electrically connected at the second end section of the channel 2104, for example, at or near the output port 2112. The first and second electrodes 2116, 2118 are electrically connected to a power supply or voltage source 2120 in order to apply a potential difference between the first and second electrodes. That is, the potential difference is applied across at least a portion of the length of the channel. When a fluid is present in the channel 2104 and is under the influence of the applied potential difference, the electrodes 2116, 2118 and the fluid create a complete electrical pathway.

The power supply or voltage source 2120 is configured to apply an electric field in a reversible manner such that a potential difference is applied in a first direction along the channel length (along the y-axis) and also in a second opposite direction (along the y-axis). In one example in which the electric field or potential difference direction is in a first direction, the positive electrode is connected at the first end section of the channel 2104, for example, at or near the input port 2110, and the negative electrode is connected at the second end section of the channel 2104, for example, at or near the output port 2112. In another example in which the electric field or potential difference direction is in a second opposite direction, the negative electrode is connected at the first end section of the channel 2104, for example, at or near the input port 2110, and the positive electrode is connected at the second end section of the channel 2104, for example, at or near the output port 2112.

The power supply or voltage source 2120 are configured to apply an AC signal in some embodiments. The frequency of the AC signal may be changed dynamically. In some aspects, the power supply or voltage source 2120 are configured to supply an electrical signal having a frequency between 10-10⁹ Hz. In some aspects, the power supply or voltage source 2120 are configured to supply an electrical signal having a frequency between 10⁵-10⁷ Hz.

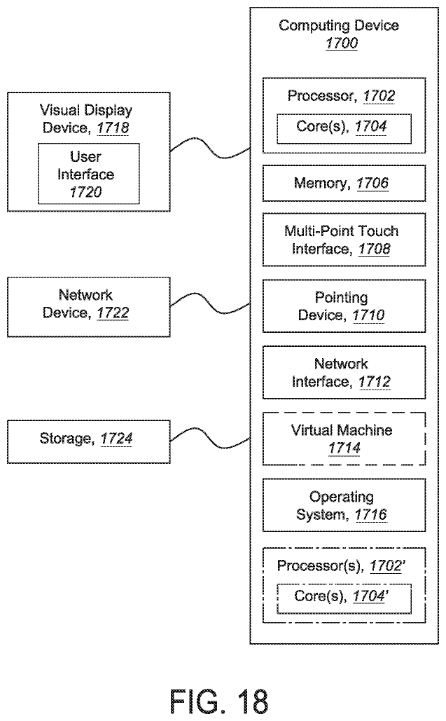

The first and second end sections of the channel 2104 (e.g., at or near the input port 2110 and the output port 2112) are electrically connected to a nucleic acid detection circuit 2122 that is programmed or configured to detect values of one or more electrical properties of the channel 2104 for determining whether the particular nucleic acid and/or nucleotide is present or absent in the channel 2104. The electrical property values are detected at a single time period (for example, a certain time period after introduction of a sample and one or more sensor compounds into the channel), or at multiple different time periods (for example, before and after introduction of both the sample and one or more sensor compound into the channel). In some aspects, the electrical property values are detected continuously for a set time period from sample introduction through LAMP amplification. Example electrical properties detected include, but are not limited to, electrical current, conductivity voltage, resistance, frequency, or waveform. Certain example nucleic acid detection circuits 2122 include or are configured as a processor or a computing device, for example as device 1700 illustrated in FIG. 18. Certain other nucleic acid detection circuits 2122 include, but are not limited to, an ammeter, a voltmeter, an ohmmeter, or an oscilloscope.

In one embodiment, the nucleic acid detection circuit 2122 comprises a measurement circuit 2123 programmed or configured to measure one or more electrical property values along at least a portion of a length of the channel 2104. The nucleic acid detection circuit 2122 also comprises an equilibration circuit 2124 that is programmed or configured to periodically or continually monitor one or more values of an electrical property of the channel over a time period, and/or to select a single one of the values after the values have reached equilibrium (e.g., have stopped varying beyond a certain threshold of variance or tolerance).

The nucleic acid detection circuit 2122 may also comprise a comparison circuit 2126 that is programmed or configured to compare two or more electrical property values of the channel, for example, a reference electrical property value (e.g., measured before a state in which both the sample and all of the sensor compounds have been introduced into the channel) and an electrical property value (e.g., measured after introduction of the sample and all of the sensor compound into the channel). The comparison circuit 2126 may use the comparison in order to determine whether the nucleic acid is present or absent in the channel. In one embodiment, the comparison circuit 2126 calculates a difference between the measured electrical property value and the reference electrical property value and compares the difference to a predetermined value indicative of the presence or absence of the nucleic acid in the channel and this information is used to diagnose or predict a disease state or the presence or absence of an infection in the subject.

In certain embodiments, upon introduction of both the sample and the sensor compound into the channel, the comparison circuit 2126 is programmed or configured to compare a first electrical property value (e.g., magnitude of electrical current) when the electric field or potential difference is applied across the channel in a first direction along the length of the channel to a second electrical property value (e.g., magnitude of electrical current) when the electric field or potential difference is applied across the channel in a second opposite direction along the length of the channel. In one embodiment, the comparison circuit 2126 calculates a difference between the magnitudes of the first and second values and compare the difference to a predetermined value (e.g., whether the difference is substantially zero) indicative of the presence or absence of a nucleic acid in the channel. For example, if the difference is substantially zero, this indicates absence of a nucleic acid, which may be in a dispersed, polymer form, or aggregate form, in the channel. If the difference is substantially non-zero, this indicates presence of a nucleic acid, which may be in dispersed form, a polymer form, or an aggregate form, in the channel.

In certain embodiments, the nucleic acid detection circuit 2122 is programmed or configured to determine an absolute concentration of the nucleic acid in a sample, and/or a relative concentration of the nucleic acid relative to one or more additional substances in a sample.

In some embodiments, the comparison circuit 2124 and the equilibration circuit 2126 is configured as separate circuits or modules, while in other embodiments, they are configured as a single integrated circuit or module.

The nucleic acid detection circuit 2122 has an output 2128 that may, in some embodiments, be connected to one or more external devices or modules. For example, the nucleic acid detection circuit 2122 may transmit a reference electrical property value and/or one or more measured electrical property values to one or more of: a processor 2130 for further computation, processing and analysis, a non-transitory storage device or memory 2132 for storage of the values, and/or a visual display device 2134 for display of the values to a user. In some embodiments, the nucleic acid detection circuit 2122 generates an indication of whether the sample includes the nucleic acid, and it transmits this indication to the processor 2130, the non-transitory storage device or memory 2132 and/or the visual display device 2134.

In an example method of using the system of FIG. 17A and FIG. 17B, one or more sensor compounds (e.g., one or more nucleic acid probes) and a sample are sequentially or concurrently introduced into the channel. When flow of the fluid and/or flow of the charged particles in the fluid is uninhibited (e.g., due to absence of an aggregate), the conductive particles or ions in the fluid travel along at least a portion of the length of the channel 2104 along the y-axis from the input port 2110 toward the output port 2112. The movement of the conductive particles or ions produce or generate a first or “reference” electrical property value or range of values (e.g., of an electrical current, conductivity, resistivity, or frequency) being detected by the nucleic acid detection circuit 2122 along at least a portion of the length of the channel 2104. In some embodiments, the equilibration circuit 2124 periodically or continually monitors electrical property values during a time period until the values reach equilibrium. The equilibration circuit 2124 then selects one of the values as the reference electrical property value to avoid the influence of transient changes in the electrical property.

As used herein, “reference” electrical property value refers to a value or range of values of an electrical property of a channel prior to introduction of a sample and all of the sensor compounds (e.g., one or more nucleic acid probes) into the channel. That is, the reference value is a value characterizing the channel prior to any interaction between the nucleic acid in the sample and all of the sensor compounds. In some cases, the reference value is detected at a time period after introduction of a sensor compound into the channel but before introduction of the sample and additional sensor compounds into the channel. In some cases, the reference value is detected at a time period after introduction of a sensor compound and the sample into the channel but before introduction of additional sensor compounds into the channel. In some cases, the reference value is detected at a time period before introduction of the sample or the sensor compounds into the channel. In some cases, the reference value is predetermined and stored on a non-transitory storage medium from which it may be accessed.

In some cases, formation of an electrically conductive aggregate, polymer, or nucleic acid complex in the channel (e.g., due to interactions between a nucleic acid of interest in the sample and one or more nucleic acid probes) enhances the electrical pathway along at least a portion of the length of the channel 2104. In this case, the nucleic acid detection circuit 2122 detects a second electrical property value or range of values (e.g., of an electrical current, conductivity, resistivity, or frequency) along at least a portion of the length of the channel 2104. In some embodiments, the nucleic acid detection circuit 2122 provides for a waiting or adjustment time period after introduction of the sample and all of the sensor compounds into the channel prior to detecting the second electrical property value. The waiting or adjustment time period allows an aggregate, polymer, or nucleic acid complex to form in the channel, preferably while being suspended in the channel, and for the aggregate, polymer, or nucleic acid complex formation to alter the electrical properties of the channel, preferably while being suspended in the channel.

In some embodiments, the equilibration circuit 2124 periodically or continually monitors electrical property values during a time period after the introduction of the sample and all of the sensor compounds until the values reach equilibrium. The equilibration circuit 2124 may then select one of the values as the second electrical property value to avoid the influence of transient changes in the electrical property.

The comparison circuit 2126 compares the second electrical property value to the reference electrical property value. If it is determined that the difference between the second value and the reference value corresponds to a predetermined range of increase in current or conductivity (or decrease in resistivity), the nucleic acid detection circuit 2122 determines that an aggregate, polymer, or nucleic acid complex is present in the channel and that, therefore, the nucleic acid target is present or detected in the sample. Based thereon, one can diagnose or identify the presence or absence of the target and a disease state or infection state in a subject.

In certain other embodiments, when flow of the fluid in the channel and/or flow of the charged particles in the fluid is partially or completely blocked (for example, by formation of an aggregate, polymer, or nucleic acid complex), the conductive particles or ions in the fluid are unable to freely travel along at least a portion of the length of the channel 2104 along the y-axis from the input port 2110 toward the output port 2112. The hindered or stopped movement of the conductive particles or ions produces or generates a third electrical property value or range of values (e.g., of an electrical current or signal, conductivity, resistivity, or frequency) is detected by the nucleic acid detection circuit 2122 along at least a portion of the length of the channel 2104. The third electrical property value is detected in addition to or instead of the second electrical property value. In some embodiments, the nucleic acid detection circuit 2122 may wait for a waiting or adjustment time period after introduction of both the sample and all of the sensor compounds into the channel prior to detecting the third electrical property value. The waiting or adjustment time period allows an aggregate, polymer, or nucleic acid complex to form in the channel and for the aggregate, polymer, or nucleic acid complex formation to alter the electrical properties of the channel.

In some embodiments, the equilibration circuit 2124 periodically or continually monitors electrical property values during a time period after the introduction of the sample and all of the sensor compounds until the values reach equilibrium. The equilibration circuit 2124 then selects one of the values as the third electrical property value to avoid the influence of transient changes in the electrical property.

The comparison circuit 2126 compares the third electrical property value to the reference electrical property value. If it is determined that the difference between the third value and the reference value corresponds to a predetermined range of decrease in current or conductivity (or increase in resistivity), the nucleic acid detection circuit 2122 determines that an aggregate, polymer, or nucleic acid complex is present in the channel and that, therefore, the target nucleic acid is identified as being present in the sample.

The fluid flow along the length of the channel depends on the size of the aggregate, polymer, or nucleic acid complex in relation to the dimensions of the channel, and the formation of an electrical double layer (EDL) at the inner surface of the channel.

In general terms, an EDL is a region of net charge between a charged solid (e.g., the inner surface of the channel, an analyte particle, an aggregate, polymer, or nucleic acid complex) and an electrolyte-containing solution (e.g., the fluid contents of the channel). EDLs exist around both the inner surface of the channel and around any nucleic acid particles and aggregates, polymers, or nucleic acid complexes within the channel. The counter-ions from the electrolyte are attracted towards the charge of the inner surface of the channel and induce a region of net charge. The EDL affects ion flow within the channel and around analyte particles and aggregates, polymers, or nucleic acid complexes of interest, creating a diode-like behavior by not allowing any of the counter-ions to pass through the length of the channel.

To mathematically solve for the characteristic length of the EDL, the Poisson-Boltzmann (“PB”) equation and/or Poisson-Nemst-Plank equations (“PNP”) are solved. These solutions are coupled to the Navier-Stokes (NS) equations for fluid flow to create a nonlinear set of coupled equations that are analyzed to understand the operation of the example system.

In view of the dimensional interplay among the channel surface, the EDLs and the aggregates, polymers, or nucleic acid complexes, example channels are configured and constructed with carefully selected dimensional parameters that ensure that flow of conductive ions is substantially inhibited along the length of the channel when an aggregate, polymer, or nucleic acid complex of a certain predetermined size is formed in the channel. In certain cases, an example channel is configured to have a depth and/or a width that is substantially equal to or smaller than the diameter of an aggregate particle formed in the channel during nucleic acid detection. In certain embodiments, the sizes of the EDLs are also taken into account in selecting dimensional parameters for the channel. In certain cases, an example channel is configured to have a depth and/or a width that is substantially equal to or smaller than the dimension of the EDL generated around the inner surface of the channel and the aggregate, polymer, or nucleic acid complex in the channel.

In certain embodiments, prior to use of the detection system, the channel is free of the sensor compounds (e.g., one or more nucleic acid probes). That is, a manufacturer of the detection system may not pre-treat or modify the channel to include the sensor compound. In this case, during use, a user will introduce one or more sensor compounds, for example in an electrolyte buffer, into the channel and detect a reference electrical property value of the channel with the sensor compound but in the absence of a sample.

In certain other embodiments, prior to use of the detection system, the channel is pre-treated or modified so that at least a portion of an inner surface of the channel includes or is coated with a sensor compound (e.g., one or more nucleic acid capture probes). In one example, the manufacturer detects a reference electrical property value of the channel modified with the sensor compound and, during use a user may use the stored reference electrical property value. That is, a manufacturer of the detection system may pretreat or modify the channel to include a sensor compound. In this case, a user will need to introduce the sample and one or more additional sensor compounds into the channel.

Certain example detection systems include a single channel. Certain other example detection systems include multiple channels provided on a single substrate. Such detection systems may include any suitable number of channels including, but not limited to, at least or equal to 2, 3, 4, 5, 6, 7, 8, 9, or 10, or a number of channels within a range defined by any two of the aforementioned numbers.

In one embodiment, a detection system includes a plurality of channels in which at least two channels operate independent of each other. The example channel 2104 and associated components of FIGS. 17A-17B are reproduced on the same substrate to achieve such a multi-channel detection system. The multiple channels are used to detect the same nucleic acid in the same sample, different nucleic acids in the same sample, the same nucleic acid in different samples, and/or different nucleic acids in different samples. In another embodiment, a detection system includes a plurality of channels in which at least two channels operate in cooperation with each other. In some aspects, the channels are shaped differently depending on the target that is sought to be detected.

### Overview of Example Devices for Point of Care Use

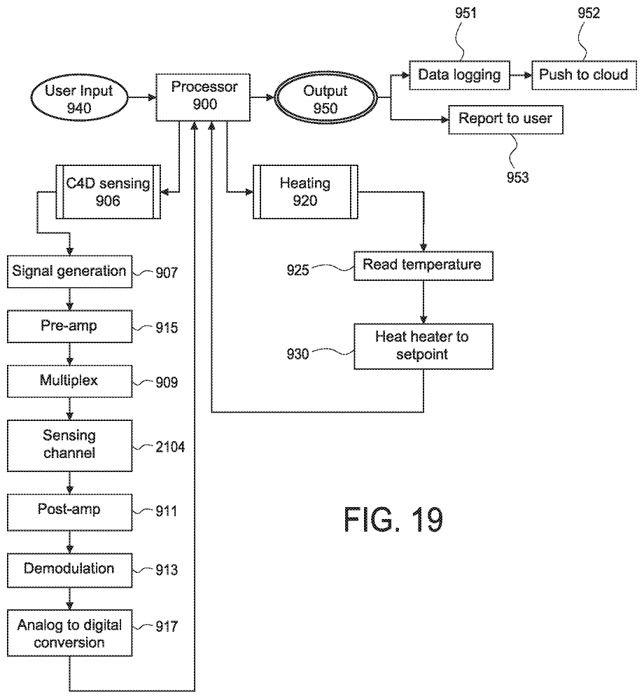

In some implementations, the device is portable and configured to detect one or more targets in a sample. As shown in FIG. 19, the device includes a processor 900 configured to control ƒC⁴D circuitry 905. The ƒC⁴D circuitry 905 includes a signal generator 907. The signal generator 907 is configured to supply one or more signals through a channel 2104 or test well as described above. The signal generator 907 is coupled to a pre-amplifier 915 to amplify the one or more signals from the signal generator 907. The one or more signals is passed through a multiplexor 909 and through the channel 2104. From the channel 2104, the signal is amplified by a post-amplifier 911 and demodulated with a demultiplexer 913. An analog to digital 917 convertor recovers the signal and forwards the digital signal to the processor 900. The processor 900 includes circuitry configured to measure, equilibrate, compare, and the like, to determine if the desired target was detected in the sample. In some embodiments, the analog to digital conversion may happen first. In some such embodiments, the induced waves can be sampled in their entirety, and demodulated digitally in software.

The processor 900 is also coupled to one or more heating elements 920 in some embodiments. The one or more heating elements 920 may be resistive heating elements. The one or more heating elements 920 are configured to heat the sample and/or the solution in the channel 2104. In some embodiments, the sample is heated to a temperature greater than or equal to 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C., 120° C., 130° C., 140° C., 150° C., 160° C., 170° C., 180° C., 190° C., 200° C., 210° C., 220° C., 230° C., 240° C., 250° C., 260° C. or any temperature, or any range of temperatures between two of the foregoing numbers. In some embodiments, the sample is cooled to a temperature less than or equal to 40° C., 35° C., 30° C., 25° C., 20° C., 15° C., 10° C., 5° C., 0° C., −5° C., −10° C., −15° C., −20° C., or any temperature or any range of temperatures between two of the foregoing numbers. In view of the foregoing, the processor 900 and/or other circuitry is configured to read the temperature 925 of the sample and/or channel 2104 and control the one or more heating elements 920 until the desired heating set point 930 is reached. In some aspects, the entire channel 2104 is configured to be heated by the one or more heating elements 920. In other aspects, only portions of the channel 2104 are configured to be heated by the one or more heating elements 920.

The processor 900 is configured to receive user input 940 from one or more user inputs such as keypads, touchscreens, buttons, switches, or microphones. Data is output 950 and logged 951, reported to a user 953, pushed to a cloud-based storage system 952, and the like. Data is sent to another device to be processed and/or further processed in some embodiments. For example, ƒC⁴D data may be pushed to the cloud and later processed to determine the presence or absence of a target(s) in the sample.

In some aspects, the device is configured to consume relatively low power. For example, the device may only require 1-10 watts of power. In some aspects, the device requires 7 watts or less of power. The device is configured to process data, wirelessly communicate with one or more other devices, send and detect signals through the channel, heat the sample/channel, and/or detect and display input/output with a touch enabled display.

In some implementations, a sample collector, sample preparer, and fluidics cartridge are formed as separate physical devices. Thus, a first sample collector device is used to collect a sample. The sample may comprise saliva, mucus, blood, plasma, stool, or cerebral spinal fluid. The sample is then transferred to a second sample preparing device. The sample preparing device includes components and reagents required for nucleic acid amplification. After the sample is prepared, it is transferred to a third device comprising a fluidics cartridge where amplification and ƒC⁴D excitations and measurements take place. In some implementations, the sample collection and sample preparation are accomplished by a single device. In some implementations, the sample preparation and fluidics cartridge are contained within a single device. In some implementations, a single device is configured to collect a sample, prepare the sample, amplify at least a portion of the sample, and analyze the sample with ƒC⁴D.

### Overview of Example Compact Fluidics Cartridges

In some aspects, the device includes a removable fluidics cartridge that is couplable to another companion device. The removable fluidics cartridge is configured to be a disposable single use cartridge. The cartridge includes a plurality of channels in some embodiments. The channels may be differently shaped. In some aspects, four shapes of channels are used and repeated to ensure accuracy. In some aspects, more than four shapes of channels are used and repeated to ensure accuracy. In some aspects, each channel is configured to detect one unique target. In other aspects each channel is configured to detect the same target. In some implementations, the cartridge includes one or more heating elements. In general, the fluidics cartridge may include at least one channel configured for ƒC⁴D analysis.

In some aspects, the cartridge includes a multi-layered laminated structure. One or more channels are stamped and/or laser cut into the substrate. The substrate includes a polypropylene film in some embodiments. One or both sides of the film are coated with an adhesive. This channel layer is secured over a polyamide heater coil in order to heat all or a portion of the channel. The channel is at least partially covered by a hydrophilic PET layer. Printed electrodes may be disposed under the PET layer. In some aspects, at least one thermistor is supplied per channel for temperature feedback.

In other aspects, the cartridge includes injected molded plastic. One or more channels are disposed within the injected molded plastic. A PET layer or PET film is coated on all or parts of the channels by laser welding the PET to the IM plastic. Injection molding may offer the benefits of rigidity and 3D structure and also allow for features such as valves, and a frame for easy handling. The cartridge may or may not include printed electronics and/or heating elements and/or thermistors depending on the particular design.

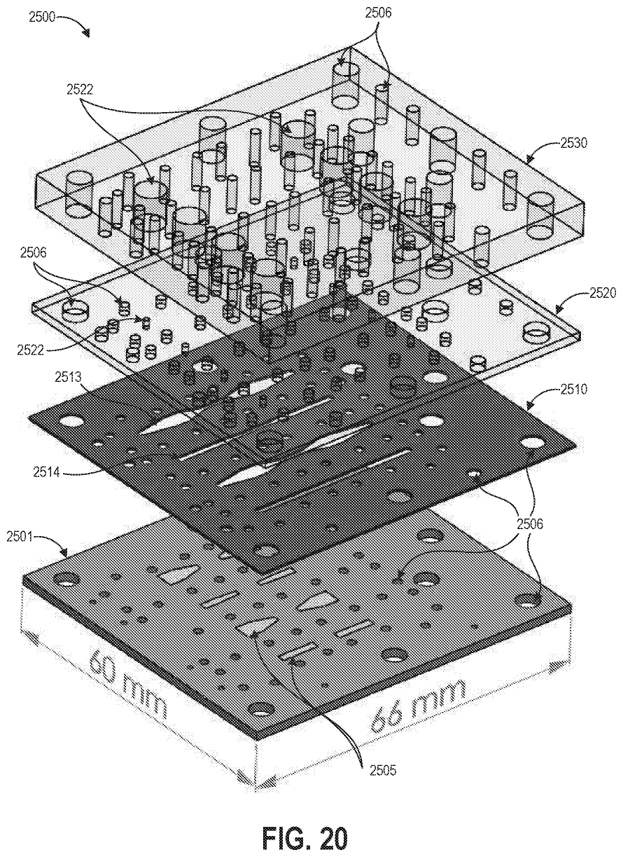

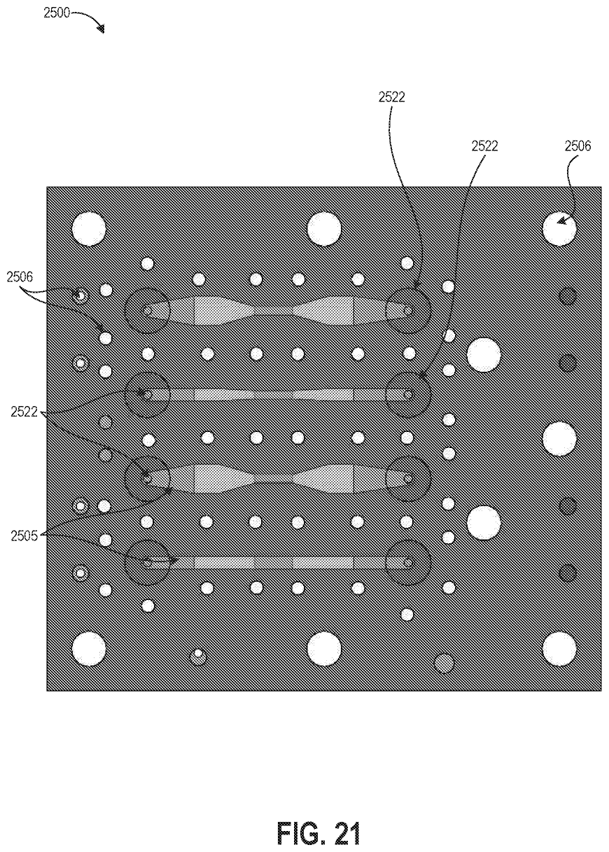

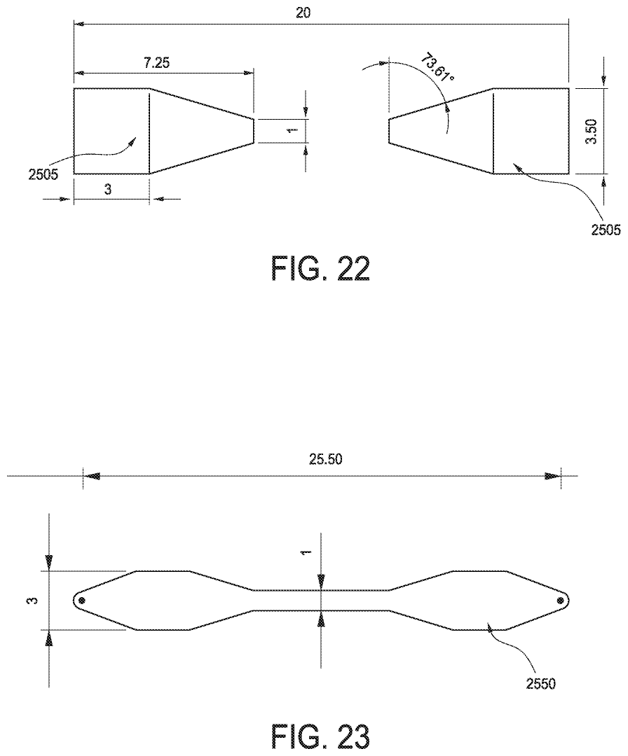

An example embodiment of a fluidics cartridge 500 is depicted in FIG. 20. As shown, the cartridge 2500 includes four layers. A PCB/PWB layer 2501 having electrodes 2505 traced thereon. The electrodes can be passivated with a 30 nm layer of titanium dioxide using methods such as atomic deposition. The PCB/PWB layer can include entry points 2506 for screws or other holding means to hold the four layers together. A power supply and detection circuitry can be in coupled to the PCB/PWB layer. A gasket layer 2510 having cutouts 2513 and 2514, and entry points 2506. The gasket layer can be manufactured from materials such as a fluorosilicone. A lower rigid substrate layer 2520 that includes entry points 2506, and inlet ports 2522. An upper rigid layer 2530 that includes entry points 2506, and inlet ports 2522. The lower and upper rigid layers can each be manufactured from materials such as acrylic. Four channels are formed when the four layers are assembled together by fixing screws or other holding means through the several entry points 2506 of the several layers. The cutouts 2513 and 2514 form the sides of the channels. The cutout 513 forms a channel having two trapezoidal ends, and the cutout 2514 forms a channel having substantially straight sides. Portions of the PCB/PWB layer 2501 including electrodes 2505 form the bottom of the channels. The lower rigid layer 2520 forms the top of the channels, and the inlet ports 2522 provide inlet and outlet ports to the channels. The inlet ports 2522 of the upper layer and inlet ports of the upper rigid layer provide a means to provide reagents to each channel. In some embodiments, a channel having two trapezoidal ends can have a volume about 30 μl to about 50 μl. In some embodiments, a channel having substantially straight sides can have a volume about 20 μl to about 30 μl. Such volumes can be adjusted by varying compression of at least the gasket layer. FIG. 21 depicts a top plan view of the fluidics cartridge 2500 of FIG. 20 and shows entry points 506 for screws or other holding means, inlet ports 2522 in communication with channels 2550, and electrodes 2505. FIG. 22 provides example dimensions for two electrodes 2505. FIG. 23 provides example dimensions for a channel 2550 having two trapezoidal ends. In some embodiments, the channel is heated to a temperature of 60° C., 61° C., 62° C., 63° C., 64° C., 65° C., 66° C., 67° C., 68° C., 69° C., 70° C., 71° C., 72° C., 73° C., 74° C., or 75° C. or within a range defined by any two of the aforementioned numbers and pressurized. In some aspects, the channel can be pressurized to 1, 2, 3, 4, 5, or 6 atmospheres or within a range defined by any two of the aforementioned pressures.

In some embodiments, a channel of a fluidics device can be adapted to or configured to hold a sample volume greater than or equal to 1 μl, 2 μl, 3 μl, 4 μl, 5 μl, 6 μl, 7 μl, 8 μl, 9 μl, 10 μl, 20 μl, 30 μl, 40 μl, 50 μl, 60 μl, 70 μl, 80 μl, 90 μl, 100 μl, 200 μl, 300 μl, 400 μl, 500 μl, 600 μl, 700 μl, 800 μl, 900 μl, or 1000 μl, or a volume or any range between any two of the foregoing volumes. In some embodiments, a channel of a fluidics device can be adapted to be pressurized. In some embodiments, the sample in a channel can be pressurized to a pressure greater than or equal to 1 atmospheres, 2 atmospheres, 3 atmospheres, 4 atmospheres, 5 atmospheres, 6 atmospheres, 7 atmospheres, 8 atmospheres, 9 atmospheres, 10 atmospheres, or any range between any two of the foregoing pressures. In some embodiments, a channel of a fluidics device can be adapted to be held at a temperature greater than or equal to −20° C., −15° C., −10° C., −5° C., 0° C., 5° C., 10° C., 15° C., 20° C., 25° C., 30° C., 35° C., 40° C., 45° C., 50° C., 55° C., 60° C., 65° C., 70° C., 85° C., 80° C., 85° C., 90° C., 95° C., 100° C., 105° C., 110° C., 115° C., 120° C., 130° C., 140° C., 150° C., 160° C., 170° C., 180° C., 190° C., 200° C., 210° C., 220° C., 230° C., 240° C., 250° C., 260° C., or any temperature or any range between any two of the foregoing temperatures.

### Overview of Example Sample Collection

In some implementations, methods, systems and device disclosed herein utilize a simplified and direct sample collection process. In this way, the number of steps from sample collection to analysis is shortened. In other words, in some implementations, it is desirable to minimize the number of times the sample is transferred and/or manipulated by the user to avoid contamination of the sample. In some aspects, the devices disclosed herein are configured to be compatible with a plurality of sample collection methods to suit all types of testing environments. Thus, homogeneous vial-to-chip interfaces are utilized in some aspects. By adjusting the sample collection systems, the detection hardware remains the same regardless of the type of sample that is collected and analyzed.

### Overview of Example Assays

Some embodiments of the methods, systems and compositions provided herein include a simple, lysis/amplification/detection of targets from crude samples in a single vessel. Some embodiments include immuno-based amplification for detection of non-nucleic acid targets. Some embodiments include reagents added to reaction which result in increased conductivity change. Some embodiments include isothermal amplification approaches, such as LAMP, SDA, and/or RCA. In some embodiments, targets for detection are biomarkers such as proteins, small molecules such as pharmaceuticals or narcotics, or biological weapons such as toxins. Detection of such targets can be achieved by conjugating immuno-based binding reagents, such as antibodies or aptamers, with nucleic acids which will participate in an isothermal amplification reaction. In some embodiments, additives to the amplification reaction can increase the solution conductivity change which is correlated with the quantification of the target. The use of additives can provide a greater sensitivity and dynamic range for detection. The use of additives can also help to reduce the formation of non specific amplification products.

Some embodiments of the methods provided herein allow for sample collection and processing to have one or more of the following desirable features: be centrifuge-free; be portable; be inexpensive; be disposable; may not require wall outlet electrics; may be easy and or intuitive to use; may require only a relatively low technical skill to use; may be able extract RNA and/or DNA from a small volume sample (e.g., 70 μL); may be able to stabilize the RNA and/or DNA until amplification; may use thermally stable reagents with no cold chain storage requirements; may be assay compatible for low level of detection samples (e.g., samples having 1,000 copies or less/mL), and/or have a dynamic range with the ability to detect viral load across, for example, at least 4 orders of magnitude; may improve the repeatability for low copy target samples.

Some embodiments of the methods, systems and compositions provided include the collection and processing of a sample for use in a diagnostic device, as described herein. Examples of a collected sample, also referred to as a biological sample, can include, for example, plant, blood, serum, plasma, urine, saliva, ascites fluid, spinal fluid, semen, lung lavage, sputum, phlegm, mucous, feces, a liquid medium comprising cells or nucleic acids, a solid medium comprising cells or nucleic acids, tissue, and the like. Methods to obtain samples can include the use of a finger stick, a heel stick, a venipuncture, an adult nasal aspirate, a child nasal aspirate, a nasopharyngeal wash, a nasopharyngeal aspirate, a nasal swab, a bulk collection in cup, a tissue biopsy or a lavage sample. More examples include environmental samples, such as soil sample, and a water sample.

### Overview of Example Amplification

Some embodiments of the methods, systems and compositions provided herein include amplification of nucleic acid targets. Methods of nucleic amplification are well known and include methods in which temperature is varied during the reaction, such as the PCR.

More examples include isothermal amplification in which the reaction can occur at a substantially constant temperature. In some embodiments, isothermal amplification of nucleic acid targets results in changes in conductivity of a solution. There are several types of isothermal nucleic acid amplification methods such as nucleic acid sequence-based amplification (NASBA), strand displacement amplification (SDA), loop-mediated amplification (LAMP), Invader assay, rolling circle amplification (RCA), signal mediated amplification of RNA technology (SMART), helicase-dependent amplification (HDA), recombinase polymerase amplification (RPA), nicking endonuclease signal amplification (NESA) and nicking endonuclease assisted nanoparticle activation (NENNA), rolling circle replication (RCA), Nicking Enzyme Amplification Reaction (NEAR), Nucleic acid sequence based amplification (NASBA), exonuclease-aided target recycling, Junction or Y-probes, split DNAZyme and deoxyribozyme amplification strategies, template-directed chemical reactions that lead to amplified signals, non-covalent DNA catalytic reactions, hybridization chain reactions (HCR) and detection via the self-assembly of DNA probes to give supramolecular structures. See e.g., Yan L., et al., Mol. BioSyst., (2014) 10: 970-1003, which is hereby expressly incorporated by reference in its entirety.

In an example of LAMP, two primers in a forward primer set are named inner (F1c-F2, c strands for “complementary”) and outer (F3) primers. At 60° C., the F2 region of the inner primer first hybridizes to the target and is extended by a DNA polymerase. The outer primer F3 then binds to the same target strand at F3c, and the polymerase extends F3 to displace the newly synthesized strand. The displaced strand forms a stem-loop structure at the 5′ end due to the hybridization of F1c and F1 region. At the 3′ end, the reverse primer set can hybridize to this strand and a new strand with stem-loop structure at both ends is generated by the polymerase. The dumbbell structured DNA enters the exponential amplification cycle and strands with several inverted repeats of the target DNA can be made by repeated extension and strand displacement. In some embodiments of the methods provided herein components for LAMP include 4 primers, DNA polymerase, and dNTPs. Examples of the application of LAMP include Viral pathogens, including dengue (M. Parida, et al., J. Clin. Microbiol., 2005, 43, 2895-2903) Japanese encephalitis (M. M. Parida, et al., J. Clin. Microbiol., 2006, 44, 4172-4178), Chikungunya (M. M. Parida, et al., J. Clin. Microbiol., 2007, 45, 351-357), West Nile (M. Parida, et al., J. Clin. Microbiol., 2004, 42, 257-263), Severe acute respiratory syndrome (SARS) (T. C. T. Hong, Q. L. Mai, D. V. Cuong, M. Parida, H. Minekawa, T. Notomi, F. Hasebe and K. Morita, J. Clin. Microbiol., 2004, 42, 1956-1961), and highly pathogenic avian influenza (HPAI) H5N1 (M. Imai, et al., J. Virol. Methods, 2007, 141, 173-180), each of the foregoing references is hereby expressly incorporated by reference herein in its entirety.

In an example of SDA, a probe includes two parts: a Hinc II recognition site at the 5′ end and another segment that includes sequences that are complementary to the target. DNA polymerase can extend the primer and incorporate deoxyadenosine 5′-[a-thio]triphosphate (dATP[aS]). The restriction endonuclease Hinc II then nicks the probe strand at the Hinc II recognition site because the endonuclease cannot cleave the other strand that includes the thiophosphate modification. The endonuclease cleavage reveals a 3′-OH, which is then extended by DNA polymerase. The newly generated strand still contains a nicking site for Hinc II. Subsequent nicking of the newly synthesized duplex, followed by DNA polymerase-mediated extension is repeated several times and this leads to an isothermal amplification cascade. In some embodiments of the methods provided herein components for SDA include 4 primers, DNA polymerase, REase HincII, dGTP, dCTP, dTTP, and dATPaS. An example of the application of SDA include Mycobacterium tuberculosis genomic DNA (M. Vincent, et al., EMBO Rep., 2004, 5, 795-800 which is hereby expressly incorporated by reference herein in its entirety).

In an example of NASBA, a forward primer 1 (P1) is composed of two parts, one of which is complementary to a 3′-end of an RNA target and the other to a T7 promoter sequence. When the P1 binds to the RNA target (RNA (+)), reverse transcriptase (RT) extends the primer into a complementary DNA (DNA (+)) of the RNA. RNase H then degrades the RNA strand of the RNA-DNA (+) hybrid. A reverse primer 2 (P2) then binds to the DNA (+), and a reverse transcriptase (RT) produces double stranded DNA (dsDNA), which contains a T7 promoter sequence. After this initial phase, the system enters the amplification phase. The T7 RNA polymerase generates many RNA strands (RNA (−)) based on the dsDNA, and the reverse primer (P2) binds to the newly formed RNA (−). RT extends the reverse primer and RNase H degrades the RNA of the RNA-cDNA duplex into ssDNA. The newly produced cDNA (DNA (+)) then becomes a template for P1 and the cycle is repeated. In some embodiments of the methods provided herein components for NASBA include 2 primers, reverse transcriptase, RNase H, RNA polymerase, dNTP, and rNTP. Examples of the application of NASBA include HIV-1 genomic RNA (D. G. Murphy, et al., J. Clin. Microbiol., 2000, 38, 4034-4041) hepatitis C virus RNA (M. Damen, et al., J. Virol. Methods, 1999, 82, 45-54), human cytomegalovirus mRNA (F. Zhang, et al., J. Clin. Microbiol., 2000, 38, 1920-1925), 16S RNA in bacterial species (S. A. Morre, et al., J. Clin. Pathol.: Clin. Mol. Pathol., 1998, 51, 149-154), and enterovirus genomic RNA (J. D. Fox, et al., J. Clin. Virol., 2002, 24, 117-130). Each of the foregoing references is hereby expressly incorporated by reference herein in its entirety.

More examples of isothermal amplification methods include: self-sustaining sequence replication reaction (3SR); 90-I; BAD Amp; cross priming amplification (CPA); isothermal exponential amplification reaction (EXPAR); isothermal chimeric primer initiated amplification of nucleic acids (ICAN); isothermal multi displacement amplification (IMDA); ligation-mediated SDA; multi displacement amplification; polymerase spiral reaction (PSR); restriction cascade exponential amplification (RCEA); smart amplification process (SMAP2); single primer isothermal amplification (SPIA); transcription-based amplification system (TAS); transcription meditated amplification (TMA); ligase chain reaction (LCR); and/or multiple cross displacement amplification (MCDA), rolling circle replication (RCA), and nicking enzyme amplification reaction (NEAR).

### Overview of Example Immuno-Isothermal Amplification

Some embodiments of the methods, systems and compositions provided herein include the use of immuno-isothermal amplification to detect non-nucleic acid targets. In some such embodiments, primers useful in an isothermal amplification method are linked to an antibody or fragment thereof, or aptamer. As used herein “aptamer” can include a peptide or oligonucleotide that binds specifically to a target molecule. In some embodiments, the antibody or aptamer can be linked to primers useful in an isothermal amplification method through covalent or non-covalent bonds. In some embodiments, primers useful in an isothermal amplification method can be linked to an antibody or aptamer through biotin and streptavidin linkers. In some embodiments, primers useful in an isothermal amplification method can be linked to an antibody or aptamer using THUNDER-LINK (Expedeon, UK).

In some embodiments, a target antigen binds to the antibody or aptamer, and the primers linked to the antibody or aptamer are substrates for isothermal amplification and/or initiate isothermal amplification. See e.g., Pourhassan-Moghaddam et al., Nanoscale Research Letters, 8:485-496 which is hereby expressly incorporated by reference herein in its entirety. In some embodiments, a target antigen is captured in a sandwich form between two antibodies or aptamers (Abs), the capture antibody and the detection antibody, which are specifically bound to the target antigen. The capture Ab, which is pre-immobilized on a solid support surface, captures the target Ag, and the detection Ab, which is linked with primers or target useful in an isothermal amplification method, attaches to the captured Ag. After washing, isothermal amplification is performed, and the presence of amplified products indicates indirectly the presence of target Ag in the sample.

### Overview of Example Recombinant Polymerase Amplification

Some embodiments include a method of nucleic acid amplification, termed recombinant polymerase amplification (RPA). In some embodiments, RPA comprises the following steps. First, a recombinase agent is contacted with a first and a second nucleic acid primer to form a first and a second nucleoprotein primer. Second, the first and second nucleoprotein primers are contacted to a double stranded target sequence to form a first double stranded structure at a first portion of said first strand and form a double stranded structure at a second portion of said second strand so the 3′ ends of said first nucleic acid primer and said second nucleic acid primer are oriented towards each other on a given template nucleic acid or DNA molecule. Third, the 3′ end of said first and second nucleoprotein primers are extended by DNA polymerases to generate first and second double stranded nucleic acids, and first and second displaced strands of nucleic acid. Finally, the second and third steps are repeated until a desired degree of amplification is reached.

Some embodiments include a method of nested RPAs. In some embodiments of a nested RPA, a first region of nucleic acid is amplified by RPA to form a first amplified region. In some embodiments, a second region of nucleic acid that is completely within the first amplified region is then amplified using RPA to form a second amplified region. This process may be repeated as often as necessary. For example, a third region of nucleic acid, which is completely within the second region, may be amplified from the second amplified region by RPA. In addition to the one, two and three rounds of RPA discussed above, at least 4 or 5 rounds of nested RPAs are also contemplated.

Some embodiments include methods of detecting a genotype using RPA. This may be useful for genotyping, for detecting a normal or diseased condition, a predisposition, or a lack of a disposition for a diseased condition. Further, RPA can be used for detecting the presence of a genome, such as for example, a genome of a pathogen. In this use, the method is useful for diagnosis and detection.

Some embodiments include recombinases, single-stranded binding proteins, polymerases, and nucleotides helpful for establishing an effective amplification reaction. Some embodiments include additional components, or the use of modified components, that contribute to establishing a recombination-polymerase amplification system that is sensitive, robust, and with optimal signal-to-noise properties. Some embodiments include engineered or modified analogues of the recombinases E. coli recA or T4 bacteriophage uvsX, polymerases such as the E. coli DNA polymerase I Klenow fragment, Bst polymerase, Sau polymerase, Phi-29 polymerase, Bacillus subtilis Pol I (Bsu), and/or single-stranded DNA binding proteins from E. coli and T4 (the gp32 protein).

Some embodiments include forms of gp32 with altered cooperativity and/or strand assimilation properties. Some embodiments include the use of T4 uvsY protein, and/or molecular crowding agents such as PEG to aid in establishing an optimal reaction environment. Some embodiments include use of other enzymes involved in DNA metabolism including topoisomerases, helicases and/or nucleases, in order to improve the amplification behavior. Some embodiments include the use of optimized conditions for repeated invasion/extension of a primer targeted to a supercoiled or linear template to generate a linear amplification, and the use of this method for DNA sequencing. Some embodiments include the use of a recombinase in detection of a specific amplified product of a reaction by directing oligonucleotides labeled in some manner to the specific product species and measuring a change in the appearance or property of the reactants as a consequence.

Some embodiments include the implementation of RPA for diagnostic applications. Some embodiments include approaches to combine oligonucleotides with distinct activities as nucleoprotein filaments to improve signal-to-noise ratios. Some embodiments include methods of product detection that obviate gel electrophoresis or fluorescent molecules such as SYBR Green. Some embodiments include the constitution of an active lyophilizate that can be stored at ambient temperature for at least 10 days and retain amplification activity when reconstituted with buffered sample only.

Some embodiments include a method to control RPA reactions, achieved by controlling the presence of supporting nucleoside triphosphate cofactors such as ATP, to limited periods during the reaction. If chemically caged nucleotide triphosphates are used, pulses of free ATP may be generated by illumination with a defined burst of light corresponding to the uncaging wavelength of the photoprotecting group. The released ATP can allow the binding of recombinase proteins to single-stranded DNA (ssDNA), and subsequent homology searching and strand exchange activity of recombinase-ssDNA complexes. In some embodiments, ATP may be periodically added to the reaction. Over time, the concentration of ATP may decrease as a consequence of hydrolysis, either recombinase hydrolysis or hydrolysis by other reaction components specifically added to hydrolyze excess ATP, and ADP. Consequently, after a period of time defined by the decreases in ATP concentration and/or an increase in the ADP concentration, recombinase molecules may cease to function and dissociate from DNA. Subsequent pulses of light at the uncaging wavelength can be delivered to release fresh ATP, or fresh ATP can be added to re-initiate recombinase activity. In this manner a series of controlled periods of homology searching and priming are enabled so that initiation of elongation can be phased.

Some embodiments include a method to control the invasion phase of RPA reactions, such as an approach to separate the activity driven by one primer from the other. In some embodiments, this method utilizes recombinase-mediated invasion at one primer target site and the completion of synthesis from that primer. In some embodiments, this generates a single-stranded displaced DNA, which can be used as a template for a second facing primer that is modified and thus unable to support recombinase-mediated initiation of synthesis. This can avoid conflicts arising through collision of polymerases.

Some embodiments include approaches to assess the polymorphic nature of amplified products without a need for size fractionation. In some embodiments, amplification reaction products are permitted to form duplex hybrids with immobilized probes of either initially single or double stranded character by the action of recombinases and/or single-stranded DNA binding proteins and other accessory molecules. Some embodiments include methods to destabilize imperfect hybrids formed between products and probes, occurring in a dynamic environment of recombinase action, and supportive of the activity of a wide variety of additional enzymatic components. In some embodiments, productive hybrids are detected by one of many standard approaches used to reveal the presence of absence of a molecular interaction.

Some embodiments include a combination of the determined in vitro conditions which support a stable persistent dynamic recombination environment with other enzymatic activities, thus permitting strand invasion and pairing between ssDNAs and duplexes to occur continuously and in the presence of other metabolic enzymes, especially those non-thermophilic enzymes that would be necessitated in conventional approaches, as well as other processes that are not equivalent, or attainable, in a system employing thermal or chemical melting.

Some embodiments include RPA, a method for the amplification of target nucleic acid polymers. Some embodiments include a general in vitro environment in which high recombinase activity is maintained in a highly dynamic recombination environment, supported by ATP. One benefit of RPA is that it may be performed without the need for thermal melting of double-stranded templates. Therefore, the need for expensive thermocyclers is also eliminated.

Leading Strand RPA (lsRPA)

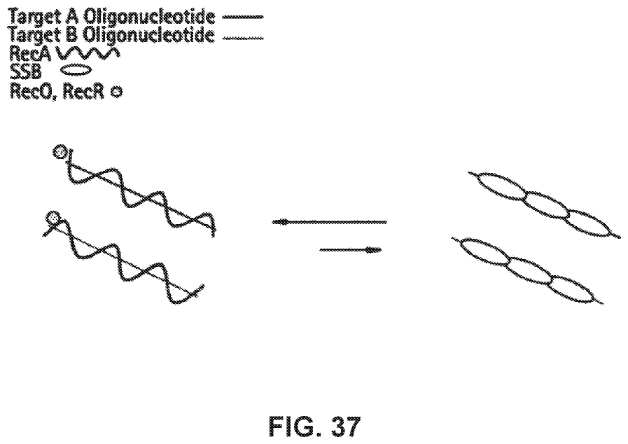

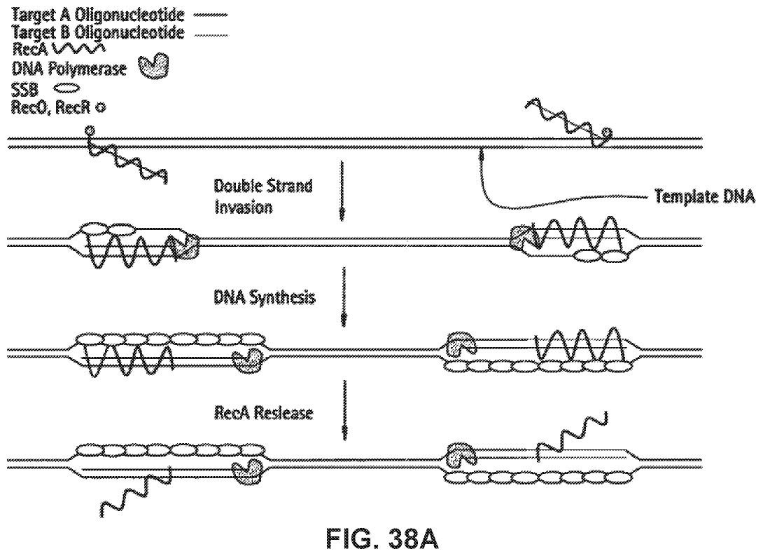

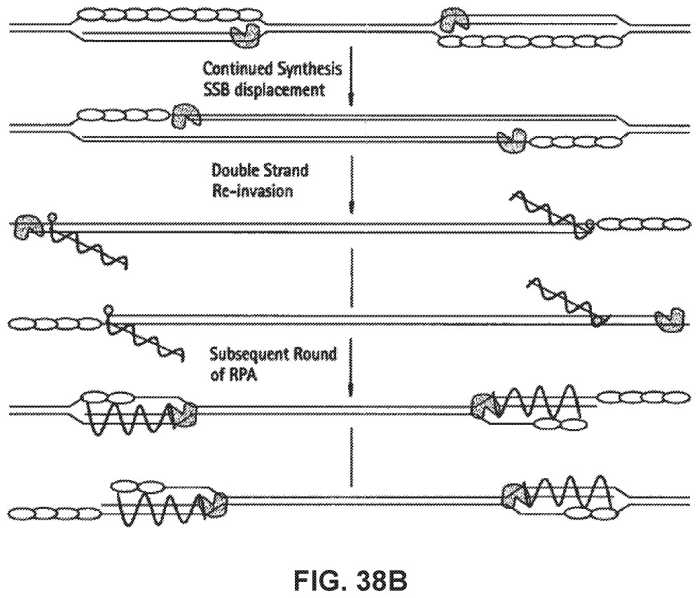

In some embodiments, RPA comprises leading strand Recombinase-Polymerase Amplification (lsRPA). In some embodiments of lsRPA, single-stranded, or partially single-stranded, nucleic acid primers are targeted to homologous double-stranded, or partially double-stranded, sequences using recombinase agents, which would form D-loop structures. In some embodiments, the invading single-stranded primers, which are part of the D-loops, are used to initiate polymerase synthesis reactions. In some embodiments, a single primer species amplifies a target nucleic acid sequence through multiple rounds of double-stranded invasion followed by synthesis. If two opposing primers are used, amplification of a fragment, can be achieved. An example of lsRPA is described briefly in FIGS. 37, 38A and 38B.

In some embodiments, the target sequence to be amplified is a double stranded DNA. However, the target sequence to be amplified not limited to double stranded DNA because other nucleic acid molecules, such as a single stranded DNA or RNA can be turned into double stranded DNA by one of skill in the arts using known methods. Suitable double stranded target DNA may be a genomic DNA or a cDNA. An RPA of the invention may amplify a target nucleic acid at least 10 fold, at least 100 fold, at least 1,000 fold, at least 10,000 fold, or at least 1,000,000 fold.

In some embodiments, the target sequence is amplified with the help of recombinase agents. A recombinase agent includes an enzyme that can coat single-stranded DNA (ssDNA) to form filaments, which can then scan double-stranded DNA (dsDNA) for regions of sequence homology. In some embodiments, when homologous sequences are located, the nucleoprotein filament (comprising the recombinase agent) strand invades the dsDNA creating a short hybrid and a displaced strand bubble known as a D-loop. Suitable recombinase agents include the E. coli RecA protein, the T4 uvsX protein, or any homologous protein or protein complex from any phyla. Eukaryotic RecA homologues are generally named Rad51 after the first member of this group to be identified. Other non-homologous recombinase agents may be utilized, for example as RecT or RecO. Recombinase agents generally require the presence of ATP, ATPγS, or other nucleoside triphosphates and their analogs. In some embodiments, recombinase agents are used in a reaction environment in which regeneration of targeting sites can occur shortly following a round of D-loop stimulated synthesis. Completed recombination events involving recombinase disassembly can avoid a stalling of amplification or inefficient linear amplification of ssDNA caused by oscillating single-sided synthesis from one end to the other.

In some embodiments, a derivative or and functional analog of a recombinase agent functions itself as a recombinase agent. For example, a small peptide from recA, which has been shown to retain some aspects of the recombination properties of recA, may be used. This peptide comprises residues 193 to 212 of E. coli recA and can mediate pairing of single stranded oligos.

Since the use of ATPγS may result in the formation of stable Recombinase-agent/dsDNA complexes that may be incompatible with efficient amplification, some embodiments use ATP and/or auxiliary enzymes to load and/or maintain the Recombinase-agent/ssDNA primer complexes. In some embodiments, the limitations of the use of ATPγS may be overcome by the use of additional reaction components capable of stripping recA bound to ATPγS from exchange complexes. In some embodiments, this role is played by a helicase such as the RuvA/RuvB complex.

Some embodiments include a method of performing RPA comprising two steps. In the first step, the following reagents are combined in a reaction: (1) at least one recombinase; (2) at least one single stranded DNA binding protein; (3) at least one DNA polymerase; (4) dNTPs or a mixture of dNTPs and ddNTPs; (5) a crowding agent; (6) a buffer; (7) a reducing agent; (8) ATP or ATP analog; (9) at least one recombinase loading protein; (10) a first primer and optionally a second primer; and (11) a target nucleic acid molecule. In the second step, the reagents are incubated until a desired degree of amplification is achieved.

In some embodiments, the recombinase is uvsX, recA or a combination of both. In some embodiments, the recombinase comprises a C terminal deletion of acidic residues to improve its activity. In some embodiments, recombinase concentrations are, for example, in the range of 0.2-12 μM, 0.2-1 μM, 1-4 μM, 4-6 μM, and 6-12 μM.

In some embodiments, the single stranded DNA binding protein is the E. coli SSB or the T4 gp32 or a derivative or a combination of these proteins. A gp32 derivative may include, at least, gp32(N), gp32(C), gp32(C)K3A, gp32(C)R4Q, gp32(C)R4T, gp32K3A, gp32R4Q, gp32R4T and a combination thereof. In some embodiments, the DNA binding protein is present at a concentration of between 1 μM and 30 μM.

In some embodiments, the DNA polymerase is a eukaryotic polymerase. Examples of eukaryotic polymerases include pol-α, pol-β, pol-δ, pol-ε and derivatives and combinations thereof. In some embodiments, the DNA polymerase is a prokaryotic polymerase. Examples of prokaryotic polymerase include E. coli DNA polymerase I Klenow fragment, bacteriophage T4 gp43 DNA polymerase, Bacillus stearothermophilus polymerase I large fragment, Phi-29 DNA polymerase, T7 DNA polymerase, Bacillus subtilis Pol I, E. coli DNA polymerase I, E. coli DNA polymerase II, E. coli DNA polymerase III, E. coli DNA polymerase IV, E. coli DNA polymerase V and derivatives and combinations thereof. In some embodiments, the DNA polymerase is at a concentration of between 10,000 units/ml to 10 units/ml, such as between 5000 units/ml to 500 units/ml. In some embodiments, the DNA polymerase lacks 3′-5′ exonuclease activity. In some embodiments, the DNA polymerase contains strand displacing properties.

Some embodiments include a derivative of a protein mentioned herein. For example, some embodiments include a derivative of a recombinase, polymerase, recombinase loading protein, single stranded DNA binding protein, accessory agents, RecA/ssDNA nucleoprotein filaments stabilizing agent and the like. The derivative of these proteins include, at least, a fusion protein comprising a C terminus tag, N terminus tag, or C and N terminus tags. Non-limiting examples of suitable sequence tags include 6-histidine (6×-His; HHHHHH), c-myc epitope (EQKLISEEDL), FLAG octapeptide (DYKDDDDK), Protein C (EDQVDPRLIDGK), Tag-100 (EETARFQPGYRS), V5 epitope (GKPIPNPLLGLDST), VSV-G (YTDIEMNRLGK), Xpress (DLYDDDDK), and hemagglutinin (YPYDVPDYA). Non-limiting examples of suitable protein tags include 0-galactosidase, thioredoxin, His-patch thioredoxin, IgG-binding domain, intein-chitin binding domain, T7 gene 10, glutathione-S-transferase (GST), green fluorescent protein (GFP), and maltose binding protein (MBP). It will be understood by those in the art that sequence tags and protein tags can be used interchangeably, e.g., for purification and/or identification purposes.

In some embodiments, the dNTPs include, for example, dATP, dGTP, dCTP, and dTTP. In leading and lagging strand RPA, ATP, GTP, CTP, and UTP may also be included for synthesis of RNA primers. In addition, ddNTPs (ddATP, ddTTP, ddGTP and ddGTP) may be used to generate fragment ladders. The dNTP may be used at a concentration of between 1 μM to 200 μM of each NTP species. A mixture of dNTP and ddNTP may be used with ddNTP concentrations at 1/100 to 1/1000 of that of the dNTP (1 μM to 200 μM).

In some embodiments, crowding agents used in RPA include polyethylene glycol (PEG), dextran and Ficoll. The crowding agent may be at a concentration of between 1% to 12% by volume or 1% to 12% by weight of the reaction. Examples of PEGs include PEG1450, PEG3000, PEG8000, PEG10000, PEG compound molecular weight 15000- to 20,000 (also known as Carbowax 20M), and combinations thereof.

The buffer solution in an RPA may be a Tris-HCl buffer, a Tris-Acetate buffer, or a combination thereof. The buffers may be present at a concentration of between 10 to 100 mM. The buffered pH may be between 6.5 to 9.0. The buffer may further contain Mg ions (e.g., in the form of Mg Acetate) at a concentration between 1 to 100 mM with a concentration of between 5 to 15 mM being preferred. One preferred Mg concentration is 10 mM (Mg concentration or Mg Acetate concentration).

In some embodiments, reducing agents to be used include DTT. In some embodiments, the DTT concentration is between 1 mM and 10 mM.

In some embodiments, the ATP or ATP analog is ATP, ATP-γ-S, ATP-β-S, ddATP or a combination thereof. In some embodiments, the ATP or ATP analog concentration is between 1 and 10 mM.

Recombinase loading protein may include, for example, T4uvsY, E. coli recO, E. coli recR and derivatives and combinations of these proteins. One preferred concentration of these proteins is between 0.2 and 8 μM.

The primers used may be made from DNA, RNA, PNA, LNA, morpholino backbone nucleic acid, phosphorothiorate backbone nucleic acid or a combination thereof. Combinations thereof in this case refers to a single nucleic acid molecules, which may contain one or more of one base connected to one of more of another base. In some embodiments, the concentration of these molecules ranges between 25 nM to 1000 nM. In some embodiments, the primers contain a non-phosphate linkage between the two bases at its 3′ end and is resistant to 3′ to 5′ nuclease activity. In some embodiments, the primers contain a locked nucleic acid at its 3′ ultimate base or 3′ penultimate base. For example, in a nucleic acid of the sequence 5′-AGT-3′, the T is the 3′ ultimate base and the G is the 3′ penultimate base. In some embodiments, the primers may be at least 20 bases in length or at least 30 bases in length. In some embodiments, the primers are between 20 to 50 bases in length. In some embodiments, the primers are between 20 to 40 bases in length such as between 30 to 40 bases in length.

In some embodiments, the primers include a 5′ sequence that is not complementary to the target nucleic acid. These 5′ sequence may contain, for example a restriction endonuclease recognition site. The primers may be partly double stranded with a single stranded 3′ end.

In some embodiments, a nucleic acid is labeled with a detectable label. A detectable label includes, for example, a fluorochrome, an enzyme, a fluorescence quencher, an enzyme inhibitor, a radioactive label and a combination thereof. In some embodiments, a nucleic acid does not have a detectable label or dye or marker.

The target nucleic acid may be single stranded or double stranded. In some embodiments, single stranded nucleic acids are converted to double stranded nucleic acid. In some embodiments, the target nucleic acid is supercoiled or linear. The sequence to be amplified (target nucleic acid) may be in between other sequences. The sequence to be amplified may also be at one end of a linear nucleic acid. In some embodiments, the target nucleic acid is linear and not connected to non-target nucleic acids. In other words, where the target nucleic acid is linear, it can be in any of the following formats:

1. [non-target nucleic acid]-[target nucleic acid]-[non-target nucleic acid]

2. [non-target nucleic acid]-[target nucleic acid]

3. [target nucleic acid]-[non-target nucleic acid]

4. [target nucleic acid]

The arrangement above is intended to represent both single stranded nucleic acids and double stranded nucleic acids. “1” may be described as a target nucleic acid molecule which is linear with two ends and wherein both ends are linked to a non-target nucleic acid molecule. “2” may be described as a target nucleic acid molecule which is linear with two ends and wherein one end is linked to a non-target nucleic acid molecule. “3” may be described as a target nucleic acid molecule which is a linear nucleic acid molecule (with no non-target nucleic acid).

In some embodiments, the target nucleic acid is a single-stranded nucleic acid which is converted to a double stranded nucleic acid by a polymerase or a double stranded nucleic acid denatured by the action of heat or chemical treatment.

The target nucleic acid may be of any concentration such as less than 10,000 copies, less than 1000 copies, less than 100 copies, less than 10 copies or 1 copy in a reaction. A reaction volume may be 5 μl, 10 μl, 20 μl, 30 μl, 50 μl, 75 μl, 100 μl, 300 μl, 1 ml, 3 ml, 10 ml, 30 ml, 50 ml or 100 ml.

The reaction may be incubated between 5 minutes and 16 hours, such as between 15 minutes and 3 hours or between 30 minutes and 2 hours. The incubation may be performed until a desired degree of amplification is achieved. The desired degree of amplification may be 10 fold, 100 fold, 1000 fold, 10,000 fold, 100,000 fold or 1,000,000 fold amplification. Incubation temperature may be between 20° C. and 50° C., between 20° C. and 40° C., such as between 20° C. and 30° C. Advantages of the methods described herein are that in some embodiments the temperature is not critical, and in some embodiments precise control is not absolutely necessary. For example, in some embodiments, in a field environment, it is sufficient to keep the RPA at room temperature, or close to body temperature (35° C. to 38° C.) by placing the sample in a body crevice. Furthermore, in some embodiments, the RPA may be performed without temperature induced melting of the template nucleic acid.

In some embodiments, the RPA comprises accessory agents. Examples of accessory agents include helicase, topoisomerase, resolvase and a combination thereof which possess unwinding, relaxing, and resolving activities respectively on DNA. The accessory agents may include RuvA, RuvB, RuvC, RecG, PriA, PriB, PriC, DnaT, DnaB, DnaC, DnaG, DnaX clamp loader, polymerase core complex, DNA ligase or a sliding clamp or a combination thereof. The sliding claim may be E. coli 3-dimer sliding clamp, the eukaryotic PCNA sliding clamp, or the T4 sliding clamp gp45 and a combination thereof. The accessory agents may include, in addition, DNA Polymerase III holoenzyme complex consisting of P-Clamp, DnaX Clamp Loader, and the Polymerase Core Complex. In some embodiments, these latter accessory agents would allow the performance of leading and lagging RPA.

In some embodiments, the RPA is performed in the presence of a RecA/ssDNA nucleoprotein filaments stabilizing agent. Examples of such stabilizing include RecR, RecO, RecF and a combination thereof. These stabilizing agents may be present at a concentration of between 0.01 μM to 20 μM. Other examples of stabilizing agents include the T4 uvsY protein which stabilizes uvsX/ssDNA nucleoprotein complexes.

In some embodiments, other components of RPA include a system for ATP regeneration (convert ADP to ATP). Such system may be, for example, phosphocreatine and creatine kinase.

The RPA reaction may also include a system to regenerate ADP from AMP and to convert pyrophosphate to phosphate (pyrophosphatase).

In some embodiments, the RPA reaction as listed above are performed with E. coli components completely by using recA, SSB, recO, recR and/or E. coli polymerase.

In some embodiments, the RPA reaction is performed with T4 components by using uvsX, gp32, uvxY, and/or T4 polymerase.

In some embodiments, RPA is performed by combining some or all of the following reagents: (1) a uvsX recombinase at a concentration of between 0.2 to 12 μM; (2) a gp32 single stranded DNA binding protein at a concentration between 1 to 30 μM; (3) a Bacillus subtilis DNA polymerase I large fragment (Bsu polymerase) at a concentration between 500 to 5000 units per ml; (4) dNTPs or a mixture of dNTPs and ddNTPs at a concentration of between 1-300 μM; (5) polyethylene glycol at a concentration of between 1% to 12% by weight or by volume; (6) Tris-acetate buffer at a concentration of between 1 mM to 60 mM; (7) DTT at a concentration of between 0.025 mM and 1 mM, or between 0.025 mM and 10 mM; (8) ATP at a concentration of between 1 mM-10 mM; (9) uvsY at a concentration of between 0.2 μM-8 μM; (10) a first primer and optionally a second primer, wherein said primers are at a concentration of between 50 nM to 1 μM; and/or (11) a target nucleic acid molecule of at least one copy. In some embodiments, after the reaction is assembled, it is incubated until a desired degree of amplification is achieved. In some embodiments, this is within 2 hours, or within 1 hour, such as, for example, in 50 minutes.

An advantage of the methods described herein is that the reagents for RPA, with the optional exception of the crowding agent and buffer, may be freeze dried (e.g., lyophilized) before use. In some embodiments, freeze dried reagents offer are desired because they do not require refrigeration to maintain activity. For example, a tube of RPA reagents may be stored at room temperature. This feature may be useful in field conditions where access to refrigeration is limited.

In some embodiments, the RPA reagents may be freeze dried onto the bottom of a tube, or on a bead (or another type of solid support). In some embodiments, to perform an RPA reaction, the reagents are reconstituted in a buffer solution and with a crowding reagent, or simply a buffered solution or water, dependent on the composition of the freeze-dried reagents. In some embodiments, a target nucleic acid, or a sample suspected to contain a target nucleic acid is added. The reconstitution liquid may also contain the sample DNA. In some embodiments, the reconstituted reaction is incubated for a period of time and the amplified nucleic acid, if present, is detected.

In some embodiments, the reagents are assembled by combining the reagents such that when constituted, they will have the following concentrations: (1) a uvsX recombinase at a concentration of between 0.2 to 12 μM; (2) a gp32 single stranded DNA binding protein at a concentration between 1 to 30 μM; (3) a T4 gp43 DNA polymerase or Bsu polymerase at a concentration between 500 to 5000 units per ml; (4) dNTPs or a mixture of dNTPs and ddNTPs at a concentration of between 1-300 μM; (5) DTT at a concentration of between 1 mM-10 mM; (6) ATP at a concentration of between 1 mM-10 mM; (7) uvsY at a concentration of between 0.2 μM-8 μM. Optionally, a first primer and optionally a second prime may be added where their concentration would be between 50 nM to 1 μM when reconstituted. In some embodiments, the reagents are freeze dried before use. Stabilizing agents such as trehalose sugar may be included in the freeze dried mixture, for example at 20 mM to 200 mM and most optimally 40 mM to 80 mM in the reconstituted reaction, in order to improve freeze-drying performance and shelf life. If desired, the freeze dried reagents may be stored for 1 day, 1 week, 1 month or 1 year or more before use.

In use, the reagents may be reconstituted with buffer (a) Tris-acetate buffer at a concentration of between 1 mM to 60 mM; and (b) polyethylene glycol at a concentration of between 1% to 12% by weight or by volume, or (c) with water. In some embodiments, if the primers were not added before freeze drying, they can be added at this stage. In some embodiments, a target nucleic acid, or a sample suspected of containing a target nucleic acid is added to begin the reaction. The target, or sample, nucleic acid may be contained within the reconstitution buffer as a consequence of earlier extraction or processing steps. In some embodiments, the reaction is incubated until a desired degree of amplification is achieved.

Any of the RPA reaction conditions discussed herein may be freeze dried. For example, the following reagents can be assembled by combining each reagent such that when constituted, they will have the following concentrations: (1) 100-200 ng/μl uvsX recombinase; (2) 600 ng/μl gp32; (3) 20 ng/μl Bsu polymerase or T4 polymerase; (4) 200 μM dNTPs; (5) 1 mM DTT (6) 3 mM ATP or an ATP analog; (7) 16 ng/μl to 60 ng/μl uvsY; (8) 50 nM to 300 nM of a first primer and 50 nM to 300 nM of a second primer; (9) 80 mM Potassium acetate; (10) 10 mM Magnesium acetate; (11) 20 mM Phosphocreatine; (12) 50 ng/μl to 100 ng/μl Creatine kinase. The reagents may be freeze dried onto the bottom of a tube or in a well of a multi-well container. The reagent may be dried or attached onto a mobile solid support such as a bead or a strip, or a well.

As another example, the following reagents can be assembled by combining each reagent such that when constituted, they will have the following concentrations: (1) 100-200 ng/μl uvsX recombinase; (2) 300-1000 ng/μl gp32; (3) 10-50 ng/μl Bsu polymerase or T4 polymerase; (4) 50-500 μM dNTPs; (5) 0.1 to 10 mM DTT; (6) 3 mM ATP or an ATP analog; (7) 16 ng/μl to 60 ng/μl uvsY; (8) 50 nM to 1000 nM of a first primer and 50 nM to 1000 nM of a second primer; (9) 40 mM to 160 mM Potassium acetate; (10) 5 mM to 20 mM Magnesium acetate; (11) 10 mM to 40 mM Phosphocreatine; (12) 50 ng/μl to 200 ng/μl Creatine kinase. In some embodiments, these reagents are freeze dried and stored. In some embodiments, in use, the reagents are reconstituted with Tris-acetate buffer at a concentration of between 1 mM to 60 mM and polyethylene glycol at a concentration of between 1% to 12% by weight or by volume. The primers, item 8 above, may be omitted before freeze drying and added after reconstitution. In some embodiments, to initiate the RPA, a target nucleic acid, or a sample suspected of containing a target nucleic acid is added. In some embodiments, the reaction is incubated until a desired degree of amplification is achieved.

Some embodiments comprise a kit for performing RPA. The kit may comprise any of the reagents discussed above for RPA and may be in the concentrations described above. The reagents of the kit may be freeze dried. For example, the kit may contain (1) 100-200 ng/μl uvsX recombinase; (2) 300 ng/μl to 1000 ng/μl gp32; (3) 10 ng/μl to 50 ng/μl Bsu polymerase or T4 polymerase; (4) 50 μM to 500 μM dNTPs; (5) 0.1 to 10 mM DTT; (6) 1 mM to 5 mM ATP or an ATP analog; (7) 16 ng/μl to 60 ng/μl uvsY; (8) 50 nM to 1000 nM of a first primer and 50 nM to 1000 nM of a second primer (optional); (9) 40 mM to 160 mM Potassium acetate; (10) 5 mM to 20 mM Magnesium acetate; (11) 10 mM to 40 mM Phosphocreatine; (12) 50 ng/μl to 200 ng/μl Creatine kinase.

In some embodiments, RPA is performed with several auxiliary enzymes that can promote efficient disassembly of Recombinase-agent/dsDNA complexes after DNA synthesis initiation. These auxiliary enzymes include those that are capable of stimulating 3′ to 5′ disassembly and those capable of supporting 5′ to 3′ disassembly.

Auxiliary enzymes can include several polymerases that can displace RecA in the 3′ to 5′ direction and can stimulate 3′ to 5′ disassembly of Recombinase-agent/dsDNA complexes. These DNA polymerases can include E. coli PolV and homologous polymerase of other species. In some embodiments, in the life of E. coli, displacement of RecA in the 3′ to 5′ direction occurs as part of SOS-lesion-targeted synthesis in concert with SSB, sliding clamps and DNA polymerase. In some embodiments, the polymerase involved in this activity in E. coli is Poly, a member of the superfamily of polymerases including UmuC, DinB, Rad30, and Rev1, whose function in vivo may be to copy DNA lesion templates. In some embodiments, the in vitro 3′ to 5′ disassembly of RecA filaments cannot be catalyzed by PolI, PolIII, or PolIV alone. In some embodiments, only PolV, in concert with SSB, has measurable ATP-independent 3′ to 5′ RecA/dsDNA disassembly activity. In effect, PolV pushes and removes RecA from DNA in a 3′ to 5′ direction ahead of the polymerase in some embodiments. Inclusion of PolV or a functional homologue may improve the amplification efficiency.

Other auxiliary enzymes can include a class of enzymes called helicases that can be used to promote the disassembly of RecA from dsDNA. In some embodiments, these promote disassembly in both the 5′ to 3′ and 3′ to 5′ directions. In some embodiments, helicases function to move the branch points of recombination intermediates from one place to another, to separate strands, and to disassemble and recycle components bound to DNA. In some embodiments, after the first round of invasion/synthesis has occurred in RPA, two new DNA duplexes are “marked” by the presence of RecA bound over the site to which primers must bind for additional rounds of synthesis. In such a situation dsDNA may tend to occupy the high affinity site in RecA, or homologs, until it is actively displaced, either by ATP hydrolysis-dependent dissociation in the 5′ to 3′ direction, which may be limiting, or by 3′ to 5′ dissociation by some other active process. In some embodiments, a helicase complex for stimulating disassembly of RecA from intermediates includes the E. coli proteins RuvA and RuvB. In some embodiments, the RuvAB complex promotes branch migration, and dissociates the RecA protein, allowing RecA to be recycled. In some embodiments, the RuvAB complex is targeted to recombination intermediates, particularly Holliday junction-like structures. As it works the RuvAB complex may encircle DNA and force RecA from the DNA in an ATP-driven translocation. In some embodiments, the RuvAB complex can recognize branched structures within the RecA coated DNA. Incorporation of RuvAB into the RPA mixture can promote the dissociation of RecA from dsDNA following strand exchange and displacement, allowing renewed synthesis of the duplicated template from the same site. Additionally, the RuvAB complex can act in concert with RuvC, which finally cuts and resolves Holliday junctions. In some embodiments, with RuvC added to the RPA reaction mixture, complicated structures such as Holliday junctions formed at invasion sites, can be resolved. In some embodiments, resolvase activity, such as that provided by RuvC, is useful when the targeting oligonucleotides are partially double-stranded. In some embodiments, reverse branch migration can generate Holliday junctions, which can then be resolved by the RuvABC complex, to generate clean separated amplification products.

In some embodiments, auxiliary enzymes include the E. coli RecG protein. In some embodiments, RecG can stimulate disassembly of branch structures. In vivo this protein may function to reverse replication forks at sites of DNA damage by unwinding both leading and lagging strands driving the replication fork back to generate a 4-way junction. In some embodiments, in vivo such junctions function as substrates for strand switching to allow lesion bypass. In vitro, RecG may bind to D-loops and lead to a decrease in D-loop structures by driving reverse branch migration. In some embodiments, RecG prefers a junction with double-stranded elements on either side, hence partly double-stranded targeting oligonucleotides, homologous to the targeting site in both single-stranded and double-stranded regions, may be useful. This may stimulate reverse branch migration and formation of a Holliday junction, which can be resolved by the RuvABC complex. In vivo RecG and RuvAB may compete to give different outcomes of recombination since branch migration will be driven in both directions. In both cases the proteins may target junction DNA coated with RecA, and disassemble it in an active manner.

In some embodiments, auxiliary enzymes useful in an RPA reaction mixture allow continual generation of RecA nucleoprotein filaments in the presence of ATP and SSB. In order to allow removal of RecA at the appropriate moments, some embodiments use ATP rather than ATPγS in an RPA reaction. In some embodiments, RecA/ssDNA filaments formed with ATP spontaneously depolymerize in the 5′ to 3′ direction, and in the presence of SSB, repolymerization will not occur at significant rates. In some embodiments, the solution to this problem is the use of the RecO, RecR, and/or possibly RecF proteins. In some embodiments, the uvsY protein is employed to stabilize the T4 uvsX nucleoprotein filaments in a similar manner. In some embodiments, in the presence of SSB and ATP, RecA/ssDNA filaments dissociate. In some embodiments, if RecA/ssDNA is incubated in the presence of RecO and RecR proteins this dissociation does not occur. In some embodiments, the RecR protein remains associated with the filament and stabilizes the structure indefinitely. In some embodiments, even if ssDNA is bound by SSB, in the presence of RecR and RecO, filaments of RecA can reassemble displacing SSB. In the T4 phage system similar properties may be attributed to the uvsY protein. Thus, it is possible in some embodiments to obviate the use of ATPγS by using ATP in the presence of RecO and RecR to maintain RecA/ssDNA filament integrity, or uvsY to maintain the uvsX/ssDNA filament integrity. In some embodiments, the RecF protein interacts with the RecO and RecR system in a seemingly opposing manner. RecF competes with RecR tending to drive filament disassembly in vitro. In some embodiments, all three components in vivo function together to control the generation of invading structures, while limiting the extent of RecA coating of ssDNA. In some embodiments, RecF is included in RPA reactions at an appropriate concentration to re-capitulate the dynamics of the in vivo processes. RecF may facilitate dissociation of RecA-coated intermediates after invasion has occurred.

In some embodiments, RPA allows the formation of short stretches of double-stranded nucleic acids bearing a free 3′-OH for extension from double-stranded templates without thermal melting. In some embodiments, this is achieved by using the RecA protein from E. coli (or a RecA relative from other phyla including the T4 uvsX protein). In the presence of ATP, dATP, ddATP, UTP, ATPγS, and possibly other types of nucleoside triphosphates and their analogs, RecA or uvsX may form a nucleoprotein filament around single-stranded DNA. In some embodiments, this filament scans double-stranded DNA. In some embodiments, when homologous sequences are located the recombinase will catalyze a strand invasion reaction and pairing of the oligonucleotide with the homologous strand of the target DNA. In some embodiments, the original pairing strand is displaced by strand invasion leaving a bubble of single stranded DNA in the region.

In some embodiments, RecA protein is obtained from commercial sources. In some embodiments, it is purified according to standard protocols. In some embodiments, a RecA homologue is purified from thermophilic organisms including Thermococcus kodakaraensis, Thermotoga maritima, Aquifex pyrophilus, Pyrococcus furiosus, Thermus aquaticus, Pyrobaculum islandicum, or Thermus thermophilus. In some embodiments, RecA is purified from a prokaryote such as Salmonella typhimurium, Bacillus subtilis, Streptococcus pneumoniae, Bacteroides fragilis, Proteus mirabilis, Rhizobium meliloti, Pseudomonas aeruginosa, from a eukaryote such as Saccharomyces cerevisiae, Ustilago maydis, including vertebrates e.g. Human Rad51 or Xenopus laevis, or from a plant such as broccoli. In some embodiments, E. coli recA, and T4 uvsX protein, is purified from overexpression cultures using a hexahistidine tag at the C terminus, and remains biologically active.

In some embodiments, leading strand Recombinase-Polymerase Amplification method (lsRPA) can be divided into four phases.

1) Sequence Targeting

In some embodiments, RPA is initiated by targeting sequences using synthetic oligonucleotides coated with RecA, or a functional homologue such as the T4 uvsX protein. In some embodiments, in order to permit exponential amplification two such synthetic oligonucleotides are employed in a manner such that their free 3-ends are orientated toward one another. In some embodiments, Nucleoprotein filaments comprising these oligonucleotides and recombinase protein identify targets in complex DNA rapidly and specifically. In some embodiments, once targeted the recombinase protein catalyses strand exchange such that D-loop structures are formed. Some embodiments use ATP rather than ATPγS in the procedure for efficient amplification. If ATP is used, RecO, RecR, and/or RecF, molecules may prove useful for efficient amplification, or uvsY protein if uvsX recombinase is employed.

2) Initiation of DNA Synthesis

In some embodiments, DNA polymerases detect and bind to the hybrid between the invading oligonucleotides and the template DNA and initiate DNA synthesis from the free 3-hydroxyl exposed in the hybrid. Some embodiments include disassembly of recombinase protein from the double-stranded hybrid formed by strand exchange after exposure of this 3-hydroxyl, and subsequent DNA synthesis. In some embodiments, to attain this disassembly, ATP is employed which may support spontaneous disassembly of recombinase from invasion complexes. Additionally, disassembly can be stimulated/enhanced by the use of other proteins contained within the reaction mixture such as RuvA, RuvB, RuvC, recG, other helicases, or other stimulatory components, which can act to strip recombinase from the strand exchange product.

3) Strand Displacement DNA Synthesis and Replicon Separation.

In some embodiments, as the DNA polymerases synthesize complementary copies of template DNAs using the free 3-hydroxyls of invading oligonucleotides, or their partly extended products, the polymerases displace single-stranded DNAs, which may be coated with single strand binding proteins (SSB) included in the reaction. In some embodiments, invasion of oligonucleotides at both ends of the target nucleic acid sequence occurs in similar timeframes, such that two polymerases on the same template nucleic acid initially progress toward one another. In some embodiments, when these extending complexes meet one another, the original template simply falls apart, and the polymerases continue to synthesize without a need for strand displacement, now copying SSB-bound ssDNA template. In some embodiments, because of steric hinderance, polymerases may become dissociated from the template temporarily when the polymerases meet to permit the separation of the two template strands

4) Completion of Synthesis and Re-Invasion.

In some embodiments, once the template strands have separated, polymerases complete the extension to the end of the template (or past the sequence acting as the second, facing, targeting site if the initial template is longer than the desired product). In some embodiments, new products are targeted and replicated in a manner similar to the original templates, that is from both targeted ends, in order to permit exponential amplification. In some embodiments, the newly synthesized targeted site are freely available to targeting recombinase/oligonucleotide filaments. In some embodiments, the site initially used to prime synthesis is freed as a consequence of the use of conditions in the reaction that favor disassembly of recombinase from strand exchange products. In some embodiments, providing the re-invasion at this latter site occurs in less time than it takes the polymerase to synthesize past the second targeting site, be primed at that second site, and return to the first site, then single-stranded DNA will not be the primary product and exponential amplification occurs. In some embodiments, having multiple synthetic complexes operating on the same template allows shorter amplification times to be achieved.

RPA Using Simultaneous Leading and Lagging Strand Synthesis

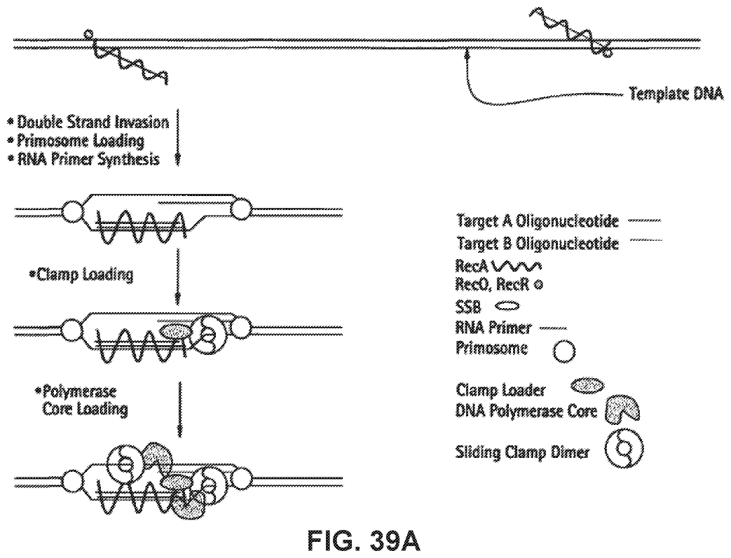

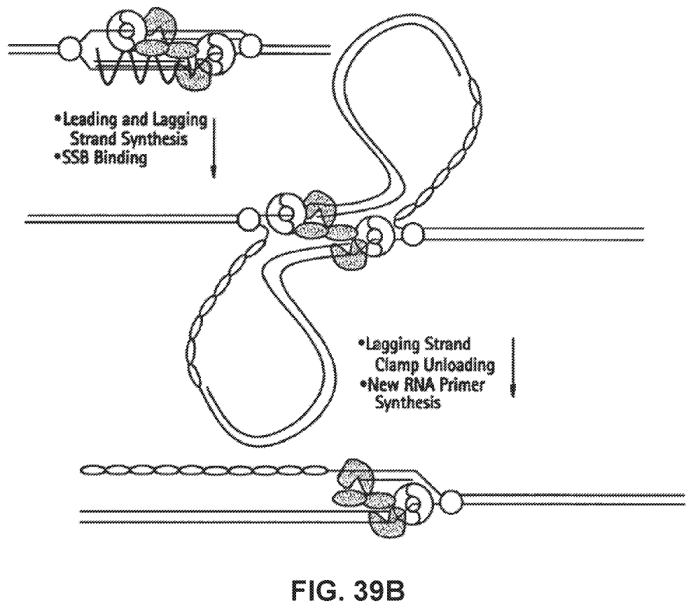

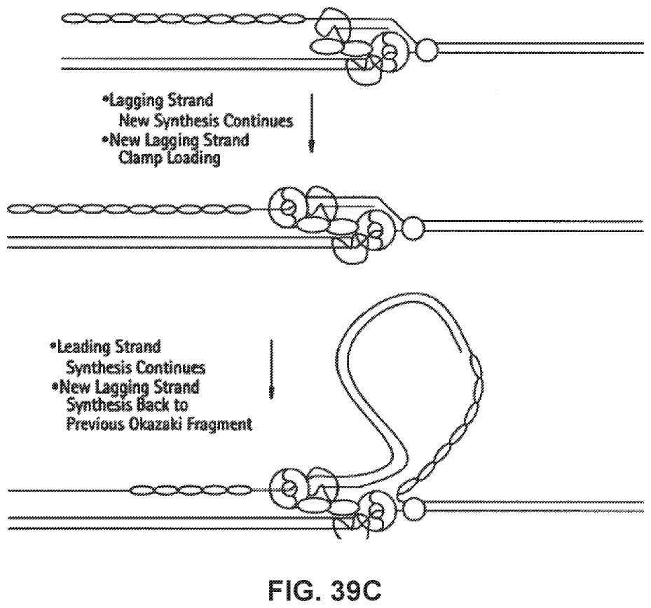

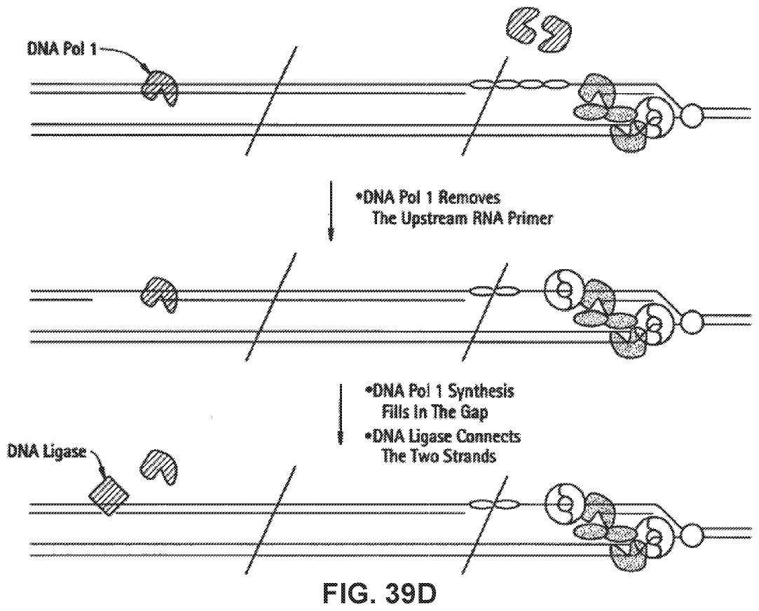

In some embodiments, lsRPA includes a multi-component system with the capacity to regenerate targeting sequences thus permitting exponential amplification of double-stranded DNA. In some embodiments, lsRPA avoids linear production of single-stranded DNA. Another approach that in some embodiments completely avoids the possibility of single-stranded products and a requirement for simultaneous end initiation involves a more complex reaction mixture. In some embodiments, this system recapitulates events occurring during the normal replication cycle of cells to permit coupled leading and lagging strand synthesis. An example of this method, leading/lagging strand RPA is described briefly in FIGS. 37 and 39A-39D.

For clarity of description, the RPA method may be divided into four phases, but in some embodiments all phases occur simultaneously in a single reaction.

1) Sequence Targeting

In some embodiments, RPA is initiated by targeting sequences using synthetic oligonucleotides coated with RecA, or T4 uvsX, or a functional homologue. In some embodiments, such nucleoprotein filaments identify targets in complex DNA rapidly and specifically. In some embodiments, once targeted, the RecA or uvsX protein catalyses strand exchange such that a D-loop structure is formed. Some embodiments include the use of ATP rather than ATPγS in the procedure for efficient amplification. In some embodiments, the linkage of leading and lagging strand syntheses however obviates rapid recombinase stripping after initiation of synthesis. In some embodiments, if ATP is used, RecO, RecR, and RecF are employed with bacterial recA recombinase, or the T4 uvsY, proteins are used for efficient amplification with T4 uvsX protein.

2) Primosome Assembly

In some embodiments, Primosomes are assembled at D-loops. In some embodiments, in E. coli, D-loop structures are formed by RecA as part of the mechanism to rescue damaged DNA in vivo, or during other forms of recombination. In some embodiments, the purpose of the combined action of RecA-mediated strand exchange and primosome assembly is to generate a replication fork. An example of a replication fork is a nucleoprotein structure comprising the separated template DNA strands and the replisome. In some embodiments, the replisome includes the polymerase holoenzyme complex, the primosome, and other components for simultaneously replicating both strands of template DNA. In some embodiments, primosomes provide DNA unwinding and the Okazaki fragment priming functions for replication fork progression. In some embodiments, similar primosome assembly occurs at recombination intermediates in T4 phage directed by gp59 and gp41 protein.

In some embodiments, the primosome assembly proteins are PriA, PriB, PriC, DnaT, DnaC, DnaB, and DnaG. In some embodiments, these proteins may assemble a primosome complex on bacteriophage phiX174 DNA in vitro. In some embodiments, PriA binds to the primosome assembly site (PAS) on the phiX174 chromosome. In some embodiments, PriB, DnaT, and PriC bind sequentially to the PriA-DNA complex. In some embodiments, PriB stabilizes PriA at the PAS and facilitate binding of DnaT. In some embodiments, omission of PriC from the reaction lowers priming 3 to 4 fold. In some embodiments, the function of PriC in the bacterium is genetically redundant to PriB. In some embodiments, DnaC loads DnaB into the complex in an ATP-dependent fashion. In some embodiments, this PriABC-DnaBT complex is competent to translocate along the chromosome. In some embodiments, the DnaG primase interacts transiently with the complex to synthesize RNA primers.

In some embodiments, during replication in E. coli, DnaB and DnaG function as a helicase and primase respectively. In some embodiments, these two components associate with the PolIII holoenzyme to synthesize primers for the Okazaki fragments. In some embodiments, other primosome components described are involved in assembly of the primosome onto DNA, and in associating a dimeric polymerase. In some embodiments, the primosome assembly proteins are involved in re-establishment of replication forks at recombination intermediates formed by RecA and strand exchange. In some embodiments, PriA initiates assembly of a replisome, competent for DNA synthesis, on recombination intermediates. In some embodiments, it is possible to target D-loops in vitro with a mixture of PriA, PriB, and DnaT, which are then competent to incorporate DnaB and DnaC. In some embodiments, once a primosome has been formed at the D-loop, all that remains to initiate replication is to load a holoenzyme complex to the site. In some embodiments, in the phage T4 system the gp59 helicase loader protein recruits and assembles the gp41 replicative helicase to D-loop structures

3) Fork Assembly and Initiation of DNA Synthesis

In some embodiments, replication forks will assemble at the site of primosome assembly. In some embodiments, in E. coli the presence of a free 3′-end on the invading strand of the D-loop stimulates the DnaX clamp loader complex detailed earlier to assemble a β-dimer at this site to act as a sliding clamp. In some embodiments, the holoenzyme and 2 core units are joined together by the scaffold T subunit. In some embodiments, the r subunit has interaction surfaces for the β-dimer, for the clamp loader, and for the DnaB helicase component of the primosome. In some embodiments, these multiple interactions coordinate synthesis of both leading and lagging strands using the 2 asymmetrically joined core polymerase complexes. In some embodiments, in T4 phage the gp59/41 proteins with uvsY and gp32 proteins, and with other components coordinate assembly of the sliding clamp gp45 aided by gp44 and gp62 proteins initiates replisome assembly.

In some embodiments, in E. coli the primosomal primase, DnaG, synthesizes a short RNA primer onto the unwound lagging strand DNA template. In some embodiments, in the presence of the holoenzyme, the clamp loader recognizes the RNA/DNA duplex and loads a second β-dimer clamp onto this site. In some embodiments, the presence of an active primosome and the interaction of the r subunit with DnaB ensures simultaneous leading/lagging strand synthesis.

In some embodiments, a replication fork is now assembled. In some embodiments, synthesis of both leading and lagging strand occur simultaneously, and the DnaB helicase separates template strands ahead of the oncoming holoenzyme. In some embodiments, the lagging strand holoenzyme core generates Okazaki fragments of 1 to 2 kilobases in length. In some embodiments, once the lagging strand polymerase encounters the previous RNA primer, it dissociates from the β-clamp and synthesis is initiated from a newly assembled clamp loaded in the vicinity of the front of the leading strand. In some embodiments, the same lagging strand holoenzyme core is re-used since it is physically tethered to leading strand core.

In some embodiments, there is a dynamic interaction between β-dimer clamps, core subunits, and clamp loaders. In some embodiments, their affinities switch depending upon the physical circumstances. In some embodiments, a β-dimer that has been ‘abandoned’ at the end of the Okazaki fragments is recycled via active removal by clamp loaders, or excess 6 subunit that may be present.

In some embodiments, the RNA primers at the ends of Okazaki fragments are removed by the 5′ to 3′ exonuclease activity of DNA polymerase I. DNA ligase then joins the Okazaki fragments together forming a continuous lagging strand.

4) Fork Meeting and Termination

In some embodiments, in RPA, replication is initiated at two distant sites and the replication forks are oriented toward each other. In some embodiments, as replication forks converge the two original template strands will dissociate from one another as they become separated entirely both behind, and in front, of each fork. In some embodiments, the leading strand core of each fork complete synthesis, the remaining RNA primers are processed, and the final products are two double-stranded molecules. In some embodiments, nucleic acids are amplified on the order of several Megabases (Mb) by such an approach. In this disclosure, megabase also encompasses megabasepairs. In some embodiments, based on the known synthetic rate of the PolIII holoenzyme replication forks proceed at a rate of approximately 1 Mb/1000 seconds, i.e., approximately 15 to 20 minutes per cycle for a I Mb fragment.

Some embodiments allow efficient reinvasion of the targeting sites by the use of mixtures of helicases, resolvases and the RecO, RecR, and RecF proteins. In some embodiments, under appropriate conditions reinvasion and primosome assembly is possible shortly after a holoenzyme has moved away from the fork-assembly site. In some embodiments, the DNA becomes branched at several points, and each branch naturally resolves as it encounters the oncoming fork. Under these conditions it may be possible to achieve enormous amplification in times similar to the time taken to replicate the DNA only once. Some embodiments limit the concentrations of targeting oligonucleotides to avoid nucleotide depletion prior to the completion of synthesis.

In addition to the holoenzyme complex, in some embodiments the replication machine employs another complex known as the primosome, which synthesizes the lagging strand and moves the replication fork forwards. In some embodiments, the primosome complex comprises a helicase encoded by DnaB and a primase encoded by DnaG. In some embodiments, in addition to the proteins of the holoenzyme and primosome, replication includes the activity of single-stranded DNA binding protein (SSB), E. coli DNA polymerase I and DNA ligase.

Nested RPA

In some embodiments, RPA includes nested RPA. In some embodiments, the nested RPA involves a first RPA of a first region of DNA. In some embodiments, the reaction mixture is diluted, for example, by 10, 20, 30, 40, 50, 75, or 100 fold or more to reduce the concentration of the first primer pair, and a second primer pair is introduced into the reaction mixture and RPA repeated. According to one embodiment of the invention, the second primer pair is designed to be internal to the first primer pair to amplify a subsequence of the first RPA product. In some embodiments, the method increases specific amplification, i.e., reduces non-specific background amplification products and therefore increases sensitivity.

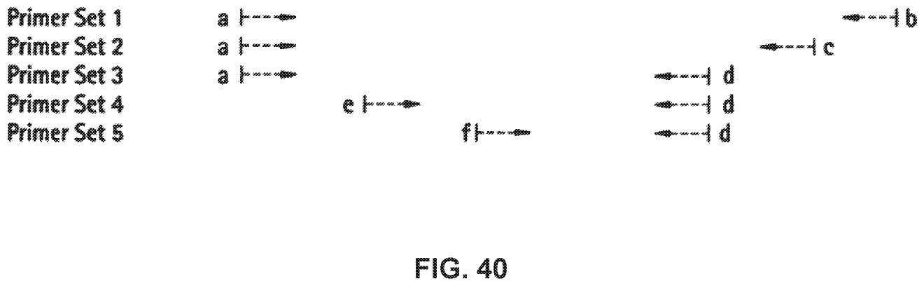

In some embodiments, nested RPA is not limited to the use of two sets of primers. In some embodiments, more sets of primers may be used to increase specificity or sensitivity. Thus, three, four, or five pairs of primers may be used. Furthermore, the different sets of primers, as another embodiment of the invention, may share common primers as illustrated in FIG. 40.

In the example in FIG. 40, the primer sets are designed to be used sequentially. For example, a first RPA is performed with primer set 1, a second RPA using the amplified product of the first RPA is performed with a primer set 2, a third RPA using the amplified product of the second RPA is performed with a primer set 3, a fourth RPA using the amplified sequence of the third RPA is performed with a primer set 4, and finally, a fifth RPA is performed using the amplified product of the fourth RPA is performed with a primer set 5. In this case, primer set 1, 2, and 3, share a common primer-primer (a). Primer 3, 4, and 5 share a common primer—primer (b).

Nested RPA may be performed using any of the two RPA methods described as well as a combination of the two methods in any particular order. That is, RPA may be performed solely by leading strand RPA, solely by leading and lagging strand RPA, or a combination of leading strand RPA and leading and lagging strand RPA in any particular order.

RPA Reagent Solutions

Some embodiments of the methods described herein relate to an RPA reagent solution. In some embodiments, the RPA reagent solution comprises a target nucleic acid, primers each being complementary to a region of said target nucleic acid, a buffer, dNTPs, a recombinase, a single-stranded DNA-binding protein (SSB), and/or a strand-displacing DNA polymerase.

In some embodiments, the recombinase comprises UvsX or RecA. In some embodiments, the SSB comprises gp32 or E. coli SSB. In some embodiments, the strand-displacing DNA polymerase comprises a Bst DNA polymerase large fragment, a Bst 2.0 polymerase, a Bst 3.0 polymerase, a Gsp polymerase, a Sau polymerase, a Bsu DNA polymerase large fragment, a Deep VentR DNA Polymerase, a Deep VentR (exo−) DNA Polymerase, a Klenow Fragment (3′→5′ exo−), a DNA Polymerase I Large (Klenow) Fragment, a phi29 DNA polymerase, a VentR DNA polymerase, or a VentR (exo−) DNA polymerase. Other recombinases, SSBs, and/or strand-displacing polymerases, such as those described herein, may also be used.

In some embodiments of the methods described herein, an RPA reagent solution or an amplification reaction solution includes RPA primers. In some embodiments, the RPA primers comprise traditional PCR primers that are about 20 bases long. In some embodiments, the RPA primers comprise longer primers (˜30-45 bases) than traditional PCR primers. RPA with longer primers may produce a more rapid amplification than RPA with traditional PCR primers.

Other RPA reagents such as those described herein may also be used in the methods described herein. In some embodiments, the RPA reagent solution comprises a reagent selected from: Tris-Acetate, polyethylene glycol, trehalose, potassium acetate, creatine kinase, phosphocreatine, adenosine triphosphate, or a recombinase loading protein, such as uvsY, or wherein said RPA reagent solution or said amplification reaction solution does not comprise a primer having a detectable label or dye. In some embodiments, the amplification reaction solution comprises a concentration of 0.5-1 mM DTT, 0.25-0.5 mM DTT, about 0.25 mM DTT, 0.25 mM DTT, 0-0.25 mM DTT, or 0 mM DTT. In some embodiments, the amplification reaction solution comprises a concentration of 1-10 mM DTT, about 5 mM DTT or 5 mM DTT, and 5-15 mM calcium chloride; about 0.9 mM calcium chloride, 0.9 mM calcium chloride, 5-15 mM Ca2+, about 0.9 mM Ca2+, or 0.9 mM Ca2+. In some embodiments, the amplification reaction solution comprises magnesium or magnesium ions at a concentration of 1-20 mM, 1-5 mM, 5-10 mM, 7-9 mM, 8 mM, about 8 mM, 10-20 mM, about 13 mM, or 13 mM. In some embodiments, the amplification reaction solution comprises a dNTP mixture at a concentration of 1-10 mM, 1-3 mM, about 1.8 mM, 1.8 mM, 5-6 mM, about 5.6 mM or 5.6 mM. In some embodiments, the amplification reaction solution further comprises a blocker oligonucleotide comprising a nucleic acid sequence that is a reverse complement to part of the nucleic acid sequence of one or more of said primers.

Blocker Oligonucleotides

Some embodiments of the methods described herein relate to the use of blocker oligonucleotides, also referred to herein as “blockers.” When testing RPA in some cases, one problem was an inability to distinguish true-positive amplification (“positives”) from false-positive amplification (“NTCs”). Some RPA primer sets showed NTCs that amplified as fast as positives, and sometimes the signal amplitude from the NTCs was greater than from the positives as well. This may be due to the low-temperature nature of RPA, which may allow more primer-primer interactions to occur. Most researchers simply use probes to get around this, as they only show signal from positives. A solution to this problem that was compatible with a sensor described herein was to use blocker oligonucleotides.

In some embodiments, the blockers comprise short oligonucleotide sequences with the following example characteristics:

(1) reverse complements of the primers used for RPA, beginning at the 3′ end. In other words, the 5′ terminal base of the blocker is hybridized to the 3′ terminal base of the primer. This may make the primer be considered partially double-stranded.

(2) a range in length from 8 nucleotides on up to 20 or more. It was found that the precise length could be primer-dependent, but generally something close to 15 nucleotides worked best.

(3) chemically modified at the 3′ end to prevent extension by polymerase. For example, they may be phosphorylated. While adequate for our purposes, phosphorylation might not work in a reaction that included an exonuclease (such as an exo+ polymerase, or a reaction that used a probe for detection), because exonucleases may remove phosphate groups from oligonucleotides. However, in these scenarios, other polymerase-blocking modifications are widely available that are resistant to exonucleases, such as 3-carbon spacers.

### Overview of Example Enhancing Changes in Conductivity

Some embodiments of the methods, systems and compositions provided herein include enhancing changes in the conductivity of a solution that result from amplification of a nucleic acid. In some embodiments, chelation of pyrophosphate (“PPi”) that results from nucleic acid amplification can be used to enhance changes in the conductivity of a solution as an amplification reaction continues. Without being bound to any one theory, conductivity changes that can occur during amplification of a nucleic may be based on precipitation of magnesium cations and PPi ions from solution. Some embodiments of the methods provided herein can include increasing the conductivity change by changing the equilibria, which otherwise results in the precipitation of magnesium cations and PPi ions. In some embodiments, this is accomplished by the addition of molecules that compete against magnesium cations for PPi. In some such embodiments, compounds are provided that have a high ionic mobility, which would result in a high contribution to net solution conductivity. Therefore, the removal of the compound from solution by precipitation of the compounds with PPi produces a dramatic change in the conductivity of the solution. In some embodiments of the methods provided herein, compounds/complexes, which may bind PPi and result in changes and/or enhanced changes in the conductivity of a solution as amplification continues, include Cd²⁺-cyclen-coumarin; Zn²⁺ complexes with a bis(2-pyridylmethyl)amine (DPA) unit; DPA-2Zn²⁺-phenoxide; acridine-DPA-Zn²⁺; DPA-Zn²⁺-pyrene; and azacrown-Cu²⁺ complexes. See e.g., Kim S. K. et al., (2008) Accounts of Chemical Research 42: 23-31; and Lee D-H, et al., (2007) Bull. Korean Chem. Soc. 29: 497-498; Credo G. M. et al., (2011) Analyst 137:1351-1362; and Haldar B. C. (1950) “Pyrophosphato-Complexes of Nickel and Cobalt in Solution” Nature 4226:744-745, each of which is hereby expressly incorporated by reference herein in its entirety.

Some embodiments include compounds such as 2-amino-6-mercapto-7-methylpurine ribonucleoside (MESG). MESG is used in kits to detect pyrophosphate such as an EnzChek® Pyrophosphate Assay Kit (ThermoFischer Scientific) in which MESG is converted by the purine nucleoside phosphorylase (PNP) enzyme to ribose 1-phosphate and 2-amino-6-mercapto-7-methylpurine in the presence of inorganic phosphate. The enzymatic conversion of MESG results in a shift in absorbance maximum from 330 nm to 360 nm. PNP catalyzes conversion of pyrophosphate into two equivalents of phosphate. The phosphate is then consumed by the MESG/PNP reaction and detected by an increase in absorbance at 360 nm. Additional sensitivity is gained by the amplification of one molecule of pyrophosphate into two molecules of phosphate. Another kit includes PIPER Pyrophosphate Assay Kit (ThermoFischer Scientific).

In some embodiments, enhancing changes in the conductivity of a solution that result from amplification of a nucleic acid include compounds that bind amplified DNA. In some such embodiments, as amplification continues a charge carrying species binds to the increasing amounts of amplified DNA resulting in a net reduction in conductivity of the solution. In some embodiments, charged carrying species can include positively charged molecules commonly used as DNA/RNA stains/dyes, such as ethidium bromide, crystal violet, SYBR, which bind to nucleic acids through electrostatic attraction. The binding of these small, charged molecular species to large and less mobile amplification products can reduce the conductivity of the solution by effectively reducing the charge mobility of the dye molecules. It should be noted that while this electrostatic attraction is the mechanism by which DNA is frequently stained for gel electrophoresis, the molecules which bind to the amplicons need not be compounds traditionally used as DNA stains. Since these molecules are being utilized for their function as a charge carrier (contributor to the solution conductivity) as well as their ability to bind to the amplicon, they need not possess any DNA staining properties. In some embodiments, the charge carrying species comprises Alizarin Red S. For example, Alizarin Red S can interact with amplified DNA molecules and change the behavior of amplified DNA voltammetrically such that it enhances detection of the amplified DNA by a system or device as described herein.

Some embodiments include the use of antibodies or aptamers linked to a nanoparticle. In some such embodiments, the presence of a target antigen results in aggregation of the antibodies and a change in conductivity of the solution. Without being bounds to any one theory, the effective electrical conductivity of colloidal nano-suspensions in a liquid can exhibit a complex dependence on the electrical double layer (EDL) characteristics, volume fraction, ionic concentrations and other physicochemical properties. See e.g., Angayarkanni S A., et al., Journal of Nanofluids, 3: 17-25 which is hereby expressly incorporated by reference herein in its entirety. Antibody-conjugated nanoparticles are well known in the art. See e.g., Arruebo M. et al., Journal of Nanomaterials 2009:Article ID 439389; and Zawrah M F., et al., HBRC Journal 2014.12.001, which are each hereby expressly incorporated by reference herein in its entirety. Examples of nanoparticles that are useful with the methods provided herein include γ-Al₂O₃, SiO₂, TiO₂ and α-Al₂O₃, and gold nanoparticles, See e.g., Abdelhalim, M A K., et al., International Journal of the Physical Sciences, 6:5487-5491 which is hereby expressly incorporated by reference herein in its entirety. The use of antibodies linked to nanoparticles may also enhance signal generated at a surface through measurements taken using electrochemical impedance spectroscopy (EIS). See e.g., Lu J., et al., Anal Chem. 84: 327-333, which is incorporated by reference herein in its entirety.

Some embodiments of the methods, systems and compositions provided herein include the use of the use of antibodies or aptamers linked to an enzyme. In some embodiments, enzyme activity produces a change in the conductivity of a solution. In some such embodiments, the change in conductivity is detected by a charge transfer to a substrate contacting the assay components.

### Overview of Example Viral Targets

Some embodiments of the methods, systems and compositions provided herein include the detection of certain viruses and viral targets. A viral target can include a viral nucleic acid, a viral protein, and/or product of viral activity, such as an enzyme or its activity. Examples of viral proteins that are detected with methods and devices provided herein include viral capsid proteins, viral structural proteins, viral glycoproteins, viral membrane fusion proteins, viral proteases or viral polymerases. Viral nucleic acid sequences (RNA and/or DNA) corresponding to at least a portion of the gene encoding the aforementioned viral proteins are also detected with the methods and devices described herein. Nucleotide sequences for such targets are readily obtained from public databases. Primers useful for isothermal amplification are readily designed from the nucleic acid sequences of desired viral targets. Antibodies and aptamers to proteins of such viruses are also readily obtained through commercial avenues, and/or by techniques well known in the art. Examples of viruses that are detected with the methods, systems and compositions provided herein include DNA viruses, such as double-stranded DNA viruses and single-stranded viruses; RNA viruses such as double-stranded RNA viruses, single-stranded (+) RNA viruses, and single-stranded (−) RNA viruses; and retro-transcribing viruses, such as single-stranded retro-transcribing RNA viruses, and double-stranded retro-transcribing DNA viruses. Viruses that are detected utilizing this technology include animal viruses, such as human viruses, domestic animal viruses, livestock viruses, or plant viruses. Examples of human viruses that are detected with the methods, systems and compositions provided herein include those listed in TABLE 2 below which also provides exemplary nucleotide sequences from which primers useful for amplification are readily designed.

TABLE 2

| | Example nucleotide |
|---|---|
| | sequence |
| Example virus | (NCBI Accession No.) |
| Adeno-associated virus | NC_001401 |
| Aichi virus | NC_001918 |
| Australian bat lyssavirus | NC_003243 |
| BK polyomavirus | NC_001538 |
| Banna virus | NC_004217 |
| Barmah forest virus | NC_001786 |
| Bunyamwera virus | NC_001925 |
| Bunyavirus La Crosse | NC_004108 |
| Bunyavirus snowshoe hare | |
| Cercopithecine herpesvirus | NC_006560 |
| Chandipura virus | |
| Chikungunya virus | NC_004162 |
| Cosavirus A | NC_012800 |
| Cowpox virus | NC_003663 |
| Coxsackievirus | NC_001612 |
| Crimean-Congo hemorrhagic fever | NC_005301 |
| virus | |
| Dengue virus | NC_001477 |
| Dhori virus | |
| Dugbe virus | |
| Duvenhage virus | NC_004159 |
| Eastern equine encephalitis virus | NC_003899 |
| Ebolavirus | NC_002549 |
| Echovirus | NC_001897 |
| Encephalomyocarditis virus | NC_001479 |
| Epstein-Barr virus | NC_007605 |
| European bat lyssavirus | NC_009527 |
| GB virus C/Hepatitis G virus | NC_001710 |
| Hantaan virus | NC_005222 |
| Hendra virus | NC_001906 |
| Hepatitis A virus | NC_001489 |
| Hepatitis B virus | NC_003977 |
| Hepatitis C virus | NC_004102 |
| Hepatitis E virus | NC_001434 |
| Hepatitis delta virus | NC_001653 |
| Horsepox virus | |
| Human adenovirus | NC_001405 |
| Human astrovirus | NC_001943 |
| Human coronavirus | NC_002645 |
| Human cytomegalovirus | NC_001347 |
| Human enterovirus 68, 70 | NC_001430 |
| Human herpesvirus 1 | NC_001806 |
| Human herpesvirus 2 | NC_001798 |
| Human herpesvirus 6 | NC_001664 |
| Human herpesvirus 7 | NC_001716 |
| Human herpesvirus 8 | NC_009333 |
| Human immunodeficiency virus | NC_001802 |
| Human papillomavirus 1 | NC_001356 |
| Human papillomavirus 2 | NC_001352 |
| Human papillomavirus 16,18 | NC_001526 |
| Human parainfluenza | NC_003461 |
| Human parvovirus B19 | NC_000883 |
| Human respiratory syncytial virus | NC_001781 |
| Human rhinovirus | NC_001617 |
| Human SARS coronavirus | NC_004718 |
| Human spumaretrovirus | |
| Human T-lymphotropic virus | NC_001436 |
| Human torovirus | |
| Influenza A virus | NC_002021 |
| Influenza B virus | NC_002205 |
| Influenza C virus | NC_006308 |
| Isfahan virus | |
| JC polyomavirus | NC_001699 |
| Japanese encephalitis virus | NC_001437 |
| Junin arenavirus | NC_005080 |
| KI Polyomavirus | NC_009238 |
| Kunjin virus | |
| Lagos bat virus | |
| Lake Victoria marburgvirus | NC_001608 |
| Langat virus | NC_003690 |
| Lassa virus | NC_004296 |
| Lordsdale virus | |
| Louping ill virus | NC_001809 |
| Lymphocytic choriomeningitis virus | NC_004294 |
| Machupo virus | NC_005078 |
| Mayaro virus | NC_003417 |
| MERS coronavirus | NC_019843 |
| Measles virus | NC_001498 |
| Mengo encephalomyocarditis virus | |
| Merkel cell polyomavirus | NC_010277 |
| Mokola virus | NC_006429 |
| Molluscum contagiosum virus | NC_001731 |
| Monkeypox virus | NC_003310 |
| Mumps virus | NC_002200 |
| Murray valley encephalitis virus | NC_000943 |
| New York virus | |
| Nipah virus | NC_002728 |
| Norwalk virus | NC_001959 |
| O'nyong-nyong virus | NC_001512 |
| Orf virus | NC_005336 |
| Oropouche virus | NC_005775 |
| Pichinde virus | NC_006439 |
| Poliovirus | NC_002058 |
| Punta toro phlebovirus | |
| Puumala virus | NC_005224 |
| Rabies virus | NC_001542 |
| Rift valley fever virus | NC_002044 |
| Rosavirus A | NC_024070 |
| Ross river virus | NC_001544 |
| Rotavirus A | NC_011506 |
| Rotavirus B | NC_007549 |
| Rotavirus C | NC_007570 |
| Rubella virus | NC_001545 |
| Sagiyama virus | |
| Salivirus A | NC_012957 |
| Sandfly fever sicilian virus | |
| Sapporo virus | NC_006554 |
| Semliki forest virus | NC_003215 |
| Seoul virus | NC_005237 |
| Simian foamy virus | NC_001364 |
| Simian virus 5 | |
| Sindbis virus | NC_001547 |
| Southampton virus | |
| St. louis encephalitis virus | NC_007580 |
| Tick-borne powassan virus | NC_003687 |
| Torque teno virus | NC_002076 |
| Toscana virus | NC_006319 |
| Uukuniemi virus | NC_005220 |
| Vaccinia virus | NC_006998 |
| Varicella-zoster virus | NC_001348 |
| Variola virus | NC_001611 |
| Venezuelan equine encephalitis virus | NC_001449 |
| Vesicular stomatitis virus | NC_001560 |
| Western equine encephalitis virus | NC_003908 |
| WU polyomavirus | NC_009539 |
| West Nile virus | NC_001563 |
| Yaba monkey tumor virus | NC_005179 |
| Yaba-like disease virus | NC_002642 |
| Yellow fever virus | NC_002031; and/or |
| Zika virus | NC_012532 |

### Overview of Example Bacterial Targets

Some embodiments of the methods, systems and compositions provided herein include the detection of certain bacteria and bacterial targets. A bacterial target includes a bacterial nucleic acid, a bacterial protein, and/or product of bacterial activity, such as toxins, and enzyme activities. Nucleotide sequences indicative of certain bacteria are readily obtained from public databases. Primers useful for isothermal amplification are readily designed from nucleic acid sequences of such bacterial targets. Antibodies and aptamers to proteins of certain bacteria are readily obtained through commercial avenues, and/or by techniques well known in the art. Examples of bacteria that are detected with the methods, systems and compositions provided herein include gram negative or gram positive bacteria. Examples of bacteria that are detected with the methods, systems and compositions provided herein include: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuarti, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides unformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and/or Staphylococcus saccharolyticus. More example include B. anthracis, B. globigii, Brucella, E. herbicola, or F. tularensis.

### Overview of Example Antigen Targets

Some embodiments of the methods, systems and compositions provided herein include the detection of certain antigen targets. Antigens are detected using antibodies, binding fragments thereof, or aptamers linked to primers that are configured for amplification, such as isothermal amplification. Antibodies and aptamers to certain antigens are readily obtained through commercial avenues, and/or by techniques well known in the art. As used herein an “antigen” includes a compound or composition that is specifically bound by an antibody, binding fragment thereof, or aptamer. Examples of antigens that are detected with the methods, systems and compositions provided herein include proteins, polypeptides, nucleic acids, and small molecules, such as pharmaceutical compounds. More examples of analytes include toxins, such as ricin, abrin, Botulinum toxin, or Staphylococcal enterotoxin B.

### Overview of Example Parasite Targets

Some embodiments of the methods, systems and compositions provided herein include the detection of certain parasite targets. A parasite target includes a parasite nucleic acid, a parasite protein, and/or a product of parasite activity, such as a toxin and/or an enzyme or enzyme activity. Nucleotide sequences indicative of certain parasites are readily obtained from public databases. Primers useful for isothermal amplification are readily designed from nucleic acid sequences of such parasite targets. Antibodies and aptamers to proteins of certain parasites are readily obtained through commercial avenues, and/or techniques well known in the art. Examples of parasites that are detected with the methods, systems and compositions provided herein include certain endoparasites such as protozoan organisms such as Acanthamoeba spp. Babesia spp., B. divergens, B. bigemina, B. equi, B. microfti, B. duncani, Balamuthia mandrillaris, Balantidium coli, Blastocystis spp., Cryptosporidium spp., Cyclospora cayetanensis, Dientamoeba fragilis, Entamoeba histolytica, Giardia lamblia, Isospora belli, Leishmania spp., Naegleria fowleri, Plasmodium falciparum, Plasmodium vivax, Plasmodium ovale curtisi, Plasmodium ovale wallikeri, Plasmodium malariae, Plasmodium knowlesi, Rhinosporidium seeberi, Sarcocystis bovihominis, Sarcocystis suihominis, Toxoplasma gondii, Trichomonas vaginalis, Trypanosoma brucei, or Trypanosoma cruzi. Certain helminth organisms such as Bertiella mucronata, Bertiella studeri, Cestoda, Taenia multiceps, Diphyllobothrium latum, Echinococcus granulosus, Echinococcus multilocularis, E. vogeli, E. oligarthrus, Hymenolepis nana, Hymenolepis diminuta, Spirometra erinaceieuropaei, Taenia saginata, or Taenia solium. Certain fluke organism such as Clonorchis sinensis; Clonorchis viverrini, Dicrocoelium dendriticum, Echinostoma echinatum, Fasciola hepatica, Fasciola gigantica, Fasciolopsis buski, Gnathostoma spinigerum, Gnathostoma hispidum, Metagonimus yokogawai, Metorchis conjunctus, Opisthorchis viverrini, Opisthorchis felineus, Clonorchis sinensis, Paragonimus westermani; Paragonimus africanus; Paragonimus caliensis; Paragonimus kellicotti; Paragonimus skrjabini; Paragonimus uterobilateralis, Schistosoma haematobium, Schistosoma japonicum, Schistosoma mansoni and Schistosoma intercalatum, Schistosoma mekongi, Schistosoma sp, Trichobilharzia regenti, or Schistosomatidae. Certain roundworm organisms such as Ancylostoma duodenale, Necator americanus, Angiostrongylus costaricensis, Anisakis, Ascaris sp. Ascaris lumbricoides, Baylisascaris procyonis, Brugia malayi, Brugia timori, Dioctophyme renale, Dracunculus medinensis, Enterobius vermicularis, Enterobius gregorii, Halicephalobus gingivalis, Loa loa filaria, Mansonella streptocerca, Onchocerca volvulus, Strongyloides stercoralis, Thelazia californiensis, Thelazia callipaeda, Toxocara canis, Toxocara cati, Trichinella spiralis, Trichinella britovi, Trichinella nelsoni, Trichinella nativa, Trichuris trichiura, Trichuris vulpis, or Wuchereria bancrofti. Other parasites such as Archiacanthocephala, Moniliformis moniliformis, Linguatula serrata, Oestroidea, Calliphoridae, Sarcophagidae, Cochliomyia hominivorax (family Calliphoridae), Tunga penetrans, Cimicidae: Cimex lectularius, or Dermatobia hominis. More examples of parasites include ectoparasites such as Pediculus humanus, Pediculus humanus corporis, Pthirus pubis, Demodex folliculorum brevis canis, Sarcoptes scabiei, or Arachnida such as Trombiculidae, or Pulex irritans, or Arachnida such Ixodidae and/or Argasidae.

Overview of Example microRNA Targets

Some embodiments of the methods, systems and compositions provided herein include the detection of certain microRNA (miRNA) targets. miRNAs include small non-coding RNA molecules that function in RNA silencing or post-transcriptional regulation of gene expression. Some miRNAs are associated with deregulation in various human diseases which are caused by abnormal epigenetic patterns, including abnormal DNA methylation and histone-modification patterns. For example, the presence or absence of a certain miRNA in a sample from a subject is indicative of a disease or disease state. Primers useful to detect miRNAs and useful for isothermal amplification are readily designed from nucleotide sequences of miRNAs. Nucleotide sequences of miRNAs are readily obtained from public databases. Examples of miRNA targets that are detected with the methods, systems and compositions provided herein include: hsa-miR-1, hsa-miR-1-2, hsa-miR-100, hsa-miR-100-1, hsa-miR-100-2, hsa-miR-101, hsa-miR-101-1, hsa-miR-101a, hsa-miR-101b-2, hsa-miR-102, hsa-miR-103, hsa-miR-103-1, hsa-miR-103-2, hsa-miR-104, hsa-miR-105, hsa-miR-106a, hsa-miR-106a-1, hsa-miR-106b, hsa-miR-106b-1, hsa-miR-107, hsa-miR-10a, hsa-miR-10b, hsa-miR-122, hsa-miR-122a, hsa-miR-123, hsa-miR-124a, hsa-miR-124a-1, hsa-miR-124a-2, hsa-miR-124a-3, hsa-miR-125a, hsa-miR-125a-5p, hsa-miR-125b, hsa-miR-125b-1, hsa-miR-125b-2, hsa-miR-126, hsa-miR-126-5p, hsa-miR-127, hsa-miR-128a, hsa-miR-128b, hsa-miR-129, hsa-miR-129-1, hsa-miR-129-2, hsa-miR-130, hsa-miR-130a, hsa-miR-130a-1, hsa-miR-130b, hsa-miR-130b-1, hsa-miR-132, hsa-miR-133a, hsa-miR-133b, hsa-miR-134, hsa-miR-135a, hsa-miR-135b, hsa-miR-136, hsa-miR-137, hsa-miR-138, hsa-miR-138-1, hsa-miR-138-2, hsa-miR-139, hsa-miR-139-5p, hsa-miR-140, hsa-miR-140-3p, hsa-miR-141, hsa-miR-142-3p, hsa-miR-142-5p, hsa-miR-143, hsa-miR-144, hsa-miR-145, hsa-miR-146a, hsa-miR-146b, hsa-miR-147, hsa-miR-148a, hsa-miR-148b, hsa-miR-149, hsa-miR-15, hsa-miR-150, hsa-miR-151, hsa-miR-151-5p, hsa-miR-152, hsa-miR-153, hsa-miR-154, hsa-miR-155, hsa-miR-15a, hsa-miR-15a-2, hsa-miR-15b, hsa-miR-16, hsa-miR-16-1, hsa-miR-16-2, hsa-miR-16a, hsa-miR-164, hsa-miR-170, hsa-miR-172a-2, hsa-miR-17, hsa-miR-17-3p, hsa-miR-17-5p, hsa-miR-17-92, hsa-miR-18, hsa-miR-18a, hsa-miR-18b, hsa-miR-181a, hsa-miR-181a-1, hsa-miR-181a-2, hsa-miR-181b, hsa-miR-181b-1, hsa-miR-181b-2, hsa-miR-181c, hsa-miR-181d, hsa-miR-182, hsa-miR-183, hsa-miR-184, hsa-miR-185, hsa-miR-186, hsa-miR-187, hsa-miR-188, hsa-miR-189, hsa-miR-190, hsa-miR-191, hsa-miR-192, hsa-miR-192-1, hsa-miR-192-2, hsa-miR-192-3, hsa-miR-193a, hsa-miR-193b, hsa-miR-194, hsa-miR-195, hsa-miR-196a, hsa-miR-196a-2, hsa-miR-196b, hsa-miR-197, hsa-miR-198, hsa-miR-199a, hsa-miR-199a-1, hsa-miR-199a-1-5p, hsa-miR-199a-2, hsa-miR-199a-2-5p, hsa-miR-199a-3p, hsa-miR-199b, hsa-miR-199b-5p, hsa-miR-19a, hsa-miR-19b, hsa-miR-19b-1, hsa-miR-19b-2, hsa-miR-200a, hsa-miR-200b, hsa-miR-200c, hsa-miR-202, hsa-miR-203, hsa-miR-204, hsa-miR-205, hsa-miR-206, hsa-miR-207, hsa-miR-208, hsa-miR-208a, hsa-miR-20a, hsa-miR-20b, hsa-miR-21, hsa-miR-22, hsa-miR-210, hsa-miR-211, hsa-miR-212, hsa-miR-213, hsa-miR-214, hsa-miR-215, hsa-miR-216, hsa-miR-217, hsa-miR-218, hsa-miR-218-2, hsa-miR-219, hsa-miR-219-1, hsa-miR-22, hsa-miR-220, hsa-miR-221, hsa-miR-222, hsa-miR-223, hsa-miR-224, hsa-miR-23a, hsa-miR-23b, hsa-miR-24, hsa-miR-24-1, hsa-miR-24-2, hsa-miR-25, hsa-miR-26a, hsa-miR-26a-1, hsa-miR-26a-2, hsa-miR-26b, hsa-miR-27a, hsa-miR-27b, hsa-miR-28, hsa-miR-296, hsa-miR-298, hsa-miR-299-3p, hsa-miR-299-5p, hsa-miR-29a, hsa-miR-29a-2, hsa-miR-29b, hsa-miR-29b-1, hsa-miR-29b-2, hsa-miR-29c, hsa-miR-301, hsa-miR-302, hsa-miR-302a, hsa-miR-302b, hsa-miR-302c, hsa-miR-302c, hsa-miR-302d, hsa-miR-30a, hsa-miR-30a-3p, hsa-miR-30a-5p, hsa-miR-30b, hsa-miR-30c, hsa-miR-30c-1, hsa-miR-30d, hsa-miR-30e, hsa-miR-30e, hsa-miR-30e-5p, hsa-miR-31, hsa-miR-31a, hsa-miR-32, hsa-miR-32, hsa-miR-320, hsa-miR-320-2, hsa-miR-320a, hsa-miR-322, hsa-miR-323, hsa-miR-324-3p, hsa-miR-324-5p, hsa-miR-325, hsa-miR-326, hsa-miR-328, hsa-miR-328-1, hsa-miR-33, hsa-miR-330, hsa-miR-331, hsa-miR-335, hsa-miR-337, hsa-miR-337-3p, hsa-miR-338, hsa-miR-338-5p, hsa-miR-339, hsa-miR-339-5p, hsa-miR-34a, hsa-miR-340, hsa-miR-340, hsa-miR-341, hsa-miR-342, hsa-miR-342-3p, hsa-miR-345, hsa-miR-346, hsa-miR-347, hsa-miR-34a, hsa-miR-34b, hsa-miR-34c, hsa-miR-351, hsa-miR-352, hsa-miR-361, hsa-miR-362, hsa-miR-363, hsa-miR-355, hsa-miR-365, hsa-miR-367, hsa-miR-368, hsa-miR-369-5p, hsa-miR-370, hsa-miR-371, hsa-miR-372, hsa-miR-373, hsa-miR-374, hsa-miR-375, hsa-miR-376a, hsa-miR-376b, hsa-miR-377, hsa-miR-378, hsa-miR-378, hsa-miR-379, hsa-miR-381, hsa-miR-382, hsa-miR-383, hsa-miR-409-3p, hsa-miR-419, hsa-miR-422a, hsa-miR-422b, hsa-miR-423, hsa-miR-424, hsa-miR-429, hsa-miR-431, hsa-miR-432, hsa-miR-433, hsa-miR-449a, hsa-miR-451, hsa-miR-452, hsa-miR-451, hsa-miR-452, hsa-miR-452, hsa-miR-483, hsa-miR-483-3p, hsa-miR-484, hsa-miR-485-5p, hsa-miR-485-3p, hsa-miR-486, hsa-miR-487b, hsa-miR-489, hsa-miR-491, hsa-miR-491-5p, hsa-miR-492, hsa-miR-493-3p, hsa-miR-493-5p, hsa-miR-494, hsa-miR-495, hsa-miR-497, hsa-miR-498, hsa-miR-499, hsa-miR-5, hsa-miR-500, hsa-miR-501, hsa-miR-503, hsa-miR-508, hsa-miR-509, hsa-miR-510, hsa-miR-511, hsa-miR-512-5p, hsa-miR-513, hsa-miR-513-1, hsa-miR-513-2, hsa-miR-515-3p, hsa-miR-516-5p, hsa-miR-516-3p, hsa-miR-518b, hsa-miR-519a, hsa-miR-519d, hsa-miR-520a, hsa-miR-520c, hsa-miR-521, hsa-miR-532-5p, hsa-miR-539, hsa-miR-542-3p, hsa-miR-542-5p, hsa-miR-550, hsa-miR-551a, hsa-miR-561, hsa-miR-563, hsa-miR-565, hsa-miR-572, hsa-miR-582, hsa-miR-584, hsa-miR-594, hsa-miR-595, hsa-miR-598, hsa-miR-599, hsa-miR-600, hsa-miR-601, hsa-miR-602, hsa-miR-605, hsa-miR-608, hsa-miR-611, hsa-miR-612, hsa-miR-614, hsa-miR-615, hsa-miR-615-3p, hsa-miR-622, hsa-miR-627, hsa-miR-628, hsa-miR-635, hsa-miR-637, hsa-miR-638, hsa-miR-642, hsa-miR-648, hsa-miR-652, hsa-miR-654, hsa-miR-657, hsa-miR-658, hsa-miR-659, hsa-miR-661, hsa-miR-662, hsa-miR-663, hsa-miR-664, hsa-miR-7, hsa-miR-7-1, hsa-miR-7-2, hsa-miR-7-3, hsa-miR-708, hsa-miR-765, hsa-miR-769-3p, hsa-miR-802, hsa-miR-885-3p, hsa-miR-9, hsa-miR-9-1, hsa-miR-9-3, hsa-miR-9-3p, hsa-miR-92, hsa-miR-92-1, hsa-miR-92-2, hsa-miR-9-2, hsa-miR-92, hsa-miR-92a, hsa-miR-93, hsa-miR-95, hsa-miR-96, hsa-miR-98, hsa-miR-99a, and/or hsa-miR-99b.

### Overview of Example Agricultural Analytes

Some embodiments of the methods, systems and compositions provided herein include the detection of certain agricultural analytes. Agricultural analytes include nucleic acids, proteins, or small molecules. Nucleotide sequences indicative of certain agricultural analytes are readily obtained from public databases. Primers useful for isothermal amplification are readily designed from nucleic acid sequences of such agricultural analytes. Antibodies and aptamers to proteins of certain agricultural analytes are readily obtained through commercial avenues, and/or techniques well known in the art.

Some embodiments of the methods and devices provided herein are used to identify the presence of an organism or product of the organism in a meat product, fish product, or yeast product such as beer, wine or bread. In some embodiments, species-specific antibodies or aptamers, or species-specific primers are used to identify the presence of a certain organism in a food product.

Some embodiments of the methods, systems and compositions provided herein include the detection of pesticides. In some embodiments, pesticides are detected in samples such as soils samples or food samples. Examples of pesticides that are detected with the devices and methods described herein include herbicides, insecticides, or fungicides. Examples of herbicides include 2,4-dichlorophenoxyacetic acid (2,4-D), atrazine, glyphosate, mecoprop, dicamba, paraquat, glufosinate, metam-sodium, dazomet, dithopyr, pendimethalin, EPTC, trifluralin, flazasulfuron, metsulfuron-methyl, diuron, nitrofen, nitrofluorfen, acifluorfen, mesotrione, sulcotrione, or nitisinone. Examples of insecticides that are detected with the devices and methods described herein include organochlorides, organophosphates, carbamates, pyrethroids, neonicotinoids, or ryanoids. Examples of fungicides that are detected with the devices and methods described herein include carbendazim, diethofencarb, azoxystrobin, metalaxyl, metalaxyl-m, streptomycin, oxytetracycline, chlorothalonil, tebuconazole, zineb, mancozeb, tebuconazole, myclobutanil, triadimefon, fenbuconazole, deoxynivalenol, or mancozeb.

### Overview of Example Biomarkers

Some embodiments of the methods, systems and compositions provided herein include the detection of certain biomarkers for certain disorders. Biomarkers can include nucleic acids, proteins, protein fragments, and antigens. Some biomarkers can include a target provided herein. Example disorders include cancers, such as breast cancers, colorectal cancers, gastric cancers, gastrointestinal stromal tumors, leukemias and lymphomas, lung cancers, melanomas, brain cancers, and pancreatic cancers. Some embodiments can include detecting the presence or absence of a biomarker, or the level of a biomarker in a sample. The biomarker can be indicative of the presence, absence or stage of a certain disorder. Example biomarkers include estrogen receptor, progesterone receptor, HER-2/neu, EGFR, KRAS, UGT1A1, c-KIT, CD20, CD30, FIP1L1-PDGFRalpha, PDGFR, Philadelphia chromosome (BCR/ABL), PML/RAR-alpha, TPMT, UGT1A1, EML4/ALK, BRAF, and elevated levels of certain amino acids such as leucine, isoleucine, and valine.

### Overview of Example Methods of Amplifying and Detecting Target Nucleic Acids

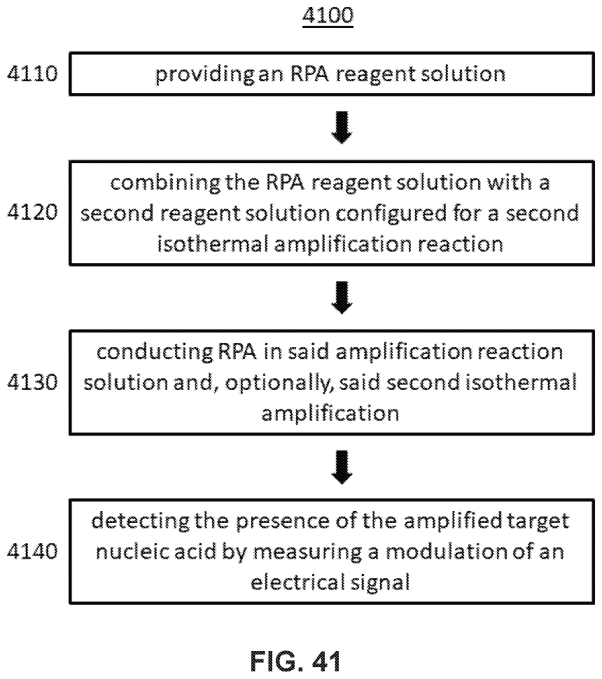

Some embodiments include a method 4100 of amplifying and detecting a target nucleic acid. An example of the method 4100 is depicted in FIG. 41. In some embodiments, the method 4100 includes providing a recombinase polymerase amplification (RPA) reagent solution comprising a target nucleic acid, primers each being complementary to a region of said target nucleic acid, a buffer, dNTPs, a recombinase, a single-stranded DNA-binding protein (SSB), and a strand-displacing DNA polymerase, preferably, in a single vessel 4110; combining the RPA reagent solution with a second reagent solution configured for a second isothermal amplification reaction, which does not utilize a recombinase, preferably in said single vessel, so as to produce an amplification reaction solution 4120; conducting RPA in said amplification reaction solution and, optionally, said second isothermal amplification, preferably in said single vessel, to produce an amplified target nucleic acid 4130; and detecting the presence of the amplified target nucleic acid by measuring a modulation of an electrical signal, such as impedance, in the amplification reaction solution when said amplification reaction is subjected to an electrical field, as compared to a control 4140. Some embodiments include conducting spiral RPA using, for example, a pair of primers with the forward and reverse primer sequences reverse complementary to each other at their 5′ end and their 3′ end sequences complementary to the target sequences.

Some embodiments of the method 4100 include providing an RPA reagent solution 4110. In some embodiments, the RPA reagent solution includes reagents compatible with RPA as described herein. In some embodiments, the RPA solution includes a target nucleic acid, primers each being complementary to a region of said target nucleic acid, a buffer, dNTPs, a recombinase, an SSB, and/or a strand-displacing DNA polymerase. In some embodiments, providing the RPA reagent solution includes providing the RPA reagents in a single vessel. In some embodiments, the RPA reagents are provided in more than one vessel.

Some embodiments of the method 4100 include combining the RPA reagent solution with a second reagent solution configured for a second isothermal amplification reaction that does not utilize a recombinase, so as to produce an amplification reaction solution 4120. In some embodiments, the RPA reagent solution is combined with the second reagent solution in said single vessel.

Some embodiments of the method 4100 include conducting RPA in the amplification reaction solution 4130. Examples of RPA reaction solutions are described herein. Some embodiments include conducting said second isothermal amplification. In some embodiments, the RPA and the second isothermal amplification are conducted in said single vessel. In some embodiments, conducting the RPA and the optional second isothermal amplification produce an amplified target nucleic acid.

Some embodiments of the method 4100 include use of a device described herein. For example, a device described herein may be used to detect the amplified nucleic acid target. Some embodiments of the method 4100 include detecting the presence of the amplified target nucleic acid by measuring a modulation of an electrical signal, for example, in the amplification reaction solution when said amplification reaction is subjected to an electrical field 4140. In some embodiments, the electrical signal comprises an impedance. In some embodiments, the electrical signal is compared to a control.

In some embodiments of the method 4100, the primers and/or the amplification reaction solution do not comprise a label or a dye. In some embodiments, the amplification and detecting are performed in the absence of a detection reagent such as a dye, a turbidity agent, a fluorophore, a double-stranded nucleic acid intercalating agent, a sequencing index, and/or a nanoparticle.

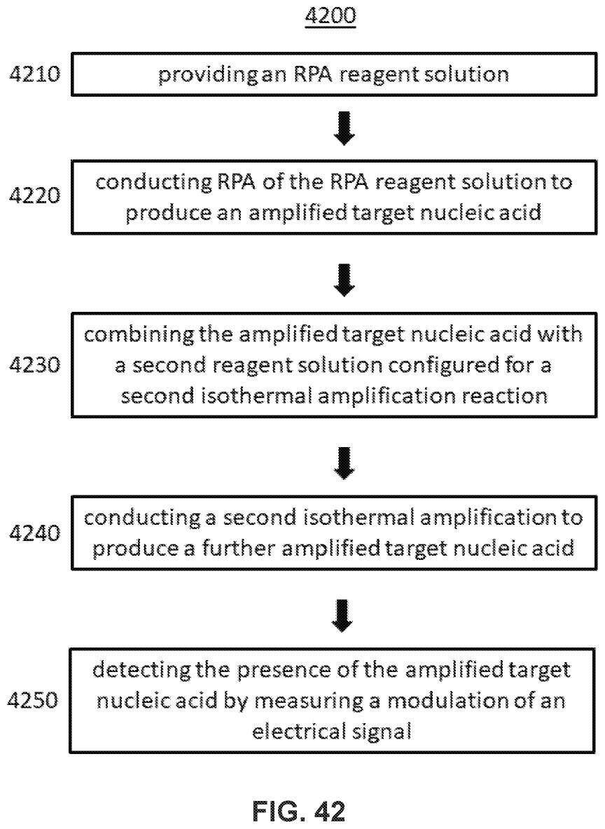

Some embodiments include a method 4200 of amplifying and detecting a target nucleic acid. An example of the method 4200 is depicted in FIG. 42. In some embodiments, the method 4200 includes providing a recombinase polymerase amplification (RPA) reagent solution comprising a target nucleic acid, primers each being complementary to a region of said target nucleic acid, a buffer, dNTPs, a recombinase, a single-stranded DNA-binding protein (SSB), and a strand-displacing DNA polymerase, preferably, in a single vessel 4210; conducting RPA of the RPA reagent solution to produce an amplified target nucleic acid 4220; combining the amplified target nucleic acid with a second reagent solution configured for a second isothermal amplification reaction, which does not utilize a recombinase, preferably in said single vessel, to produce a second amplification reaction solution 4230; conducting a second isothermal amplification of said second amplification reaction solution to produce a further amplified target nucleic acid 4240; and detecting the presence of the amplified target nucleic acid by measuring a modulation of an electrical signal, such as impedance, in the amplification reaction solution when said amplification reaction is subjected to an electrical field, as compared to a control 4250.

Some embodiments of the method 4200 include providing a recombinase polymerase amplification (RPA) reagent solution 4210. In some embodiments, the RPA reagent solution includes reagents compatible with RPA as described herein. In some embodiments, the reagent solution comprises a target nucleic acid, primers each being complementary to a region of said target nucleic acid, a buffer, dNTPs, a recombinase, a single-stranded DNA-binding protein (SSB), and/or a strand-displacing DNA polymerase. In some embodiments, the reagents are in a single vessel.

Some embodiments include conducting RPA of the RPA reagent solution to produce an amplified target nucleic acid 4220. Examples of RPA are described herein.

Some embodiments include combining the amplified target nucleic acid with a second reagent solution configured for a second isothermal amplification reaction, which does not utilize a recombinase, preferably in said single vessel, to produce a second amplification reaction solution 4230. Examples of isothermal amplification reactions that do not utilize a recombinase are provided herein.

Some embodiments include conducting a second isothermal amplification of said second amplification reaction solution to produce a further amplified target nucleic acid 4240.

Some embodiments of the method 4200 include use of a device described herein. For example, a device described herein may be used to detect the amplified nucleic acid target. Some embodiments of the method 4200 include detecting the presence of the amplified target nucleic acid by measuring a modulation of an electrical signal, for example, in the amplification reaction solution when said amplification reaction is subjected to an electrical field 4250. In some embodiments, the electrical signal comprises an impedance. In some embodiments, the electrical signal is compared to a control.

In some embodiments of the method 4200, the primers and/or the amplification reaction solution do not comprise a label or a dye. In some embodiments, the amplification and detecting are performed in the absence of a detection reagent such as a dye, a turbidity agent, a fluorophore, a double-stranded nucleic acid intercalating agent, a sequencing index, and/or a nanoparticle.

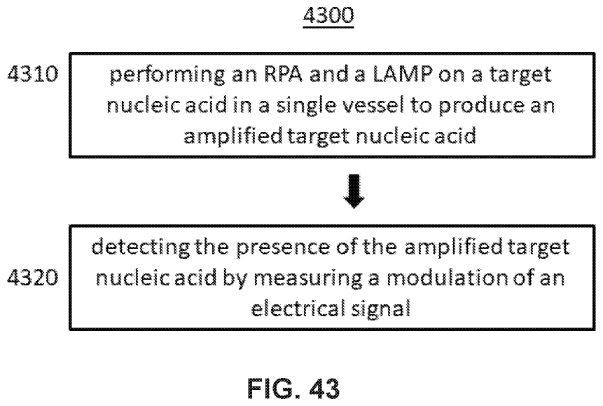

Some embodiments include a method 4300 of amplifying and detecting a target nucleic acid. An example of the method 4300 is depicted in FIG. 43. In some embodiments, the method 4300 includes performing a recombinase polymerase amplification (RPA) and a Loop-Mediated Isothermal Amplification (LAMP) on a target nucleic acid in a single vessel to produce an amplified target nucleic acid, preferably without isolating or purifying the amplified target nucleic acid between said RPA amplification and said LAMP amplification, such as in a single vessel 4310; and detecting the presence of the amplified target nucleic acid by measuring a modulation of an electrical signal, such as impedance, in the amplification reaction solution when said amplification reaction is subjected to an electrical field, as compared to a control 4320.

Some embodiments of the method 4300 include performing a recombinase polymerase amplification (RPA) and a Loop-Mediated Isothermal Amplification (LAMP) on a target nucleic acid in a single vessel to produce an amplified target nucleic acid, preferably without isolating or purifying the amplified target nucleic acid between said RPA amplification and said LAMP amplification, such as in a single vessel 4310. Some embodiments include detecting the presence of the amplified target nucleic acid by measuring a modulation of an electrical signal, such as impedance, in the amplification reaction solution when said amplification reaction is subjected to an electrical field, as compared to a control 4320.

In some embodiments of the method 4300, the primers in the RPA and/or LAMP amplifications do not comprise a label or a dye. In some embodiments, the RPA and/or LAMP amplifications and detecting are performed in the absence of a detection reagent such as a dye, a turbidity agent, a fluorophore, a double-stranded nucleic acid intercalating agent, a sequencing index, and/or a nanoparticle.

In some embodiments of the methods described herein, the detecting of the amplified target nucleic acid is performed in a device comprising a test well. In some embodiments, the test well comprises an excitation electrode and a sensor electrode. In some embodiments, said detecting further comprises applying an excitation signal from a reader device to the excitation electrode. In some embodiments, said detecting further comprises sensing a signal from the test well using the excitation electrode. In some embodiments, the signal represents the impedance of the amplification reaction solution. In some embodiments, said detecting further comprises transmitting the signal to the reader device, wherein the reader device analyzes the signal. In some embodiments, the detecting of the amplified target nucleic acid is performed in a device comprising a test well, which comprises an excitation electrode and a sensor electrode, and wherein said detecting further comprises: applying an excitation signal from a reader device to the excitation electrode; sensing a signal from the test well using the excitation electrode, wherein the signal represents the impedance of the amplification reaction solution; and transmitting the signal to the reader device, wherein the reader device analyzes the signal.

In some embodiments of the methods described herein, the amplification and detection steps are performed in the presence of about 0.25 mM dithiothreitol (DTT) or less, or in the absence of DTT.

In some embodiments of the methods described herein, the RPA comprises leading strand RPA (lsRPA), simultaneous leading and lagging strand synthesis, or nested RPA. In some embodiments, the second isothermal amplification comprises self-sustaining sequence replication reaction (3SR), 90-I, BAD Amp, cross priming amplification (CPA), isothermal exponential amplification reaction (EXPAR), isothermal chimeric primer initiated amplification of nucleic acids (ICAN), isothermal multi displacement amplification (IMDA), ligation-mediated SDA; multi displacement amplification; polymerase spiral reaction (PSR), restriction cascade exponential amplification (RCEA), smart amplification process (SMAP2), single primer isothermal amplification (SPIA), transcription-based amplification system (TAS), transcription meditated amplification (TMA), ligase chain reaction (LCR), or multiple cross displacement amplification (MCDA), rolling circle replication (RCA), Nicking Enzyme Amplification Reaction (NEAR) or Nucleic acid sequence based amplification (NASBA).

In some embodiments of the methods described herein, the second isothermal amplification comprises Loop-Mediated Isothermal Amplification (LAMP). In some embodiments, the amplification reaction solution comprises a primer oligonucleotide compatible with LAMP. In some embodiments, the amplification reaction solution comprises FIP, BIP, LF, or LB primer oligonucleotides compatible with LAMP, and primers compatible with RPA. In some embodiments, the amplification reaction solution comprises FIP, BIP, LF, LB, F3, or B3 primer oligonucleotides compatible with LAMP.

In some embodiments of the methods described herein, the RPA and the second isothermal amplification are conducted at the same or substantially the same temperature. In some embodiments, the RPA or the second isothermal amplification are conducted at 25° C., 30° C., 37° C., 40° C., 45° C., 50° C., 60° C., 65° C., 70° C., 75° C., or at a temperature that is within a range defined by any two of the aforementioned temperatures. In some embodiments, the RPA is conducted at a lower or higher temperature than the second isothermal amplification. In some embodiments, the RPA is conducted at 37° C., about 37° C., 40° C., or about 40° C., and the second isothermal amplification is conducted at 60° C., about 60° C., 65° C., or about 65° C.

In some embodiments, of the methods described herein, providing an RPA reagent solution, combining the RPA reagent solution with a second reagent solution, and/or conducting RPA in said amplification solution are performed at 5224 and/or 5228 of FIG. 52D or at 5718 and/or 5740 of any of FIGS. 57A-58D. In some embodiments, conducting RPA in the RPA reagent solution to produce an amplified target nucleic acid, combining the amplified target nucleic acid with a second reagent solution configured for a second isothermal amplification reaction, and/or conducting a second isothermal amplification of said second amplification reaction solution to produce a further amplified target nucleic acid are performed at 5224 and/or 5228 of FIG. 52D or at 5718 and/or 5740 of any of FIGS. 57A-58D. In some embodiments, performing an RPA and a LAMP on a target nucleic acid in a single vessel to produce an amplified target nucleic acid is performed at 5224 and/or 5228 of FIG. 52D or at 5718 and/or 5740 of any of FIGS. 57A-58D.

### EXAMPLES

### Example 1—ƒC4D LAMP Pre/Post Amplification Detection in PDMS

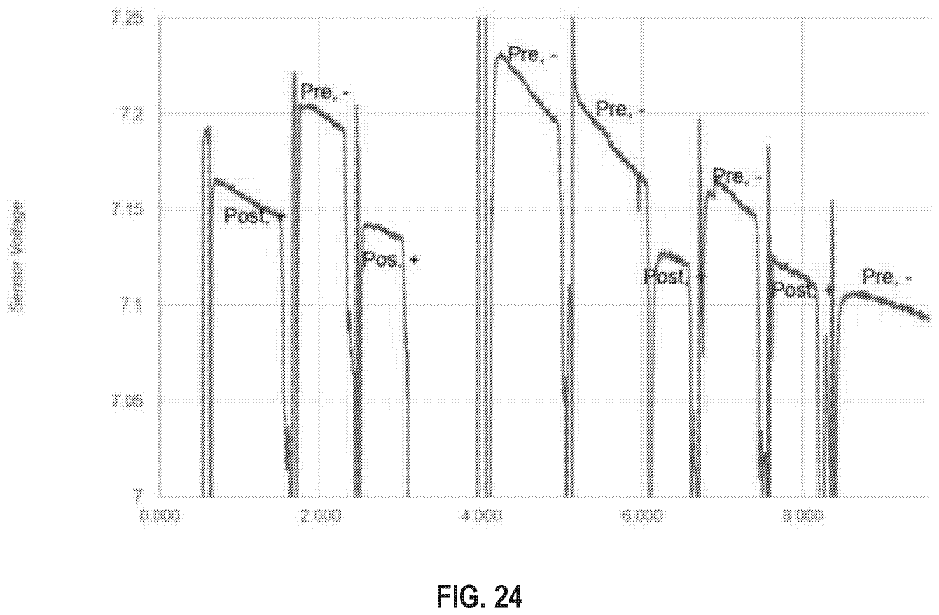

A LAMP reaction mix was prepared according to NEB's standard protocol using the 5′ untranslated region of the genome of H. influenzae as the target. The mix was aliquoted into a pre-amplification vial (− control), and post-amplification vial (+ control). The pre-amplification vial was heat-inactivated at 85° C. for 20 minutes to prevent amplification. The post-amplification vial was amplified at 63° C. for 60 minutes. Aliquots from each vial were loaded sequentially, alternating between the two vials at room temperature on to the PDMS/Glass Chip v.1.1 while real time data collection was performed. FIG. 24 is a graph depicting sensor voltage over time.

### Example 2—ƒC4D Pre/Post Amplification Detection with Whole Blood in PDMS

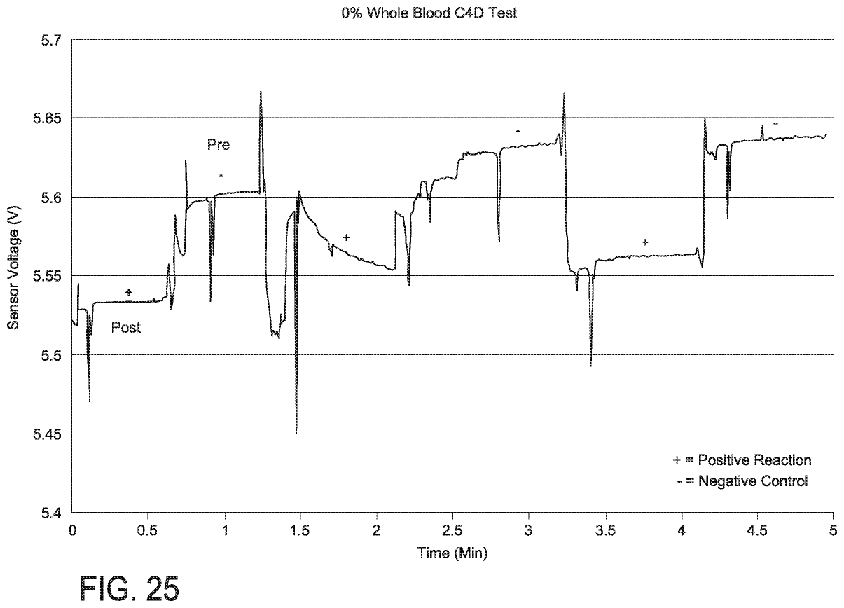

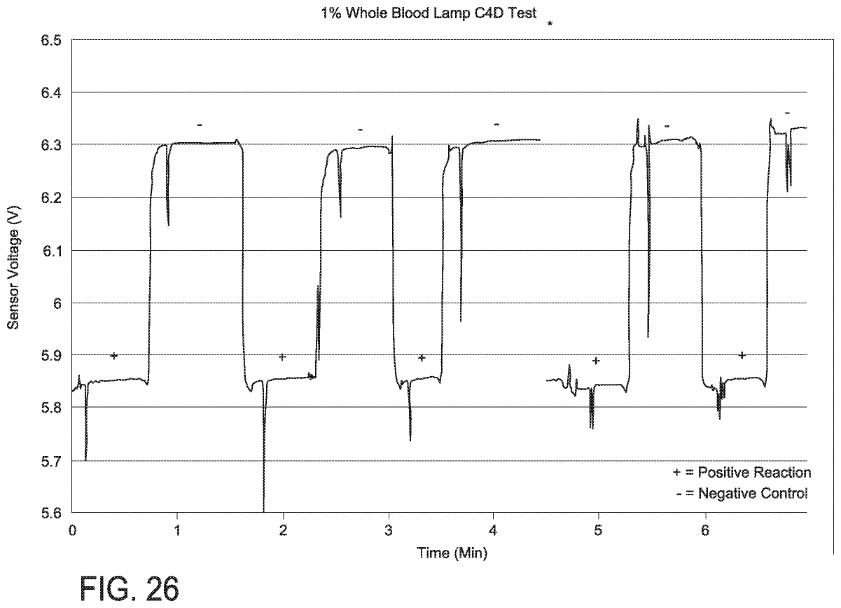

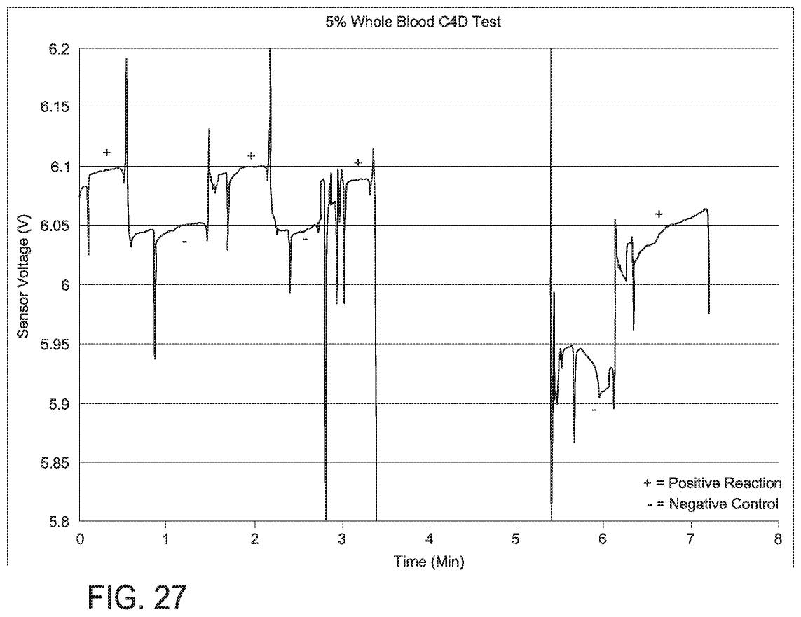

A reaction mix was prepared using the 5′ untranslated region of the genome of H. influenzae as the target with 0%, 1%, and 5% whole blood (v/v). The mix was aliquoted into a pre-amplification vial (− control), and post-amplification vial (+ control). The pre-amplification vial was heat-inactivated at 85° C. for 20 minutes to prevent amplification. The post-amplification vial was amplified at 63° C. for 60 minutes. Aliquots from each vial were loaded sequentially, alternating between the two vials at room temperature on to the PDMS/Glass Chip v.1.1 while real time data collection was performed. FIG. 25, FIG. 26 and FIG. 27 are graphs depicting sensor voltage over time for pre-amplification (− control), and post-amplification (+ control) for 0%, 1%, and 5% whole blood, respectively.

### Example 3—Filtering LAMP Pre/Post Amplification

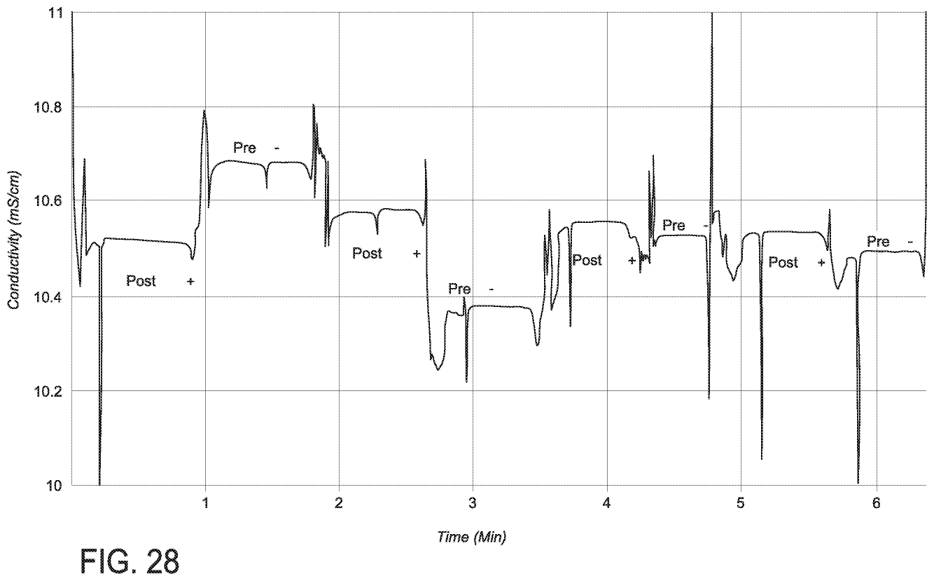

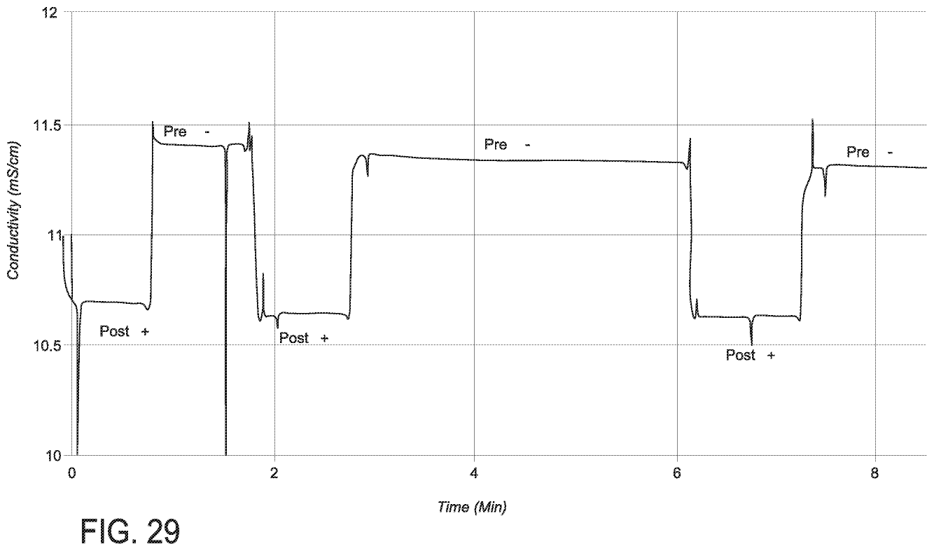

Samples were prepared as in Example 1. Prior to measurement, all samples (minus one as a control) were spin-filtered using a 50 kD filter. Aliquots from each vial were loaded sequentially, alternating between the two vials at room temperature on to the PDMS/Glass Chip v.1.1 while real time data collection was performed. Filtration improved S/N and conductivity change. FIG. 28 and FIG. 29 are graphs depicting sensor voltage over time for pre-amplification (− control), and post-amplification (+ control) with 0% whole blood, for unfiltered sample and filtered sample, respectively.

### Example 4-Conductivity Detection of 1 k-1M Target Copies

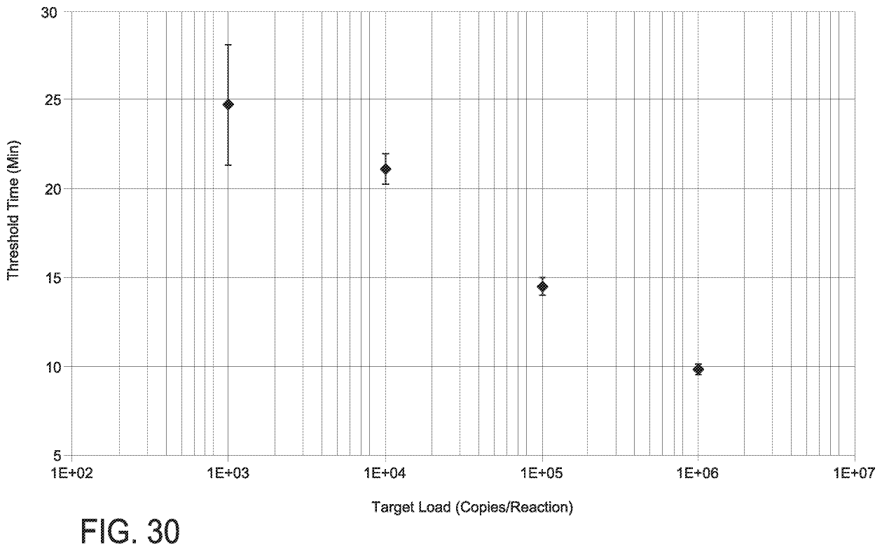

A reaction mix was prepared using the 5′ untranslated region of the genome of H. influenzae as the target. Detection was performed using a ƒC⁴D instrument. Data was averaged for 3 replicates. FIG. 30 depicts a graph of time over target load with error bars showing standard deviation. No template negative controls showed no signal at 60 minutes heating.

### Example 5—ƒC4D Pre/Post Amplification Detection with Whole Blood in PDMS

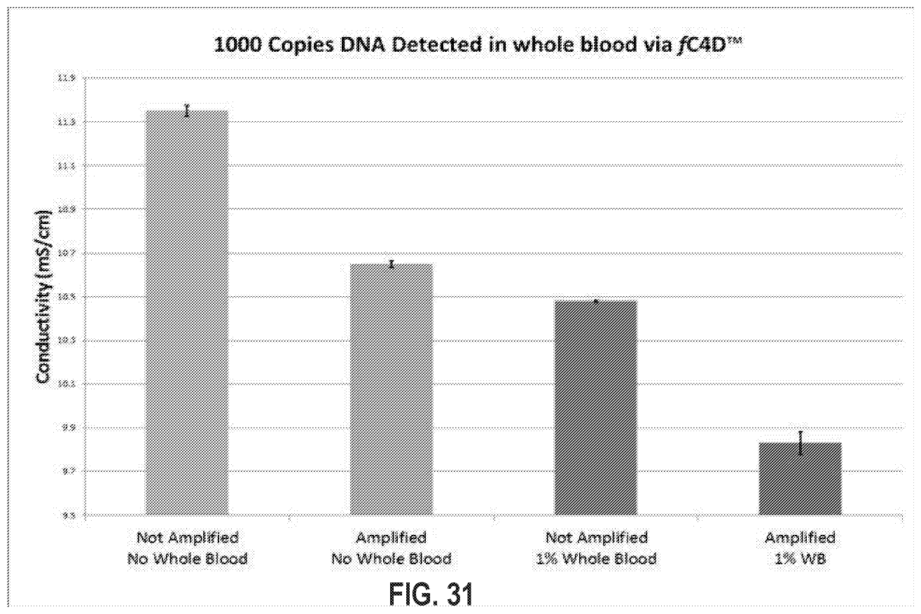

A reaction mix was prepared using the 5′ untranslated region of the genome of H. influenzae as the target with 0% or 1% whole blood (v/v). The mix was aliquoted into a pre-amplification vial (− control), and post-amplification vial (+ control). The pre-amplification vial was heat-inactivated at 85° C. for 20 minutes to prevent amplification. The post-amplification vial was amplified at 63° C. for 60 minutes. Aliquots from each vial were loaded sequentially, alternating between the two vials at room temperature on to the PDMS/Glass Chip v.1.1 while real time data collection was performed. FIG. 31 depicts a graph of conductivity for various samples from pre-amplification vial (− control), and post-amplification vial (+ control).

### Example 6—Detection of Hepatitis B Surface Antigen Using MAIA

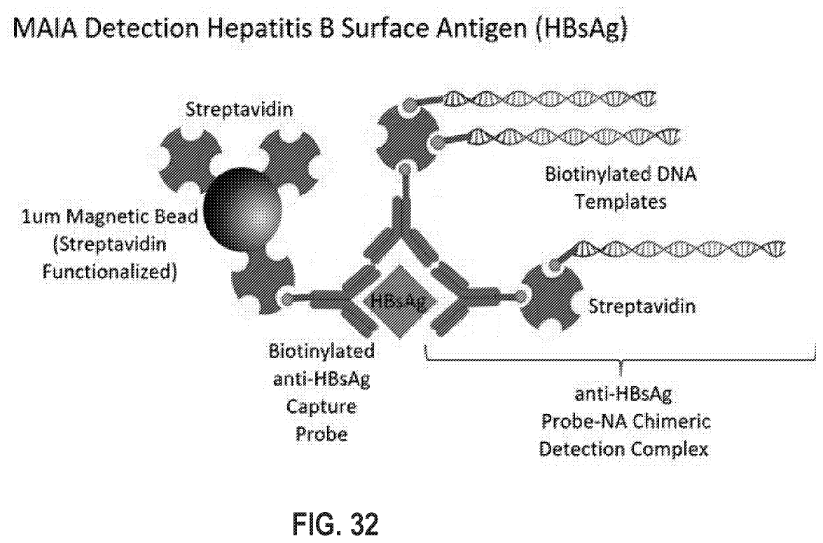

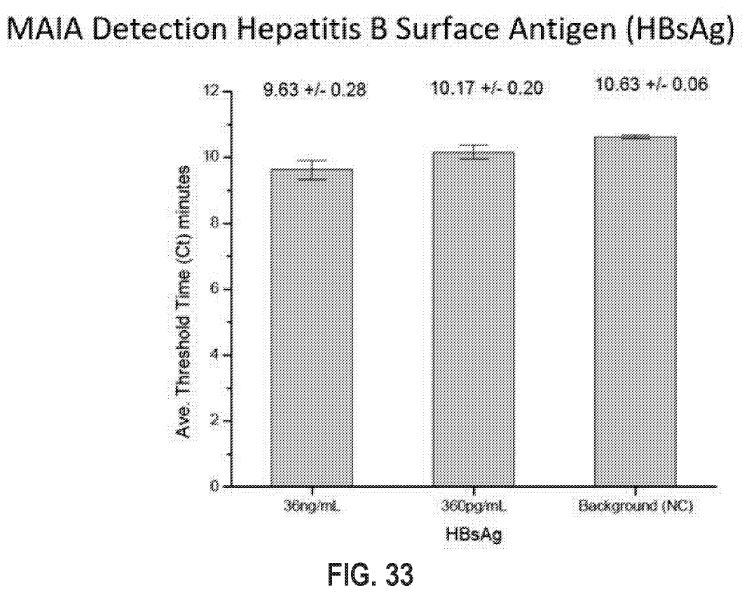

Biotinylated, polyclonal antibody capture probe (anti-HBsAg) was conjugated to streptavidin functionalized 1 micron magnetic microspheres (Dynal T1). Chimeric detection complexes were synthesized by conjugating biotinylated, polyclonal capture probe (anti-HBsAg) to streptavidin, and conjugating to the streptavidin-Antibody complex to biotinylated DNA target. The Antibody functionalized beads captured HBsAntigen from solution. The HBsAntigen was detected by the binding of the chimera Ab-DNA complex followed by amplification of the DNA template portion of the chimera complex. FIG. 32 depicts binding between antigen, antibody conjugated with nucleic acids. FIG. 33 depicts a graph showing detection of hepatitis B surface antigen.

### Example 7—Detection with Low Ionic Strength Buffer

A commercial amplification solution, and a T10 amplification solution were prepared with the reagents listed in TABLE 2 and TABLE 3, respectively. The commercial amplification solution would typically be used in general amplification reactions. The T10 amplification solution had a reduced content of Tris-HCl, and ammonium sulfate was absent. 400 μL of each solution was prepared, and about 15 μL of each solution was loaded into a different channel of an experimental cartridge. The solutions were heated to 63.0° C. Data was collected using a data collection board.

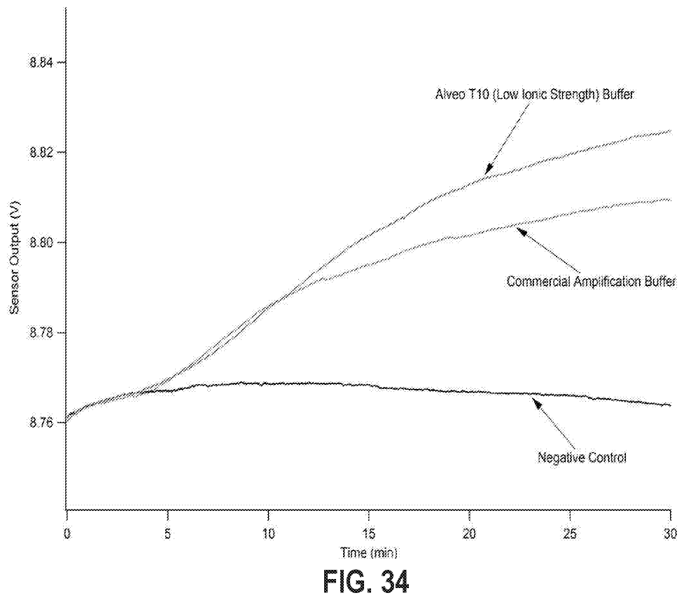

The results are depicted in FIG. 34. The T10 amplification buffer provided at least 30% greater signal compared to the signal provided by the commercial amplification solution.

TABLE 2

| | | | Final |
|---|---|---|---|
| | Volume | Final reagent | added |
| Reagent | ratio | concentration | volume |
| Isothermal amplification | 0.1 | 1x (contains 2 mM | 40 |
| buffer (10x; NEB) | | MgSO₄) | |
| MgSO₄ (100 mM; NEB) | 0.06 | 6 mM (8 mM Total) | 24 |
| dNTP mix (10 mM each; | 0.14 | 1.4 mM each | 56 |
| NEB) | | | |
| 10x H. inf. primer mix | 0.1 | 1x (1.6 μM FIP/BIP, | 40 |
| | | 0.2 μM F3/B3, | |
| | | 0.4 mM LoopF/B | |
| Bst 2.0 WarmStart | 0.04 | 320 U/L | 16 |
| polymerase (8000 U/L; | | | |
| NEB) | | | |
| H. inf. DNA Sample (1 | 0.04 | | 16 |
| Mc/uL) | | | |
| Ultra Pure Water | 0.52 | | 208 |
| Total | | | 400 |

TABLE 3

| | 1x | 10x | | mg to add |
|---|---|---|---|---|
| | concentration | concentration | | for 10 mL |
| Reagent | (mM) | (mM) | FW | 10x |
| Tris-HCl | 2 | 20 | 157.6 | 31.52 |
| KCl | 50 | 500 | 74.55 | 372.75 |
| MgSO₄ | 2 | 20 | 246.48 | 49.30 |
| Tween 20 | 0.10% | 1% | 100% | 0.1 mL |
| DI Water | | | | 9.9 mL |

### Example 8—Impedance Characteristics of a Fluidics Cartridge

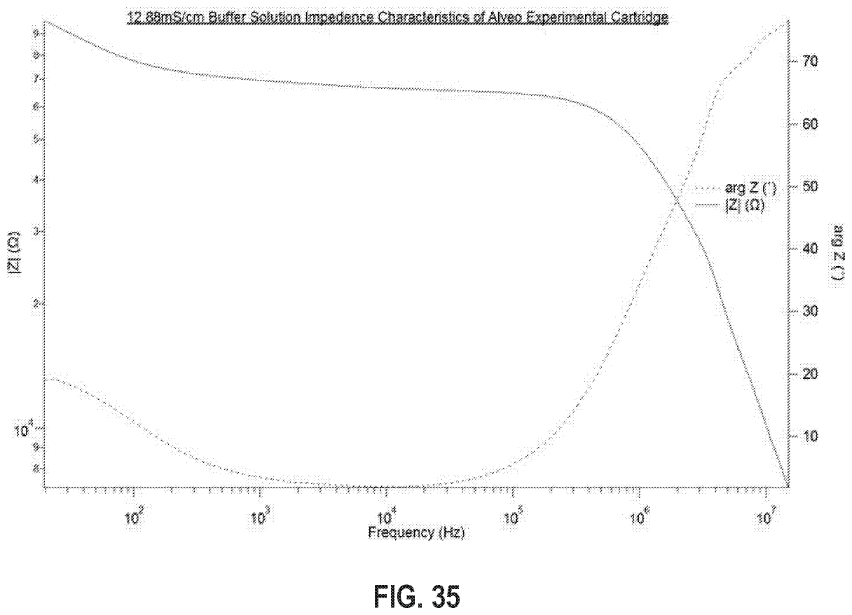

The channels of a fluidics cartridge depicted in FIG. 17A were filled with a 1288 mS/cm reference buffer, and an excitation frequency was swept from less than about 100 Hz to greater than about 1 MHz, and the impedance (“|Z|”) or arg Z over frequency were measured. The results are shown in FIG. 35 which depicts either |Z| or arg Z over frequency.

### Example 9—Amplification of Nucleic Acids Containing HCV Sequences

Samples containing nucleic acids comprising Hepatitis C virus (HCV) sequences were amplified in a series of experiments by LAMP under various conditions, and threshold cycle (Ct) values along with standard deviations (SD) and % relative standard deviations (RSD) were determined. Nucleic acids included synthetic nucleic acids comprising an HCV sequence; synthetic RNA comprising an HCV sequence. All reactions contained 5% Tween-20. For experiments with reactions containing about a million copies of synthetic nucleic acids comprising an HCV sequence, the average Ct was 856 s, with a SD of 15 s, and a RSD of 1.72%.

Samples of plasma containing synthetic RNA comprising an HCV sequence were amplified by LAMP under various conditions including: untreated, treated by heating before addition of the synthetic RNA, by heating after addition of the synthetic RNA, and by adding 100 mM DTT. Each reaction contained about 25 k copies of the nucleic acid. TABLE 4 summarizes the results.

TABLE 4

| | | Heat-treated before | Heat-treated after | 100 nM |
|---|---|---|---|---|
| | | addition of the | addition of the | DTT |
| Parameter | Untreated | synthetic RNA | synthetic RNA | added |
| Average Ct | 1043 | 983 | 1190 | 999 |
| (s) | | | | |
| SD (s) | 53 | 26 | 145 | 19 |
| RSD (%) | 5.12 | 2.64 | 12.22 | 1.93 |
| n | 12 | 16 | 8 | 4 |

Addition of 100 mM DTT, or heat-treating plasma before addition of the synthetic RNA improved amplification as shown by relative standard deviation (RSD) compared to untreated samples. Adding DTT, or heat-treating plasma before addition of the synthetic RNA also produced faster amplification (about 50 s faster) compared to untreated samples (P=0.03 and 0.002, respectively).

Samples of plasma containing HCV (SeraCare, Milford Mass.) were amplified by LAMP under various conditions including: heat-treating the plasma, adding 100 mM DTT, adding SDS and/or DTT. TABLE 5 summarizes the results.

TABLE 5

| | | | | | | 0.05% | 0.05% |
|---|---|---|---|---|---|---|---|
| | | | 100 nM | 0.05% | 0.1% | SDS + 100 | SDS + 100 |
| Parameter | Untreated | Heat-treated | DTT | SDS | SDS | mM DTT | mM DTT |
| Average Ct (s) | 2020 | 1081 | 1117 | 2032 | 2793 | 1190 | 1288 |
| SD (s) | 1368 | 111 | 130 | 2052 | 1617 | 230 | 278 |
| RSD (%) | 67.72 | 10.23 | 11.63 | 100.96 | 57.89 | 19.36 | 21.57 |
| n | 15 | 18 | 16 | 16 | 15 | 16 | 16 |

Heat treating the plasma or adding DTT improved amplification results, compared to untreated plasma, as shown by RSD values. Adding either 0.05% or 0.1% SDS reduced the reproducibility and speed of the amplification compared to plasma that was untreated, heat-treated, or DTT was added.

### Example 10—Amplification of Clinical Samples Containing HCV

Clinical plasma samples containing HCV were amplified by LAMP with various concentrations of DTT. WarmStart LAMP master mix (New England Biolabs) was used to prepare samples in quadruplicates. Samples included: 5% plasma (SeraCare, Milford Mass.) containing about ˜20 k copies of HCV/reaction, 50 U/reaction murine RNase inhibitor, with various concentrations of Tween and DTT. Samples containing synthetic nucleic acids comprising an HCV sequence (1M copies/r×n) were tested with 10 and 5 Tween-20. No target controls (NTCs) were also tested. LAMP was carried out at 67° C. for 60 m on an Applied Biosystems QuantStudio 3 (QS3) system. Fluorescence was measured once per minute to assess reaction progress. Results are summarized in TABLE 6.

TABLE 6

| | Average | | |
|---|---|---|---|
| Sample | Cₜ (s) | SD (s) | RSD (%) |
| Synthetic nucleic acids + 1% Tween | 1214 | 15 | 1.22 |
| Synthetic nucleic acids + 5% Tween | 1123 | 54 | 4.84 |
| Plasma + 1% Tween | 1754 | 1040 | 59.32 |
| Plasma + 1% Tween + 5 mM DTT | 1728 | 1030 | 59.61 |
| Plasma + 1% Tween + 10 mM DTT | 1202 | 213 | 17.76 |
| Plasma + 1% Tween + 25 mM DTT | 1467 | 609 | 41.53 |
| Plasma + 1% Tween + 50 mM DTT | 1576 | 543 | 34.43 |
| Plasma + 1% Tween + 100 mM | 1391 | 165 | 11.84 |
| DTT | | | |
| Plasma + 5% Tween | 1038 | 48 | 4.64 |
| Plasma + 5% Tween + 5 mM DTT | 961 | 52 | 5.43 |
| Plasma + 5% Tween + 10 mM DTT | 979 | 68 | 6.94 |
| Plasma + 5% Tween + 25 mM DTT | 983 | 38 | 3.89 |
| Plasma + 5% Tween + 50 mM DTT | 965 | 122 | 12.66 |
| Plasma + 5% Tween + 100 mM | 1111 | 102 | 9.18 |

| | |
|---|---|
| DTT | |
| No template control | No amplification detected |

Samples containing 500 Tween had improved amplification compared to samples containing 100 Tween, as shown by RSD values. A similar study was carried out further varying the concentrations of Tween in reaction tubes. The results are summarized in TABLE 7.

TABLE 7

| | Average | | |
|---|---|---|---|
| Sample | Cₜ (s) | SD (s) | RSD (%) |
| Synthetic nucleic acids + 2% Tween | 957 | 3 | 0.27 |
| Synthetic nucleic acids + 5% Tween | 842 | 12 | 1.37 |
| Plasma + 2% Tween | 2163 | n/a | n/a |
| Plasma + 2% Tween + 0.5 mM | 1671 | 989 | 59.16 |
| DTT | | | |
| Plasma + 2% Tween + 1 mM DTT | 1512 | 623 | 41.17 |
| Plasma + 2% Tween + 5 mM DTT | 1234 | 154 | 12.45 |
| Plasma + 2% Tween + 10 mM DTT | 1042 | 56 | 5.38 |
| Plasma + 3% Tween | 1995 | 1004 | 50.34 |
| Plasma + 3% Tween + 0.5 mM | 1119 | 63 | 5.65 |
| DTT | | | |
| Plasma + 3% Tween + 1 mM DTT | 1581 | 948 | 59.87 |
| Plasma + 3% Tween + 5 mM DTT | 1067 | 107 | 10.03 |
| Plasma + 3% Tween + 10 mM DTT | 1237 | 120 | 9.73 |
| Plasma + 4% Tween | 1182 | 71 | 6.04 |
| Plasma + 4% Tween + 0.5 mM | 1112 | 117 | 10.56 |
| DTT | | | |
| Plasma + 4% Tween + 1 mM DTT | 1229 | 301 | 24.50 |
| Plasma + 4% Tween + 5 mM DTT | 1076 | 114 | 10.64 |
| Plasma + 4% Tween + 10 mM DTT | 1017 | 57 | 5.61 |
| Plasma + 5% Tween | 1142 | 62 | 5.42 |
| Plasma + 5% Tween + 0.5 mM | 1104 | 93 | 8.46 |
| DTT | | | |
| Plasma + 5% Tween + 1 mM DTT | 1510 | 800 | 52.99 |
| Plasma + 5% Tween + 5 mM DTT | 1020 | 65 | 6.34 |
| Plasma + 5% Tween + 10 mM DTT | 1014 | 59 | 5.79 |

| | |
|---|---|
| No template control | No amplification detected |

Reaction volumes with greater concentrations of Tween and DTT had better reproducibility of amplification results for the HCV samples, specifically, in replicated reactions there were fewer extreme outliers, fewer failed amplifications, and lower RSD values for amplified replicates. At 5 mM DTT and 10 mM DTT, there were no replicates that did not amplify for any concentration of Tween. Likewise, at 4% and 5% Tween, there were no failed replicates or extreme outliers, except for the low (1 mM and below) DTT concentrations.

### Example 11—Amplification of Targets with Cartridges

A series of three experiments were performed using a cartridge substantially similar to the cartridge depicted in FIG. 2 having six wells, each well having an annular ring electrode. Each well was associated with a measured channel. Samples included targets nucleic acids comprising sequences from Haemophilus influenzae (Hinf), or Hepatitis B virus (HBV). Samples were amplified by LAMP, and changes in impedance were measured.

Wells were prepared by pre-heating the cartridge to 72° C. for 20 minutes, filling each well with 25 μl ‘no template and primer control’ (NTPC) buffer, capping the buffer with mineral oil, heating the cartridge to 72° C. for 20 minutes, removing bubbles from the wells, cooling the cartridge at room temperate for 10 minutes. Samples were injected at the bottom of the prefilled wells, and the cartridge was placed at 67° C., or 76.5° C. to carry out the LAMP for a particular experiment. The frequency used for the Hinf studies was 60 kHz. Samples and corresponding wells/channels for each cartridge are listed in TABLE 8. Target sequences and primers are listed in TABLE 9. Reaction components are listed in TABLE 10.

TABLE 8

| Well/channel | Sample |
|---|---|
| 1 | Synthetic |
| | HBV |
| 2 | Synthetic |
| | HBV |
| 3 | Synthetic |
| | HBV |
| 4 | NTPC |
| 5 | Hinf |
| 6 | Hinf |

TABLE 9

| SEQ ID NO: | Sequence |
|---|---|
| SEQ ID NO: 01 | GACAAGAATCCTCACAATACCGCAGAGTCTAGACTC |
| (HBV target) | GTGGTGGACTTCTCTCAATTTTCTAGGGGGATCACC |
| | CGTGTGTCTTGGCCAAAATTCGCAGTCCCCAACCTC |
| | CAATCACTCACCAACCTCCTGTCCTCCAATTTGTCCT |
| | GGTTATCGCTGGATGTGTCTGCGGCGTTTTATCATAT |
| | TCCTCTTCATCCTGCTGCTATGCC |
| SEQ ID NO: 02 | TCCTCACAATACCGCAGAGT |
| (HBV F3 primer) | |
| SEQ ID NO: 03 | GCATAGCAGCAGGATGAAGA |
| (HBV B3 primer) | |
| SEQ ID NO: 04 | GTTGGGGACTGCGAATTTTGGCCTCGTGGTGGACTTCTCTCA |
| (HBV FIP primer) | |
| SEQ ID NO: 05 | TCACCAACCTCCTGTCCTCCAAATAAAACGCCGCAGACACAT |
| (HBV BIP primer) | |
| SEQ ID NO: 06 | CACGGGTGATCCCCCTA |
| (HBV LF primer) | |
| SEQ ID NO: 07 | TTTGTCCTGGTTATCGCTGG |
| (HBV LB primer) | |
| SEQ ID NO: 08 | TGGTACGCCAATACATTCAACAAGAAATTAATCCAA |
| (Hinf target) | AAGAAAAATTTGCGTTTGTTGAATTCTGGGGGCGAG |
| | GCTATACACAAGATACCTTTGGTCGTCTGCTAAATG |
| | ATGCCTTTGGTAAAGAAGTAAAAAACCCATTCTATT |
| | ATGTCAGAAGTTTTACTGATGATATGGGTACATCTG |
| | TTCGCCATAACTTCATCTTAGCACCACAAAACTTCT |
| | CATTCTTCGAGCCTATTTTTGCACAAACCCCATACG |
| | ACAGTATTCCTGATTACTACGAAGAAAAAGGCAGA |
| | ATTGAACCAATTA |
| SEQ ID NO: 09 | GCAGACGACCAAAGGTATCTTG |
| (Hinf LF primer) | |
| SEQ ID NO: 10 | CGTATGGGGTTTGTGCA |
| (Hinf B3 primer) | |
| SEQ ID NO: 11 | CGCCAATACATTCAACAAGA |
| (Hinf F3 primer) | |
| SEQ ID NO: 12 | CTGATGATATGGGTACATCTGTTCGCGAAGAATGAG |
| (Hinf BIP primer) | AAGTTTTGTGG |
| SEQ ID NO: 13 | ACTTCTTTACCAAAGGCATCATTTTGCGTTTGTTGAC |
| (Hinf FIP primer | GCCAAATTCTGG |
| sequence) | |

TABLE 10

| | | Volume |
|---|---|---|
| Mix | Component | (μl) |
| Master mix 1 | LAMP master mix (2X; NEB) | 12.5 |
| | dUTP Additive (100 mM; Sigma) | 0.175 |
| NTPC mix | Master mix 1 | 12.675 |
| | Water | 12.325 |
| Hinf mix | Master mix 1 | 12.675 |
| | Hinf primers (25X) | 1 |
| | Hinf target (1M/μl) | 1 |
| | Water | 10.325 |
| Master mix 2 | Master mix 1 | 12.675 |
| | UDG | 0.5 |
| | HBV primers (25X) | 1 |
| Synthetic | Master mix 2 | 13.175 |
| HBV | HBV target (10e10 c/μl) | 1 |
| | Water | 10.825 |

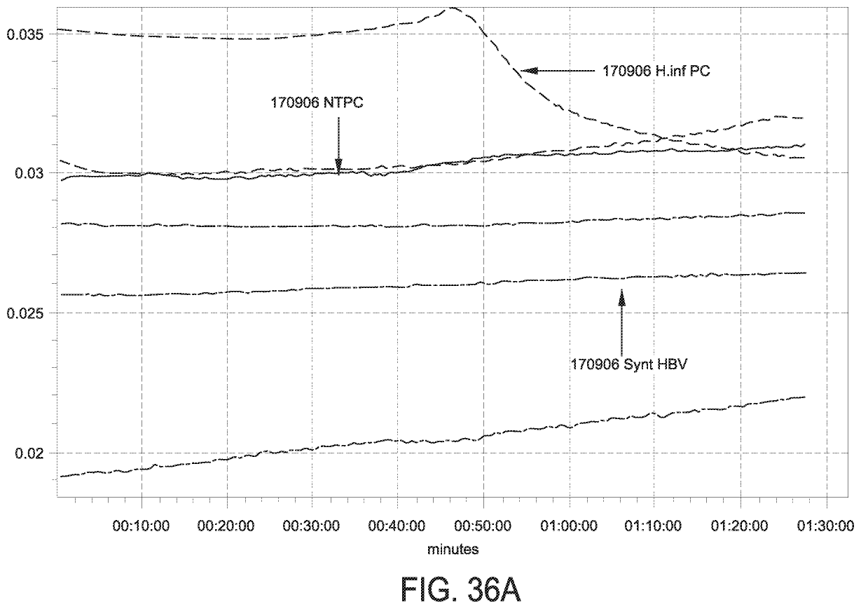

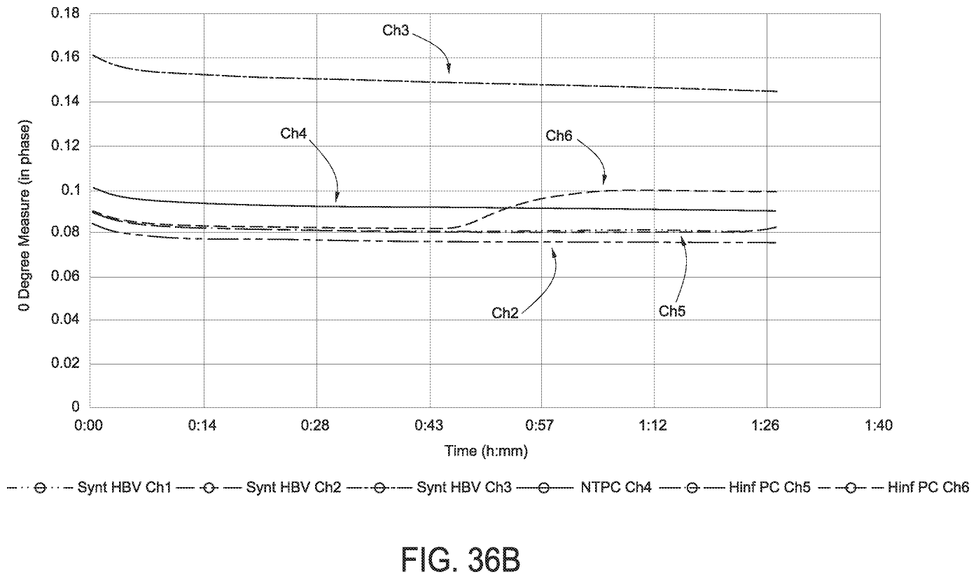

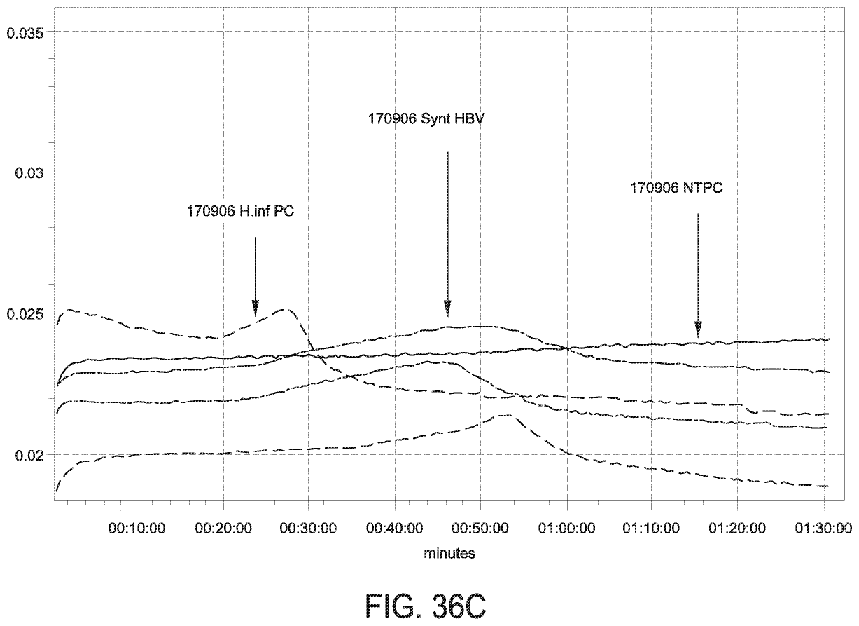

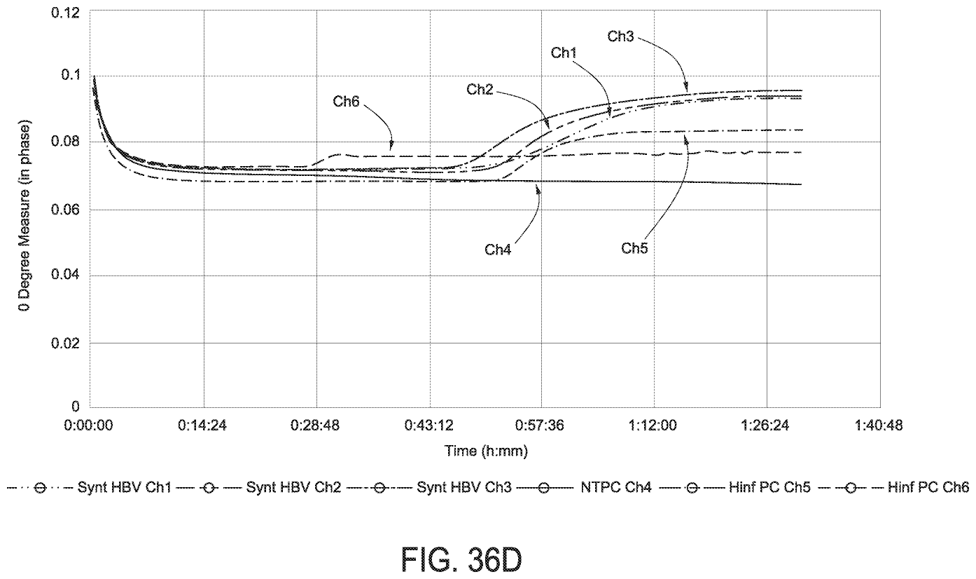

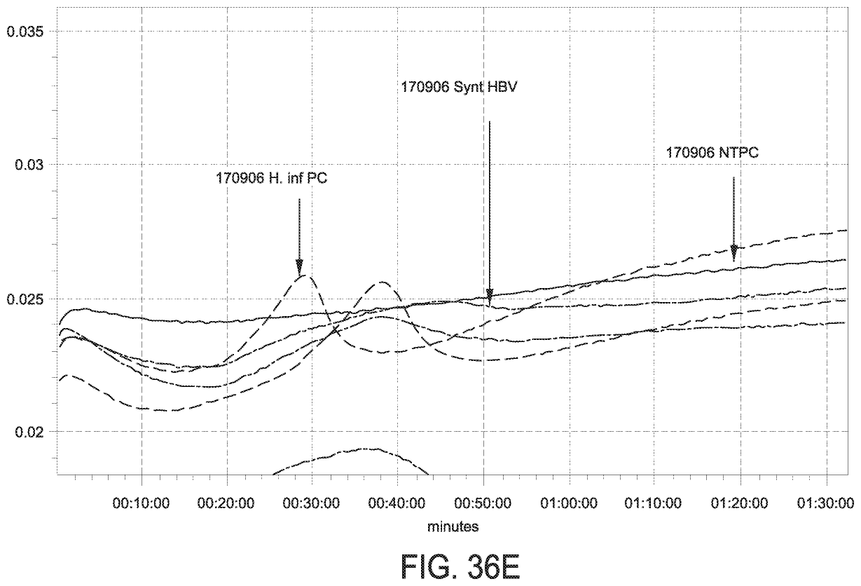

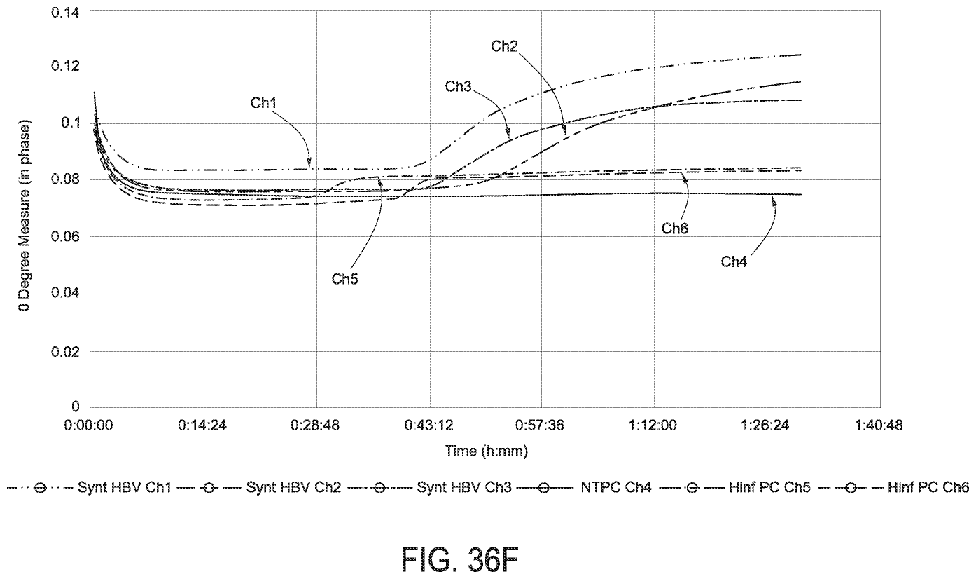

Data for LAMP carried out on the cartridge at 65° C. are shown in FIGS. 36A and 36B. FIG. 36A is a graph of the out of phase portion of an attenuated excitation signal sensed in a test well of the cartridge of FIG. 2, in which the x-axis is time, and lines representing LAMP on samples for NTPC, and examples of Hinf and synthetic HBV are labelled. FIG. 36B is a graph of the in phase portion of an attenuated excitation signal sensed in a test well of the cartridge of FIG. 2, with lines representing synthetic HBV (channels 1-3), NTPC (channel 4) and Hinf (channels 5-6). Samples containing synthetic HBV were not amplified on the cartridge at 65° C. The labeled Hinf sample shows an example signal cliff indicative of a positive sample.

Data for LAMP carried out on the cartridge at 67° C. are shown in FIGS. 36C and 36D. FIG. 36C is a graph of the out of phase portion of an attenuated excitation signal sensed in a test well of the cartridge of FIG. 2, in which the x-axis is time, and lines representing LAMP on samples for NTPC, and examples of Hinf and synthetic HBV are labelled. FIG. 36D is a graph of the in phase portion of an attenuated excitation signal sensed in a test well of the cartridge of FIG. 2 with lines representing synthetic HBV (channels 1-3), NTPC (channel 4) and Hinf (channels 5-6). Samples containing synthetic HBV amplified on the cartridge at 67° C. at about 49 minutes. The labeled Hinf sample shows an example signal cliff indicative of a positive sample.

Data for LAMP carried out on the cartridge at 67° C. is shown in FIGS. 36E and 36F. FIG. 36E is a graph of the out of phase portion of an attenuated excitation signal sensed in a test well of the cartridge of FIG. 2, in which the x-axis is time, and lines representing LAMP on samples for NTPC, and examples of Hinf and synthetic HBV are labelled. FIG. 36F is a graph of the in-phase portion of an attenuated excitation signal sensed in a test well of the cartridge of FIG. 2, with lines representing synthetic HBV (channels 1-3), NTPC (channel 4) and Hinf (channels 5-6). Samples containing synthetic HBV amplified on the cartridge at 67° C. at about 46 minutes.

Samples were also tested by LAMP using a QS3 at 67° C. TABLE 11 lists average Ct values for samples containing Hinf, or synthetic HBV. Data in the graph are shown in seconds (s).

TABLE 11

| Sample (target | Average | | |
|---|---|---|---|
| concentration) | Ct (s) | SD (s) | RSD (%) |
| Hinf PC (1M copies) | 1704.5 | 10.4 | 0.6 |
| HBV Synt (10B | 380.4 | 5.5 | 1.5 |
| copies) | | | |

### Example 12—RPA Master Mix and Protocol

The following is an example of a Basic Master Mix for an RPA reaction: 50 mM Tris-Acetate, pH 7.8; 5.5% polyethylene glycol-35 k; 5% trehalose; 100 mM potassium acetate; 6 μg T4 UvsX recombinase; 2.5 μg T4 UvsY; 6 μg T4 gp32 SSB; 2.5 μg creatine kinase; 5 mM dithiothreitol (DTT); 50 mM phosphocreatine (from disodium salt); 1 U of polymerase from Staph aureus (Sau polymerase) or Bacillus subtilis (Bsu polymerase). In some embodiments, the Basic Master Mix used in a 25-μL reaction. Additional disclosure related to RPA and the Basic Master Mix are provided in U.S. Pat. No. 9,057,097 B2. The entire contents of U.S. Pat. No. 9,057,097 B2 are incorporated herein by reference. In some embodiments, an amplification reaction solution or an RPA reagent solution comprises the Basic Master Mix, or one or more components of the Basic Master Mix.

The following is an example RPA experimental protocol:
(1) Mix all RPA reagents together (buffer, enzymes, primers, etc.) except for the target DNA/RNA and magnesium acetate (activates the enzymes used in the reaction)
(2) Pipet reaction mixes into separate 25-μL PCR tubes
(3) Pipet a mixture of the target and activator into the lid of each tube
(4) Close lid, invert several times to mix, then briefly centrifuge the tubes
(5) Place tubes in thermocycler
(6) Run RPA reaction at 40° C., taking data every minute.

Some embodiments of the methods provided herein include performing RPA and/or another isothermal amplification such as LAMP. According to some embodiments, the Basic Master Mix and/or the RPA experimental protocol described in Example 12, or certain aspects thereof, are used to perform the RPA and/or LAMP or another isothermal amplification.

### Example 13—RPA with Blockers

Initial attempts at performing RPA without optical probes (i.e., forward and reverse primers only +dsDNA-intercalating reporter dye) found that curves from NTCs overlapped substantially or completely with those from positive samples. This was the case for a variety of primer sets and targets. It was hypothesized that this was due to the relatively low RPA reaction temperature enabling polymerase-extensible interactions between primers at their 3′ termini. It was therefore tested whether blocking oligonucleotides could mitigate this problem by making the 3′ termini of the primers double-stranded until they were fully annealed to their target sequences. TwistAmp Liquid Basic Master Mix (the Basic Master Mix described in Example 12) was used, and 3 replicates of a target (Synt H inf at 1 million copies/reaction) (SEQ ID NO: 08) and 3 replicates of NTC reactions were made with the following combination of blocking oligos as follows with the concentrations in parentheses: POSITIVE CONTROL (no blockers), F1_B10+R3_B10 (2400 nM and 4800 nM each), F1_B12+R3_B12 (2400 nM and 4800 nM each), F1_B15+R3_B15 (2400 nM and 4800 nM each), F1_B10+R3_B15 (4800 nM each), F1_B15+R3_B10 (4800 nM each).

The RPA experiment was run on a QuantStudio 5 thermocycler (Thermo Fisher Scientific) for 60 minutes at 65° C., with fluorescence recorded once per minute to assess reaction progress. The samples are named in the following format: (P or N_length_F−lengthR_concentration) so, the positive with both 10 @ 2400 nM would be P_10-10_2400, and the NTC for F1_B15+R3_B10 would be N15-10_4800.

Results are shown in TABLE 12. Oligonucleotide sequences are included in TABLE 13.

TABLE 12

| | Ct Mean | Ct SD | % | # Replicates |
|---|---|---|---|---|
| Sample Name | (min) | (min) | RSD | Amplified |
| PC No Blocker | 4.88 | 0.15 | 3.14 | 3 out of 3 |
| Control | | | | |
| P_15-15_4800 | 13.17 | 0.42 | 3.16 | 3 out of 3 |
| P_15-15_2400 | 8.15 | 0.74 | 9.09 | 3 out of 3 |
| P_15-10_4800 | 8.02 | 0.00 | 0.01 | 3 out of 3 |
| (1 outlier eliminated) | | | | |
| P_12-12_2400 | 4.34 | 0.33 | 7.66 | 3 out of 3 |
| P_10-15_4800 | 5.88 | 0.10 | 1.78 | 3 out of 3 |
| P_10-10_4800 | 4.36 | 0.28 | 6.43 | 3 out of 3 |
| P_10-10_2400 | 3.62 | 0.33 | 8.99 | 3 out of 3 |
| NTC No Blocker | 6.10 | 0.52 | 8.51 | 3 out of 3 |
| Control | | | | |
| N_15-15_4800 | 51.20 | 3.99 | 7.78 | almost |
| | | | | amplified |
| | | | | 3 out of 3 |
| N_15-15_2400 | 25.40 | 1.11 | 4.35 | 3 out of 3 |
| N_15-10_4800 | 24.54 | 0.33 | 1.36 | 3 out of 3 |
| N_12-12_4800 | 8.37 | 0.49 | 5.88 | 3 out of 3 |
| N_12-12_2400 | 5.74 | 0.40 | 6.90 | 3 out of 3 |
| N_10-15_4800 | 7.47 | 0.31 | 4.15 | 3 out of 3 |
| N_10-10_4800 | 4.21 | 0.61 | 14.59 | 3 out of 3 |
| N_10-10_2400 | 3.63 | 0.02 | 0.44 | 3 out of 3 |

TABLE 13

| SEQ ID | | | |
|---|---|---|---|
| NO: | Type | Name | Sequence |
| 14 | RPA | H Inf_180430 RPA_F1 | CGCCAATACATTCAACAAGAAATTAA |
| | Primer | (“F1”) | TCCAAAAG |
| 15 | RPA | H Inf_180430_F1_B10 | CTTTTGGATT/3Phos/ |
| | Blocker | (“F1_B10”) | |
| 16 | RPA | H Inf_180430_F1_B12 | CTTTTGGATTAA/3Phos/ |
| | Blocker | (“F1_B12”) | |
| 17 | RPA | H Inf_180430_F1_B15 | CTTTTGGATTAATTT/3Phos/ |
| | Blocker | (“F1_15”) | |
| 18 | RPA | 180709 B17_F1_H Inf | CTTTTGGATTAATTTCT/3Phos/ |
| | Blocker | 180430 RPA (“F1_17”) | |
| 19 | RPA | 180709 B19_F1_H Inf | CTTTTGGATTAATTTCTTG/3Phos/ |
| | Blocker | 180430 RPA (“F1_19”) | |
| 20 | RPA | 180709 B21_F1_H Inf | CTTTTGGATTAATTTCTTGTT/3Phos/ |
| | Blocker | 180430 RPA (“F1_21”) | |

As shown in TABLE 12, the samples with F5._B9+R3_B15 (4800 nM each) worked well; while the no-blocker positive and negative samples were separated in Ct by approximately one minute, the P_15-15_4800 samples and N_15-15_4800 samples were separated by nearly 40 minutes of amplification time. These results show that according to some embodiments, inclusion of blocker oligonucleotides in an RPA reaction solution enhances RPA or another amplification reaction by increasing specificity. The inventors next assessed the dynamic range of our assay with this blocker combination using decade dilutions of target from 1 million copies per reaction down to 0 copies per reaction. Results are shown in TABLE 14.

TABLE 14

| Copies of | Ct Mean | Ct SD | |
|---|---|---|---|
| H inf Target | (min) | (min) | % RSD |
| 10\hat{\hat{ }}6 | 14.08 | 0.46 | 3.24 |
| 10\hat{ }5 | 18.00 | 0.46 | 2.54 |
| 10\hat{ }4 | 25.04 | 0.46 | 1.85 |
| 10\hat{ }3 | 37.22 | 2.23 | 5.98 |
| 10\hat{ }2 | 38.90 | 1.58 | 4.05 |
| 10\hat{ }1 | 40.62 | 0.72 | 1.78 |
| 0 (NTC) | 43.13 | 3.71 | 8.61 |

As shown in TABLE 14, the template was detected down to 10 copies per reaction but at 10 copies per reaction, it appeared within the NTC range. NTCs were successfully inhibited by the Blockers. These results show that according to some embodiments, inclusion of blocker oligonucleotides in an RPA reaction solution enhances RPA or another amplification reaction by increasing specificity.

### Example 14—Isothermal Amplification Such as LAMP with Low Levels of DTT, or in the Absence of DTT

Another problem that was encountered in some cases was a difficulty with the inclusion of dithiothreitol (DTT) in commercial RPA kits. DTT is a reducing agent whose thiol groups have a strong affinity for gold. Some embodiments of the devices and methods used herein relate to electrodes comprising gold. For example, some embodiments of the cartridge electrodes contain gold. In some embodiments, some concentrations of DTT prevent detection on a cartridge or with a device described herein. It was found, for example, that in some cases DTT concentrations above ˜1 mM prevented detection on a cartridge, but that amounts of DTT at about 0.5 mM were acceptable.

DTT was found to not have deleterious effects on optical detection methods with thermocyclers, indicating that the DTT indeed was interfering with a cartridge's method of electrical detection, but not the amplification reaction itself. To circumvent this issue, in some embodiments, where a 5 mM or 1-10 mM DTT concentration would typically be used without a detection method or device described herein, a lower concentration of DTT may be substituted (for example, 0.25 mM). In other words, if a master mix with a higher amount (such as 5 mM) is described, a similar master mix may be made in some embodiments that includes, for example, 0.25 mM DTT may be used instead. For example, some embodiments include a Basic Master Mix such as the one in Example 12, but have about 0.25 mM DTT instead of 5 mM DTT.

An experiment was performed to determine whether in some cases the reducing agents, DTT and tris(2-carboxyethyl)phosphine (TCEP), react with the cartridge electrode in such a way that the amplification signal is not properly detected. If one or both of these reducing agents is shown to not affect signaling, they may be used in cartridge assays for both viral lysis and for mitigating negative effects of nasal fluid sample testing. For this experiment, samples were made up with H. Influenzae target and LAMP primers (SEQ ID NOS: 08-13) with 5 mM of either DTT or TCEP in a LAMP reaction solution. The samples were loaded directly into a preheated cartridge and run for 60 minutes at 65° C. with electrical detection. Identical samples were also run on a QS3 thermocycler to optically assess the effect of DTT/TCEP on the reactions. Ct values from amplification plots are shown in TABLE 15.

TABLE 15

| | | Avg | Ct SD | |
|---|---|---|---|---|
| Instrument | Additive | Ct (min) | (min) | % RSD |
| QS3 | TCEP | 18.9 | 0.008 | 0.04 |
| Cartridge | TCEP | 13.3 | 1.1 | 8.3 |
| QS3 | DTT | 18.7 | 0.3 | 1.6 |

| | | |
|---|---|---|
| Cartridge | DTT | no amplification |

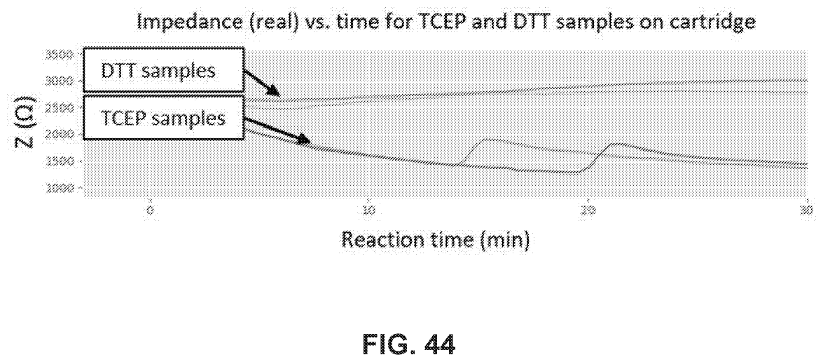

Cartridge test results are shown in FIG. 44. The results of the experiment showed that the H. Influenzae samples with 5 mM TCEP added amplified normally in the thermocycler, indicating that it did not interfere with LAMP chemistry. It also produced a normal amplification signal when samples were run in the cartridges with electrical detection. With these results, we should expect TCEP to be compatible with isothermal amplification assays such as LAMP in a cartridge according to some embodiments, and that isothermal amplification assays such as LAMP may proceed in the absence of DTT. In some embodiments of the methods described herein, an amplification reaction solution comprises TCEP. In some embodiments, the TCEP in the amplification reaction solution is at 5 mM, about 5 mM, 1-10 mM, or 3-7 mM.

The results of the experiment also showed H. Influenzae samples with 5 mM DTT amplified normally in the thermocycler showing that DTT did not interfere with LAMP chemistry. When the H. Influenzae with 5 mM DTT samples were run in the cartridge with electrical detection, no amplification was detected. Because the DTT sample amplified in the thermocycler, it is likely that there was amplification in the cartridge, but the signal was not properly reported due to possible corrosion of the electrode. This result may have been due to an affinity of DTT's thiol groups for gold.

### Example 15—Isothermal Amplification Such as LAMP or RPA with or without Calcium in the Amplification Reaction Solution

Experiments were performed to test the detection of H Inf with a low amount of dNTP (1.8 mM in this experiment) with and without CaCl₂), which the inventors found to be signal-enhancing in previous experiments. The experiments were conducted according to a procedure using the Basic Master Mix in Example 12, but with modifications including changing the dNTP concentration to 1.8 mM and differing the CaCl₂) amount as follows.
H. Inf LAMP primers F3 and B3 (SEQ ID NOS: 23 and 25)+no CaCl₂)+synthetic H. Inf (SEQ ID NO: 08) at 1M copies/r×n (LAMP F3+B3 for short)
H. Inf LAMP primers F3 and B3+0.9 mM CaCl₂)+synthetic H. Inf at 1M copies/r×n (LAMP F3+B3 and 0.9 mM CaCl₂))
H Inf_180430_RPA F1 (SEQ ID NO: 14)+R1 (SEQ ID NO: 27: GTAATCAGGAATACTGTCGTATGGGGTTTGTGCA)+no CaCl₂)+synthetic H. Inf at 1M copies/r×n (RPA F1+R1 for short)
H Inf_180430_RPA F1+R1+0.9 mM CaCl₂)+synthetic H. Inf at 1M copies/r×n (RPA F1+R1 and 0.9 mM CaCl2)
NTC for LAMP F3+B3 and no CaCl2
NTC for LAMP F3+B3 and 0.9 mM CaCl2
NTC for RPA F1+R1 and no CaCl2
NTC for RPA F1+R1 and 0.9 mM CaCl2
NPC for no CaCl2
NPC for 0.9 mM CaCl2

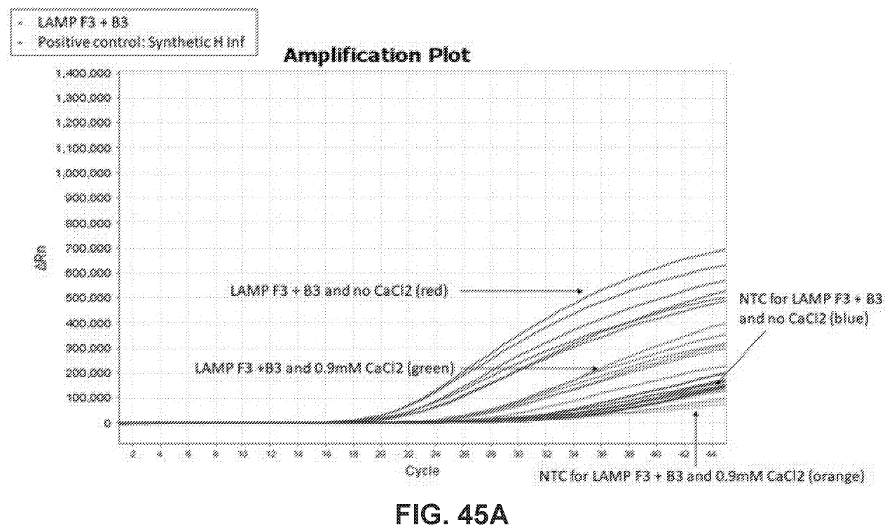

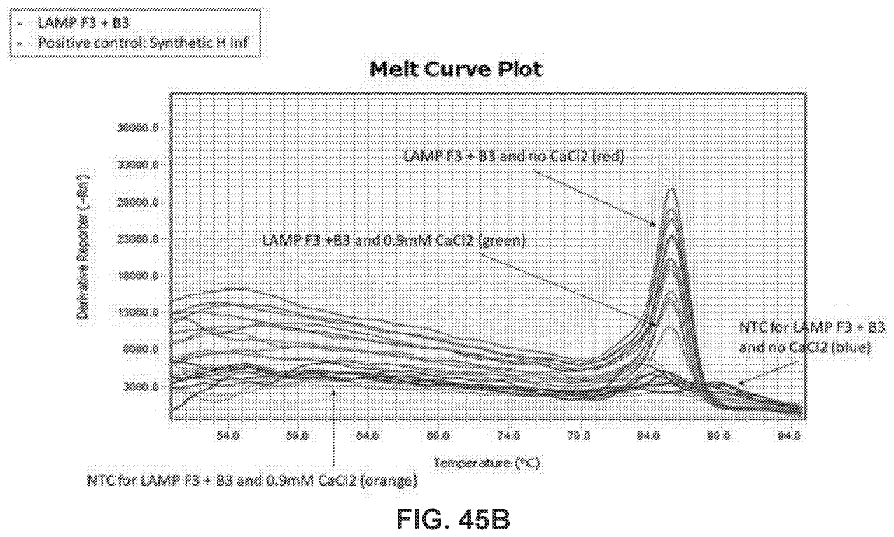

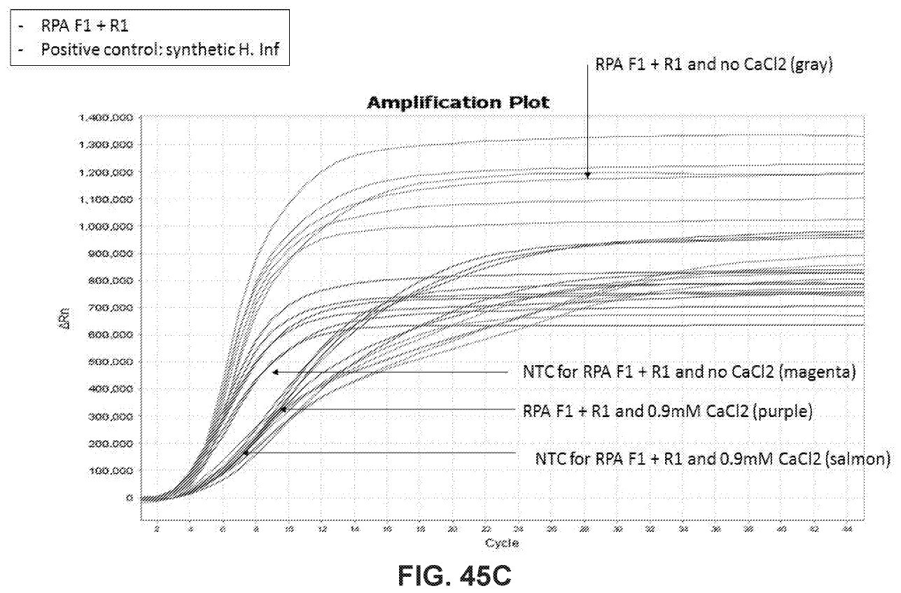

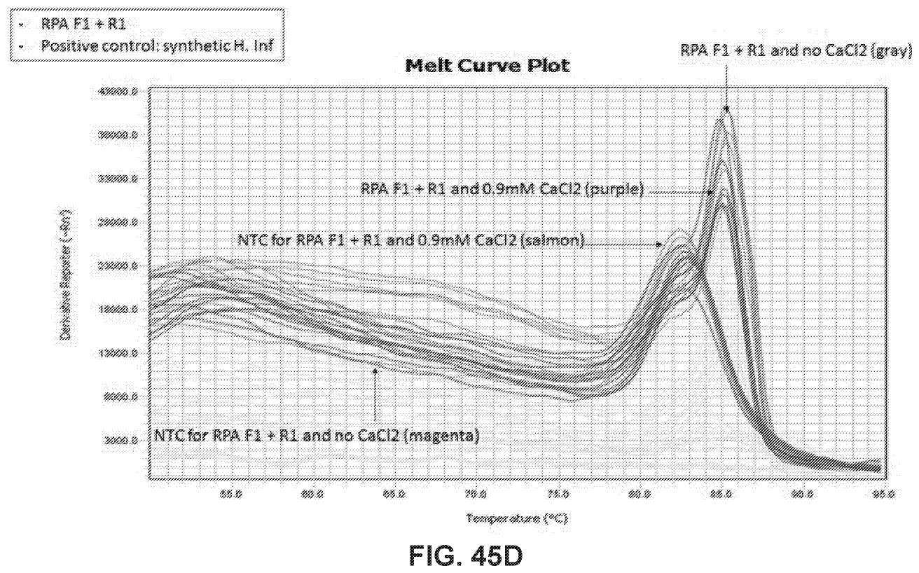

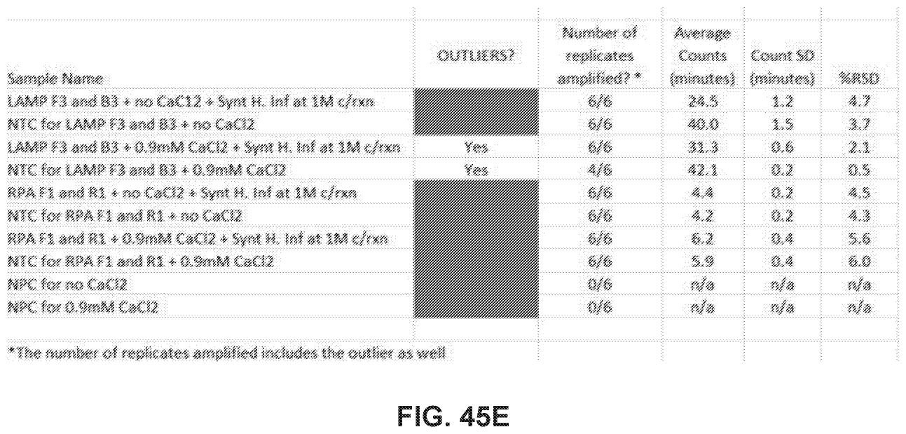

Amplification was performed on 6 replicates of each group. Results are shown in FIGS. 45A-45E. FIG. 45A includes amplification curves for LAMP F3+B3 without CaCl₂) and with 0.9 mM CaCl₂) with synthetic H Inf at 1M copies/r×n and NTC. FIG. 45B shows melt curves for LAMP F3+B3 without CaCl₂) and with 0.9 mM CaCl₂) with synthetic H Inf at 1M copies/r×n and NTC. FIG. 45C includes amplification curves for RPA F1+R1 without CaCl₂) and with 0.9 mM CaCl₂) with synthetic H Inf at 1M copies/r×n and NTC. FIG. 45D includes melt curves for RPA F1+R1 without CaCl₂) and with 0.9 mM CaCl₂) with synthetic H Inf at 1M copies/r×n and NTC. There was a lack of amplification for no-primer control (NPC). Ct values are shown in FIG. 45E. There was a distinction from the positive controls to the NTC. The positive control with no CaCl₂) amplified at 24.5 minutes, whereas the NTC with no CaCl₂) amplified at 40.0 minutes. The positive control with 0.9 mM CaCl₂) was a little more delayed and amplified at 31.3 minutes, whereas the NTC with 0.9 mM CaCl₂) amplified at 42.1 minutes.

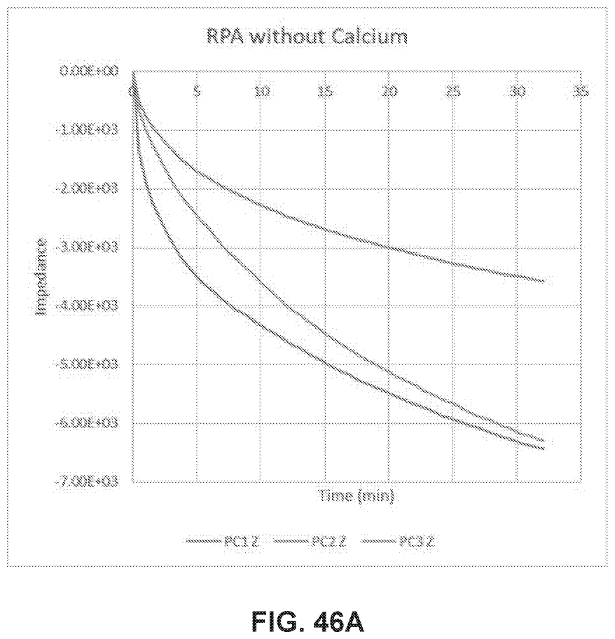

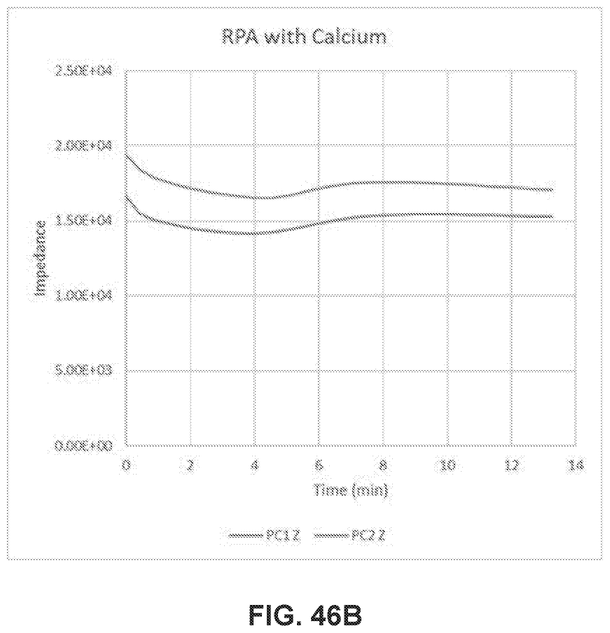

Similar experiments were performed using a cartridge described herein for detection of amplification products. FIG. 46A shows RPA curves for samples without calcium. No amplification signal is detectable in these RPA reactions without calcium. (Note: The drop in impedance during the early phase of the reaction is normal.) FIG. 46B shows RPA curves for samples with 0.9 mM calcium. A signal is evident at approximately 5 min. These results indicate that detection methods using a cartridge according to some embodiments are compatible with isothermal amplification assays such as RPA, and that isothermal amplification such as RPA may work in a higher level of DTT (such as about 5 mM). In some embodiments, calcium (such as CaCl₂) at, for example, 0.5-1.5 mM or about 0.9 mM) or calcium ions are included in an amplification reaction solution or in a reagent solution such as an RPA reagent solution, and the calcium may allow the amplification reaction to proceed in the higher level of DTT.

In another example, similar RPA reactions are performed as in this Example, but in the absence of DTT or at low levels of DTT (such as at 0.1-0.5 mM, 0.5-1.0 mM, or about 0.25 mM). The RPA reactions are performed in and detected by a cartridge detection system as described herein. In such an example, (in the absence of DTT or at low levels of DTT), calcium or calcium ions are not needed to allow the RPA reaction to proceed and be detectable in the cartridge detection system.

### Example 16—Protocol for RPA as Pre-Amp (RAMP) Prior to Another Amplification Such as LAMP

In some embodiments of the methods described herein, RPA is performed as a pre-amplification followed by LAMP in the same reaction container. In some embodiments where RPA is used as a pre-amp prior to LAMP, the LAMP primers comprise FIP/BIP and LF/LB. In some embodiments, the outer LAMP primers (F3/B3) can be replaced by RPA primers, so long as they produce amplicons of the desired length (i.e., the LAMP-specific primers must be spatially nested inside the RPA primers along the target sequence). In some embodiments of RAMP, RPA primers were used in an RPA phase, and then FIP/BIP and LF/LB were added before the LAMP phase. Adding all six LAMP primers (effectively having RPA primers+LAMP F3/B3) also works but is unnecessary in some embodiments.

The following is an example protocol for performing RPA as a pre-amplification followed by LAMP:
(1) Use the Basic Master Mix of Example 12, except:
   Include 8 U of New England Biolabs's WarmStart Bst 2.0 LAMP polymerase. Note that this is not active at the 40° C. RPA reaction temperature.
   Increase dNTP concentration to 5.6 mM total (or 1.8 mM of each dNTP)
   Reduce magnesium activator amount to 8 mM from the 13+mM typically used in RPA
(2) Run steps 2-6 as in RPA. Step 6 is run as pre-amplification for the desired length of time.
(3) After pre-amp, open tubes and pipet in LAMP primers (may be just FIP/BIP and LF/LB; the RPA primers can take the place of F3/B3)
(4) Run the LAMP phase of the reaction at 65° C.

### Example 17—RPA as Pre-Amp (RAMP) Prior to Another Amplification Such as LAMP

Experiments where an RPA reagent solution was prepared according to the Basic Master Mix of Example 12, but including 8 U of NEB's WarmStart Bst 2.0, and dNTPs at 5.6 mM instead of the 1.8 mM, and varying amounts of magnesium. The RPA phase contained only RPA primers, then LAMP FIP/BIP and LF/LB were added after the RPA phase was complete.

A purpose of these experiments were performed to add the NEB WarmStart Bst 2.0 to the RPA phase, as a step toward a one-pot RAMP reaction because all components, except for the LAMP primers and additional dNTPs were included in the RPA phase. The total volume in the RPA phase was 23 μL and 2 μL of LAMP primers and dNTPs were added following the RPA run.

A LAMP reagent solution was provided, which included 1 μL of H Inf LAMP primers and 1 μL of a 95 mM dNTP mix. 2 μL of this mix was added to each 23 μL RPA reaction after its run. This volume can be added to the lid or side of the RPA reaction tubes, then inverted and spun down, similar to an RPA activation step. The LAMP stage was run in the same strips used for the RPA stage since the LAMP reagent solution was added directly to the tubes instead of RPA reaction being removed and added to a new tube. LAMP primer sequences are shown in TABLE 17. The RPA primers were H Inf RPA F1 (SEQ ID NO: 14) and H Inf RPA R3 (SEQ ID NO: 28: ATTGGTTCAATTCTGCCTTTTTCTTCGTAGTAATC). The experiments were run on a QS3 instrument. The target was Synthetic H Inf (10⁶ copies/reaction).

TABLE 17

| SEQ | | | |
|---|---|---|---|
| ID | | | |
| NO: | Type | Name | Sequence |
| 21 | LAMP | H Inf | ACTTCTTTACCAAAGGCATCATTT |
| | Primer | 180430_FIP | AATTTGCGTTTGTTGAATTCTGGG |
| 22 | LAMP | H Inf | TTTTACTGATGATATGGGTACATCT |
| | Primer | 180430_BIP | GGGCTCGAAGAATGAGAAGTTTTGT |
| 23 | LAMP | H Inf | TGGTACGCCAATACATTCAA |
| | Primer | F3_180430 | |
| 24 | LAMP | H Inf | CGCCATAACTTCATCTTAGCACC |
| | Primer | LB_180430 | |
| 10 | LAMP | | |
| (Table | Primer | H Inf B3 | CGTATGGGGTTTGTGCA |
| 9) | | | |

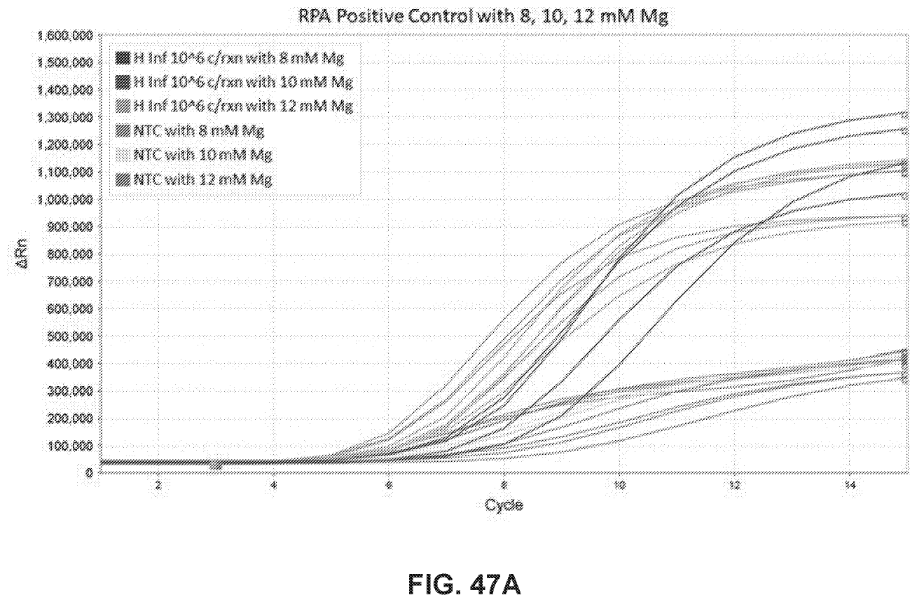

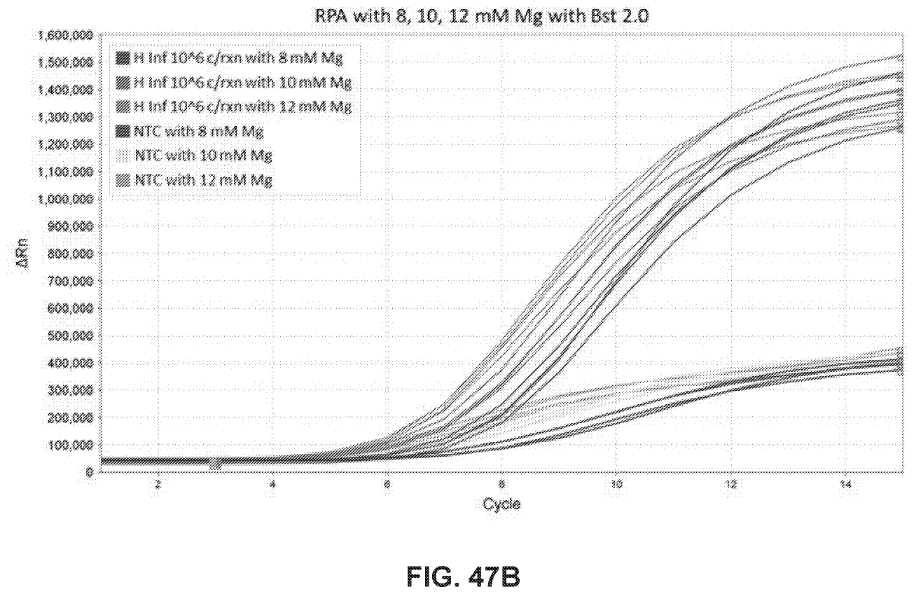

FIGS. 47A and 47B depict RPA stage data (the RPA pre-amp stage before the LAMP primers were added). FIG. 47A is an amplification plot for an RPA positive control with 8 mM, 10 mM, and 12 mM Mg with no added Bst 2.0. FIG. 47B is an amplification plot for the RPA positive control with 8 mM, 10 mM, and 12 mM Mg with NEB's WarmStart Bst 2.0.

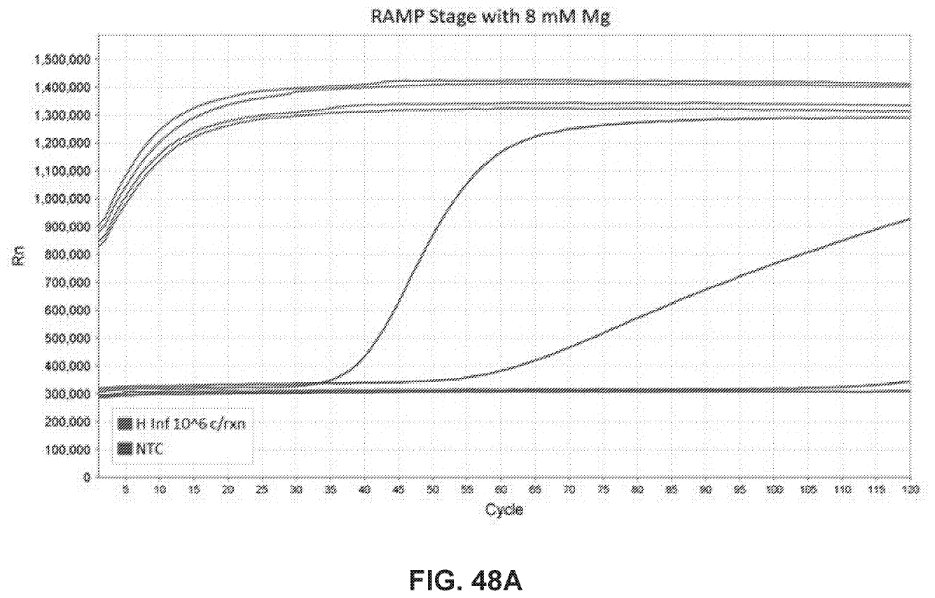

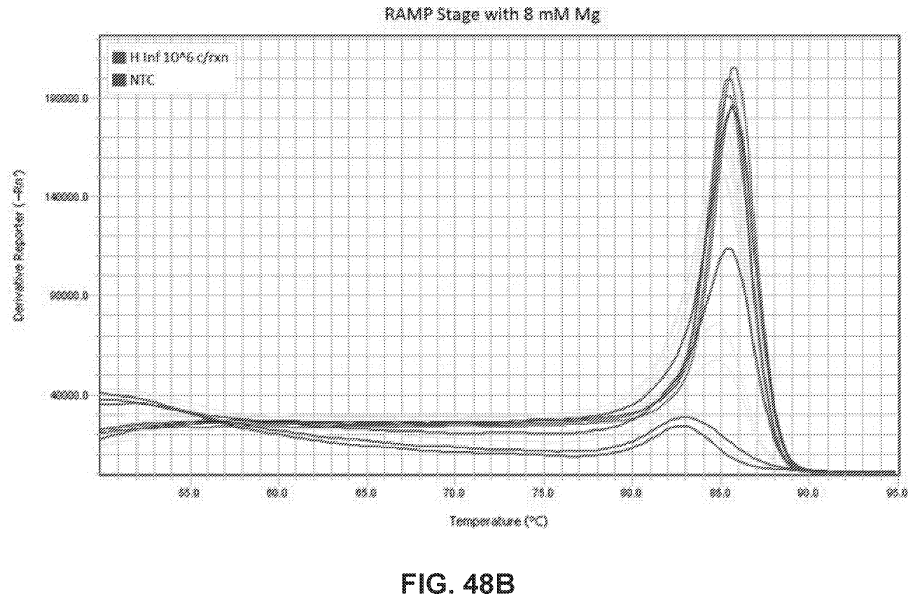

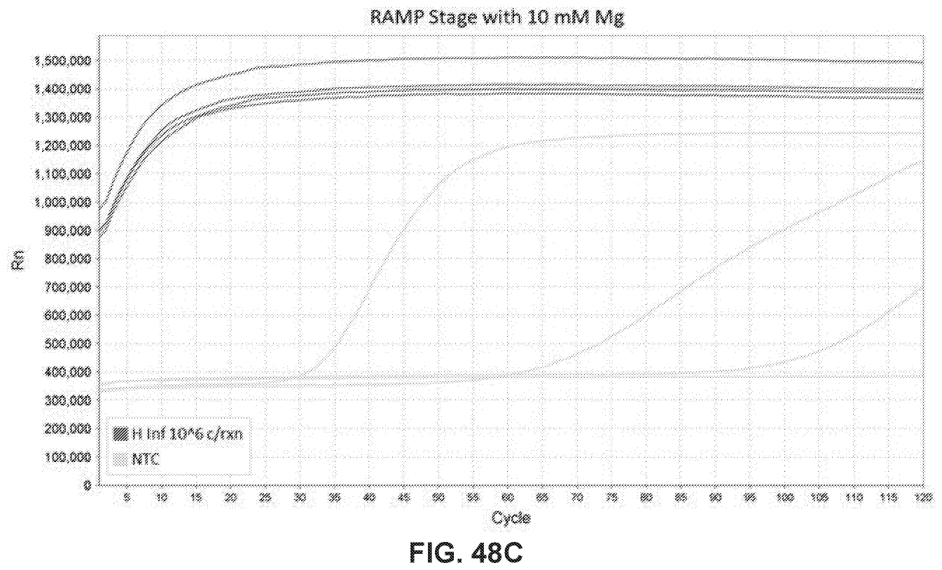

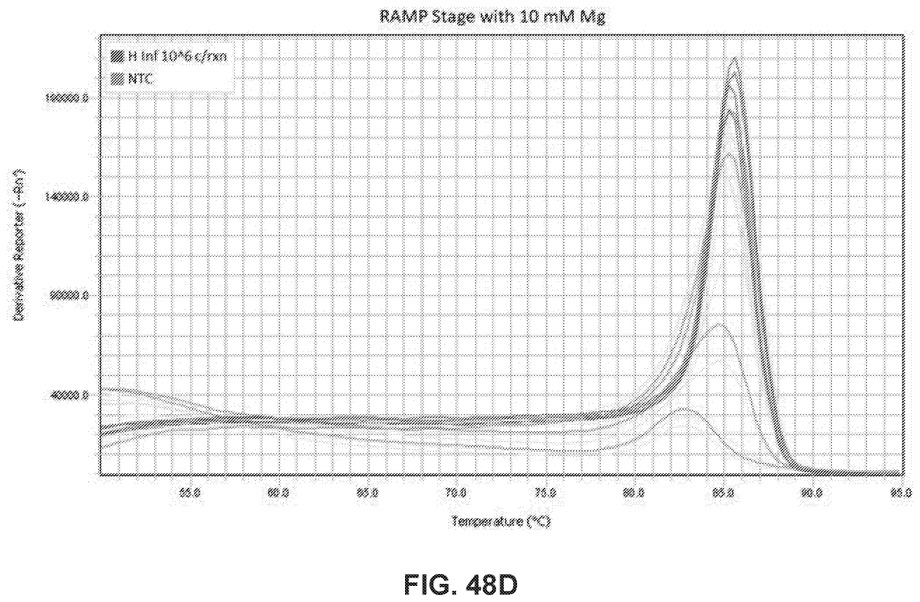

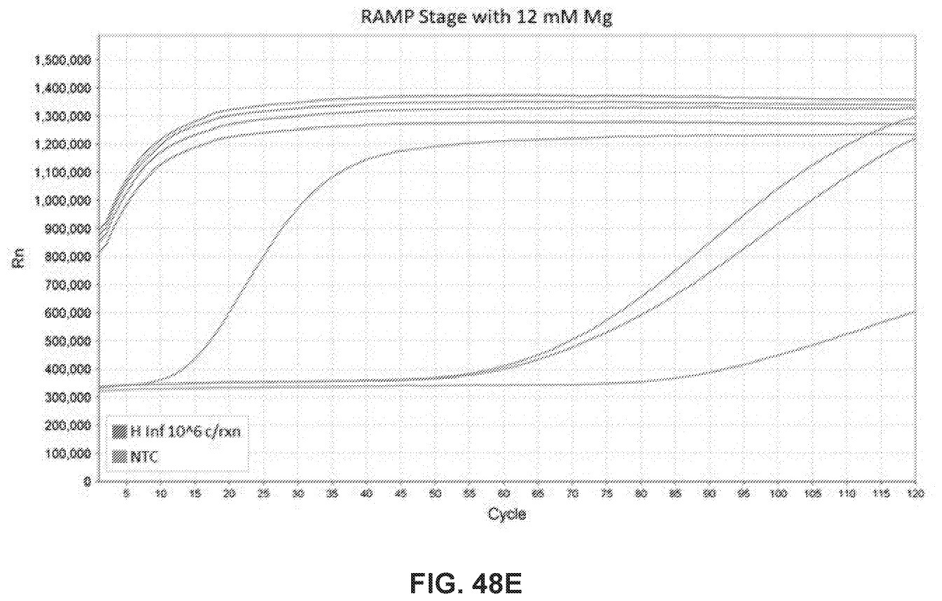

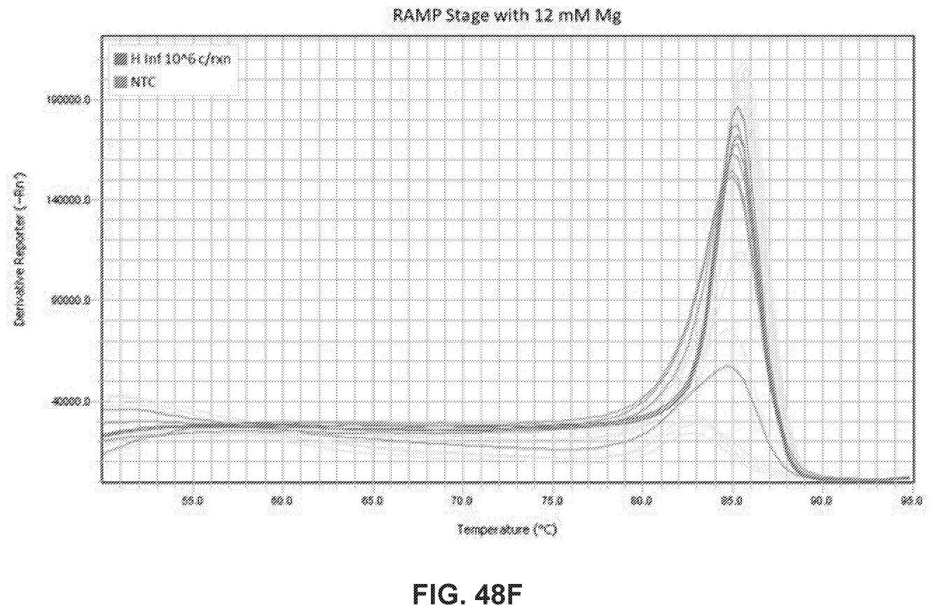

FIG. 48A-48F depict LAMP stage amplification data (after LAMP primers were added). FIG. 48A includes an amplification plot for the LAMP stage of H Inf 10⁶ c/r×n and NTC with 8 mM Mg. FIG. 48B includes a melt curve plot for the LAMP stage of H Inf 10⁶ c/r×n and NTC with 8 mM Mg. FIG. 48C includes an amplification plot for the LAMP stage of H Inf 10⁶ c/r×n and NTC with 10 mM Mg. FIG. 48D includes a melt curve plot for the LAMP stage of H Inf 10⁶ c/r×n and NTC with 10 mM Mg. FIG. 48E includes an amplification plot for the LAMP stage of H Inf 10⁶ c/r×n and NTC with 12 mM Mg. FIG. 48F includes a melt curve plot for the LAMP stage of H Inf 10⁶ c/r×n and NTC with 12 mM Mg.

The data in FIGS. 47A-48F indicate that the RAMP method was successful at all magnesium concentrations used. Accordingly, some embodiments include a method comprising RAMP, or RPA as a pre-amplification reaction prior to another amplification such as LAMP.

### Example 18—Two-Pot RPA and Another Amplification Such as LAMP on a Cartridge

Experiments were performed to test RAMP with serial dilutions of H Inf in the cartridge (10⁶-10⁰ copies/reaction) to determine a sensitivity, specificity, and limit of detection in a cartridge.

RPA was performed in a similar manner (with the modified Basic Master Mix of Example 12) to the RPA in Example 17, except that LAMP primers were not added into the RPA reaction at the end of the RPA stage, and the reaction was performed on a cartridge. The RPA stage was run for 15 minutes at 40° C. on the cartridge. No data was taken during the RPA stage. 2.5-μL aliquots were transferred from the RPA reactions to a LAMP master mix. The LAMP stage was run for 60 minutes at 65° C. on the cartridge. Effectively, the products of the RPA reaction were used as templates in LAMP.

Because the RPA product was diluted 10× when it was transferred to the LAMP reaction, the target concentrations in the RPA stage are a decade higher than the target concentrations in the LAMP phase. The target concentration listed in the data refers to the final concentration after the dilution. Because of this, the initial RPA target concentrations were 10⁷, 10⁶, 10⁵, 10⁴, 10³, 10², and 10¹ copies per reaction (c/r×n). The reason for the 10× dilution from the RPA phase to the LAMP phase was that the RPA reaction mix contained 5 mM of DTT, which may in some cases be incompatible with an electrical detection sensor. By diluting it 10×, the final DTT concentration became 0.5 mM, which allowed the cartridge to detect amplification signals.

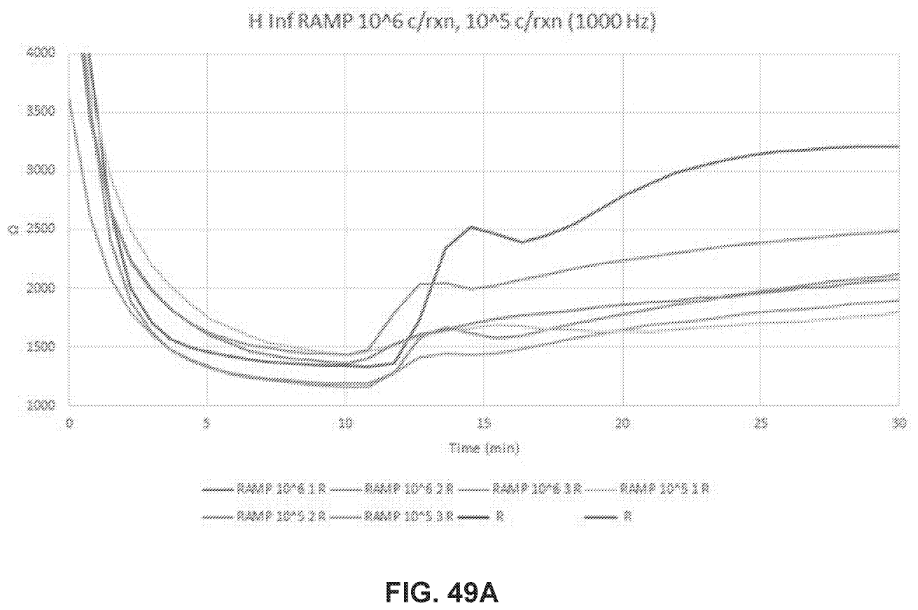

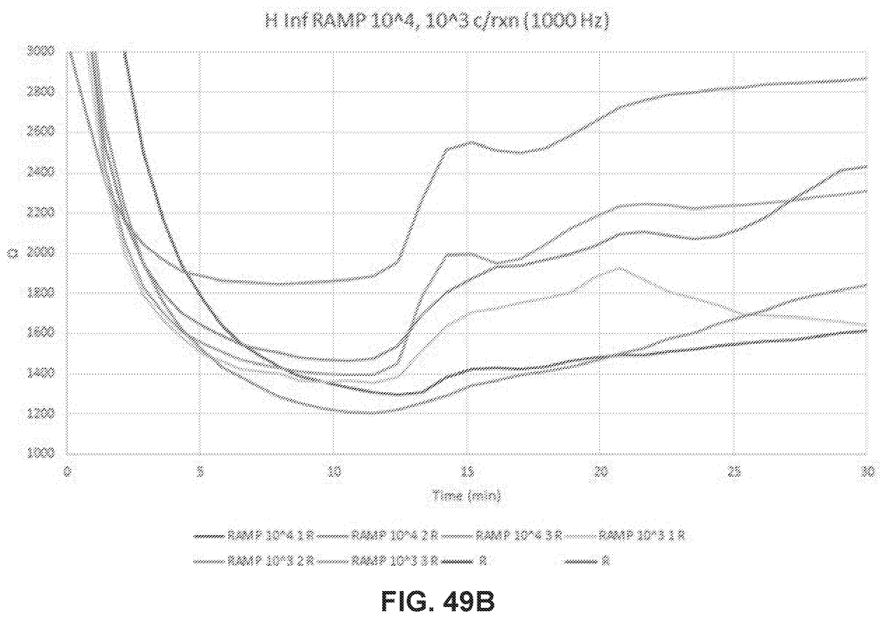

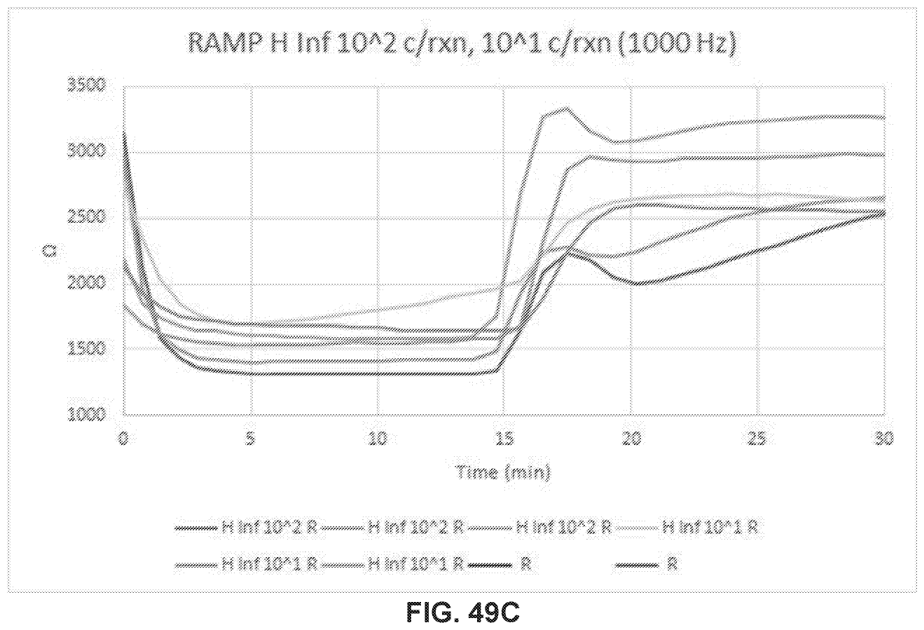

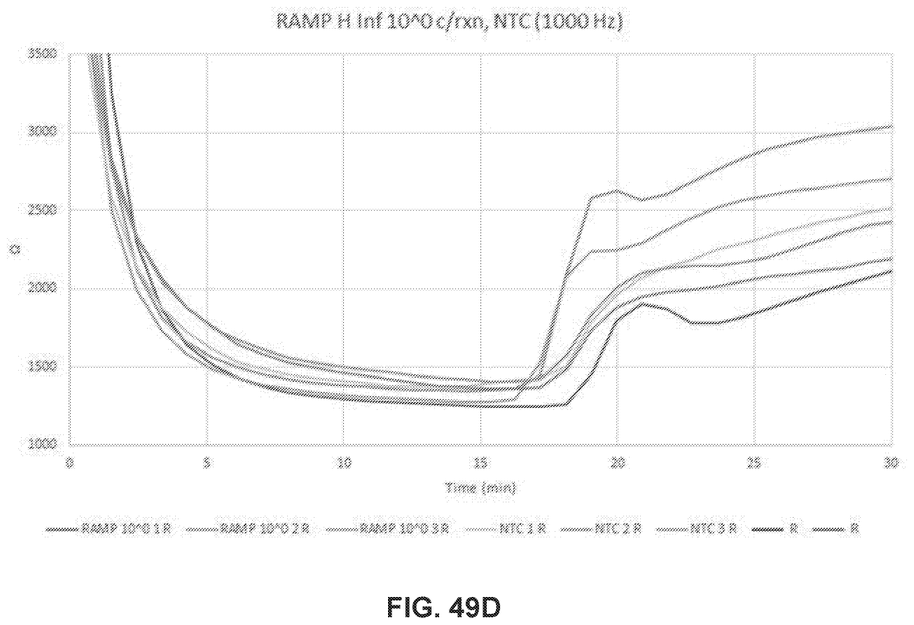

Data are depicted in FIGS. 49A-49D. FIG. 49A includes amplification data with H Inf 10⁶ c/r×n and 10⁵ c/r×n at 1000 Hz. FIG. 49B includes amplification data with H Inf 10⁴ c/r×n and 10³ c/r×n at 1000 Hz. FIG. 49C includes amplification data with H Inf 10² c/r×n and 10¹ c/r×n at 1000 Hz. FIG. 49D includes amplification data with H Inf 10⁰ c/r×n and NTC c/r×n at 1000 Hz.

Amplification was seen for almost all replicates, except for a few replicates on the cartridge with 10⁴ and 10³ c/r×n. Overall, the Cts were slower than what was seen in the thermocycler; 10⁶ c/r×n came up almost immediately in the LAMP stage, while 10⁶ came up at closer to 10 min in the cartridge. These data indicate that according to some embodiments, LAMP may be performed on an RPA reaction product as a template.

### Example 19—One-Pot RPA with Another Amplification Such as LAMP

Experiments were conducted to determine whether RPA and LAMP could be performed together in one reaction mix with blockers included in the reaction solution. An amplification reaction solution was provided that included Bst 2.0 in MM, 5.6 mM dNTPs and 10 mM Mg2+, using TwistAmp Liquid Basic Master Mix. Experiments included 3 replicates of Synt H inf and 3 replicates of NTC with the following conditions:
1. One-pot: FIP/BIP @ 0.32 μM each, no blockers
2. One-pot: FIP/BIP @ 0.32 μM each, 0.1× (0.032 μM each) of LAMP blockers
3. One-pot: FIP/BIP @ 0.32 μM each, 1× (0.32 μM each) of LAMP blockers
4. One-pot: FIP/BIP @ 0.32 μM each, 2× (0.64 μM each) of LAMP blockers
5. One-pot: FIP/BIP @ 0.32 μM each, 5× (1.6 μM each) of LAMP blockers

The experiment was run on a QS3 for 15 minutes at 40° C., followed by 45 minutes at 65° C. Fluorescence was measured once per minute to assess reaction progress. The RPA primers used in each reaction were H Inf_180430 RPA_F1 (SEQ ID NO: 14) and H Inf_180430 RPA_R3 (SEQ ID NO: 28), at 480 nM each in all cases. The LAMP primers used in each reaction were H Inf 180430_FIP (SEQ ID NO: 22) and H Inf 180430_BIP (SEQ ID NO: 21). No F3/B3 or LF/LB primers were included, just H Inf 180430_FIP and H Inf 180430_BIP. Blocker sequences are shown in Table 18. The blockers were designed to bind directly to the LAMP primers to prevent their action during the RPA phase.

TABLE 18

| SEQ | | | |
|---|---|---|---|
| ID | | | |
| NO: | Type | Name | Sequence |
| 25 | LAMP | 180706 | CCCAGAATTCAACAA/3Phos/ |
| | Blocker | B15_FIP | |
| | | H Inf | |
| 26 | LAMP | 180706 | ACAAAACTTCTCATTC/3Phos/ |
| | Blocker | B16_BIP | |
| | | H Inf | |

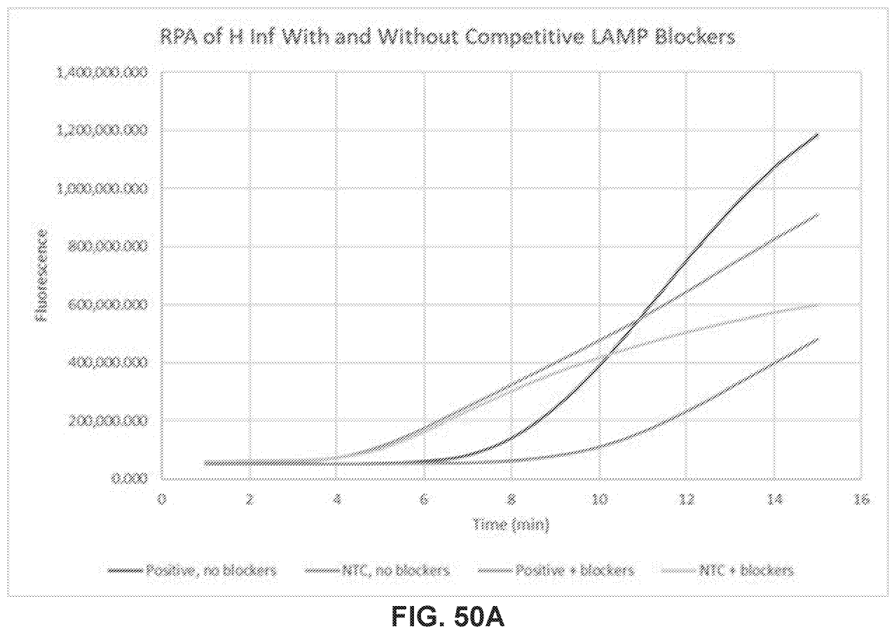

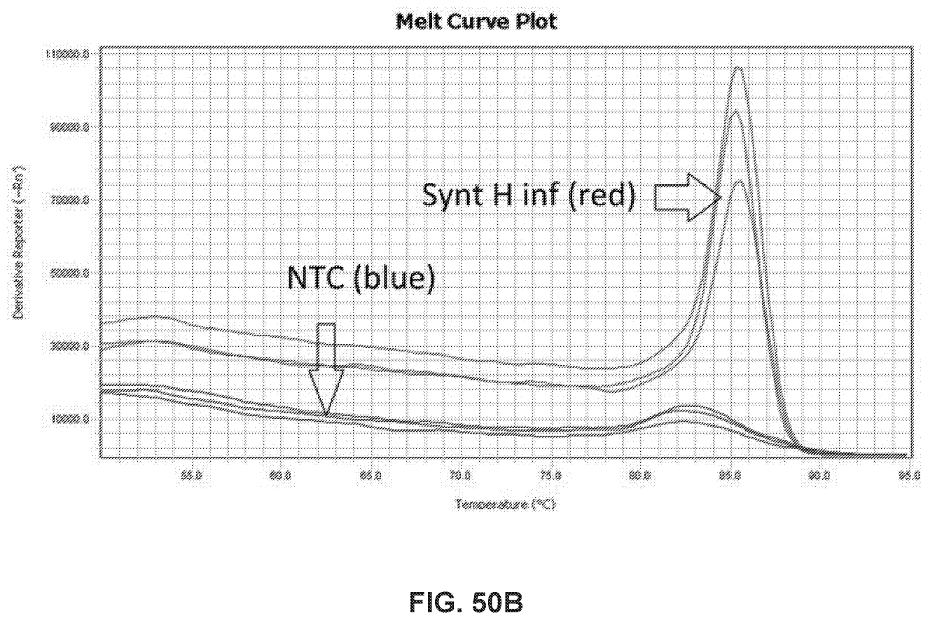

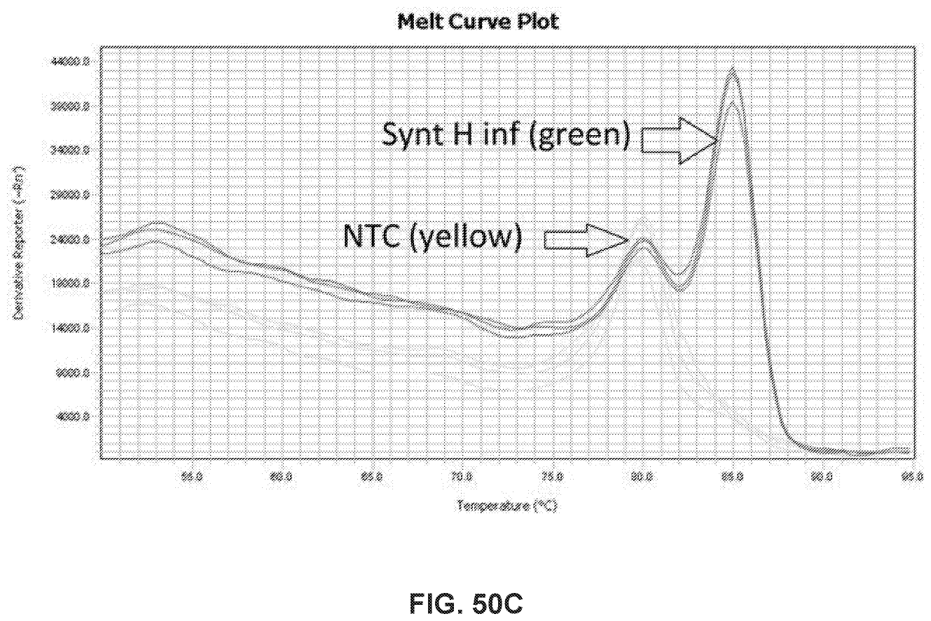

Data are depicted in FIGS. 50A-50C. As shown in FIG. 50A, inclusion of LAMP blockers resulted in faster amplification, indicating that the blockers prevented the LAMP primers from inhibiting RPA. FIG. 50B includes a melt curve plot of one-pot: FIP/BIP @ 0.32 μM each, no blockers. FIG. 50C includes a melt curve plot of one-pot: FIP/BIP @ 0.32 μM each, 0.1× (0.032 μM each) of LAMP blockers. The melt curves indicate that different species were produced in the presence of the blockers. These experiments indicate that blockers may be used according to some embodiments as part of, for example, an amplification reaction solution to improve the specificity of a nucleic acid amplification.

### Implementing Systems and Terminology

Implementations disclosed herein provide systems, methods and apparatus for detection of the presence and/or quantity of a target analyte. One skilled in the art will recognize that these embodiments may be implemented in hardware or a combination of hardware and software and/or firmware.

The signal processing and reader device control functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. The term “computer-program product” refers to a computing device or processor in combination with code or instructions (e.g., a “program”) that may be executed, processed or computed by the computing device or processor. As used herein, the term “code” may refer to software, instructions, code or data that is/are executable by a computing device or processor.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. A computing environment can include any type of computer system, including, but not limited to, a computer system based on a microprocessor, a mainframe computer, a digital signal processor, a portable computing device, a personal organizer, a device controller, and a computational engine within an appliance, to name a few.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A method of amplifying and detecting a target nucleic acid, comprising:
providing a recombinase polymerase amplification (RPA) reagent solution configured for a first isothermal amplification reaction comprising a target nucleic acid, primers each being complementary to a region of said target nucleic acid, a buffer, dNTPs, a recombinase, a single-stranded DNA-binding protein (SSB), and a strand-displacing DNA polymerase in a single vessel;
combining the RPA reagent solution with a second reagent solution configured for a second isothermal amplification reaction, which does not utilize a recombinase in said single vessel, to produce an amplification reaction solution;
conducting RPA in said amplification reaction solution to produce an amplified target nucleic acid;
conducting spiral RPA using a pair of primers with the forward and reverse primer sequences reverse complementary to each other at their 5′ end and their 3′ end sequences complementary to the target sequences; and
detecting the presence of the amplified target nucleic acid by measuring a modulation of an electrical signal in the amplification reaction solution when said amplification reaction is subjected to an electrical field, as compared to a control.

2-55. (canceled)

56. The method of claim 1, wherein the detecting the presence of the amplified target nucleic acid is performed in a device comprising a test well, which comprises an excitation electrode and a sensor electrode, and wherein said detecting further comprises:
applying an excitation signal from a reader device to the excitation electrode;
sensing a signal from the test well using the excitation electrode, wherein the signal represents the impedance of the amplification reaction solution; and
transmitting the signal to the reader device, wherein the reader device analyzes the signal.

57. The method of claim 1, wherein the strand-displacing DNA polymerase comprises at least one of a Bst DNA polymerase large fragment, a Bst 2.0 polymerase, a Bst 3.0 polymerase, a Gsp polymerase, a Sau polymerase, a Bsu DNA polymerase large fragment, a Deep VentR DNA Polymerase, a Deep VentR (exo−) DNA Polymerase, a Klenow Fragment (3′→5′ exo−), a DNA Polymerase I Large (Klenow) Fragment, a phi29 DNA polymerase, a VentR DNA polymerase, or a VentR (exo−) DNA polymerase.

58. The method of claim 1, wherein the RPA reagent solution further comprises a reagent selected from the group consisting of Tris-Acetate, polyethylene glycol, trehalose, potassium acetate, creatine kinase, phosphocreatine, adenosine triphosphate, and a recombinase loading protein.

59. The method of claim 1, wherein the amplification reaction solution further comprises a blocker oligonucleotide comprising a nucleic acid sequence that is a reverse complement to part of the nucleic acid sequence of one or more of said primers.

60. The method of claim 1, wherein the second isothermal amplification comprises at least one of: self-sustaining sequence replication reaction (3SR), 90-I, BAD Amp, cross priming amplification (CPA), isothermal exponential amplification reaction (EXPAR), isothermal chimeric primer initiated amplification of nucleic acids (ICAN), isothermal multi displacement amplification (IMDA), ligation-mediated SDA, multi displacement amplification, polymerase spiral reaction (PSR), restriction cascade exponential amplification (RCEA), smart amplification process (SMAP2), single primer isothermal amplification (SPIA), transcription-based amplification system (TAS), transcription meditated amplification (TMA), ligase chain reaction (LCR), or multiple cross displacement amplification (MCDA), and comprises: rolling circle replication (RCA), Nicking Enzyme Amplification Reaction (NEAR), or Nucleic acid sequence based amplification (NASBA).

61. The method of claim 1, wherein the RPA and the second isothermal amplification are conducted at the same temperature.

62. The method of claim 1, wherein the second isothermal amplification comprises Loop-Mediated Isothermal Amplification (LAMP).

63. The method of claim 62, wherein the amplification reaction solution comprises:
a primer oligonucleotide compatible with LAMP; and
FIP, BIP, LF, or LB primer oligonucleotides compatible with LAMP, and primers compatible with RPA.

64. The method of claim 63, wherein the amplification reaction solution comprises FIP, BIP, LF, LB, F3, or B3 primer oligonucleotides compatible with LAMP.

65. A method of amplifying and detecting a target nucleic acid, comprising:
providing a recombinase polymerase amplification (RPA) reagent solution configured for a first isothermal amplification reaction comprising a target nucleic acid, primers each being complementary to a region of said target nucleic acid, a buffer, dNTPs, a recombinase, a single-stranded DNA-binding protein (SSB), and a strand-displacing DNA polymerase in a single vessel;
conducting RPA in the RPA reagent solution to produce an amplified target nucleic acid;
combining the amplified target nucleic acid with a second reagent solution configured for a second isothermal amplification reaction, which does not utilize a recombinase in said single vessel, to produce an amplification reaction solution;
conducting a second isothermal amplification of said second amplification reaction solution to produce a further amplified target nucleic acid; and
detecting the presence of the amplified target nucleic acid by measuring a modulation of an electrical signal in the amplification reaction solution when said amplification reaction is subjected to an electrical field, as compared to a control.

66. The method of claim 65, wherein combining the amplified target nucleic acid with a second reagent solution configured for a second isothermal amplification reaction comprises adding the second reagent solution to the RPA reagent solution after conducting RPA to produce an amplified target nucleic acid.

67. The method of claim 65, wherein combining the amplified target nucleic acid with a second reagent solution configured for a second isothermal amplification reaction comprises adding the RPA reagent solution or a portion of the RPA reagent solution to the second reagent solution after conducting RPA to produce the amplified target nucleic acid.

68. The method of claim 65, wherein the strand-displacing DNA polymerase comprises at least one of a Bst DNA polymerase large fragment, a Bst 2.0 polymerase, a Bst 3.0 polymerase, a Gsp polymerase, a Sau polymerase, a Bsu DNA polymerase large fragment, a Deep VentR DNA Polymerase, a Deep VentR (exo−) DNA Polymerase, a Klenow Fragment (3′→5′ exo−), a DNA Polymerase I Large (Klenow) Fragment, a phi29 DNA polymerase, a VentR DNA polymerase, or a VentR (exo−) DNA polymerase.

69. The method of claim 65, wherein the RPA reagent solution further comprises a reagent selected from the group consisting of Tris-Acetate, polyethylene glycol, trehalose, potassium acetate, creatine kinase, phosphocreatine, adenosine triphosphate, and a recombinase loading protein.

70. The method of claim 65, wherein the amplification reaction solution further comprises a blocker oligonucleotide comprising a nucleic acid sequence that is a reverse complement to part of the nucleic acid sequence of one or more of said primers.

71. The method of claim 65, wherein the second isothermal amplification comprises at least one of: self-sustaining sequence replication reaction (3SR), 90-I, BAD Amp, cross priming amplification (CPA), isothermal exponential amplification reaction (EXPAR), isothermal chimeric primer initiated amplification of nucleic acids (ICAN), isothermal multi displacement amplification (IMDA), ligation-mediated SDA; multi displacement amplification, polymerase spiral reaction (PSR), restriction cascade exponential amplification (RCEA), smart amplification process (SMAP2), single primer isothermal amplification (SPIA), transcription-based amplification system (TAS), transcription meditated amplification (TMA), ligase chain reaction (LCR), or multiple cross displacement amplification (MCDA), and comprises: rolling circle replication (RCA), Nicking Enzyme Amplification Reaction (NEAR) or Nucleic acid sequence based amplification (NASBA).

72. The method of claim 65, wherein the RPA is conducted at a lower or higher temperature than the second isothermal amplification.

73. The method of claim 65, wherein the second isothermal amplification comprises Loop-Mediated Isothermal Amplification (LAMP).

74. The method of claim 65, wherein the amplification reaction solution comprises at least one of FIP, BIP, LF, or LB primer oligonucleotides compatible with LAMP, and primers compatible with RPA, or
FIP, BIP, LF, LB, F3, or B3 primer oligonucleotides compatible with LAMP.
